(19) United States
(12) Patent Application Publication
(10) Pub. No.: US 2025/0393386 A1
(43) Pub. Date: Dec. 25, 2025

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Changwoo KIM, Suwon-si (KR); Youngkyoung JO, Suwon-si (KR); Byungku KIM, Suwon-si (KR); Dong Min KANG, Suwon-si (KR); Wonho NOH, Suwon-si (KR); Kipo JANG, Suwon-si (KR); Min Seok SEO, Suwon-si (KR); Dalho HUH, Suwon-si (KR); Hyung Sun KIM, Suwon-si (KR)

(21) Appl. No.: 19/238,785

(22) Filed: Jun. 16, 2025

(30) Foreign Application Priority Data
Jun. 20, 2024 (KR) 10-2024-0080485

(51) Int. Cl. H10K 50/11; C07D 209/82; H10K 85/40; H10K 85/60

(52) U.S. Cl. CPC H10K 50/11; C07D 209/82; H10K 85/40; H10K 85/622; H10K 85/626; H10K 85/633; H10K 85/636; H10K 85/654; H10K 85/6572; H10K 85/6574; H10K 85/6576

(57) ABSTRACT

A compound for an organic optoelectronic device, a composition for an organic optoelectronic device including the compound, an organic optoelectronic device including the compound or the composition for an organic optoelectronic device, and a display device including the organic optoelectronic device, the compound being represented by a combination of Chemical Formula 1a and Chemical Formula 1b:

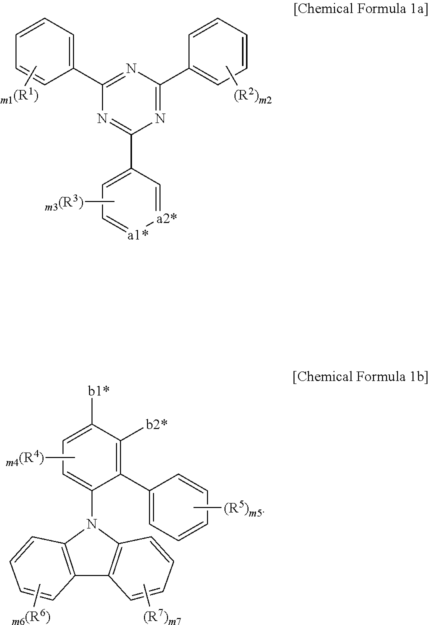

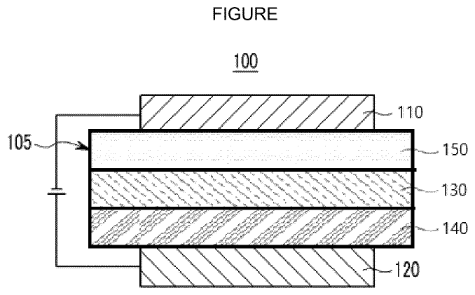

### CROSS-REFERENCE TO RELATED APPLICATION

This application claims priority to and the benefit of Korean Patent Application No. 10-2024-0080485 filed in the Korean Intellectual Property Office on Jun. 20, 2024, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is a light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting more attention in recent years due to increasing demand for flat panel display devices. The organic light emitting diode is a device that converts electrical energy into light, and the performance of the organic light emitting diode may be greatly influenced by an organic material between electrodes.

### SUMMARY

The embodiments may be realized by providing a compound for an organic optoelectronic device, the compound being represented by a combination of Chemical Formula 1a and Chemical Formula 1b:

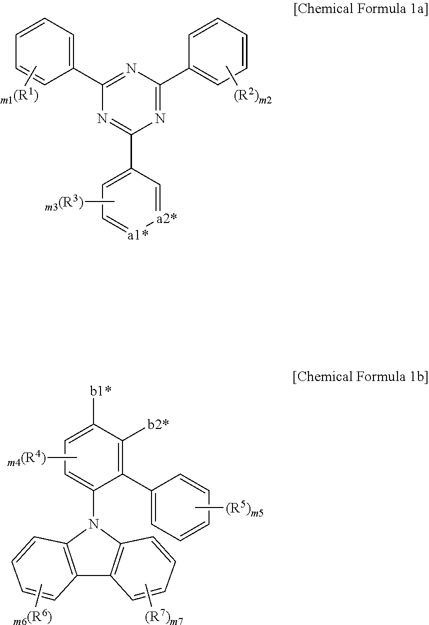

wherein, in Chemical Formula 1a and Chemical Formula 1b, a1* and a2* are each independently a linking carbon or CR^{a}, b1* and b2* are each independently linking points, that link at a1* or a2*, or R^{b}, R¹ and R² are each independently hydrogen, deuterium, or a phenyl group substituted with deuterium, R^{a}, R^{b}, and R³ to R⁷ are each independently hydrogen or deuterium, m1, m2, and m5 are each independently an integer of 1 to 5, m3 is an integer of 1 to 3, m4 is an integer of 1 or 2, m6 and m7 are each independently an integer of 1 to 4, when m1 is 2, 3, 4, or 5, each R¹ is the same or different from each other, when m2 is 2, 3, 4, or 5, each R² is the same or different from each other, when m3 is 2 or 3, each R³ is the same or different from each other, when m4 is 2, each R⁴ is the same or different from each other, when m5 is 2, 3, 4, or 5, each R⁵ is the same or different from each other, when m6 is 2, 3, or 4, each R⁶ is the same or different from each other, and when m7 is 2, 3, or 4, each R⁷ is the same or different from each other.

The embodiments may be realized by providing a composition for an organic optoelectronic device, the composition including a first compound; and a second compound, wherein the first compound may be the compound for an organic optoelectronic device according to an embodiment, and the second compound may be represented by Chemical Formula 2, a combination of Chemical Formula 3 and Chemical Formula 4, or Chemical Formula 5:

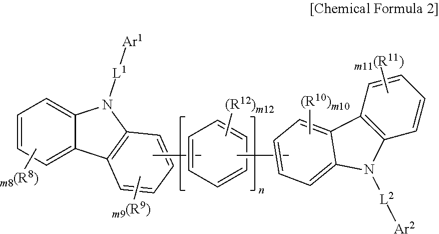

in Chemical Formula 2, R⁸ to R¹² may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar¹ and Ar² may each independently be a substituted or unsubstituted C6 to C20 aryl group or a substituted or unsubstituted C2 to C30 heterocyclic group, L¹ and L² may each independently be a single bond or a substituted or unsubstituted C6 to C20 arylene group, m8, m11, and m12 may each independently be an integer of 1 to 4, m9 and m10 may each independently be an integer of 1 to 3, when m8 is 2, 3, or 4, each R⁸ may be the same or different from each other, when m9 is 2 or 3, each R⁹ may be the same or different from each other, when m10 is 2 or 3, each R¹⁰ may be the same or different from each other, when m11 is 2, 3, or 4, each R¹¹ may be the same or different from each other, when m12 is 2, 3, or 4, each R¹² may be the same or different from each other, and n may be an integer of 0 to 2;

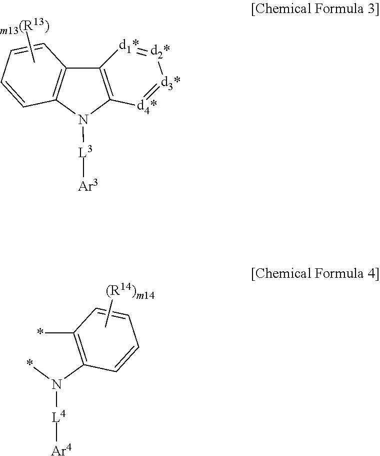

in Chemical Formula 3 and Chemical Formula 4, two adjacent ones of d₁* to d₄* in Chemical Formula 3, may each be linking carbons linked to * of Chemical Formula 4, the remaining two of d₁* to d₄* in Chemical Formula 3, not linked at * of Chemical Formula 4, are C-L^{a}-R^{c}, L^{a}, L³, and L⁴ may each independently be a single bond or a substituted or unsubstituted C6 to C20 arylene group, R^{c}, R¹³, and R¹⁴ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar³ and Ar⁴ may each independently be a substituted or unsubstituted C6 to C20 aryl group or a substituted or unsubstituted C2 to C30 heterocyclic group, m13 and m14 may each independently be an integer of 1 to 4, when m13 is 2, 3, or 4, each R¹³ may be the same or different from each other, and when m14 is 2, 3, or 4, each R¹⁴ may be the same or different from each other;

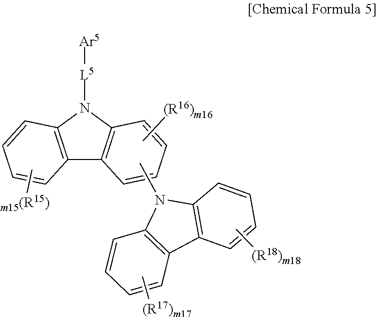

in Chemical Formula 5, L⁵ may be single bond or a substituted or unsubstituted C6 to C20 arylene group, R¹⁵ to R¹⁸ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar⁵ may be a substituted or unsubstituted C6 to C20 aryl group or a substituted or unsubstituted C2 to C30 heterocyclic group, m15, m17, and m18 may each independently be an integer of 1 to 4, m16 may be an integer of 1 to 3, when m15 is 2, 3, or 4, each R¹⁵ may be the same or different from each other, when m16 is 2 or 3, each R¹⁶ may be the same or different from each other, when m17 is 2, 3, or 4, each R¹⁷ may be the same or different from each other, and when m18 is 2, 3, or 4, each R¹⁸ may be the same or different from each other.

The embodiments may be realized by providing an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing an organic optoelectronic device including an anode and a cathode facing each other, and at least one organic layer between the anode and the cathode, wherein the organic layer includes the composition for an organic optoelectronic device according to an embodiment.

The embodiments may be realized by providing a display device including the organic optoelectronic device according to an embodiment.

### BRIEF DESCRIPTION OF THE DRAWINGS

Features will become apparent to those of skill in the art by describing in detail exemplary embodiments with reference to the attached drawing in which:

the FIGURE is a cross-sectional view showing an organic light emitting diode according to some example embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figure, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present embodiments, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present embodiments, “substituted” refers to replacement of at least one hydrogen of a substituent or a compound by deuterium, a C1 to C20 alkyl group, a C1 to C5 alkylsilyl group, a C6 to C20 aryl group, a C2 to C20 heteroaryl group, or a cyano group. In specific example of the present embodiments, “substituted” refers to replacement of at least one hydrogen of a substituent or a compound by deuterium, a C1 to C5 alkyl group, a C1 to C5 alkylsilyl group, a C6 to C18 aryl group, a C2 to C18 heteroaryl group, or a cyano group. In specific example of the present embodiments, the “substituted” refers to replacement of at least one hydrogen of a substituent or a compound by deuterium, a cyano group, a methyl group, an ethyl group, an n-propyl group, an iso-propyl group, an n-butyl group, an iso-butyl group, a sec-butyl group, a tert-butyl group, a trimethylsilyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).” For example, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, e.g., a phenyl group, a naphthyl group, or the like, two or more hydrocarbon aromatic moieties may be linked by a sigma bond and may be, e.g., a biphenyl group, a terphenyl group, a quarterphenyl group, or the like, and two or more hydrocarbon aromatic moieties may be fused directly or indirectly to provide a non-aromatic fused ring, e.g., a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

In an implementation, “heteroaryl group” may refer to an aryl group including at least one heteroatom selected from N, O, S, P, and Si. Two or more heteroaryl groups may be linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to some example embodiments is described.

A compound for an organic optoelectronic device according to some example embodiments may be represented by a combination of Chemical Formula 1a and Chemical Formula 1b.

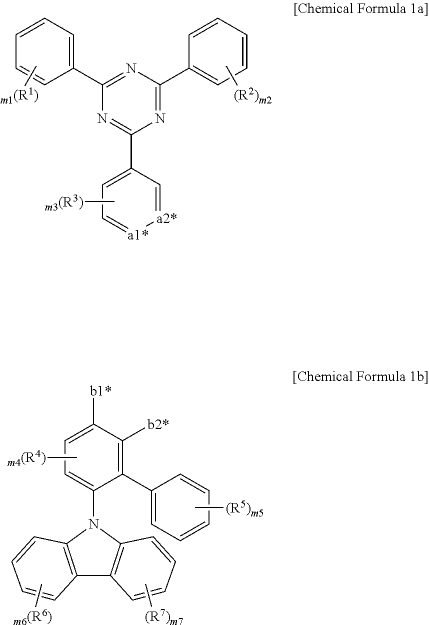

In Chemical Formula 1a and Chemical Formula 1b, a1* and a2* may each independently be, e.g., a linking carbon or CR^{a}.

b1* and b2* may each independently be, e.g., linking points, that link at a1* or a2*, or R^{b}.

R¹ and R² may each independently be, e.g., hydrogen, deuterium, or a phenyl group substituted with deuterium.

R^{a}, R^{b}, and R³ to R⁷ may each independently be, e.g., hydrogen or deuterium.

m1, m2, and m5 may each independently be, e.g., an integer of 1 to 5.

m3 may be, e.g., an integer of 1 to 3.

m4 may be, e.g., an integer of 1 or 2.

m6 and m7 may each independently be, e.g., an integer of 1 to 4.

When any one of m1 to m7 is 2 or more, each respective one of R¹ to R⁷ may be the same or different from each other.

The compound represented by the combination of Chemical Formula 1a and Chemical Formula 1b is a compound in which a biphenyl linker linked to a triazine and a carbazole are linked para- or meta-directly, and an additional phenyl group is substituted in the ortho position to the phenylene linked to the carbazole in the biphenyl linker, thereby improving the efficiency characteristics and life-span characteristics of an organic light emitting diode to which it is applied.

In particular, the compound represented by the combination of the above chemical formulas 1a and 1b has a relatively excellent efficiency characteristic because the terphenyl linker connecting the triazine and the carbazole becomes a folded structure such as a meta structure or an ortho structure, thereby shortening the conjugation length compared to the structure in which the terphenyl linker is connected in a para structure, and forming a Tl energy level suitable for a green host.

In addition, when the phenyl group is substituted at the ortho position of the carbazole, the dihedral angle increases due to the steric hindrance between the carbazole and the ortho phenyl, and the carbazole moiety and the ortho phenyl moiety twist with each other, thereby increasing the dihedral angle. This means that the electron clouds of the HOMO level and LUMO level are mostly separated without overlapping, and because it has a small ΔEst, rapid energy transfer is possible, and it exhibits high efficiency characteristics, especially when applied as a phosphorescent host. In addition, the side reaction path in the excited state is reduced, and thus the life-span extension effect can be further obtained.

In an implementation, m1 may be 2, 3, 4, or 5, and each of R¹ may be the same or different from each other.

In an implementation, m2 may be 2, 3, 4, or 5, and each of R² may be the same or different from each other.

In an implementation, m3 may be 2 or 3, and each of R³ may be the same or different from each other.

In an implementation, m4 may be 2, and each of R⁴ may be the same or different from each other.

In an implementation, m5 may be 2, 3, 4, or 5, and each of R⁵ may be the same or different from each other.

In an implementation, m6 may be 2, 3, or 4, and each of R⁶ may be the same or different from each other.

In an implementation, m7 may be 2, 3, or 4, and each of R⁷ may be the same or different from each other.

In an implementation, the combination of Chemical Formula 1a and Chemical Formula 1b may be represented by one of Chemical Formula 1-1 to Chemical Formula 1-4.

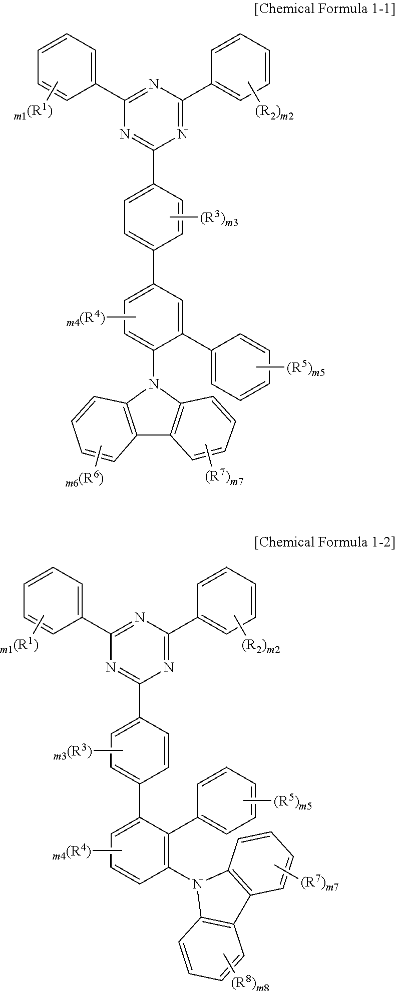

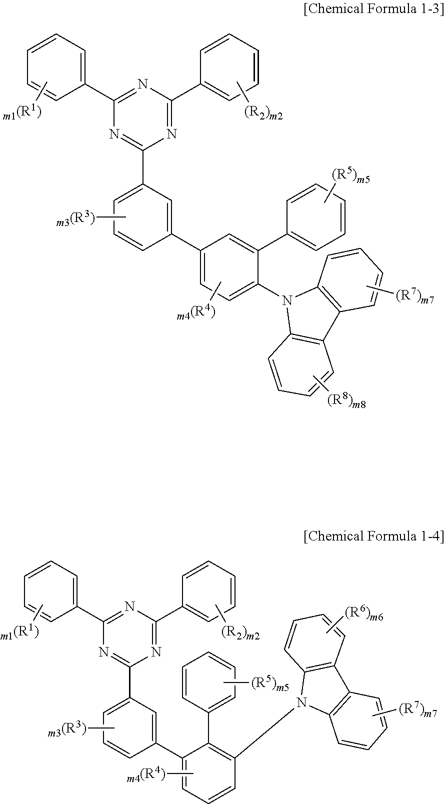

In Chemical Formula 1-1 to Chemical Formula 1-4, R¹ to R⁷ and m1 to m7 may be defined the same as described above.

In an implementation, the compound represented by the combination of Chemical Formula 1a and Chemical Formula 1b may be a compound of Group 1.

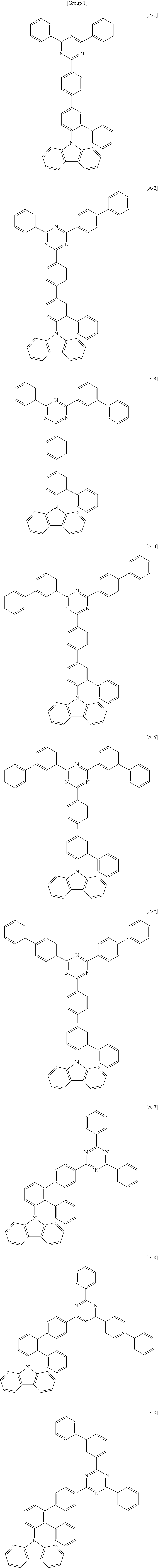

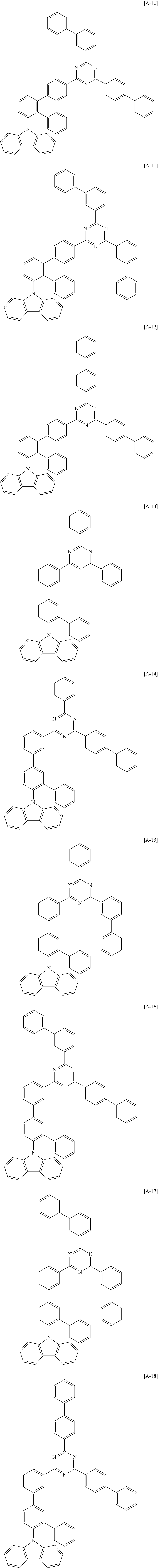

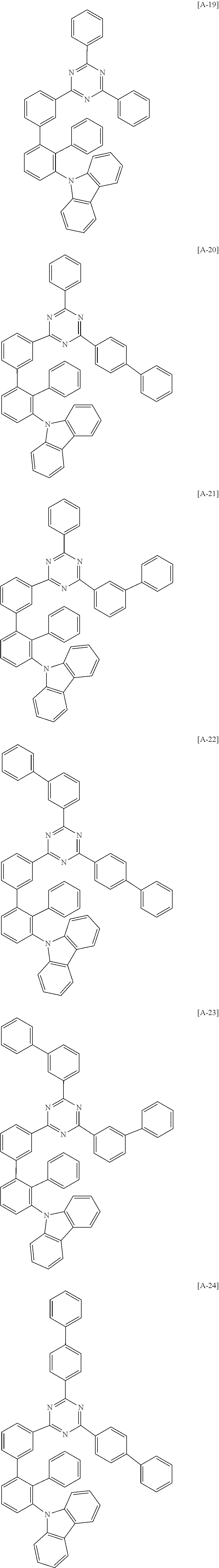

In addition, the form in which deuterium is substituted in the compounds listed in Group 1 may be one selected from the compounds listed in Group 1-1.

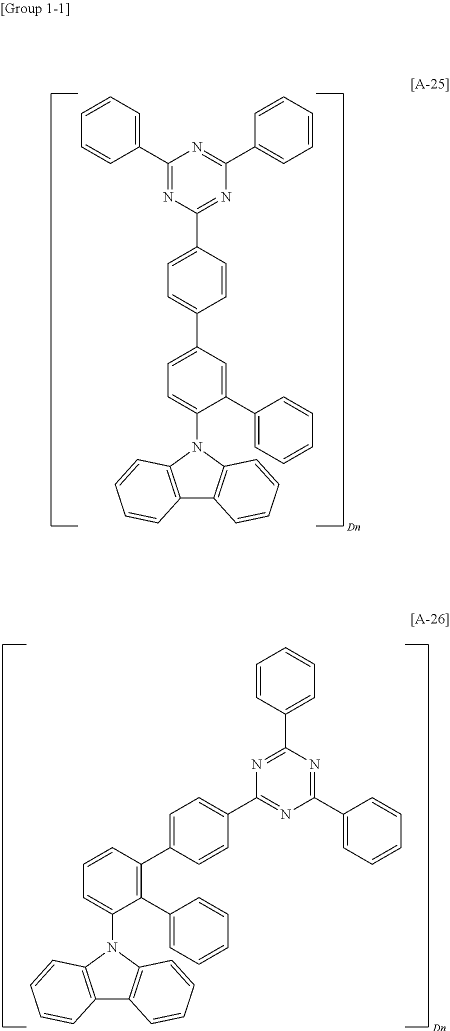

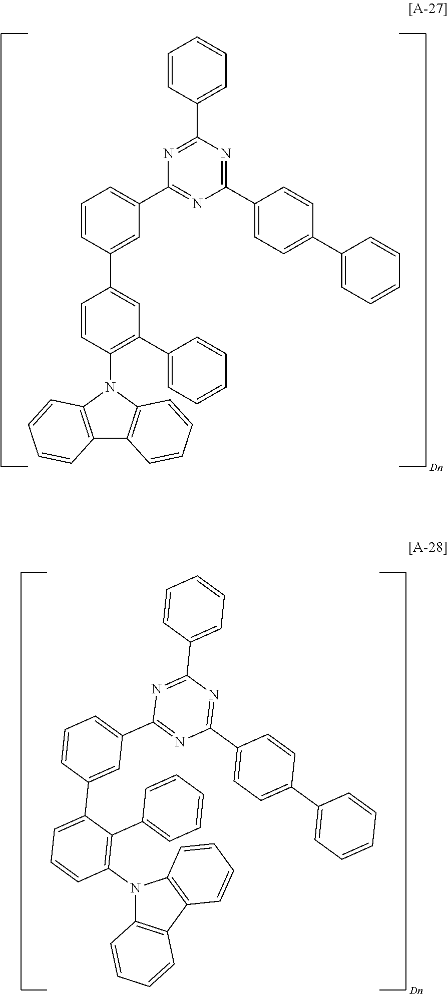

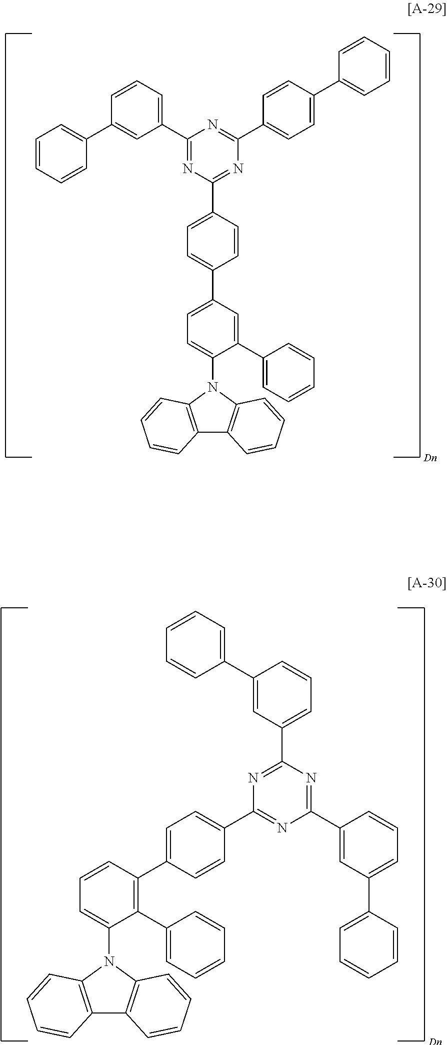

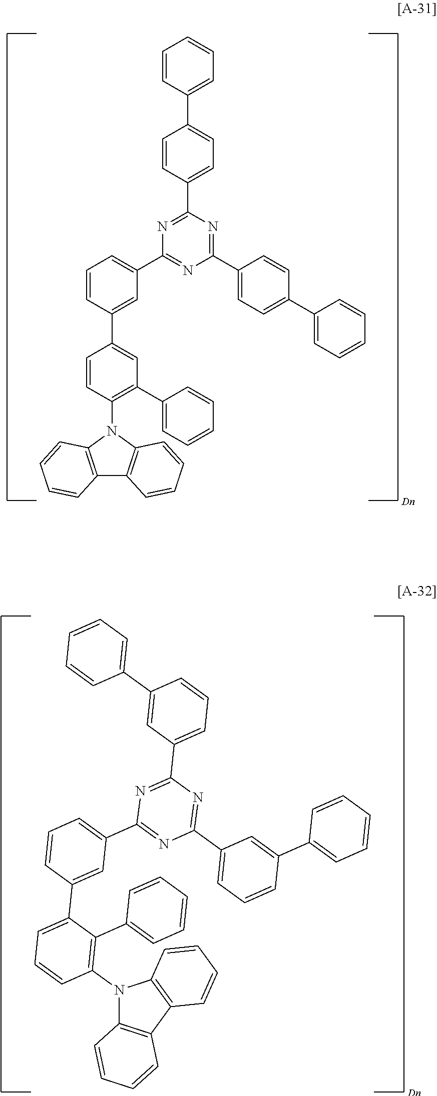

(Dn refers to the number of deuterium substitutions and indicates a structure substituted with one or more deuterium atoms).

More specific examples of the compounds listed in Group 1-1 may be selected from the compounds listed in Group 1-2.

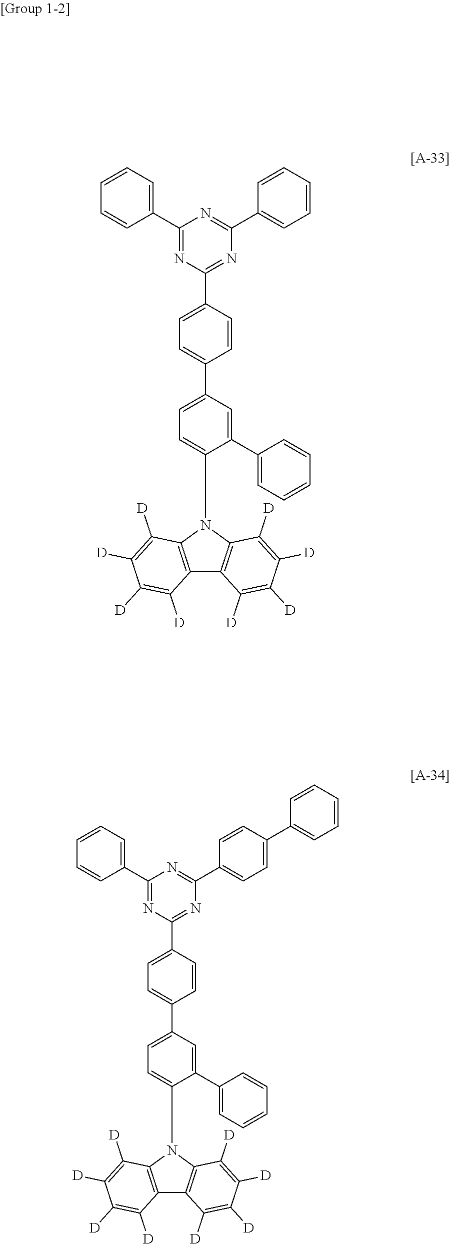 [SMILES: [2H]c1c([2H])c([2H])c2c(c1[2H])c1c([2H])c([2H])c([2H])c([2H])c1n2-c1ccc(-c2ccc(-c3nc(-c4ccccc4)nc(-c4ccccc4)n3)cc2)cc1-c1ccccc1 ; [2H]c1c([2H])c([2H])c2c(c1[2H])c1c([2H])c([2H])c([2H])c([2H])c1n2-c1ccc(-c2ccc(-c3nc(-c4ccccc4)nc(-c4ccc(-c5ccccc5)cc4)n3)cc2)cc1-c1ccccc1]

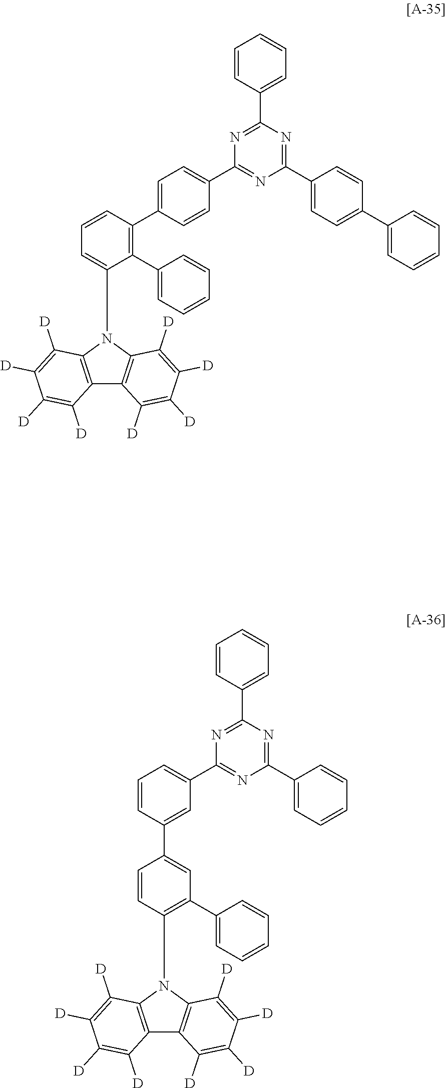 [SMILES: [2H]c1c([2H])c([2H])c2c(c1[2H])c1c([2H])c([2H])c([2H])c([2H])c1n2-c1cccc(-c2ccc(-c3nc(-c4ccccc4)nc(-c4ccc(-c5ccccc5)cc4)n3)cc2)c1-c1ccccc1 ; [2H]c1c([2H])c([2H])c2c(c1[2H])c1c([2H])c([2H])c([2H])c([2H])c1n2-c1ccc(-c2cccc(-c3nc(-c4ccccc4)nc(-c4ccccc4)n3)c2)cc1-c1ccccc1]

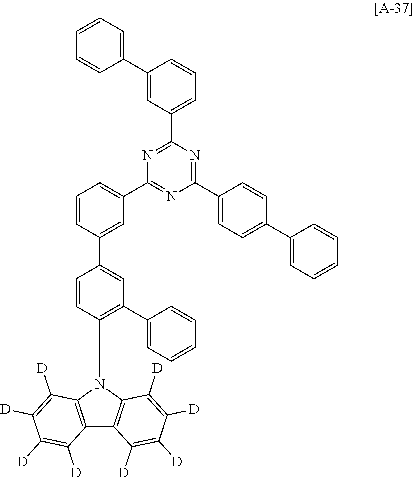 [SMILES: [2H]c1c([2H])c([2H])c2c(c1[2H])c1c([2H])c([2H])c([2H])c([2H])c1n2-c1ccc(-c2cccc(-c3nc(-c4ccc(-c5ccccc5)cc4)nc(-c4cccc(-c5ccccc5)c4)n3)c2)cc1-c1ccccc1]

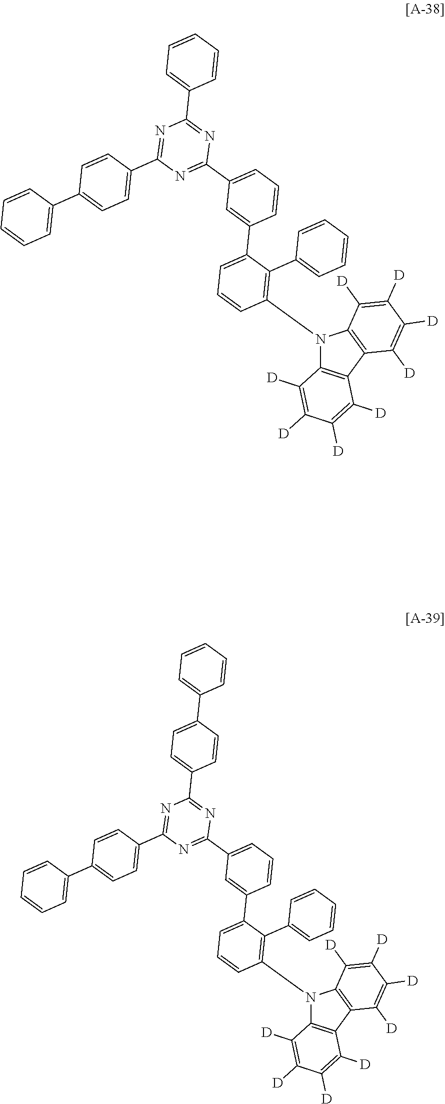 [SMILES: [2H]c1c([2H])c([2H])c2c(c1[2H])c1c([2H])c([2H])c([2H])c([2H])c1n2-c1cccc(-c2cccc(-c3nc(-c4ccccc4)nc(-c4ccc(-c5ccccc5)cc4)n3)c2)c1-c1ccccc1 ; [2H]c1c([2H])c([2H])c2c(c1[2H])c1c([2H])c([2H])c([2H])c([2H])c1n2-c1cccc(-c2cccc(-c3nc(-c4ccc(-c5ccccc5)cc4)nc(-c4ccc(-c5ccccc5)cc4)n3)c2)c1-c1ccccc1]

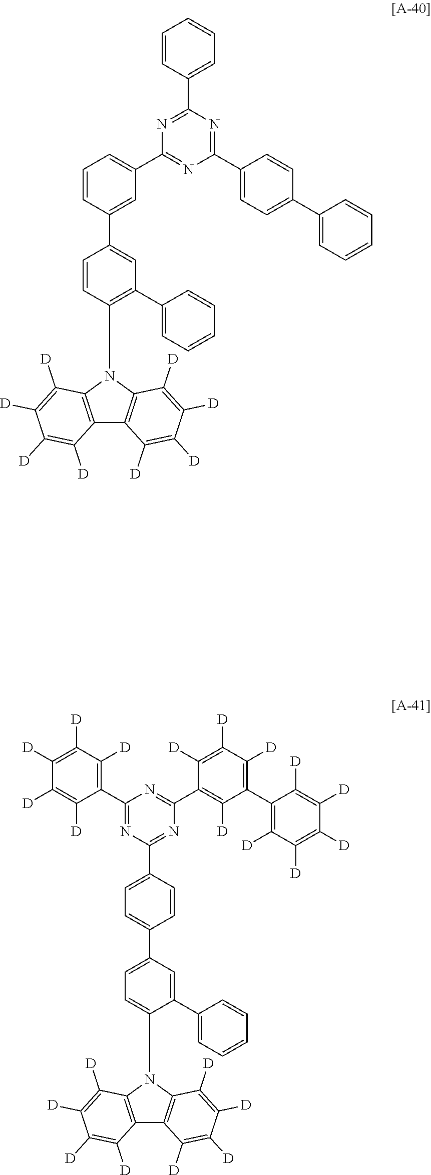 [SMILES: [2H]c1c([2H])c([2H])c2c(c1[2H])c1c([2H])c([2H])c([2H])c([2H])c1n2-c1ccc(-c2cccc(-c3nc(-c4ccccc4)nc(-c4ccc(-c5ccccc5)cc4)n3)c2)cc1-c1ccccc1 ; [2H]c1c([2H])c([2H])c(-c2nc(-c3ccc(-c4ccc(-n5c6c([2H])c([2H])c([2H])c([2H])c6c6c([2H])c([2H])c([2H])c([2H])c65)c(-c5ccccc5)c4)cc3)nc(-c3c([2H])c([2H])c([2H])c(-c4c([2H])c([2H])c([2H])c([2H])c4[2H])c3[2H])n2)c([2H])c1[2H]]

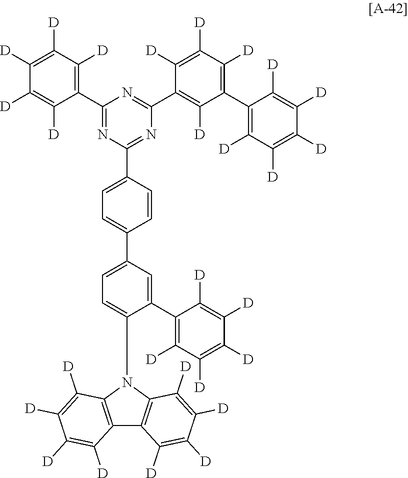 [SMILES: [2H]c1c([2H])c([2H])c(-c2nc(-c3ccc(-c4ccc(-n5c6c([2H])c([2H])c([2H])c([2H])c6c6c([2H])c([2H])c([2H])c([2H])c65)c(-c5c([2H])c([2H])c([2H])c([2H])c5[2H])c4)cc3)nc(-c3c([2H])c([2H])c([2H])c(-c4c([2H])c([2H])c([2H])c([2H])c4[2H])c3[2H])n2)c([2H])c1[2H]]

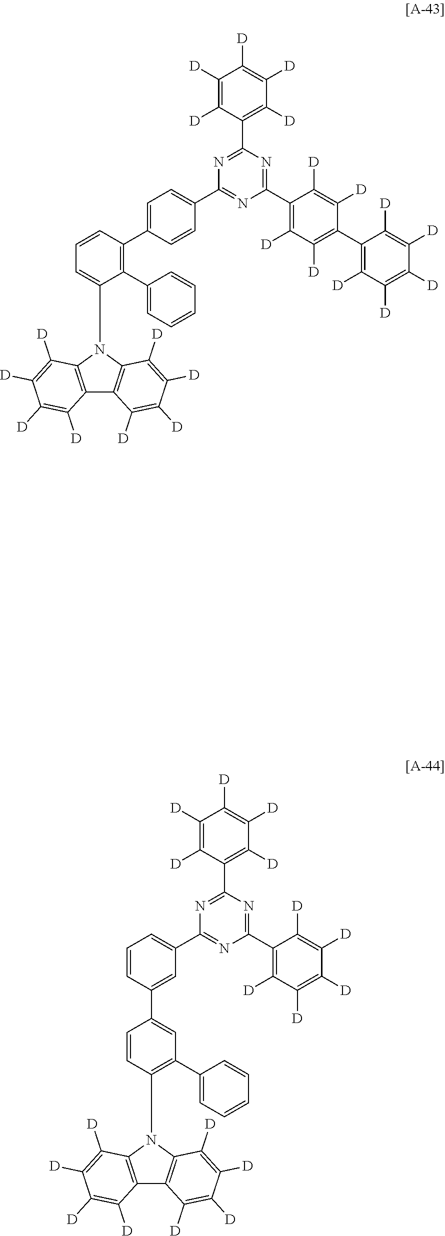 [SMILES: [2H]c1c([2H])c([2H])c(-c2nc(-c3ccc(-c4cccc(-n5c6c([2H])c([2H])c([2H])c([2H])c6c6c([2H])c([2H])c([2H])c([2H])c65)c4-c4ccccc4)cc3)nc(-c3c([2H])c([2H])c(-c4c([2H])c([2H])c([2H])c([2H])c4[2H])c([2H])c3[2H])n2)c([2H])c1[2H] ; [2H]c1c([2H])c([2H])c(-c2nc(-c3cccc(-c4ccc(-n5c6c([2H])c([2H])c([2H])c([2H])c6c6c([2H])c([2H])c([2H])c([2H])c65)c(-c5ccccc5)c4)c3)nc(-c3c([2H])c([2H])c([2H])c([2H])c3[2H])n2)c([2H])c1[2H]]

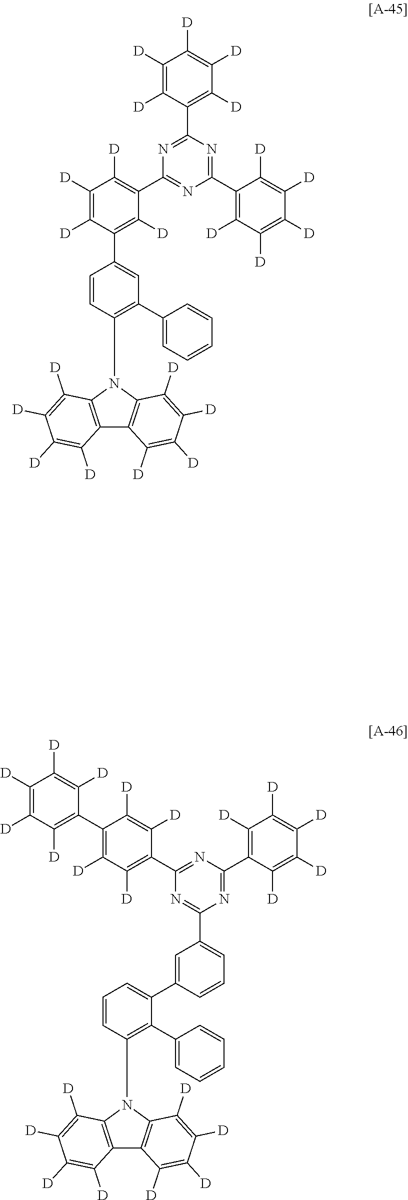 [SMILES: [2H]c1c([2H])c([2H])c(-c2nc(-c3c([2H])c([2H])c([2H])c([2H])c3[2H])nc(-c3c([2H])c([2H])c([2H])c(-c4ccc(-n5c6c([2H])c([2H])c([2H])c([2H])c6c6c([2H])c([2H])c([2H])c([2H])c65)c(-c5ccccc5)c4)c3[2H])n2)c([2H])c1[2H] ; [2H]c1c([2H])c([2H])c(-c2nc(-c3cccc(-c4cccc(-n5c6c([2H])c([2H])c([2H])c([2H])c6c6c([2H])c([2H])c([2H])c([2H])c65)c4-c4ccccc4)c3)nc(-c3c([2H])c([2H])c(-c4c([2H])c([2H])c([2H])c([2H])c4[2H])c([2H])c3[2H])n2)c([2H])c1[2H]]

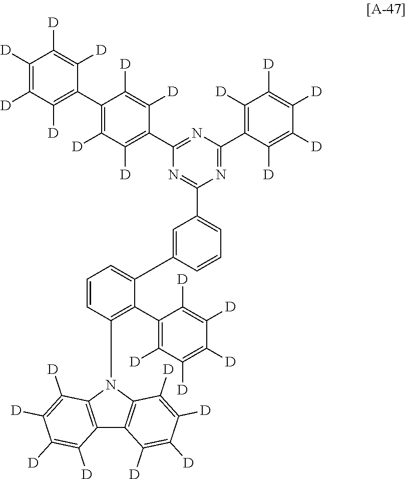 [SMILES: [2H]c1c([2H])c([2H])c(-c2nc(-c3cccc(-c4cccc(-n5c6c([2H])c([2H])c([2H])c([2H])c6c6c([2H])c([2H])c([2H])c([2H])c65)c4-c4c([2H])c([2H])c([2H])c([2H])c4[2H])c3)nc(-c3c([2H])c([2H])c(-c4c([2H])c([2H])c([2H])c([2H])c4[2H])c([2H])c3[2H])n2)c([2H])c1[2H]]

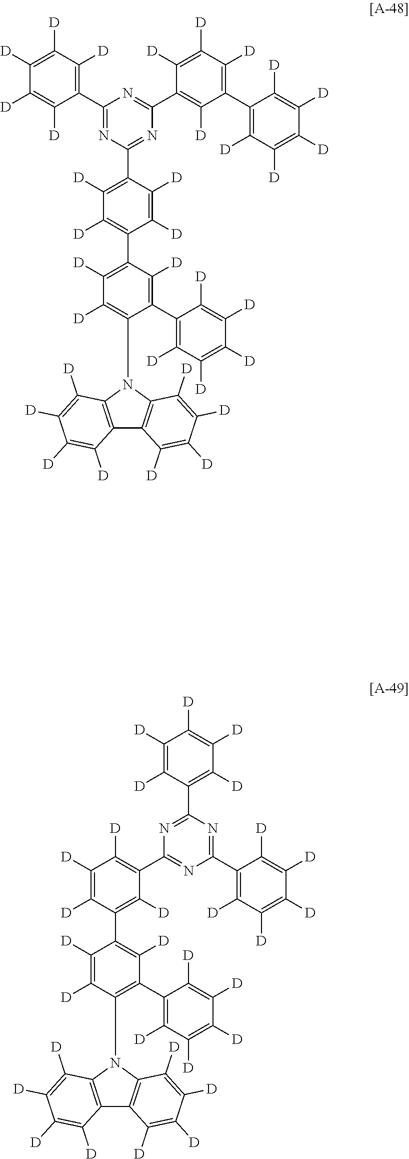

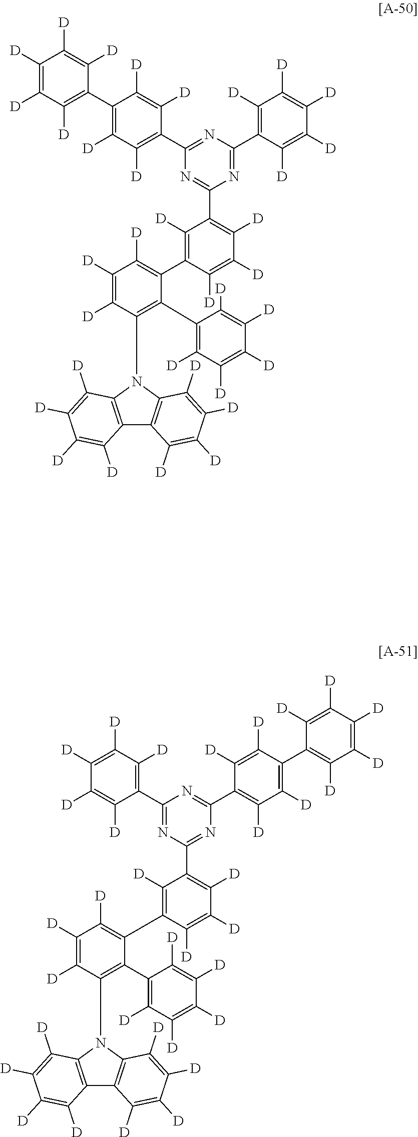

A composition for an organic optoelectronic device according to some example embodiments may include a first compound and a second compound. The first compound may be the aforementioned compound for an organic optoelectronic device and the second compound may be represented by Chemical Formula 2; a combination of Chemical Formula 3 and Chemical Formula 4; or Chemical Formula 5.

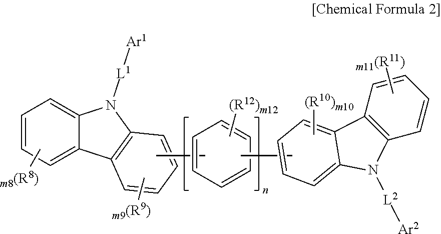

In Chemical Formula 2, R⁸ to R¹² may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar¹ and Ar² may each independently be, e.g., a substituted or unsubstituted C6 to C20 aryl group or a substituted or unsubstituted C2 to C30 heterocyclic group.

L¹ and L² may each independently be, e.g., a single bond or a substituted or unsubstituted C6 to C20 arylene group.

m8, m11, and m12 may each independently be, e.g., an integer of 1 to 4.

m9 and m10 may each independently be, e.g., an integer of 1 to 3.

n may be, e.g., an integer of 0 to 2.

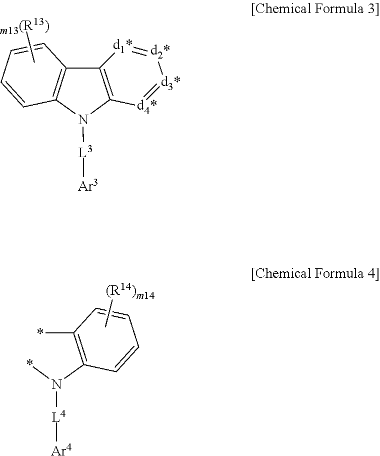

In Chemical Formula 3 and Chemical Formula 4, two adjacent ones of d₁* to d₄* in

Chemical Formula 3, may each be linking carbons linked to * of Chemical Formula 4.

The remaining two of d₁* to d₄* in Chemical Formula 3, not linked at * of Chemical Formula 4 may be, e.g., C-L^{a}-R^{c}.

L^{a}, L³, and L⁴ may each independently be, e.g., a single bond or a substituted or unsubstituted C6 to C20 arylene group.

R^{c}, R¹³, and R¹⁴ may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar³ and Ar⁴ may each independently be, e.g., a substituted or unsubstituted C6 to C20 aryl group or a substituted or unsubstituted C2 to C30 heterocyclic group.

m13 and m14 may each independently be, e.g., an integer of 1 to 4.

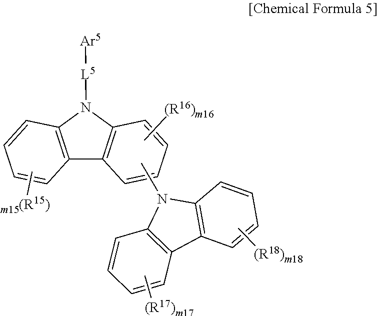

In Chemical Formula 5, L⁵ may be, e.g., a single bond or a substituted or unsubstituted C6 to C20 arylene group.

R¹⁵ to R¹⁸ may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar⁵ may be, e.g., a substituted or unsubstituted C6 to C20 aryl group or a substituted or unsubstituted C2 to C30 heterocyclic group.

m15, m17, and m18 may each independently be, e.g., an integer of 1 to 4.

m16 may be, e.g., an integer of 1 to 3.

The second compound can be used in the light emitting layer together with the first compound to improve luminous efficiency and life-span characteristics by increasing charge mobility and stability.

In an implementation, m8 may be 2, 3, or 4, and each R⁸ may be the same or different from each other.

In an implementation, m9 may be 2 or 3, and each R⁹ may be the same or different from each other.

In an implementation, m10 may be 2 or 3, and each R¹⁰ may be the same or different from each other.

In an implementation, m11 may be 2, 3, or 4, and each R¹¹ may be the same or different from each other.

In an implementation, m12 may be 2, 3, or 4, and each R¹² may be the same or different from each other.

In an implementation, m13 may be 2, 3, or 4, and each R¹³ may be the same or different from each other.

In an implementation, m14 may be 2, 3, or 4, and each R¹⁴ may be the same or different from each other.

In an implementation, Re may be present two or more times, and each Re may be the same or

different from each other.

In an implementation, m15 may be 2, 3, or 4, and each R¹⁵ may be the same or different from each other.

In an implementation, m16 may be 2 or 3, and each R¹⁶ may be the same or different from each other.

In an implementation, m17 may be 2, 3, or 4, and each R¹⁷ may be the same or different from each other.

In an implementation, m18 may be 2, 3, or 4, and each R¹⁸ may be the same or different from each other.

In an implementation, in Chemical Formula 2, Ar¹ and Ar² may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, in Chemical Formula 2, L¹ and L² may each independently be a single bond, a substituted or unsubstituted phenylene group, or a substituted or unsubstituted biphenylene group.

In an implementation, in Chemical Formula 2, R⁸ to R¹² may each independently be hydrogen, deuterium, or a substituted or unsubstituted C6 to C12 aryl group.

In an implementation, in Chemical Formula 2, n may be 0 or 1.

In an implementation, in Chemical Formula 2, “substituted” may refer to replacement of at least one hydrogen by deuterium, a C1 to C4 alkyl group, a C6 to C18 aryl group, or a C2 to C30 heteroaryl group.

In an implementation, in Chemical Formula 2, Ar¹ and Ar² may each independently be a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, Chemical Formula 2 may be represented by one of Chemical Formula 2-1 to Chemical Formula 2-15.

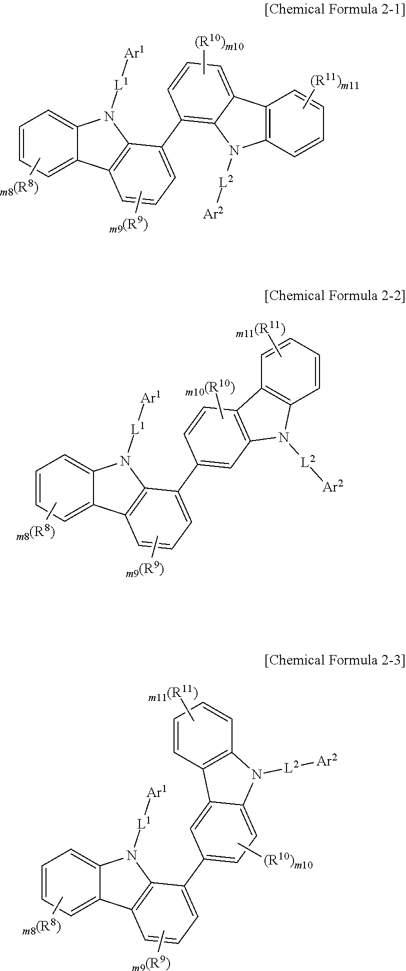

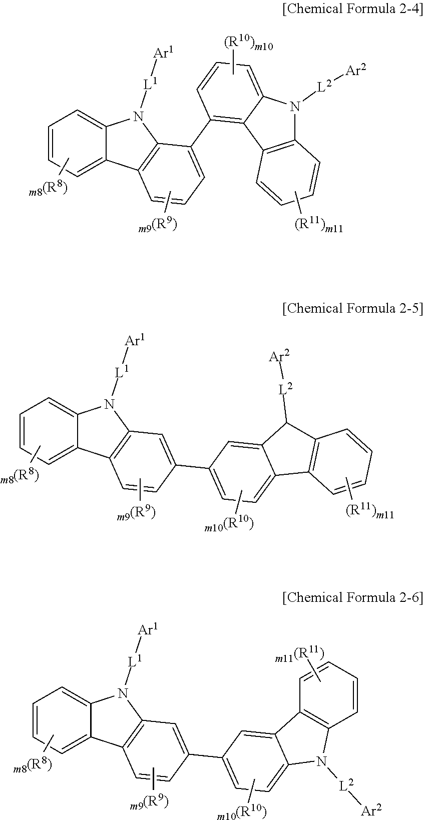

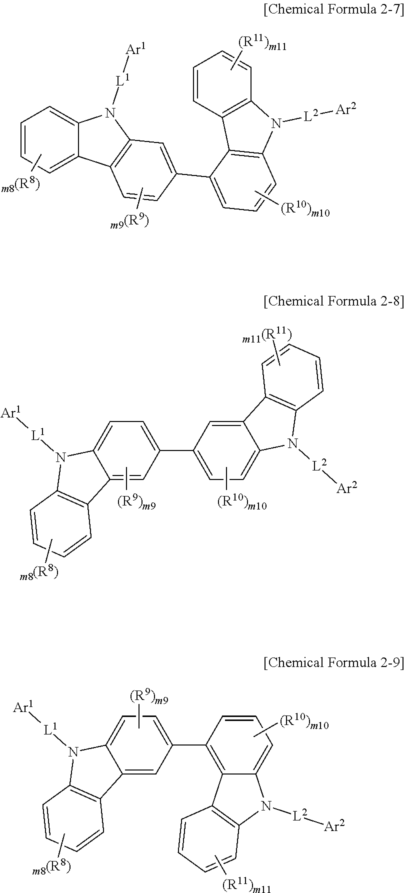

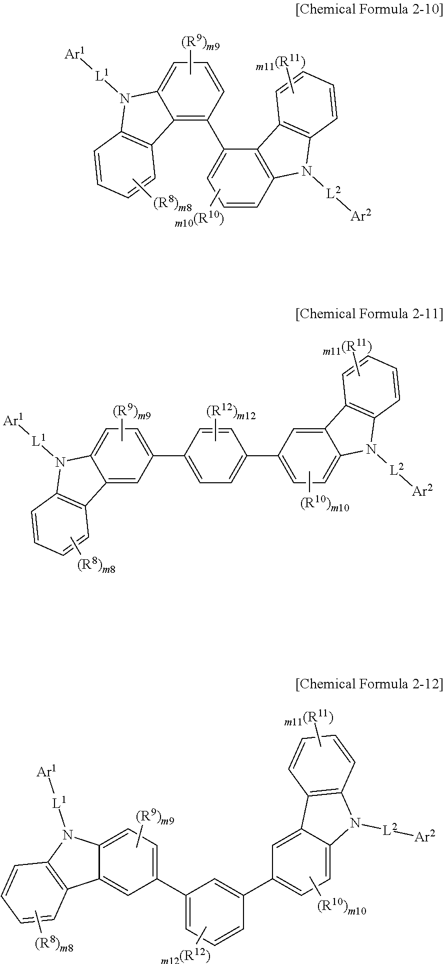

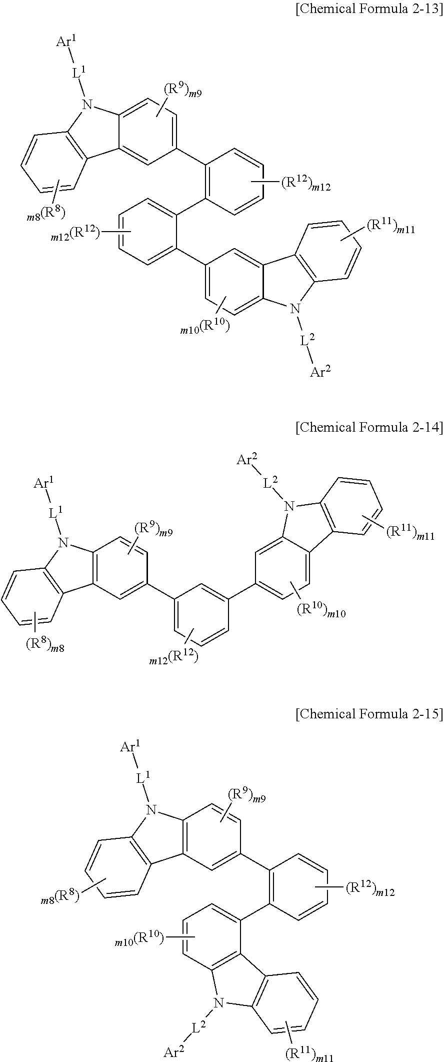

In Chemical Formula 2-1 to Chemical Formula 2-15, R⁸ to R¹² may each independently be, e.g., hydrogen, deuterium, or a substituted or unsubstituted C6 to C12 aryl group, and moieties L¹-Ar¹ and L²-Ar² may each independently be a moiety of Group I.

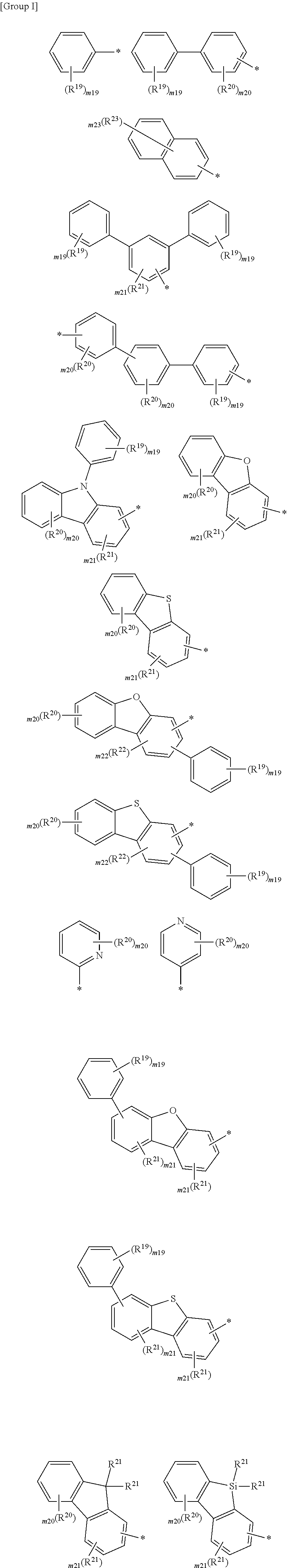

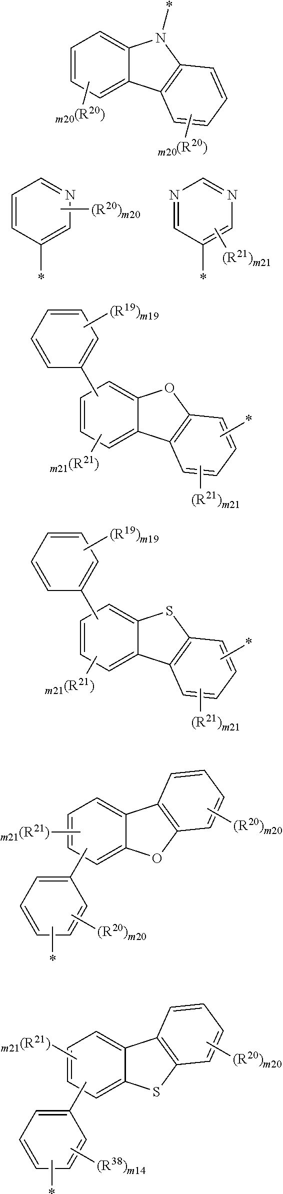

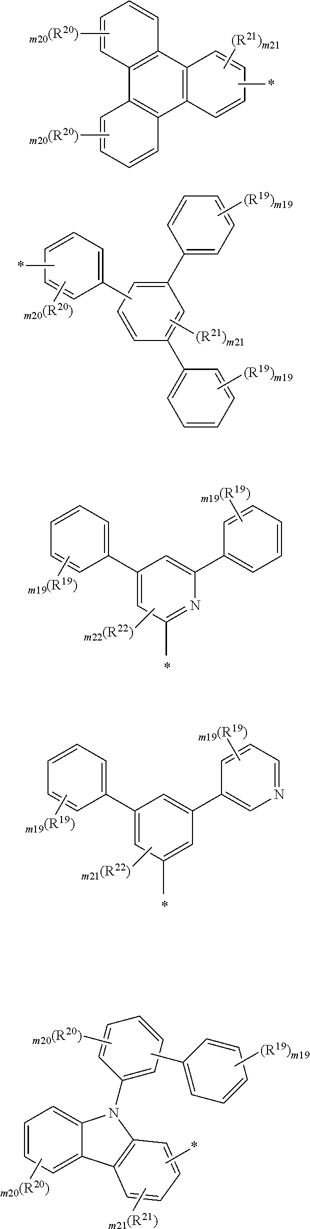

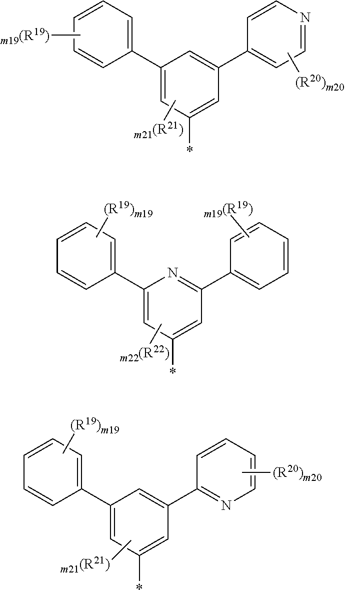

In Group I, R¹⁹ to R²³ may each independently be, e.g., hydrogen, deuterium, a cyano group, a substituted or unsubstituted C1 to C10 alkyl group, or a substituted or unsubstituted C6 to C12 aryl group.

m19 may be, e.g., an integer of 1 to 5.

m20 may be, e.g., an integer of 1 to 4.

m21 may be, e.g., an integer of 1 to 3.

m22 may be, e.g., an integer of 1 or 2.

m23 may be, e.g., an integer of 1 to 7.

* is a linking point.

In an implementation, m19 may be 2, 3, 4, or 5, and each R¹⁹ may be the same or different from each other.

In an implementation, m20 may be 2, 3, or 4, and each R²⁰ may be the same or different from each other.

In an implementation, m21 may be 2 or 3, and each R²¹ may be the same or different from each other.

In an implementation, m22 may be 2, and each R²² may be the same or different from each other.

In an implementation, m23 may be 2, 3, 4, 5, 6, or 7, and each R²³ may be the same or different from each other.

The combination of Chemical Formula 3 and Chemical Formula 4 may be represented, e.g., by one of Chemical Formula 3A, Chemical Formula 3B, Chemical Formula 3C, Chemical Formula 3D, or Chemical Formula 3E.

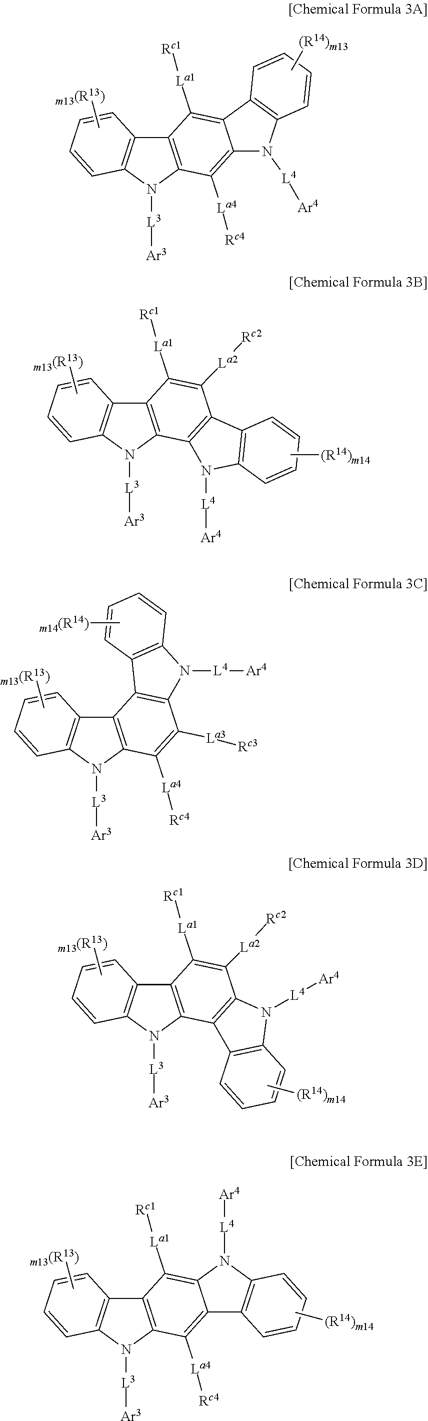

In Chemical Formula 3A to Chemical Formula 3E, L³, L⁴, Ar³, Ar⁴, R¹³, R¹⁴, m13, and m14 may be defined the same as described above.

L^{a1} to L^{a4} may be defined the same as L³ and L⁴ described above.

R^{c1} to R^{c4} may be defined the same as R¹³ and R¹⁴ described above.

In an implementation, in Chemical Formulae 3 and 4, Ar³ and Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, R^{c1} to R^{c4}, R¹³, and R¹⁴ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formulae 3 and 4, moieties L³-Ar³ and L⁴-Ar⁴ may each independently be a moiety of Group I.

In an implementation, R^{c1} to R^{c4}, R¹³, and R¹⁴ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{c1} to R^{c4}, R¹³, and R¹⁴ may each independently be hydrogen, deuterium, a cyano group, or a substituted or unsubstituted phenyl group.

In an implementation, R^{c1} to R^{c4}, R¹³, and R¹⁴ may each independently be hydrogen, deuterium, or a substituted or unsubstituted phenyl group.

In an implementation, the second compound may be represented by Chemical Formula 2-8, and in Chemical Formula 2-8, Ar¹ and Ar² may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L¹ and L² may each independently be a single bond, or a substituted or unsubstituted C6 to C20 arylene group, and R⁸ to R¹¹ may each independently be a hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 2-8, R⁸ to R¹¹ may each independently be hydrogen, deuterium, or a substituted or unsubstituted C6 to C12 aryl group, and moieties L¹-Ar¹ and L²-Ar² may each independently be a moiety of Group I.

In an implementation, the second compound may be represented by Chemical Formula 3C, and in Chemical Formula 3C, L^{a3} and L^{a4} may each independently be a single bond, L³ and L⁴ may each independently be a single bond or a substituted or unsubstituted C6 to C12 arylene group, R¹³, R¹⁴, R^{c3}, and R^{c4} may each independently be hydrogen, deuterium, or a substituted or unsubstituted phenyl group, and Ar³ and Ar⁴ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 3C, L^{a3} and L^{a4} may each be a single bond, R¹³, R¹⁴, R^{c3}, and R^{c4} may each independently be hydrogen, deuterium, or a substituted or unsubstituted C6 to C12 aryl group, and moieties L³-Ar³ and L⁴-Ar⁴ may each independently be a moiety of Group I.

Chemical Formula 5 may be for example represented by one of Chemical Formula 5-1 to Chemical Formula 5-4.

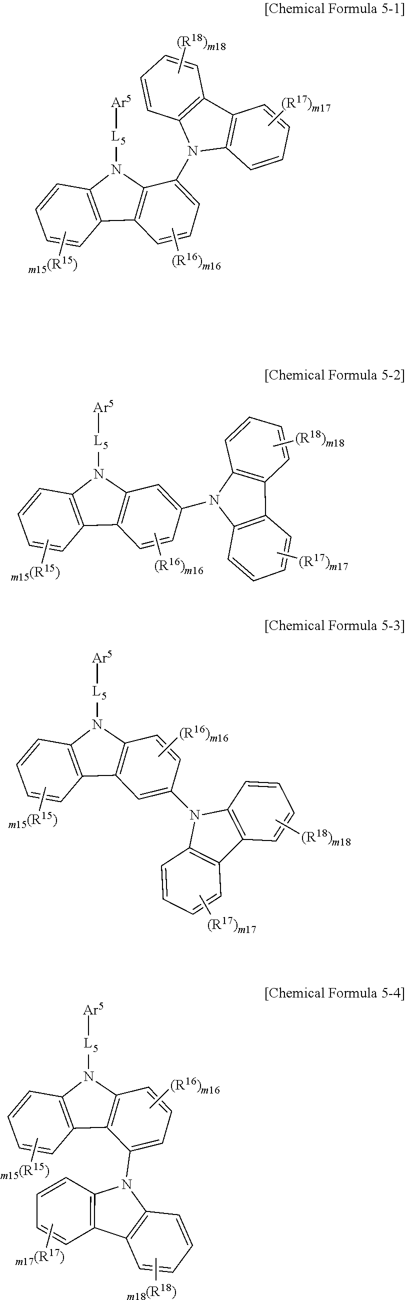

In Chemical Formula 5-1 to Chemical Formula 5-4, L⁵, Ar⁵, R¹⁵ to R¹⁸, and m15 to m18 may be defined the same as described above.

In an implementation, in Chemical Formula 5, Ar⁵ may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, R¹⁵ to R¹⁸ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 5, moiety L⁵-Ar⁵ may be a moiety of Group I.

In an implementation, R¹⁵ to R¹⁸ may each independently be hydrogen, deuterium, a cyano group, or a substituted or unsubstituted phenyl group.

In an implementation, the second compound for an organic optoelectronic device may be, e.g., a compound of Group 2.

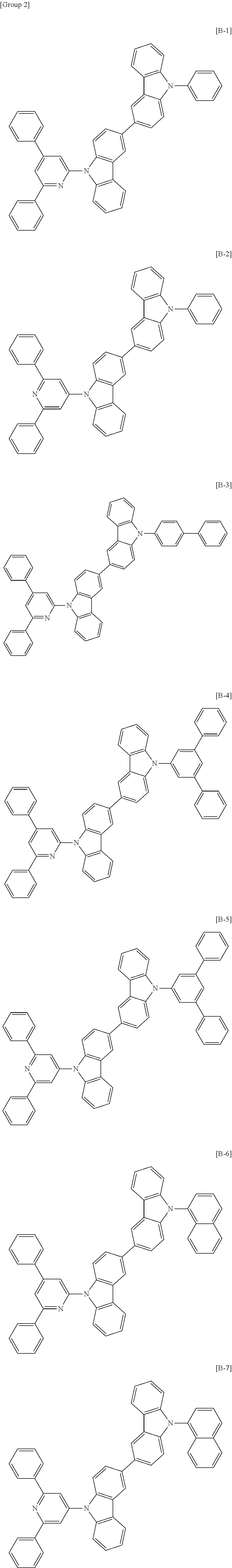

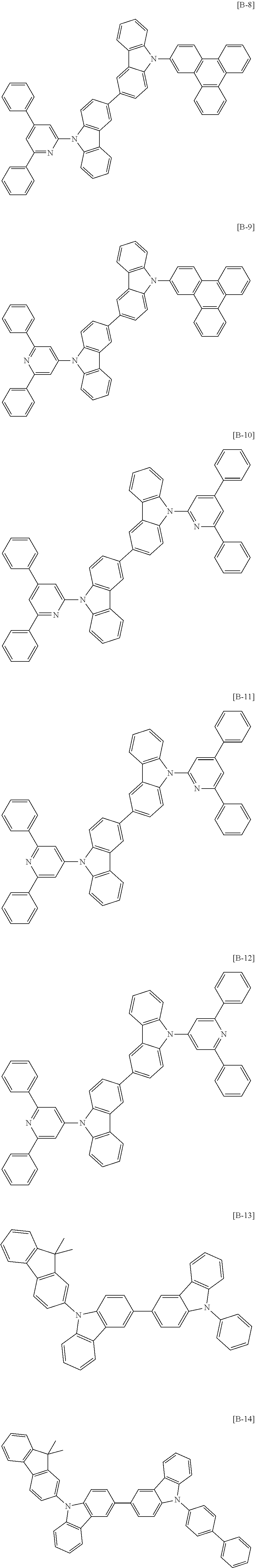

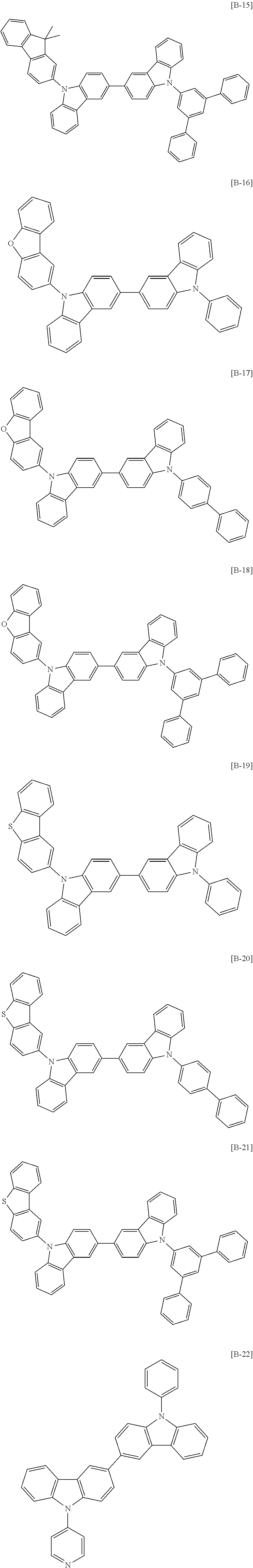

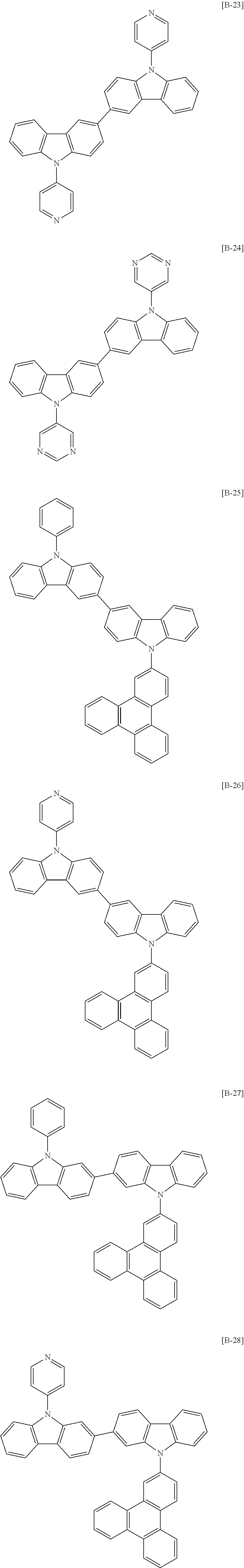 [SMILES: c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21]

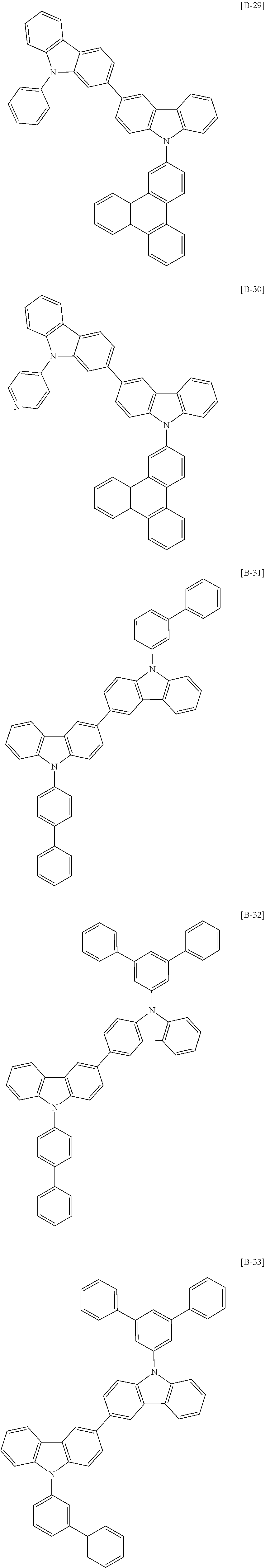 [SMILES: c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

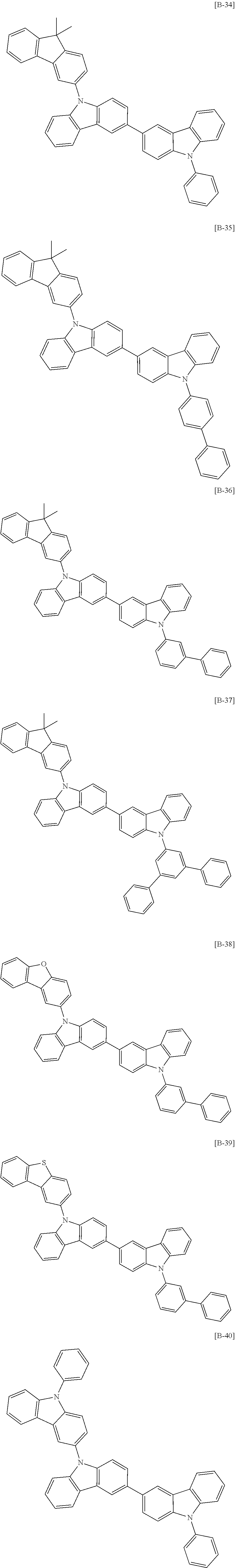

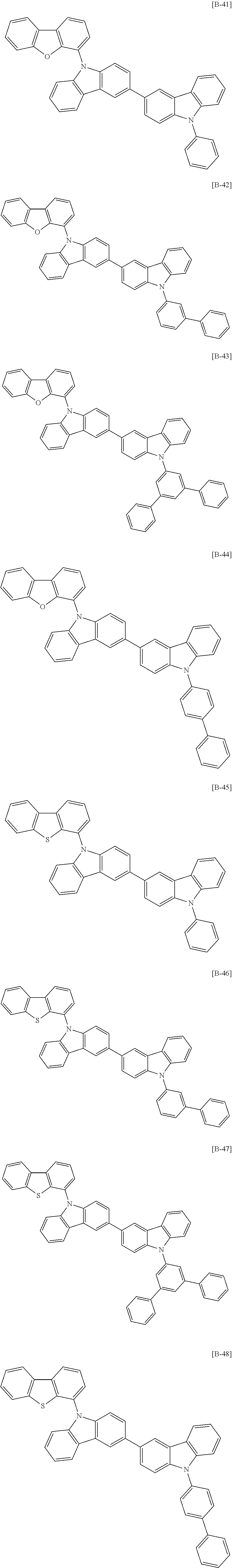

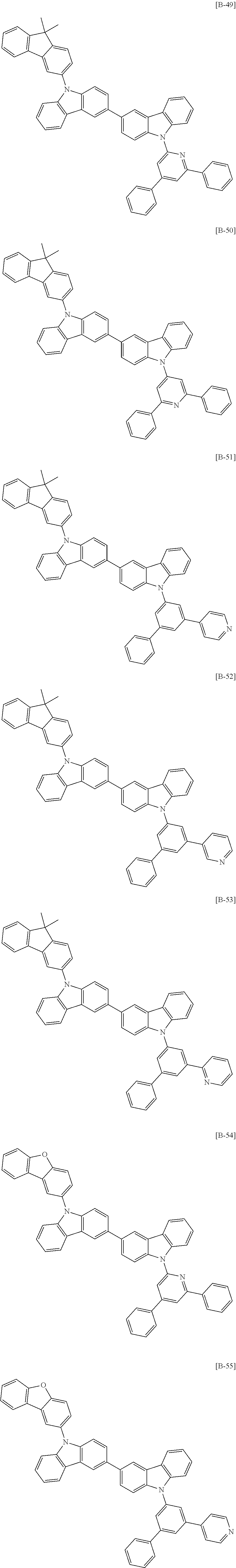

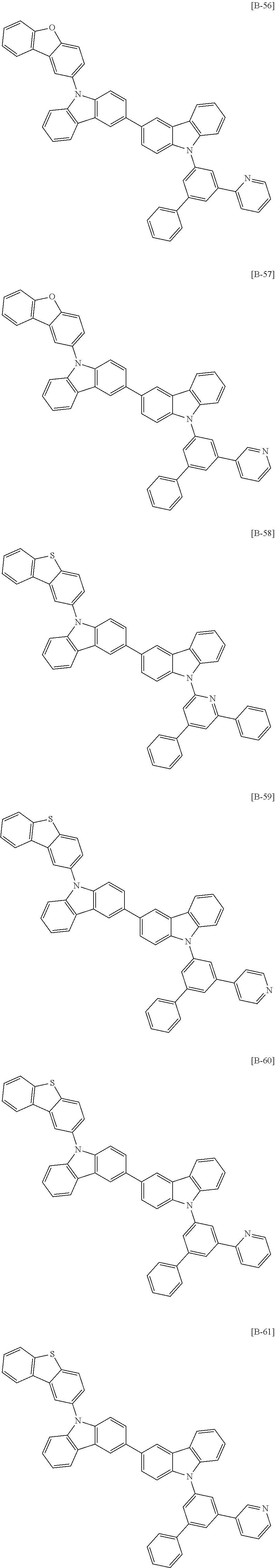

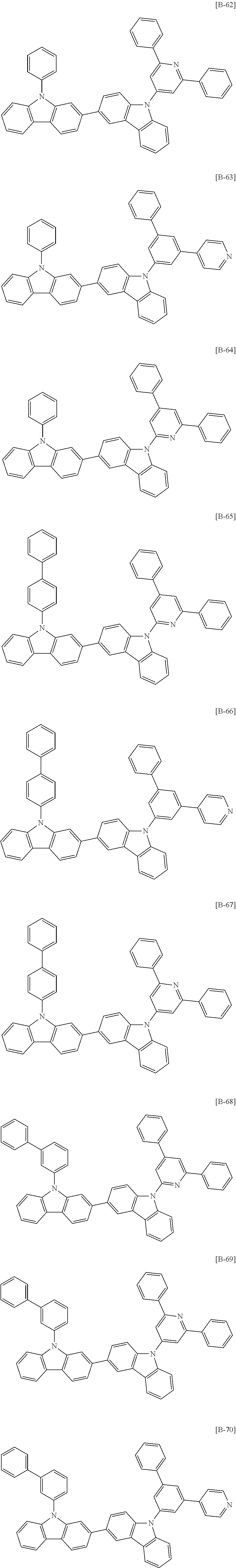

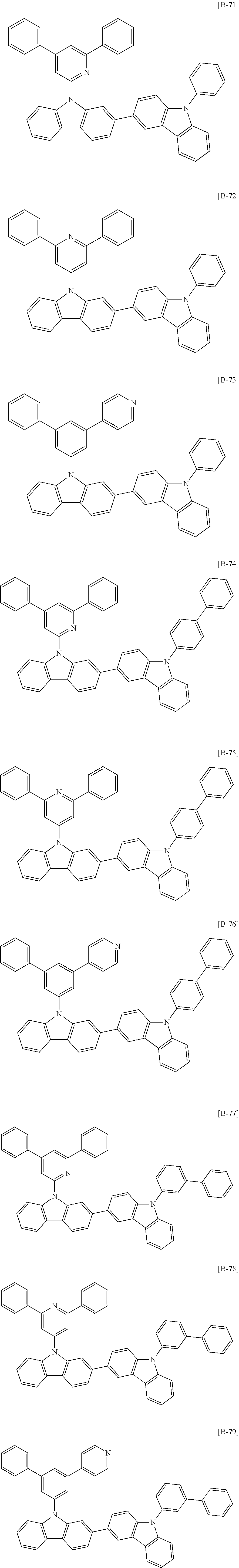

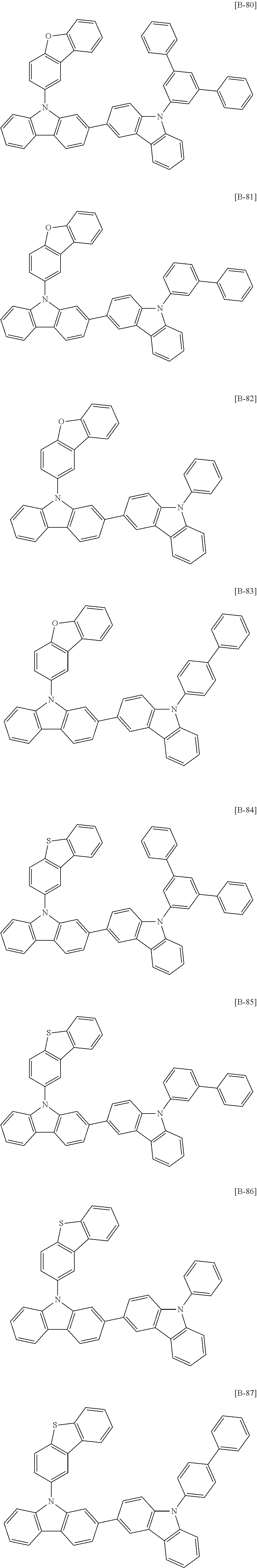

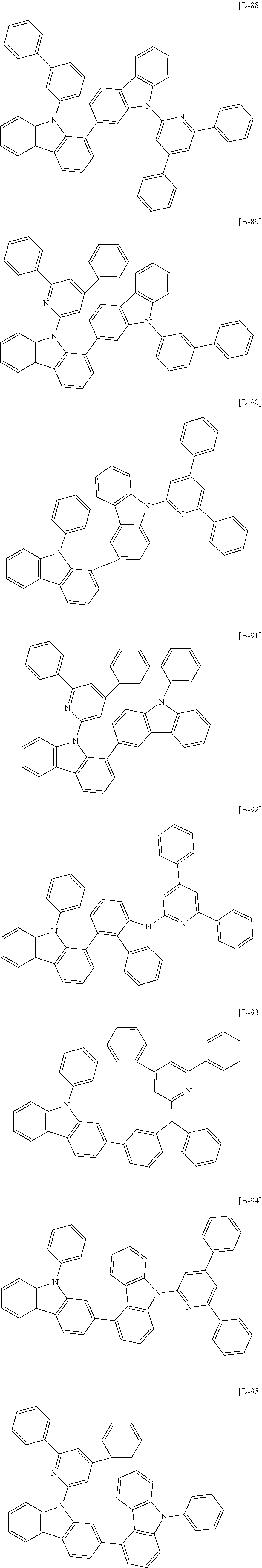

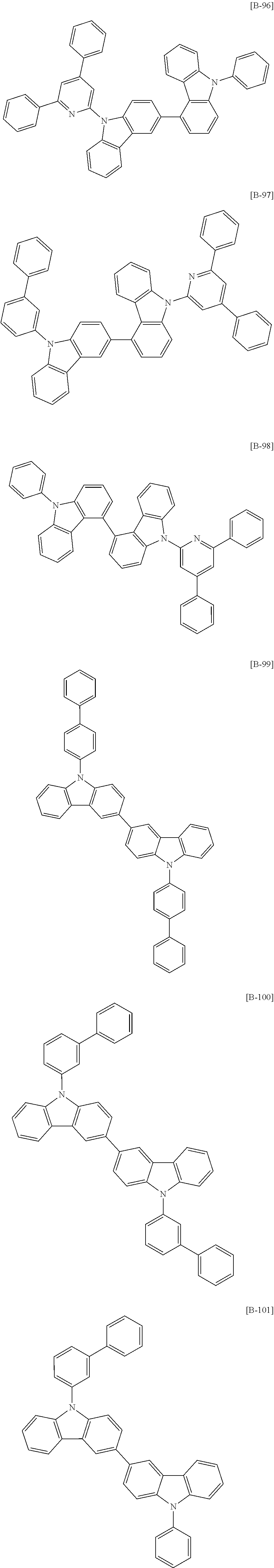

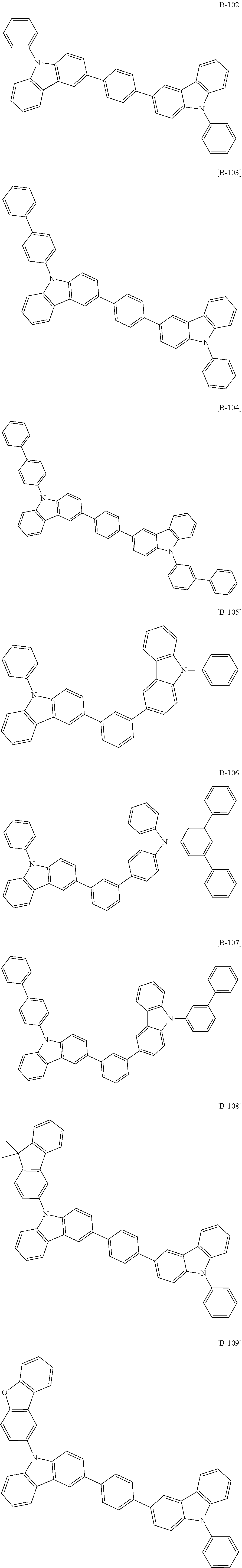

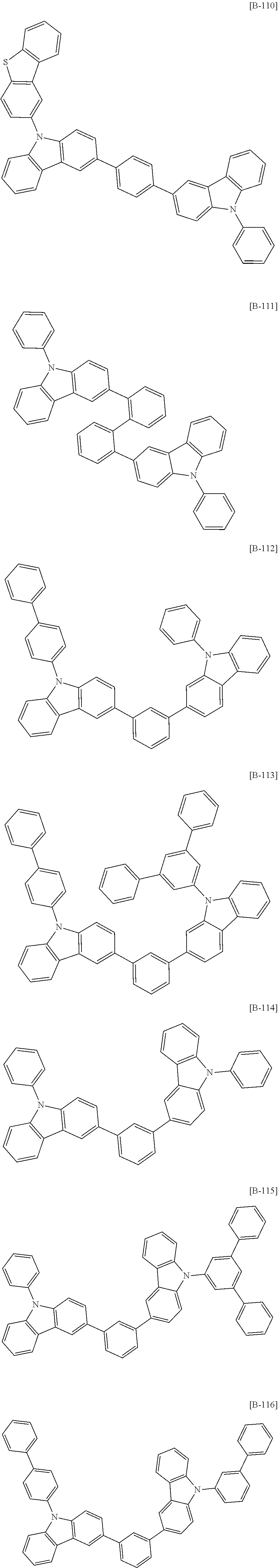

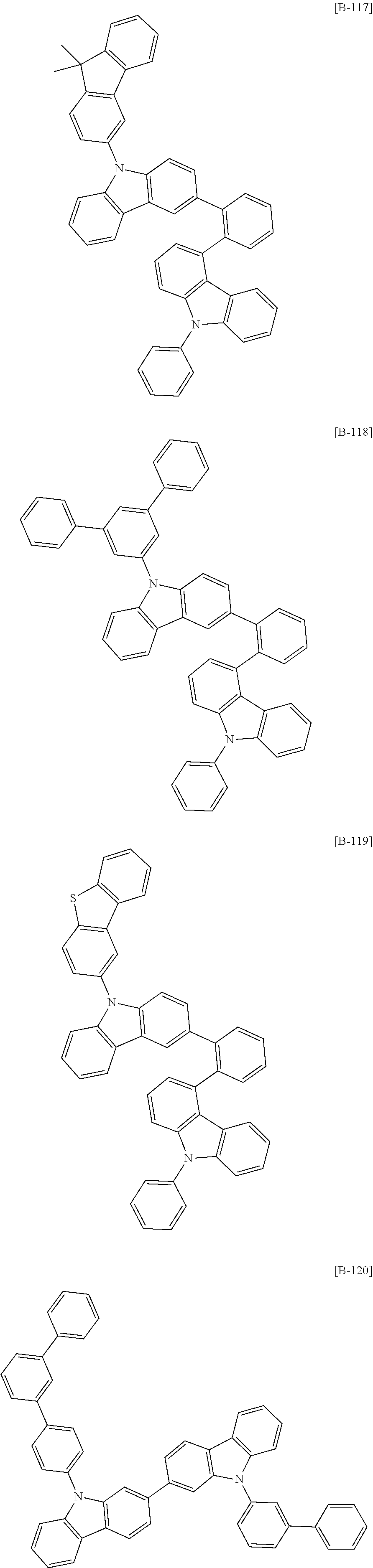 [SMILES: CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

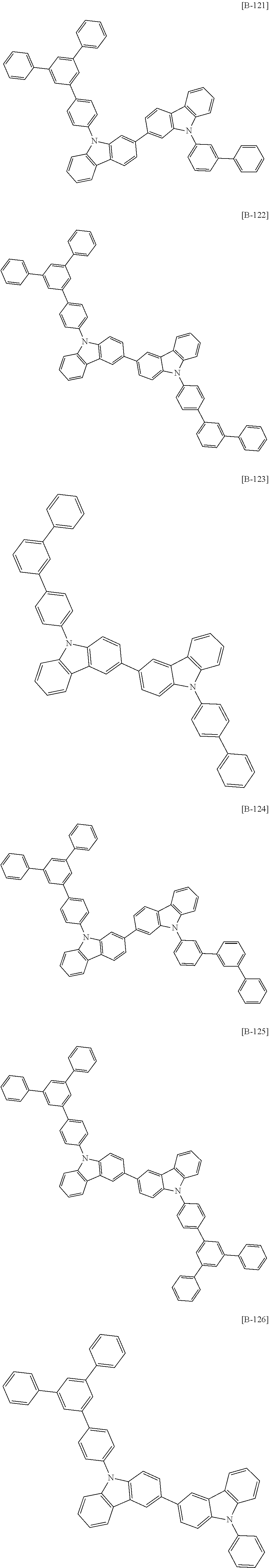

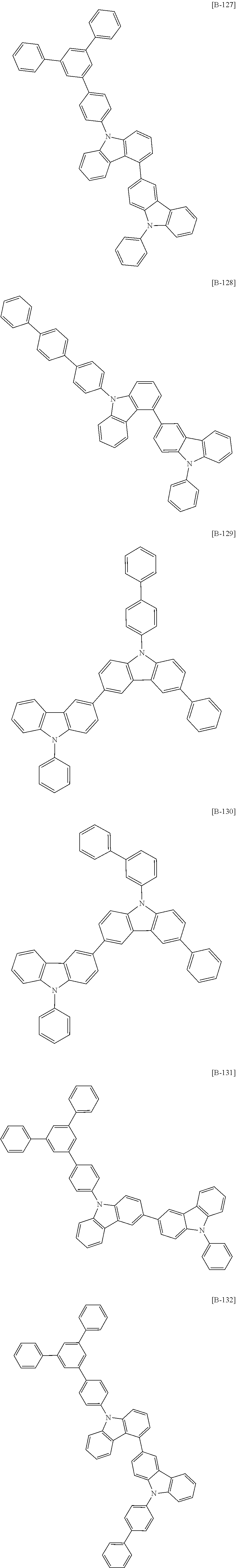

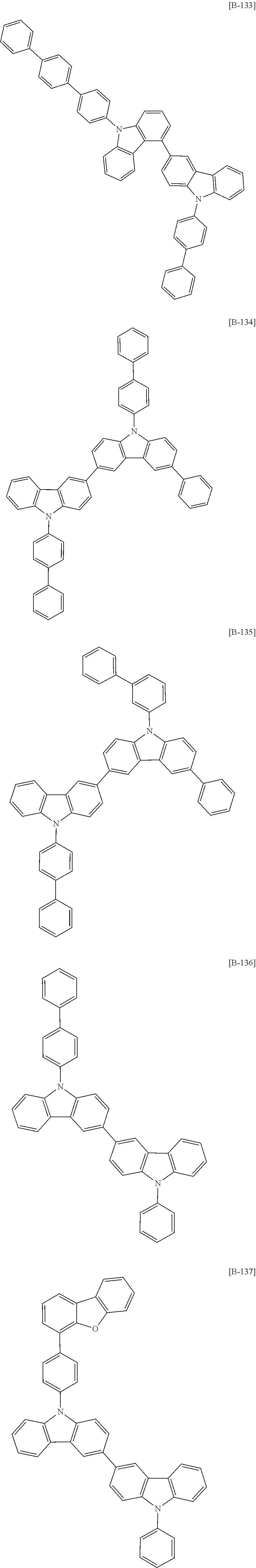 [SMILES: c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

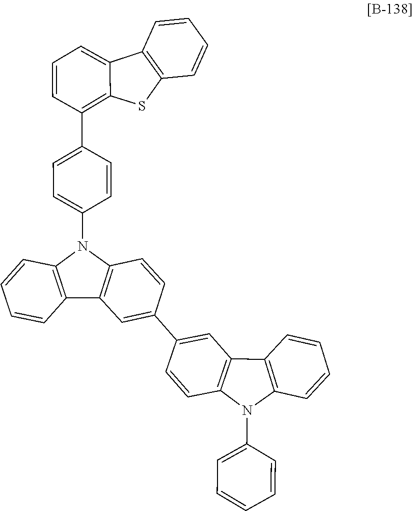 [SMILES: c1ccc(-n2c3ccccc3c3cc(-c4ccc5c(c4)c4ccccc4n5-c4ccc(-c5cccc6c5sc5ccccc56)cc4)ccc32)cc1]

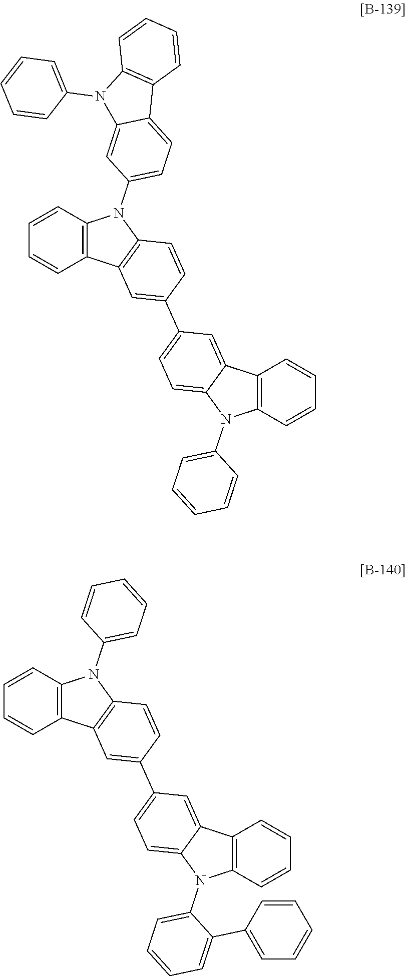 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1]

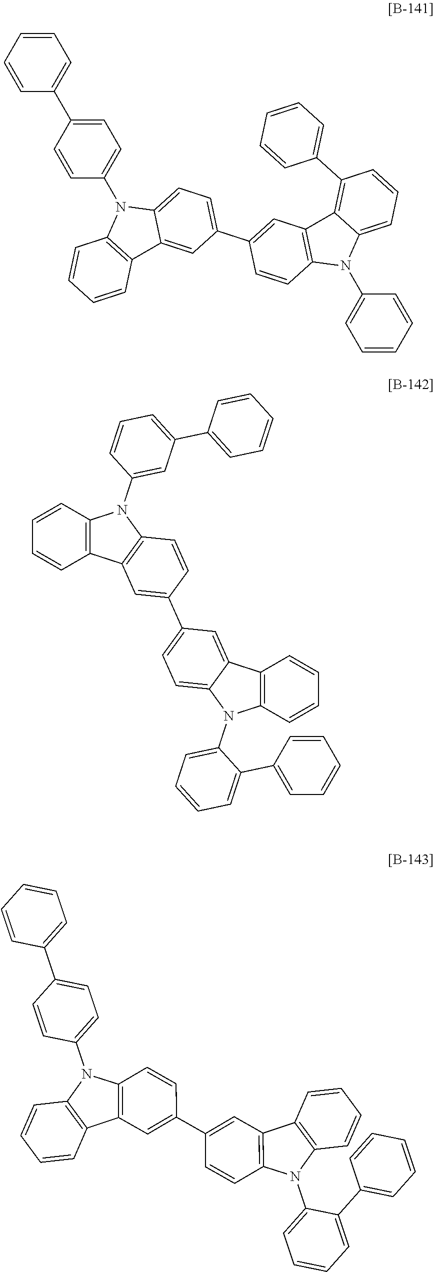 [SMILES: c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

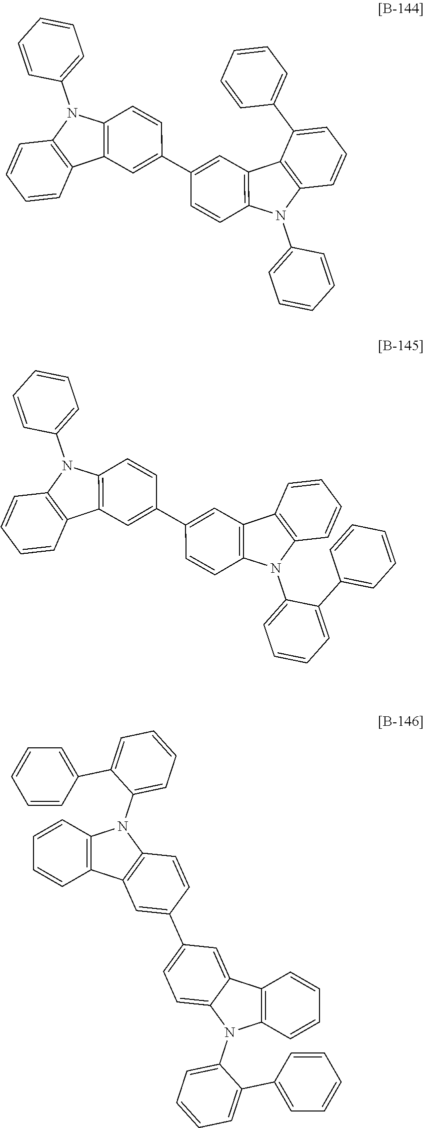 [SMILES: c1ccc(-c2cccc3c2c2cc(-c4ccc5c(c4)c4ccccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4-c4ccccc4)ccc32)cc1]

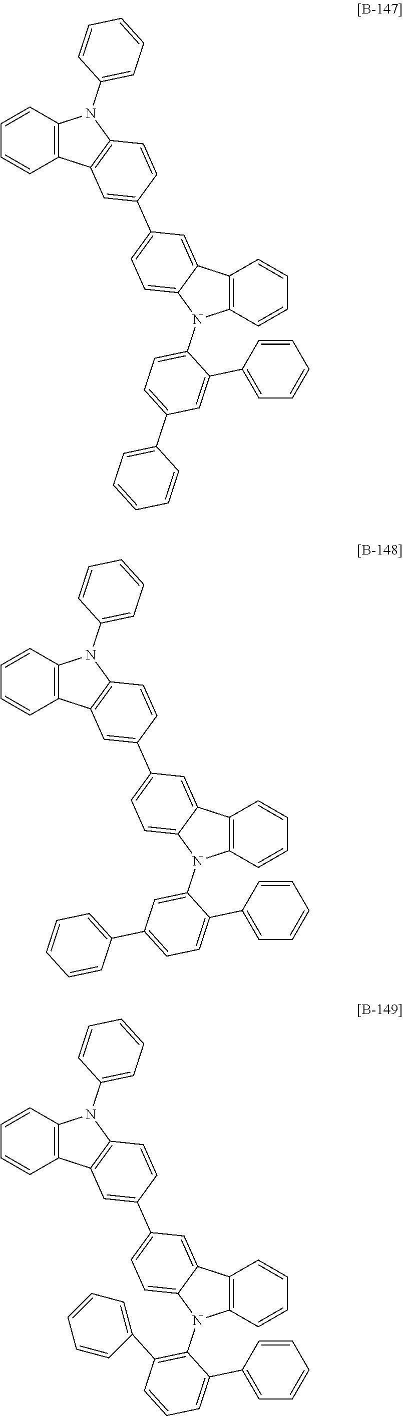 [SMILES: c1ccc(-c2ccc(-n3c4ccccc4c4cc(-c5ccc6c(c5)c5ccccc5n6-c5ccccc5)ccc43)c(-c3ccccc3)c2)cc1 ; c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2cccc(-c3ccccc3)c2-n2c3ccccc3c3cc(-c4ccc5c(c4)c4ccccc4n5-c4ccccc4)ccc32)cc1]

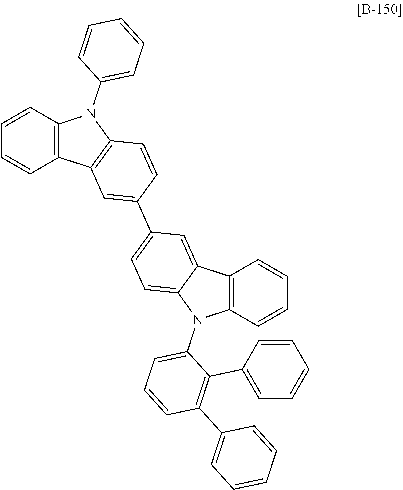 [SMILES: c1ccc(-c2cccc(-n3c4ccccc4c4cc(-c5ccc6c(c5)c5ccccc5n6-c5ccccc5)ccc43)c2-c2ccccc2)cc1]

Additionally, examples of Compound B-1 to Compound B-150, listed in Group 2, in which at least one hydrogen is replaced with deuterium are given below.

In an implementation, deuterium may be substituted, e.g., as shown in Compound B-151 to Compound B-195, exemplified below.

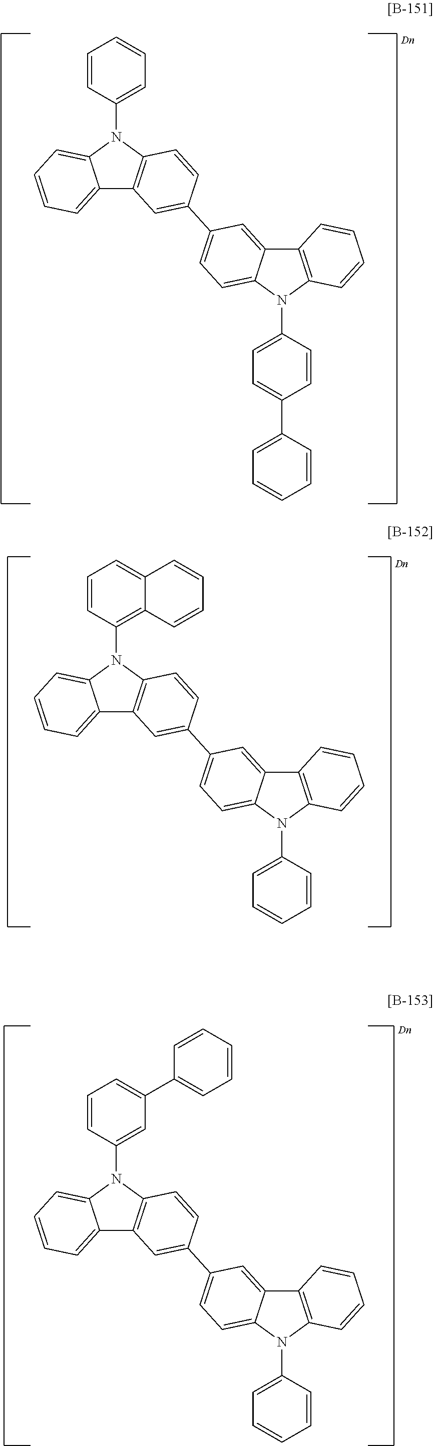

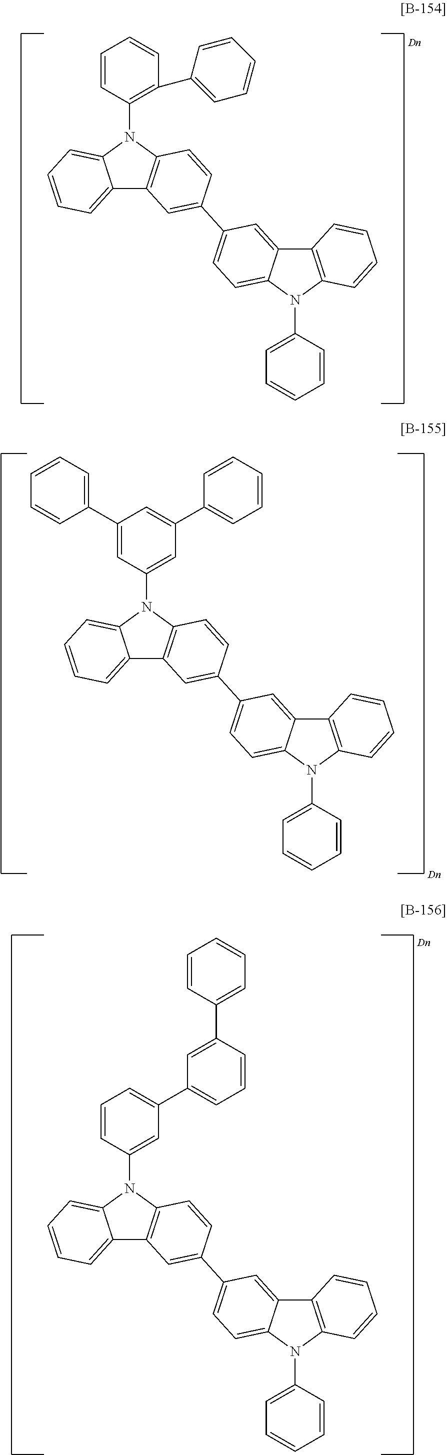

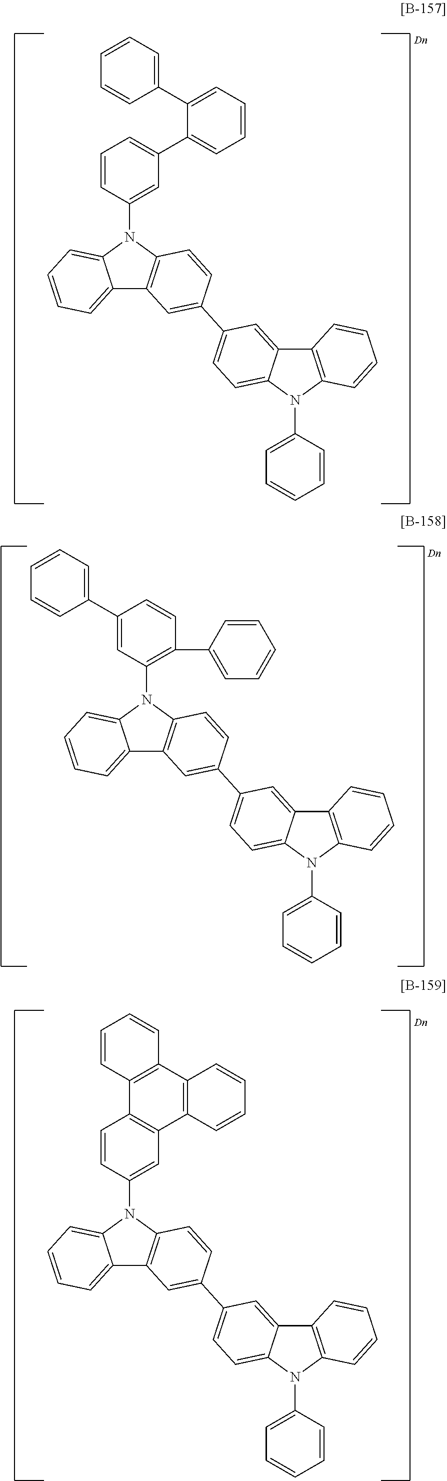

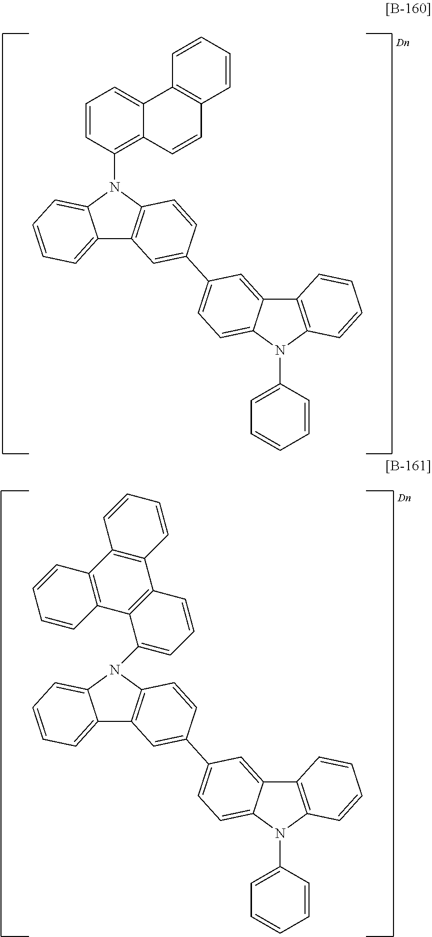

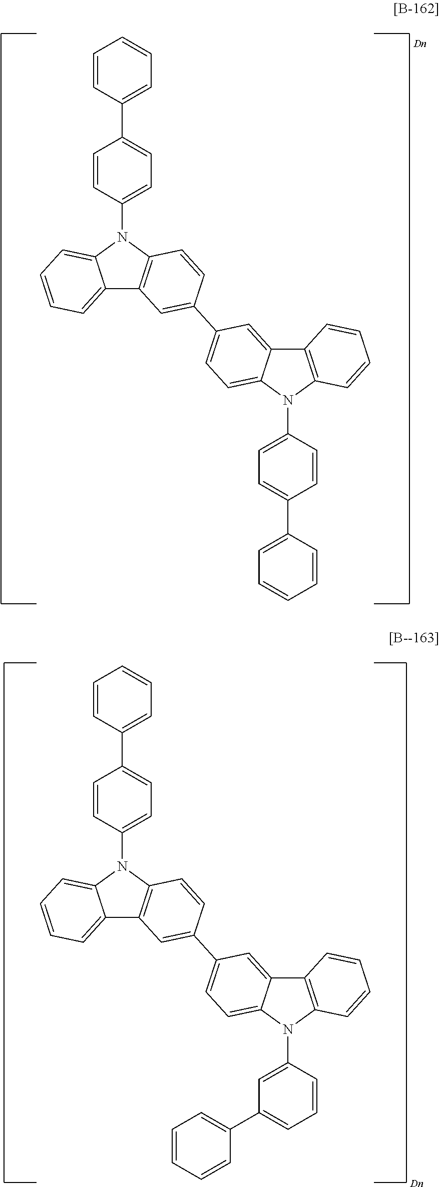

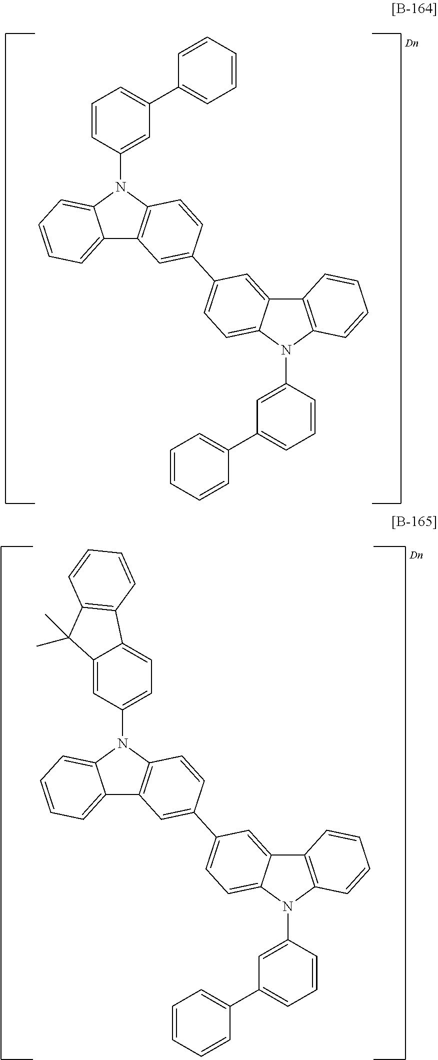

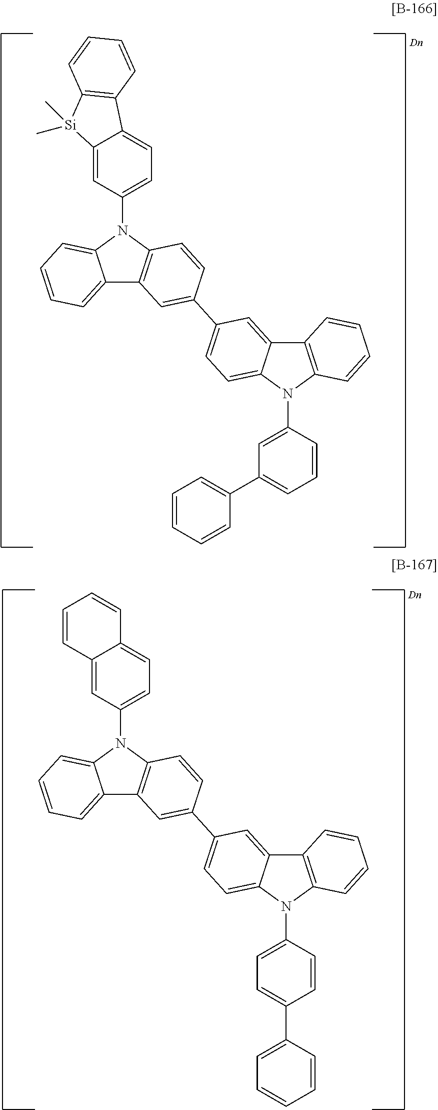

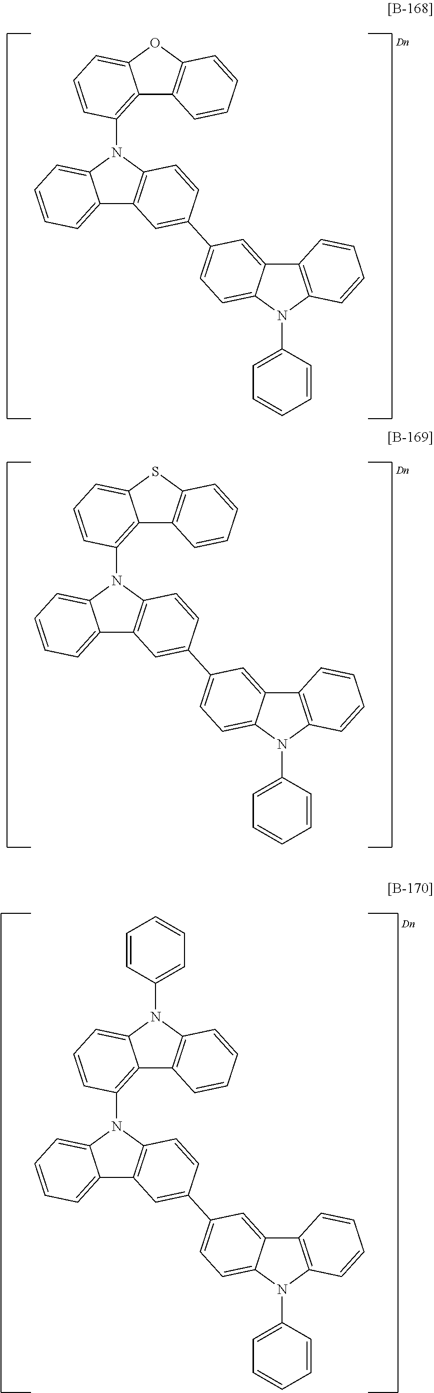

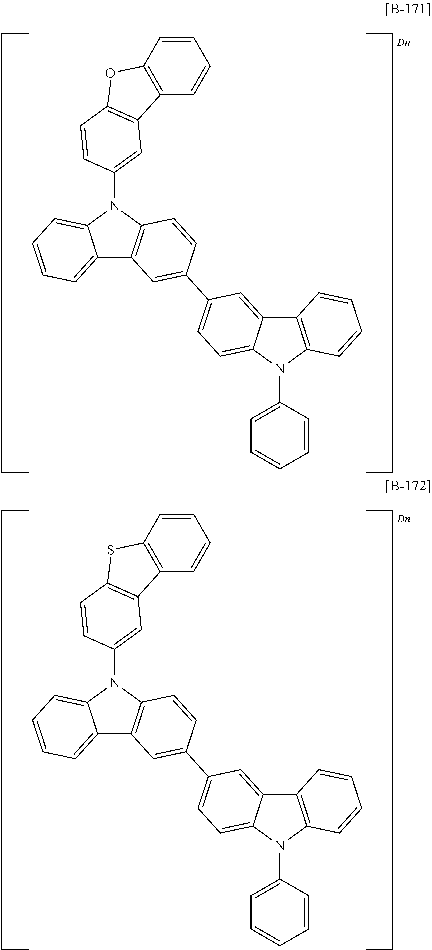

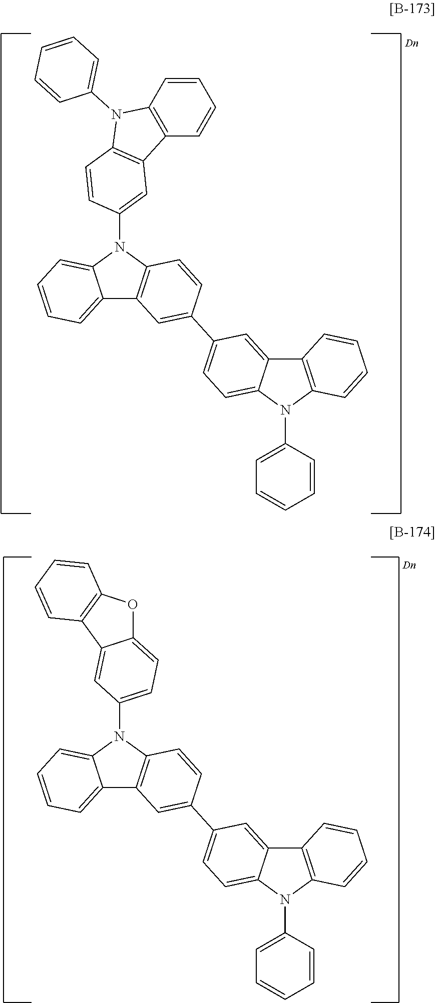

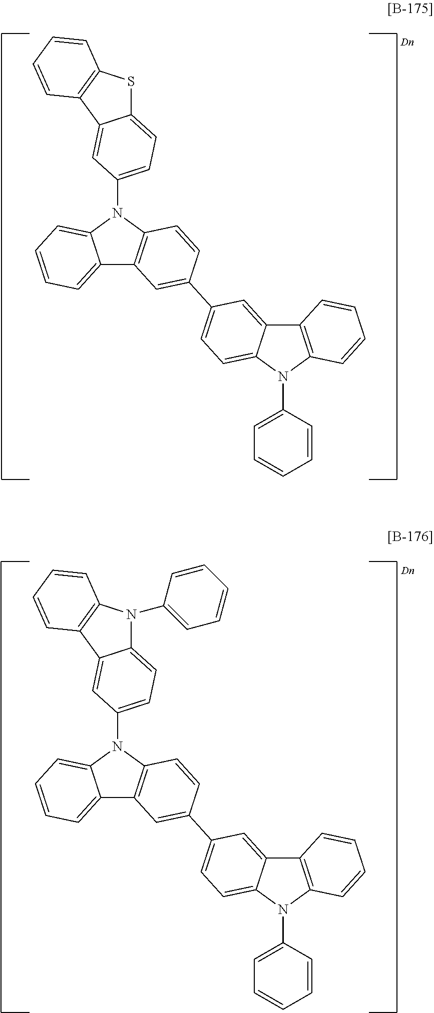

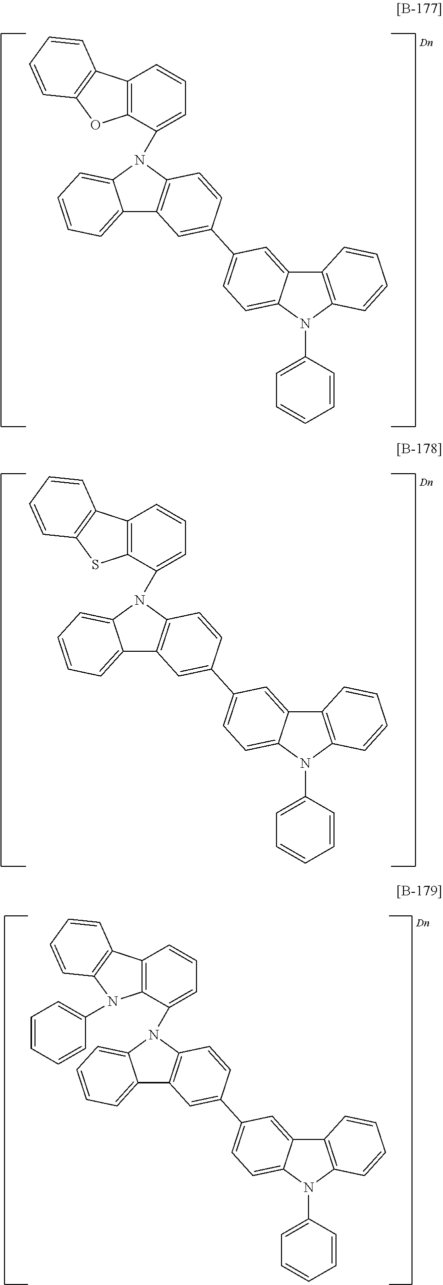

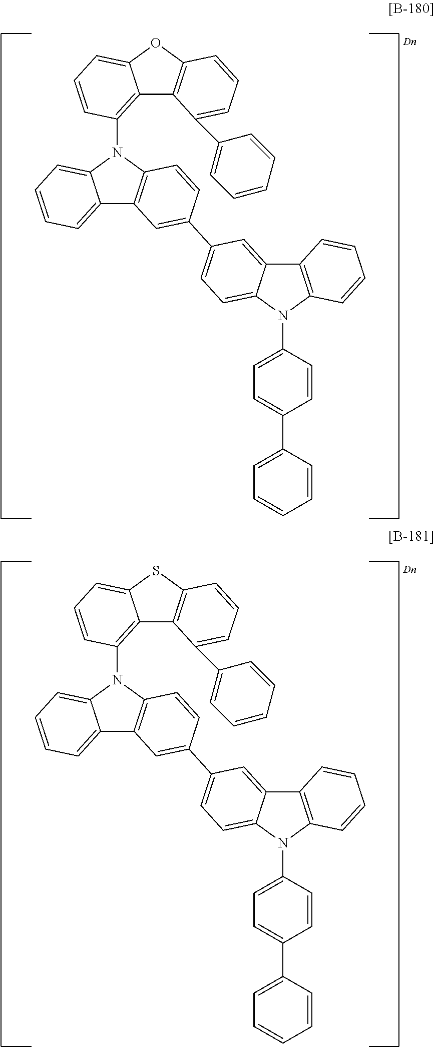

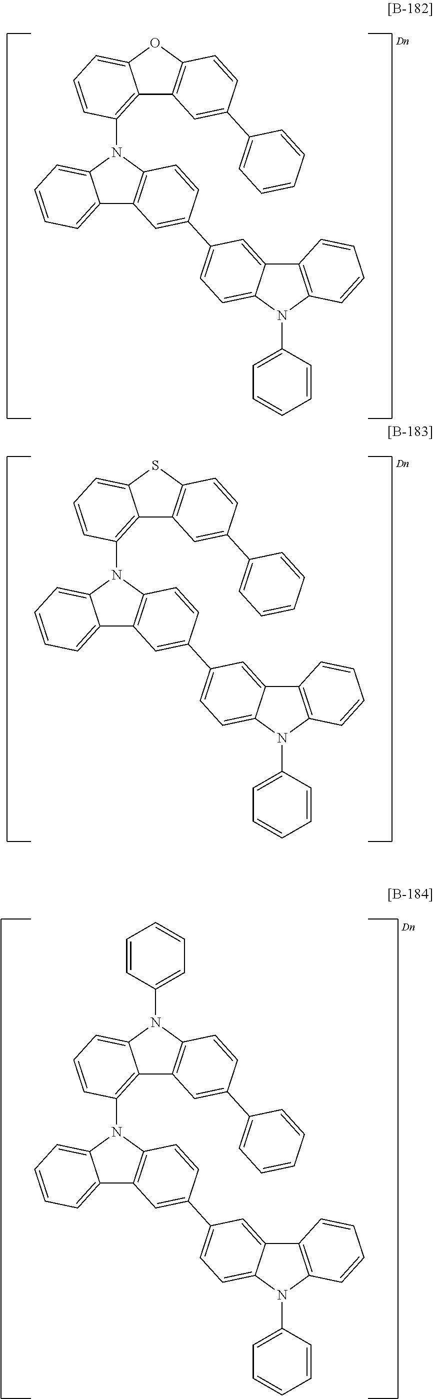

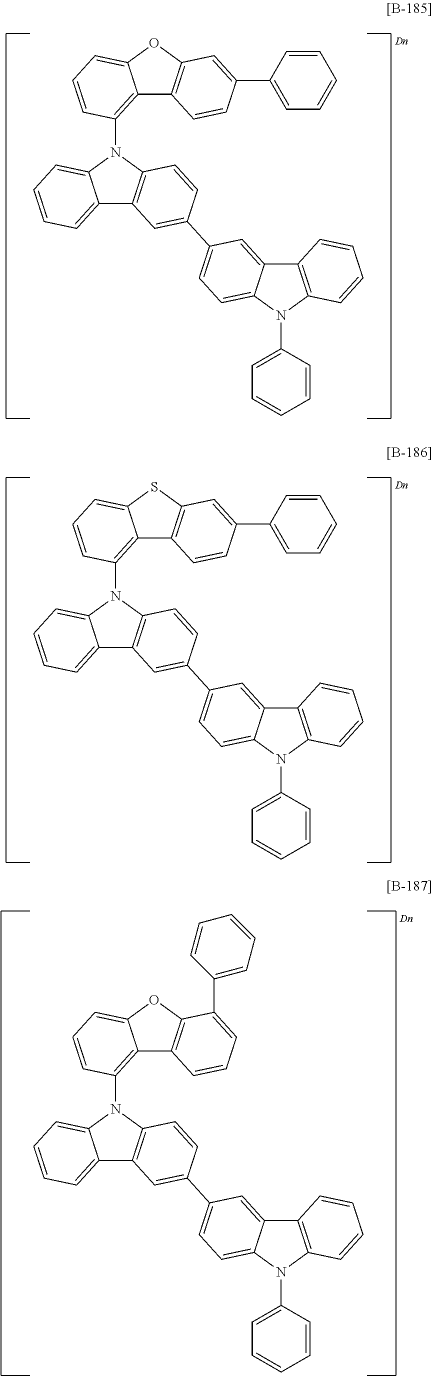

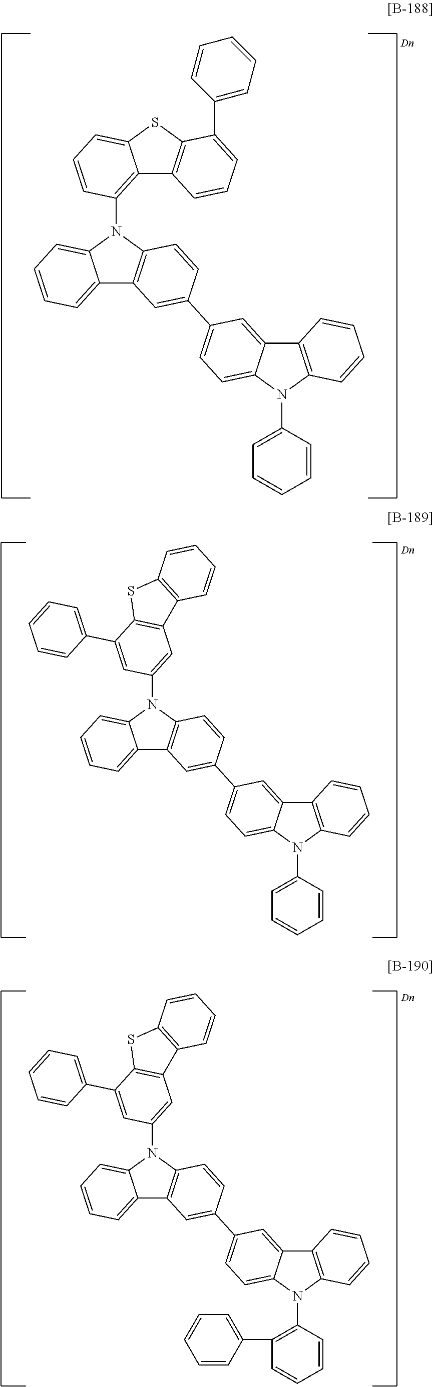

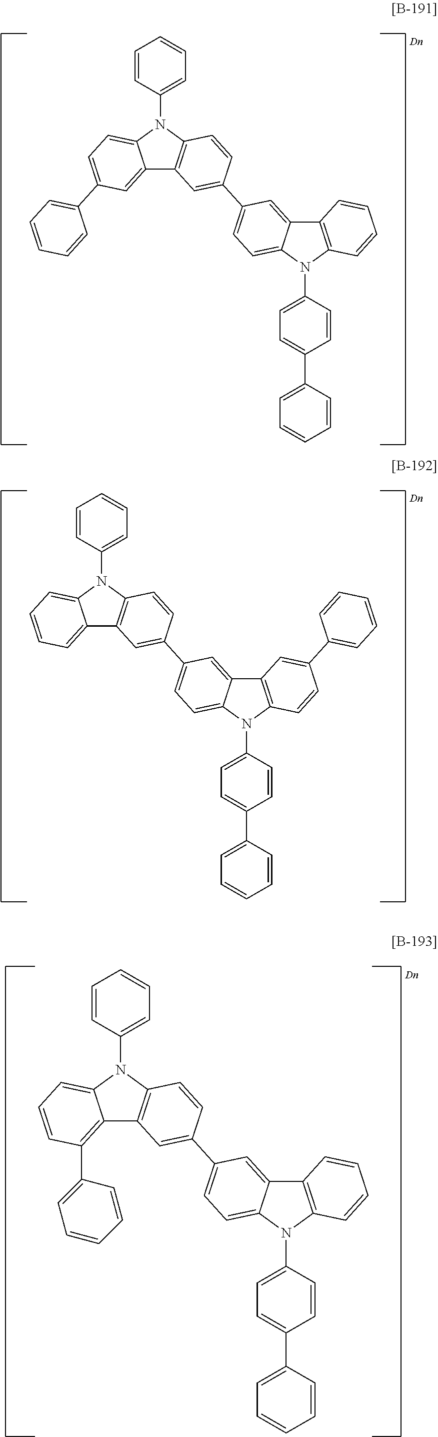

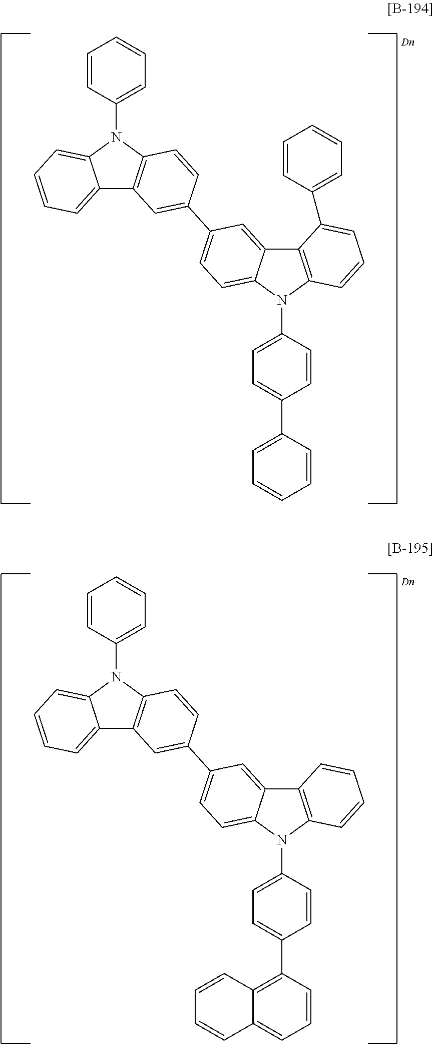

(Dn refers to the number of deuterium substitutions and indicates a structure substituted with one or more deuterium atoms)

The deuterium substitution position and deuterium substitution ratio may include all changeable ranges within the range of Compound B-1 to Compound B-195 (e.g., any hydrogen in any compound may be a protium or a deuterium).

The most specific structures for Compound B-151 to Compound B-195 of Group 2 are presented below as examples according to the position and substitution rate of deuterium substitution.

In an implementation, deuterium may be substituted, e.g., as shown in Compound B-196 to Compound B-234.

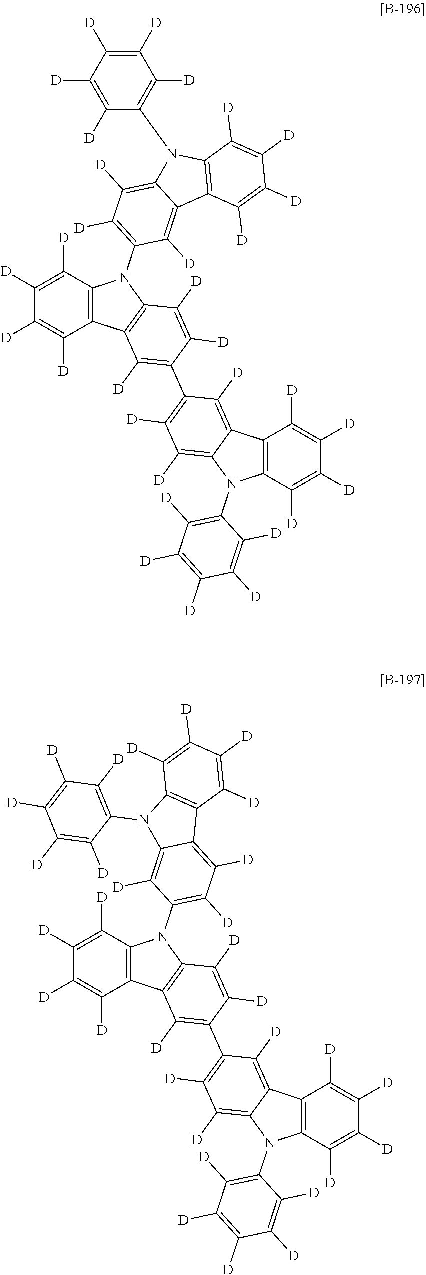

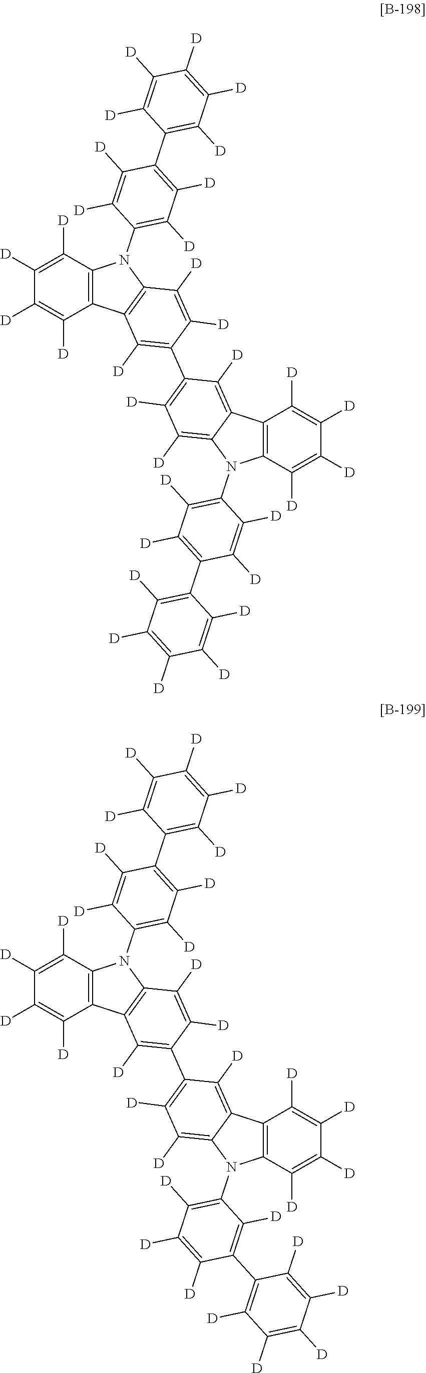

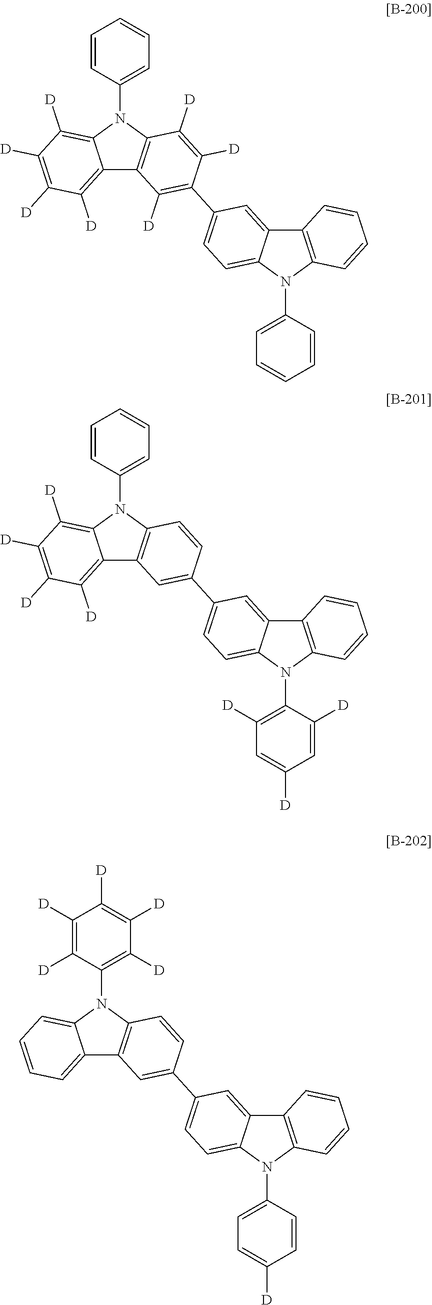 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccccc1 ; [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1]

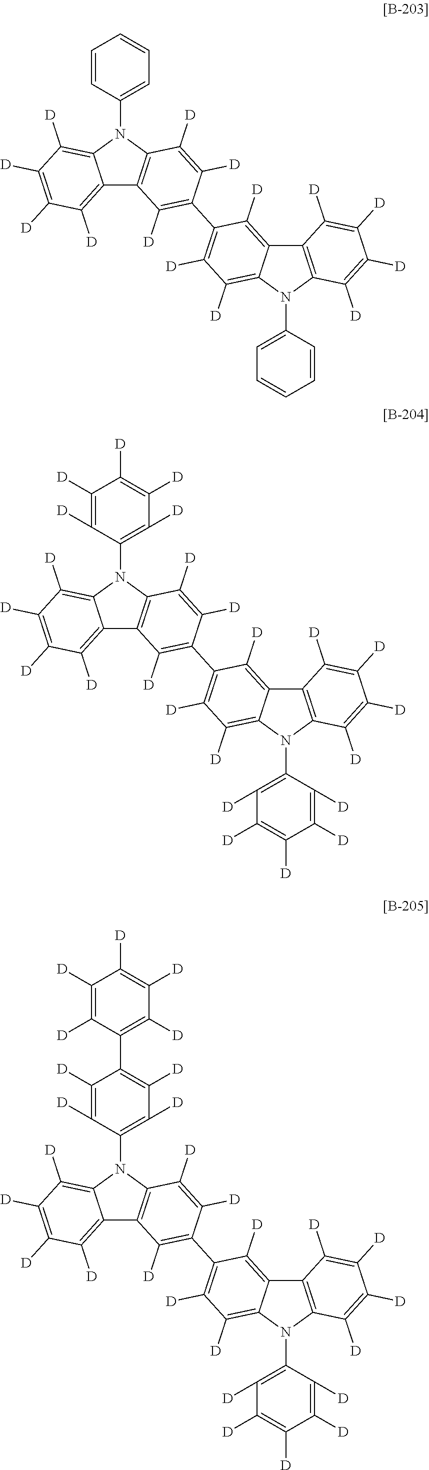

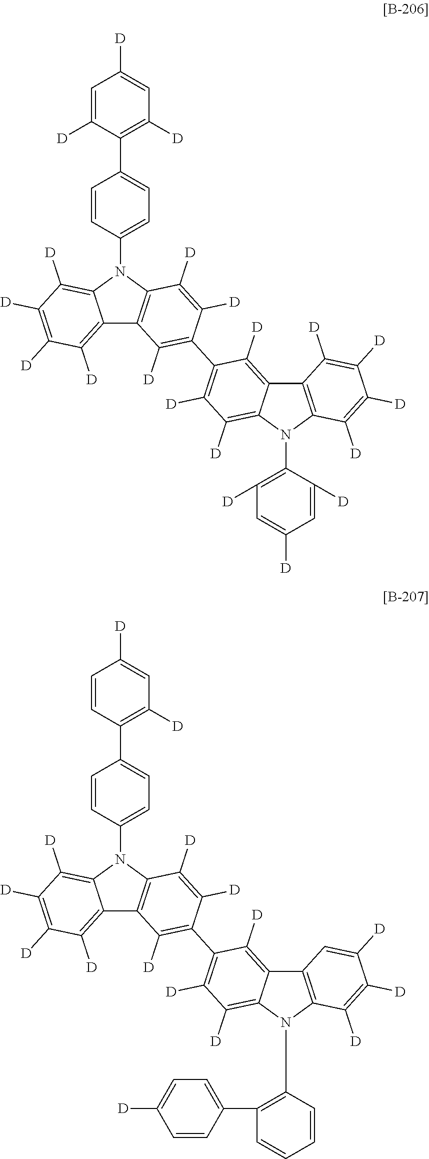 [SMILES: [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1 ; [2H]c1ccc(-c2ccccc2-n2c3c([2H])c([2H])c([2H])cc3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1]

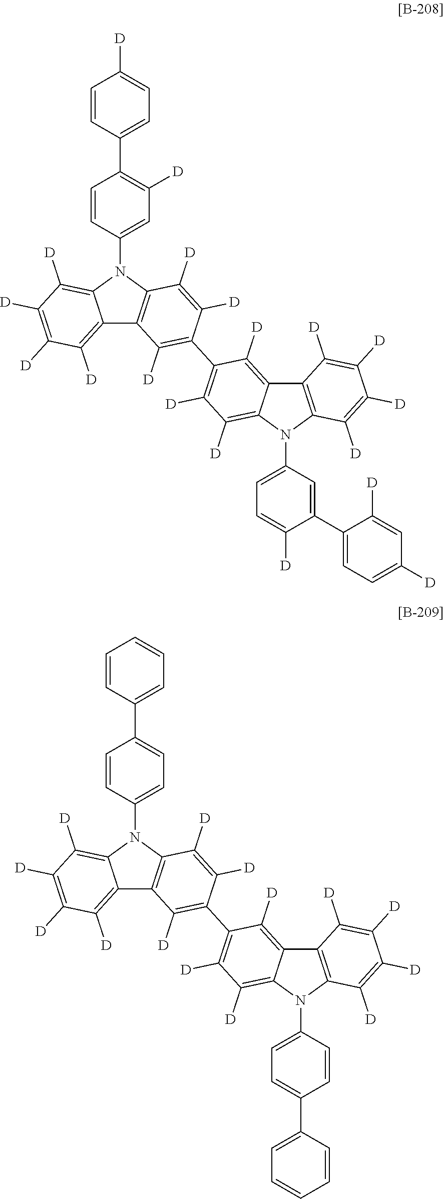 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

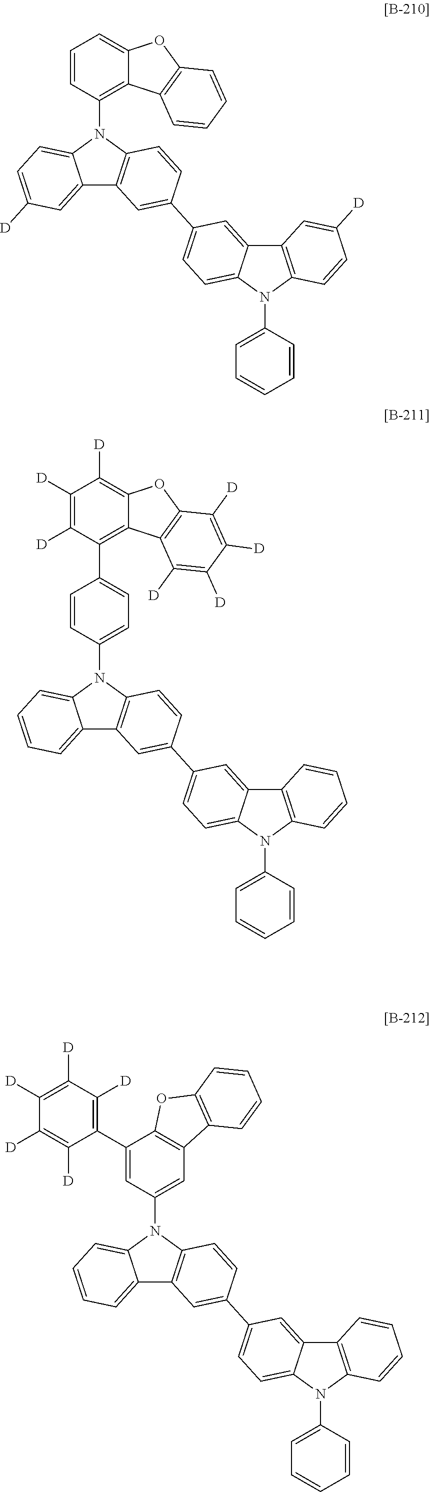 [SMILES: [2H]c1ccc2c(c1)c1cc(-c3ccc4c(c3)c3cc([2H])ccc3n4-c3cccc4oc5ccccc5c34)ccc1n2-c1ccccc1 ; [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H] ; [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

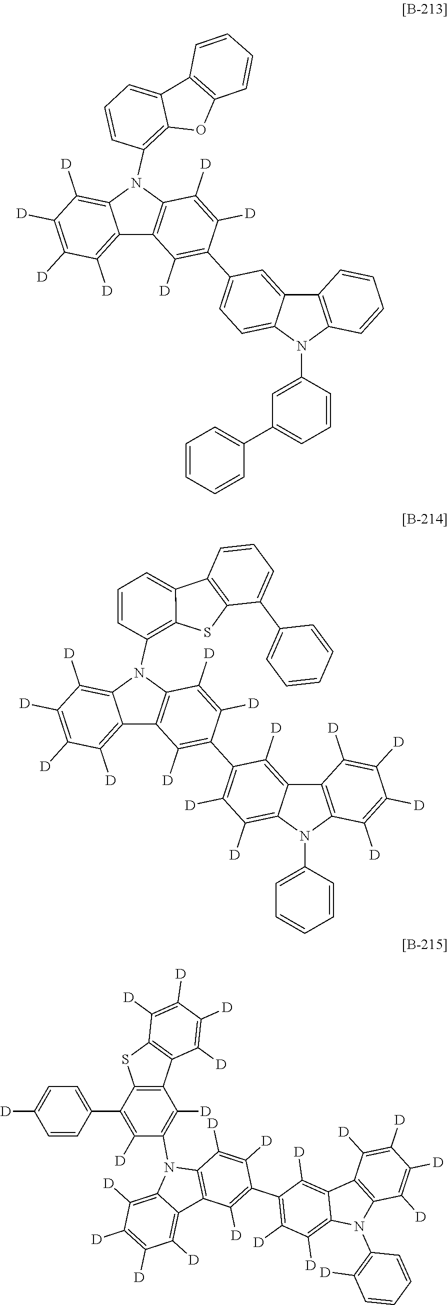

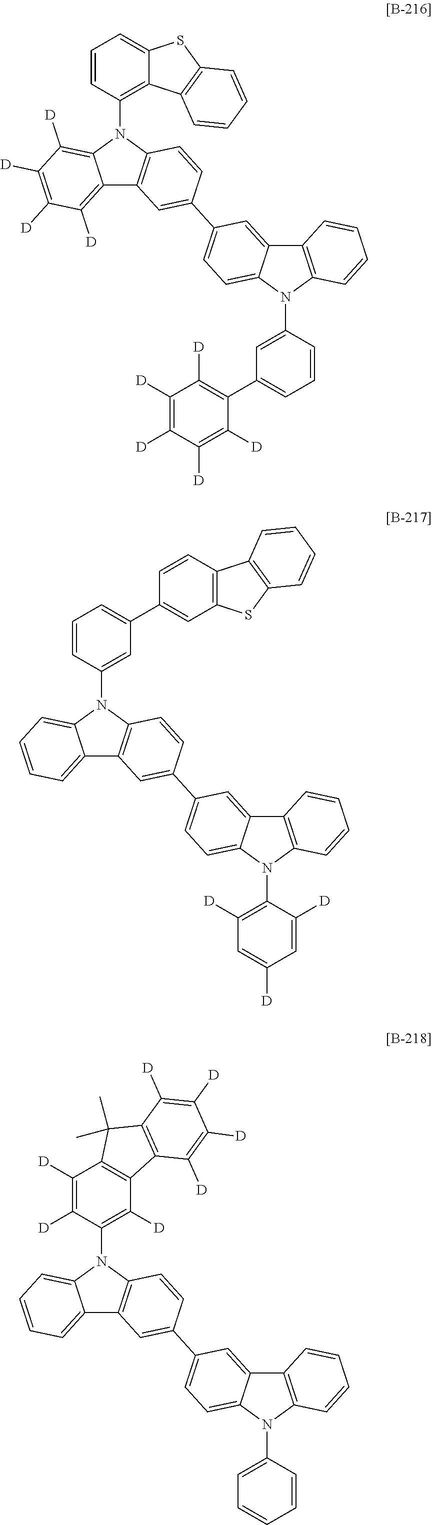 [SMILES: [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1 ; [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C]

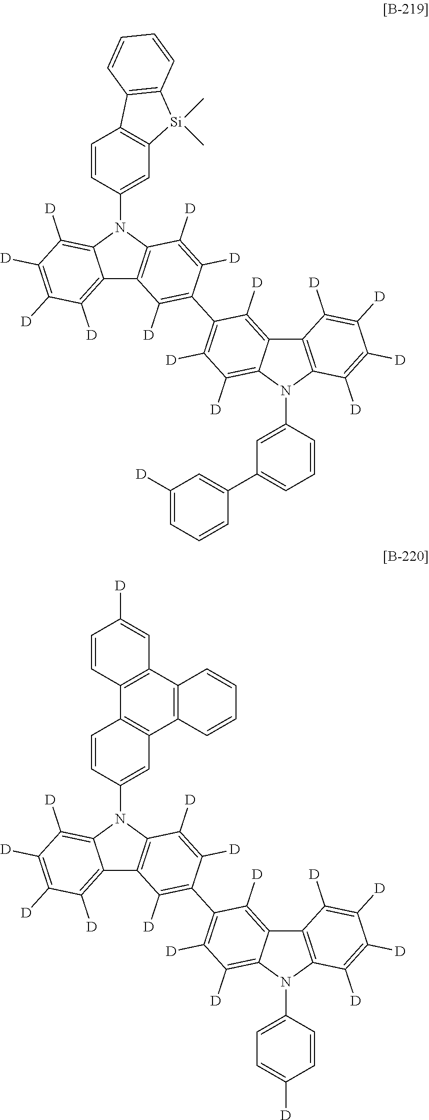 [SMILES: [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1 ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1]

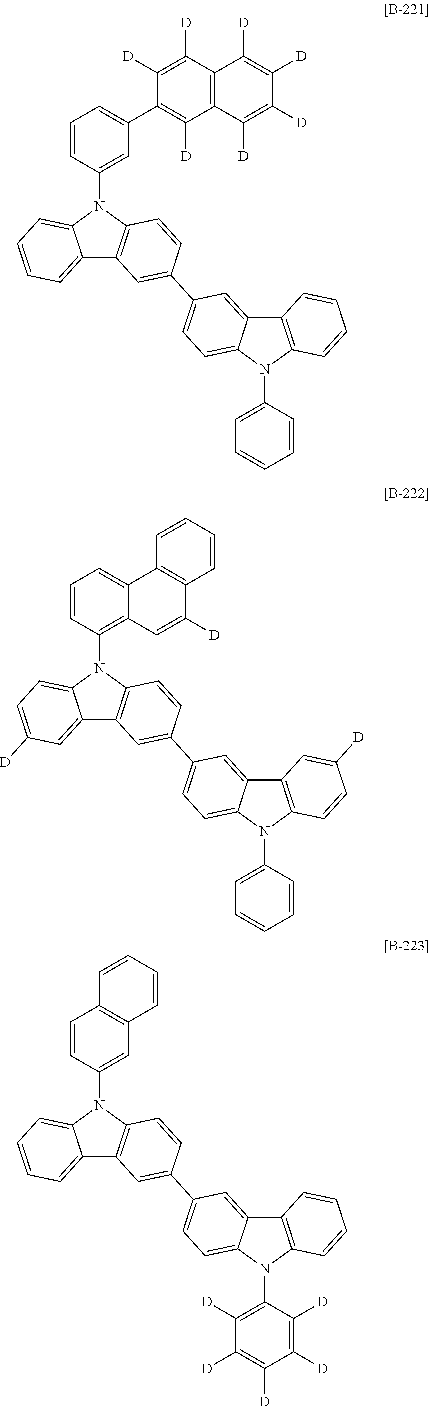 [SMILES: [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H] ; [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H]]

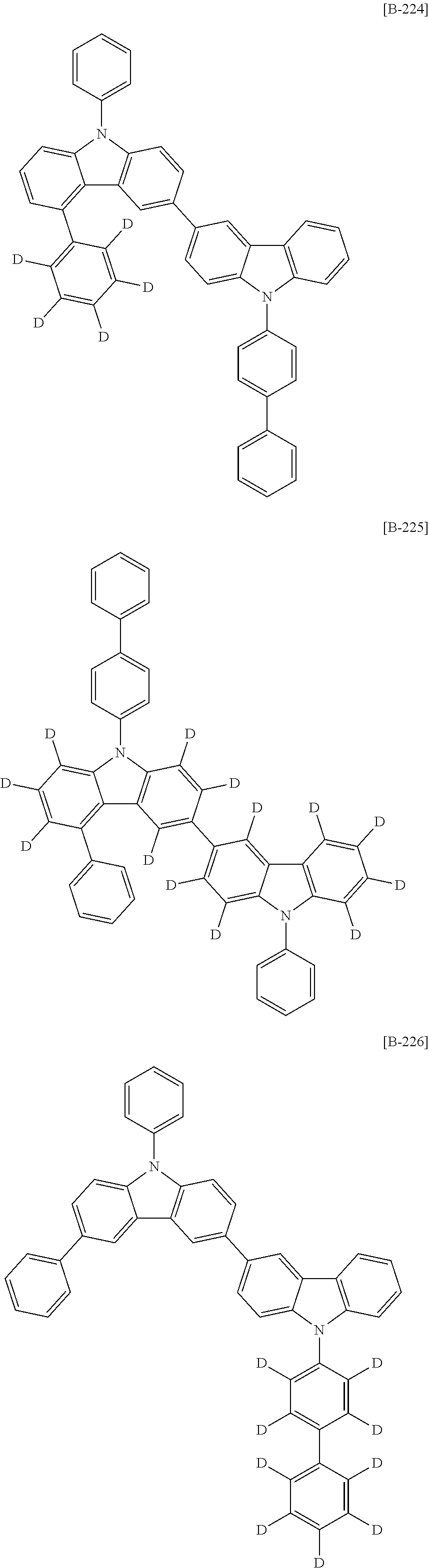 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1 ; [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H]]

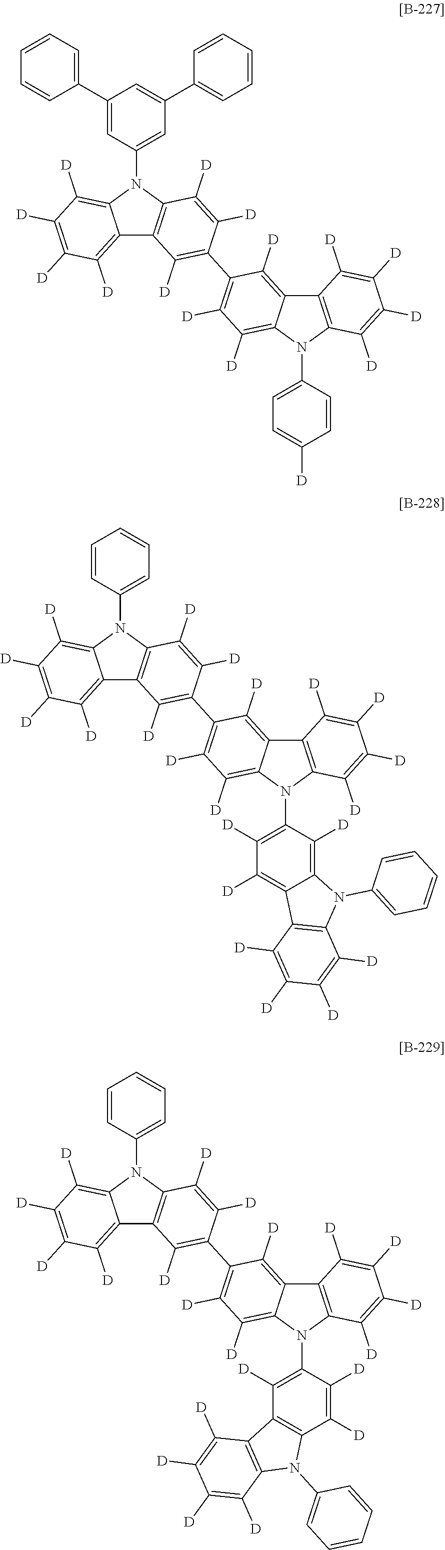

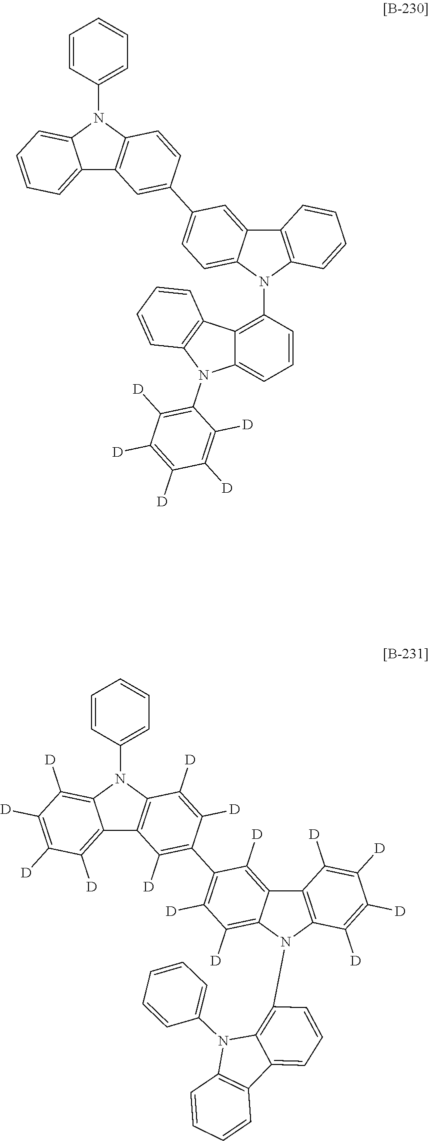 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1]

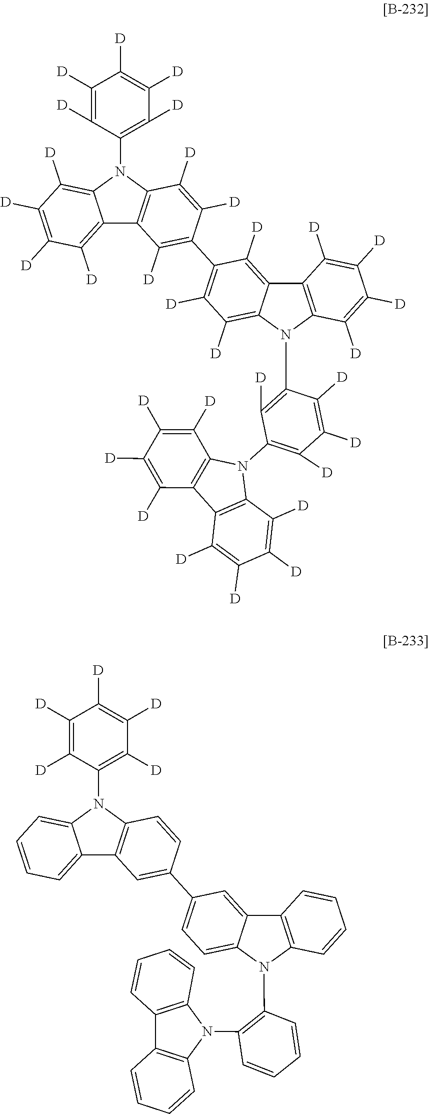 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H] ; [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H]]

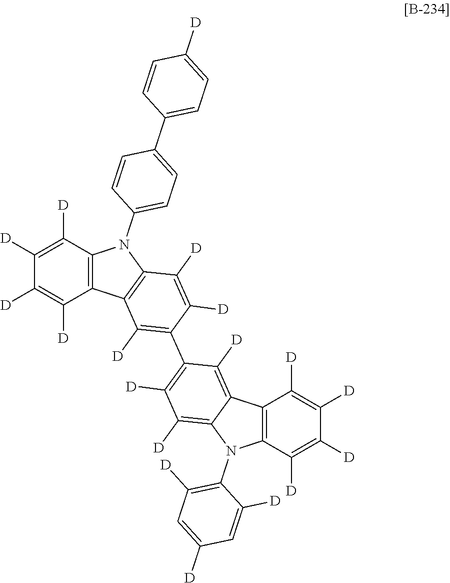 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1]

In an implementation, the second compound for organic optoelectronic device may be, e.g., a compound of Group 3.

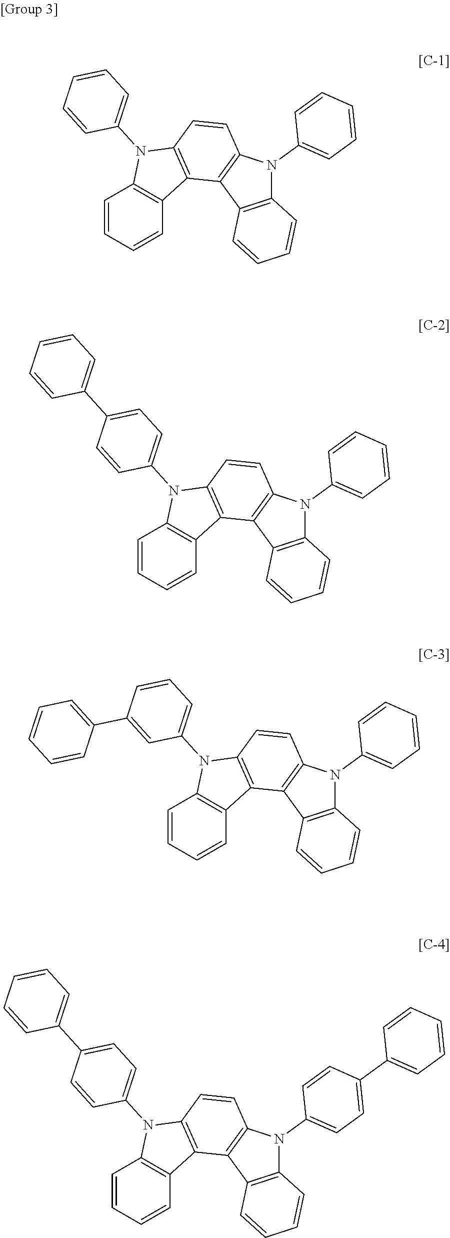 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

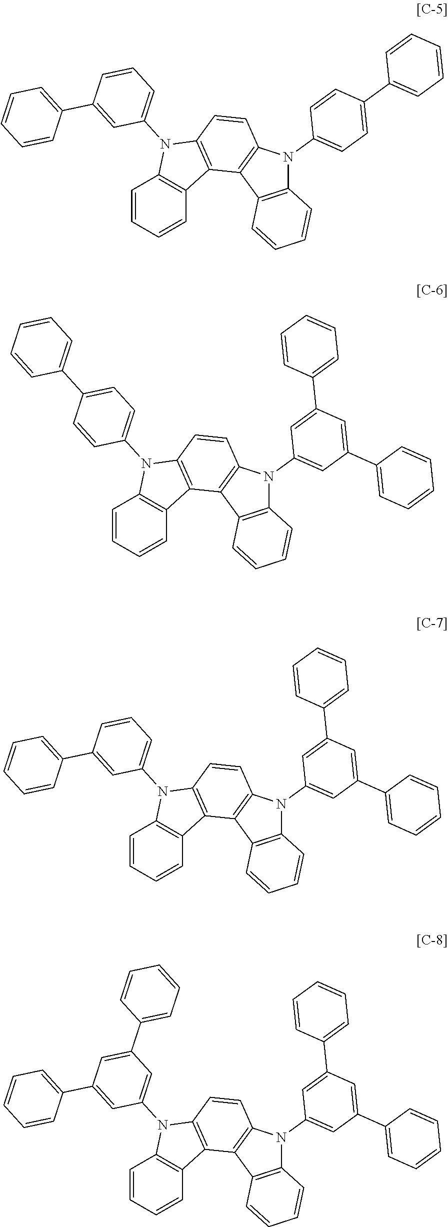 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

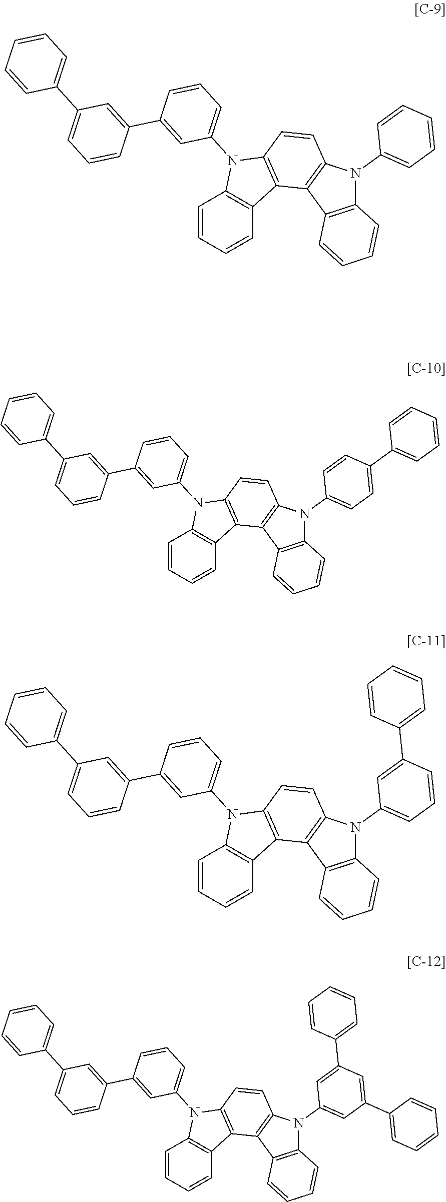 [SMILES: c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

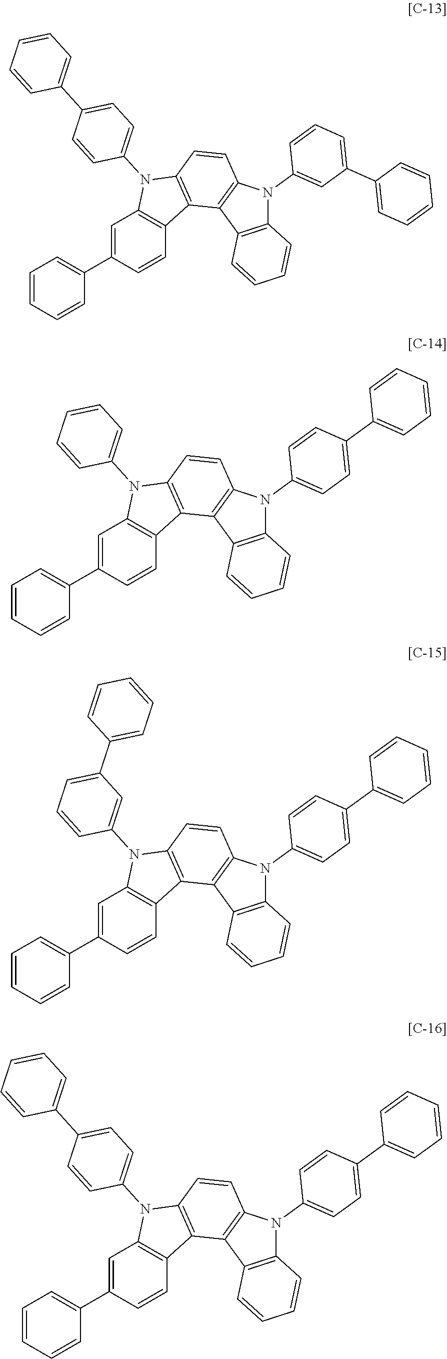 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

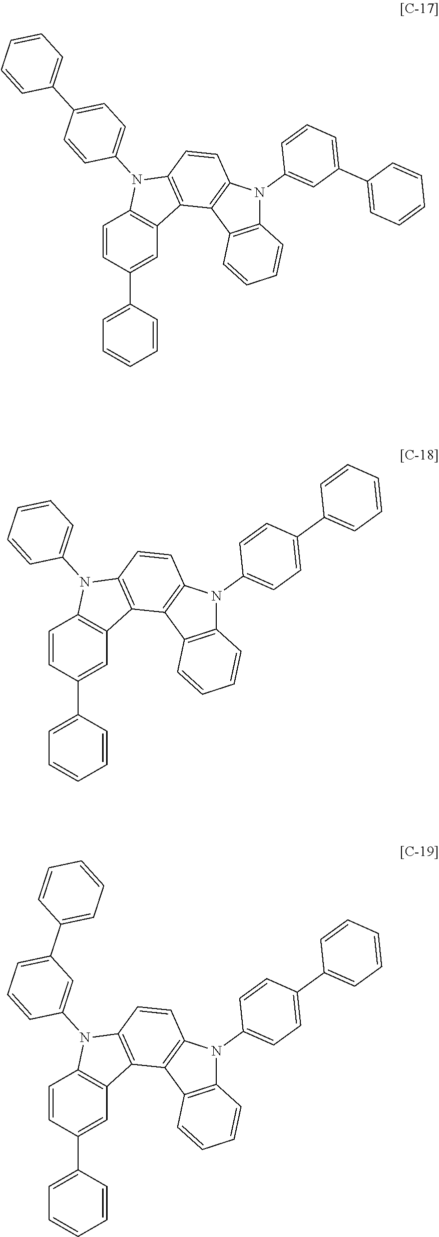 [SMILES: c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1]

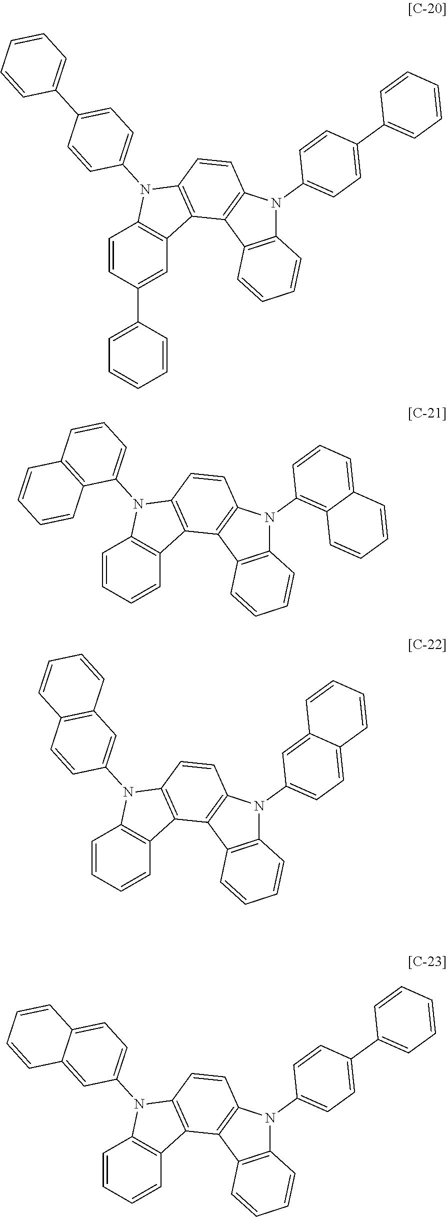 [SMILES: c1ccc(-c2ccc(-n3c4ccccc4c4c5c6cc(-c7ccccc7)ccc6n(-c6ccc(-c7ccccc7)cc6)c5ccc43)cc2)cc1 ; c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1]

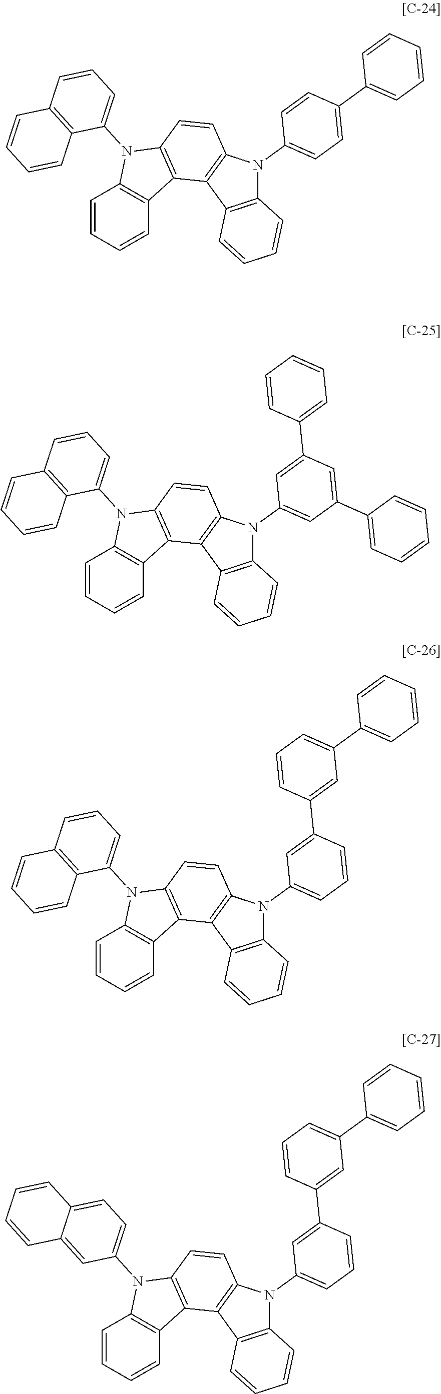 [SMILES: c1ccc(-c2ccc(-n3c4ccccc4c4c5c6ccccc6n(-c6cccc7ccccc67)c5ccc43)cc2)cc1 ; c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1]

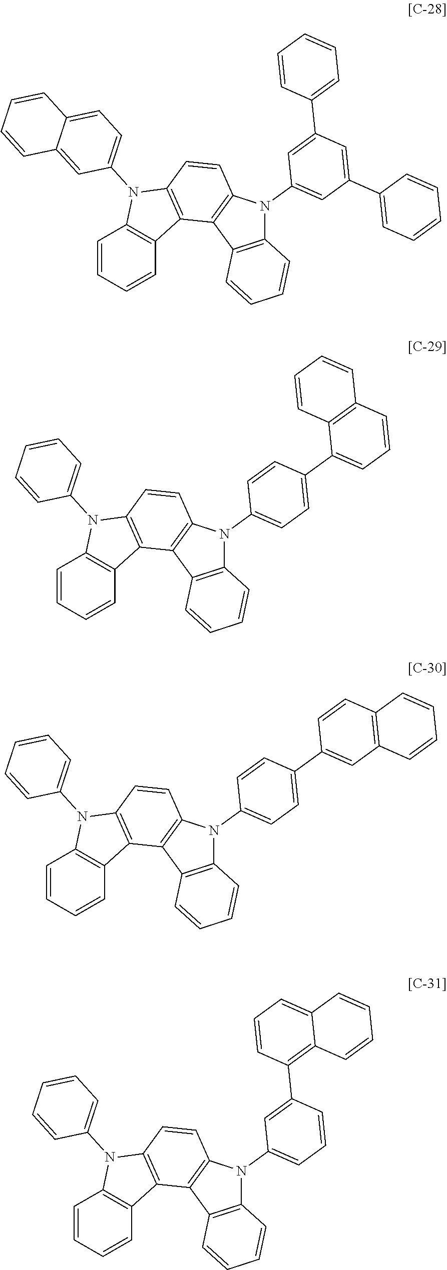 [SMILES: c1ccc(-c2cc(-c3ccccc3)cc(-n3c4ccccc4c4c5c6ccccc6n(-c6ccc7ccccc7c6)c5ccc43)c2)cc1 ; c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1]

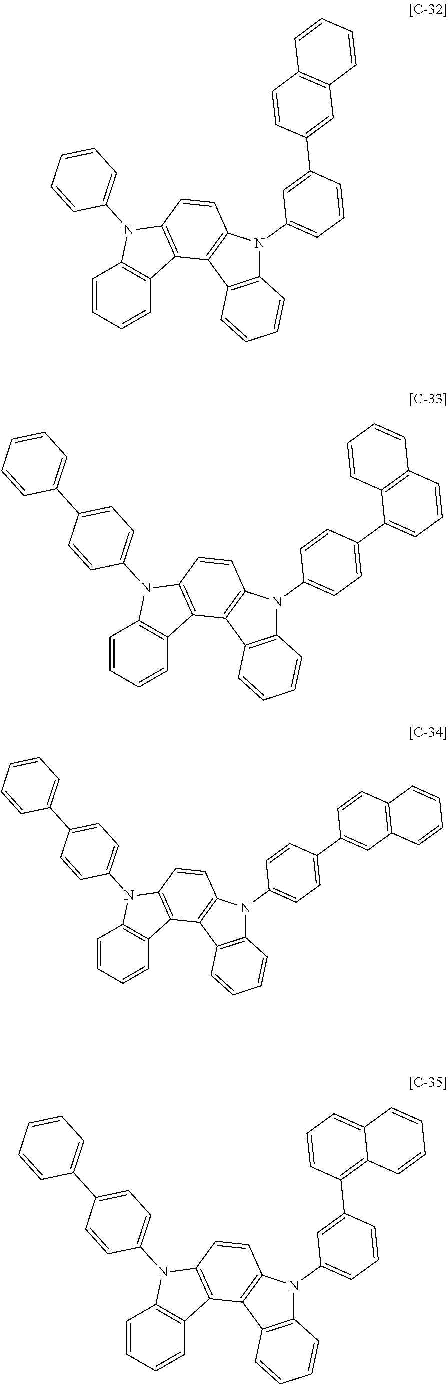 [SMILES: c1ccc(-n2c3ccccc3c3c4c5ccccc5n(-c5cccc(-c6ccc7ccccc7c6)c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1]

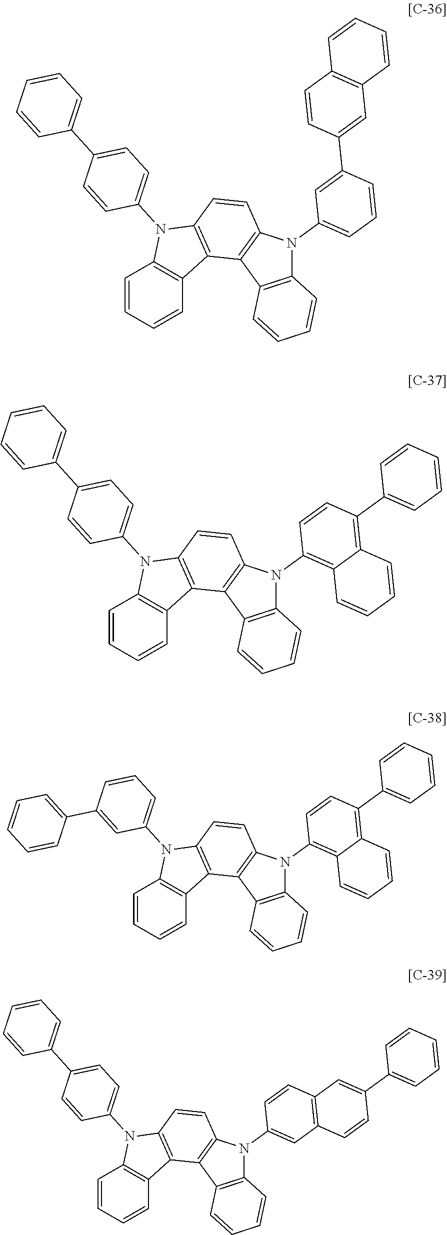 [SMILES: c1ccc(-c2ccc(-n3c4ccccc4c4c5c6ccccc6n(-c6cccc(-c7ccc8ccccc8c7)c6)c5ccc43)cc2)cc1 ; c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1]

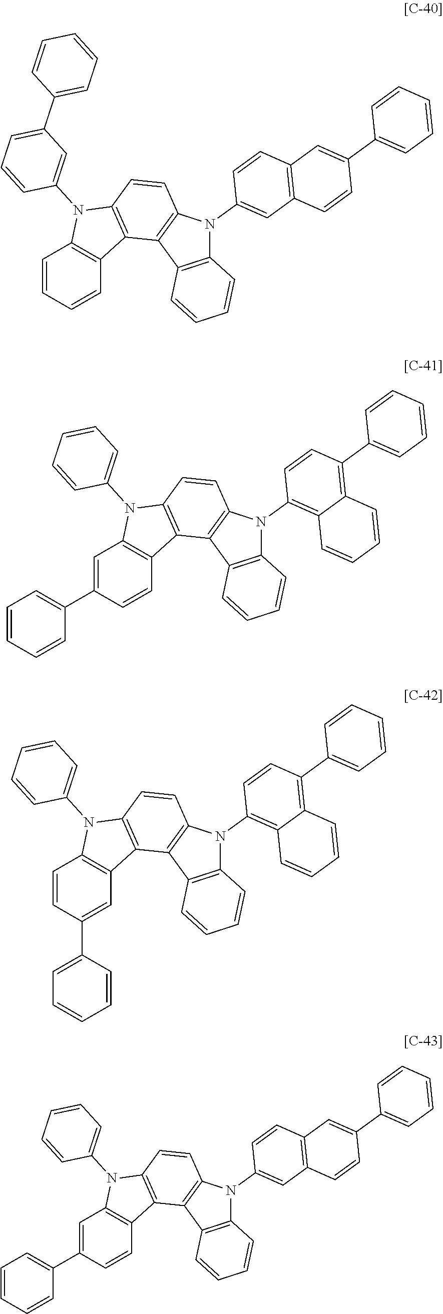 [SMILES: c1ccc(-c2cccc(-n3c4ccccc4c4c5c6ccccc6n(-c6ccc7cc(-c8ccccc8)ccc7c6)c5ccc43)c2)cc1 ; c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1]

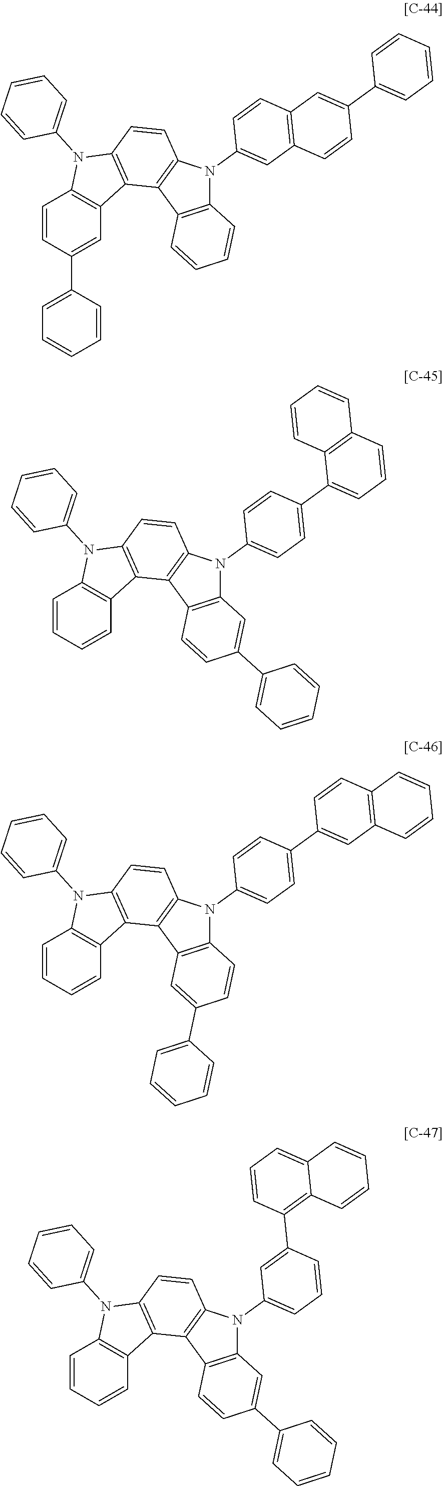 [SMILES: c1ccc(-c2ccc3cc(-n4c5ccccc5c5c6c7cc(-c8ccccc8)ccc7n(-c7ccccc7)c6ccc54)ccc3c2)cc1 ; c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1 ; c1ccc(-c2ccc3c4c5c6ccccc6n(-c6ccccc6)c5ccc4n(-c4cccc(-c5cccc6ccccc56)c4)c3c2)cc1]

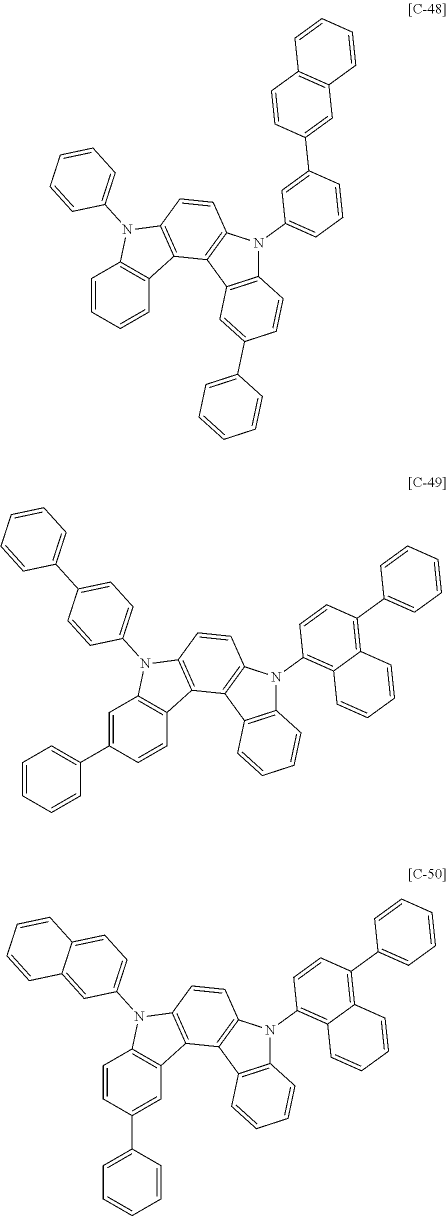 [SMILES: c1ccc(-c2ccc3c(c2)c2c4c5ccccc5n(-c5ccccc5)c4ccc2n3-c2cccc(-c3ccc4ccccc4c3)c2)cc1 ; c1ccc(-c2ccc(-n3c4cc(-c5ccccc5)ccc4c4c5c6ccccc6n(-c6ccc(-c7ccccc7)c7ccccc67)c5ccc43)cc2)cc1 ; c1ccc(-c2ccc3c(c2)c2c4c5ccccc5n(-c5ccc(-c6ccccc6)c6ccccc56)c4ccc2n3-c2ccc3ccccc3c2)cc1]

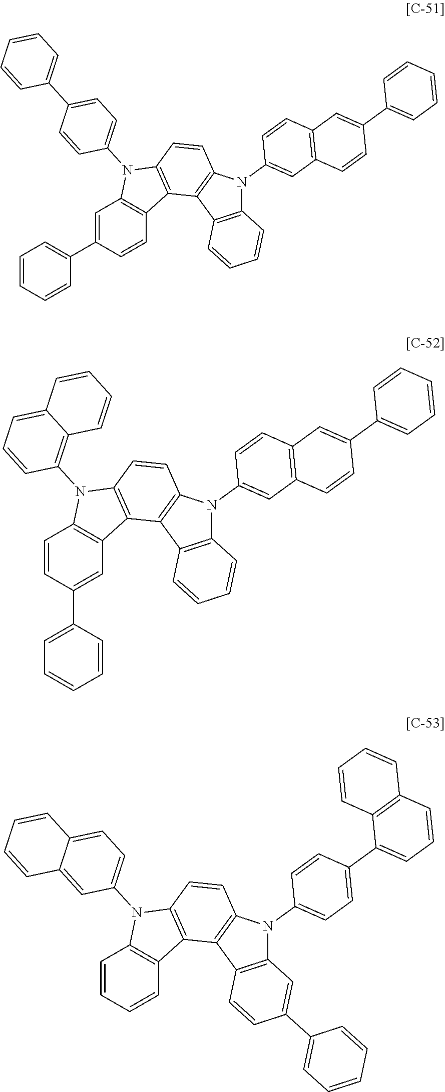 [SMILES: c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1 ; c1ccc(-c2ccc3c4c5c6ccccc6n(-c6ccc7ccccc7c6)c5ccc4n(-c4ccc(-c5cccc6ccccc56)cc4)c3c2)cc1]

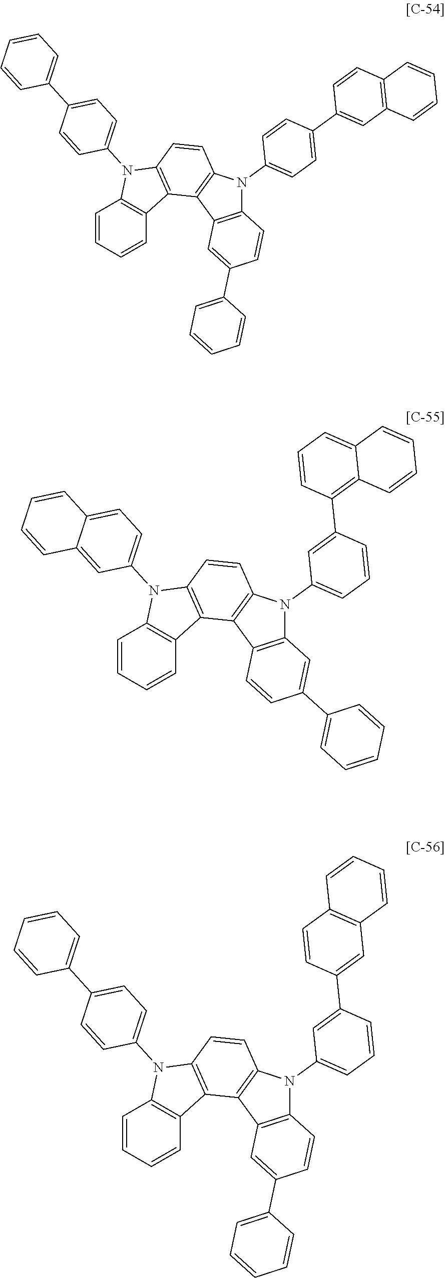 [SMILES: c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1 ; c1ccc(-c2ccc(-n3c4ccccc4c4c5c6cc(-c7ccccc7)ccc6n(-c6cccc(-c7ccc8ccccc8c7)c6)c5ccc43)cc2)cc1]

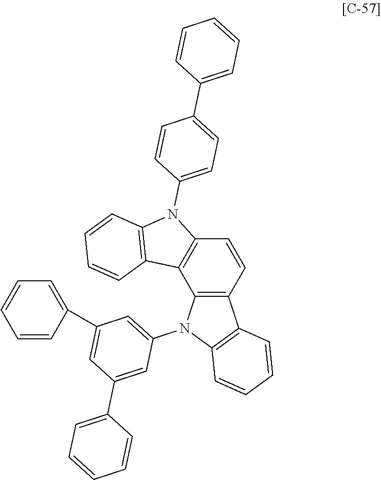 [SMILES: c1ccc(-c2ccc(-n3c4ccccc4c4c3ccc3c5ccccc5n(-c5cc(-c6ccccc6)cc(-c6ccccc6)c5)c34)cc2)cc1]

In an implementation, examples of Compound C-1 to Compound C-57, listed in Group 3, in which at least one hydrogen is replaced with deuterium are given below. In an implementation, deuterium may be substituted, e.g., as shown in Compound C-58 to Compound C-72, exemplified below.

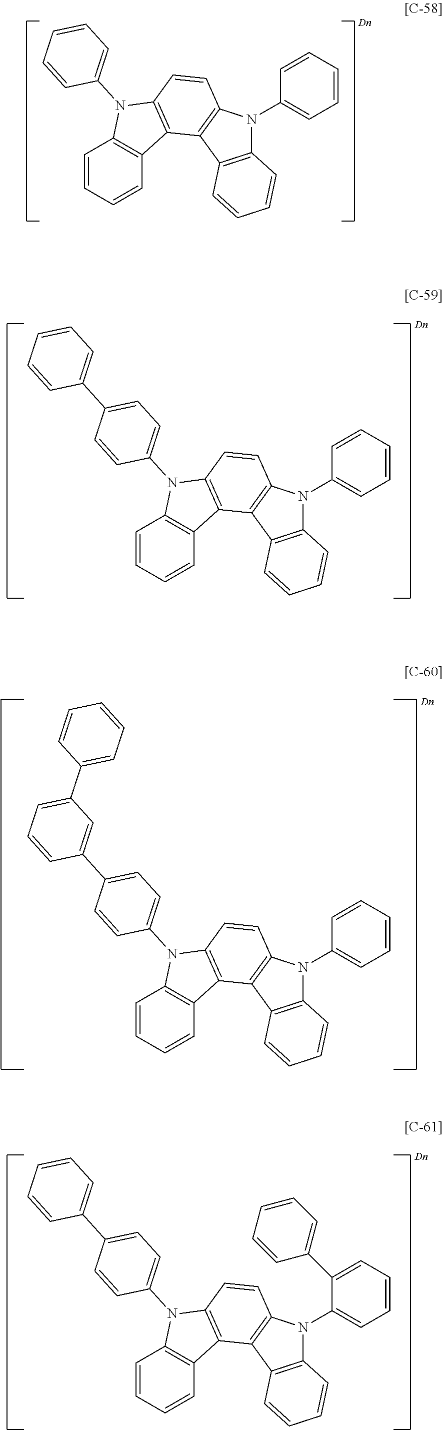

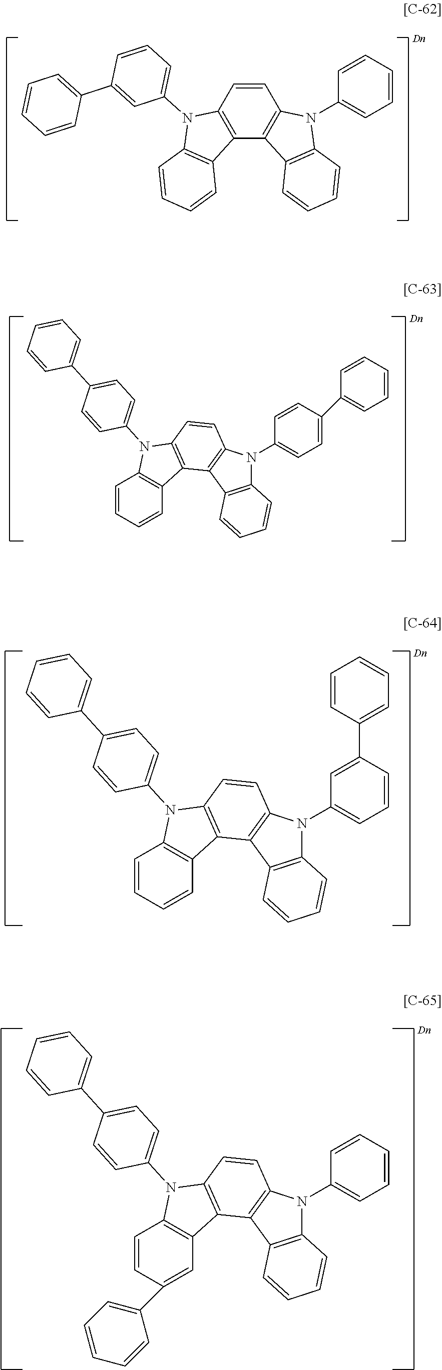

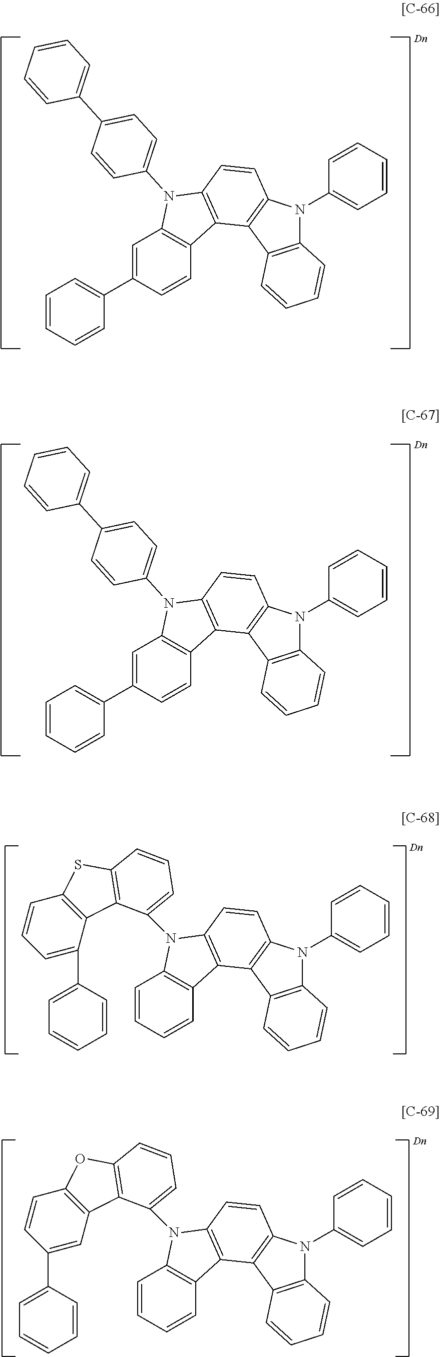

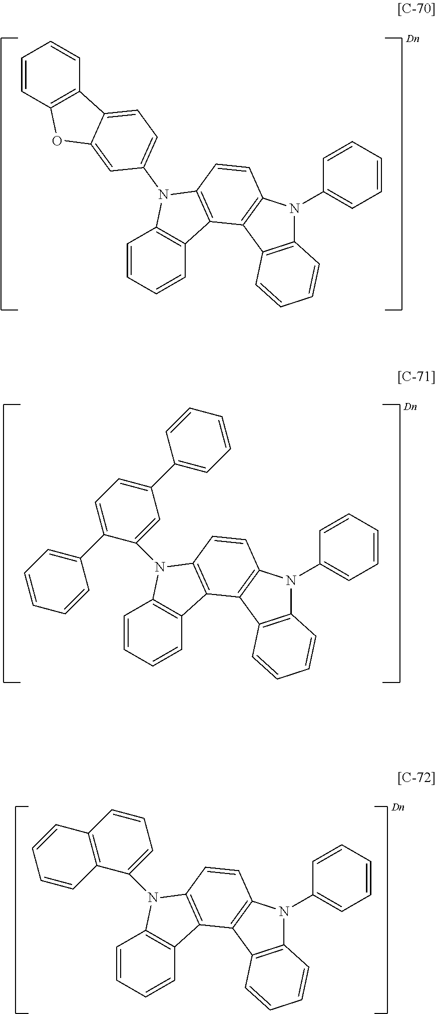

(Dn refers to the number of deuterium substitutions and indicates a structure substituted with one or more deuterium atoms)

The deuterium substitution position and deuterium substitution ratio may include all changeable ranges within the range of Compound C-1 to Compound C-72 (e.g., any hydrogen in any compound may be a protium or a deuterium).

The most specific structures for Compound C-58 to Compound C-72 of Group 3 are presented below as examples according to the position and substitution rate of deuterium substitution.

In an implementation, deuterium may be substituted, as shown in Compound C-73 to Compound C-102.

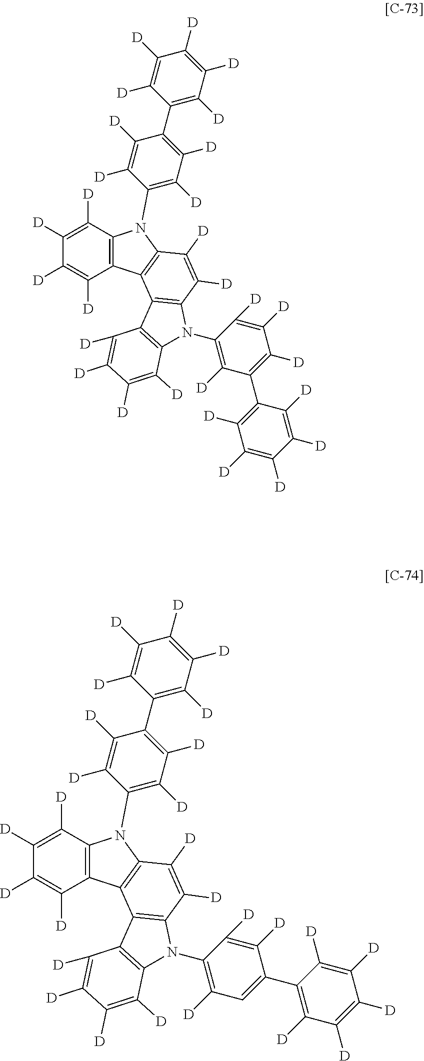 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1cc(-c2c([2H])c([2H])c([2H])c([2H])c2[2H])c([2H])c([2H])c1-n1c2c([2H])c([2H])c([2H])c([2H])c2c2c3c4c([2H])c([2H])c([2H])c([2H])c4n(-c4c([2H])c([2H])c(-c5c([2H])c([2H])c([2H])c([2H])c5[2H])c([2H])c4[2H])c3c([2H])c([2H])c21]

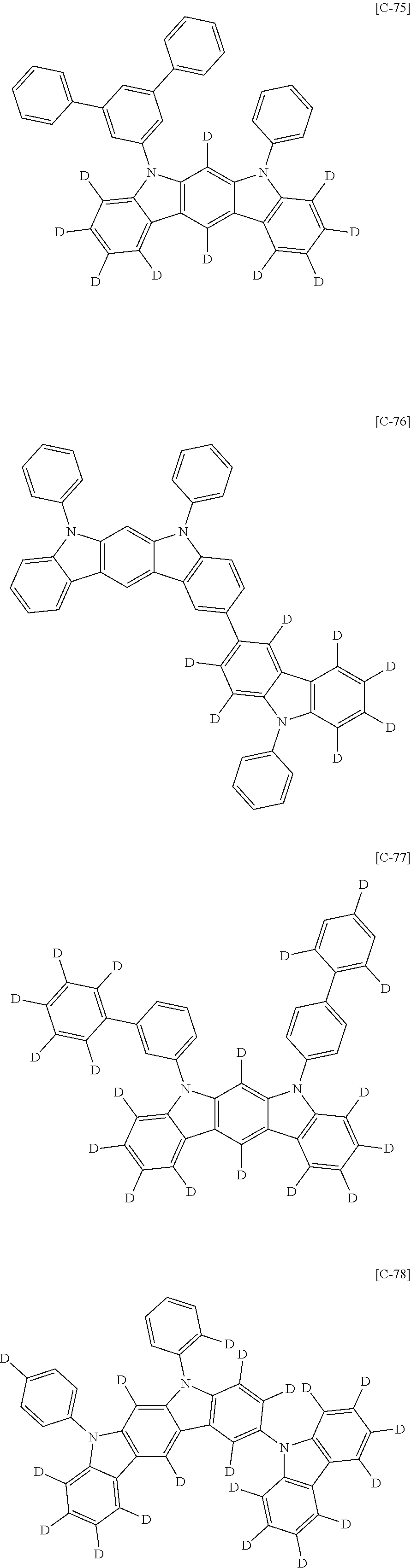

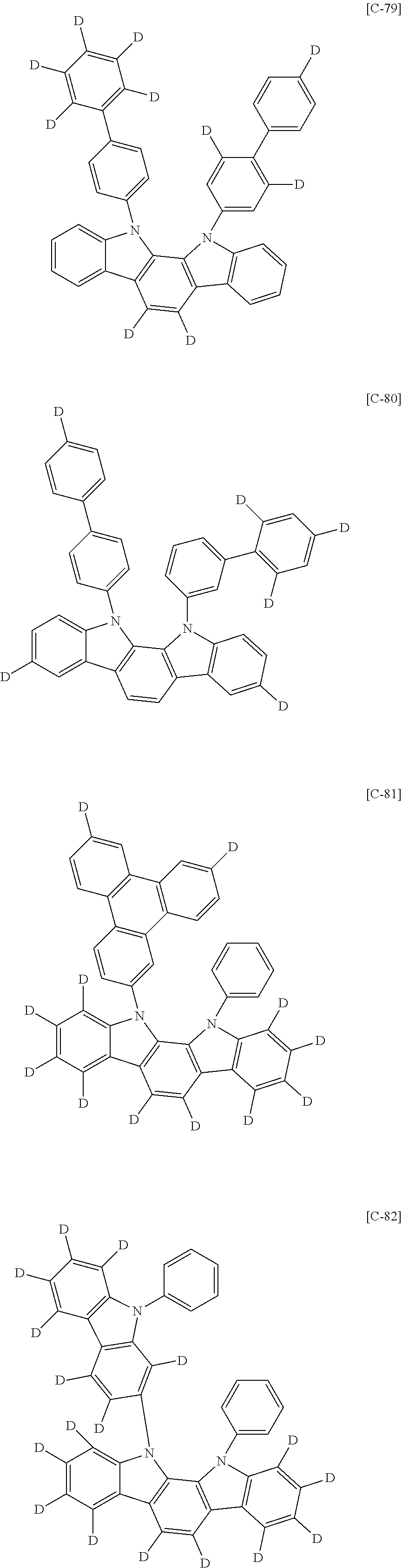

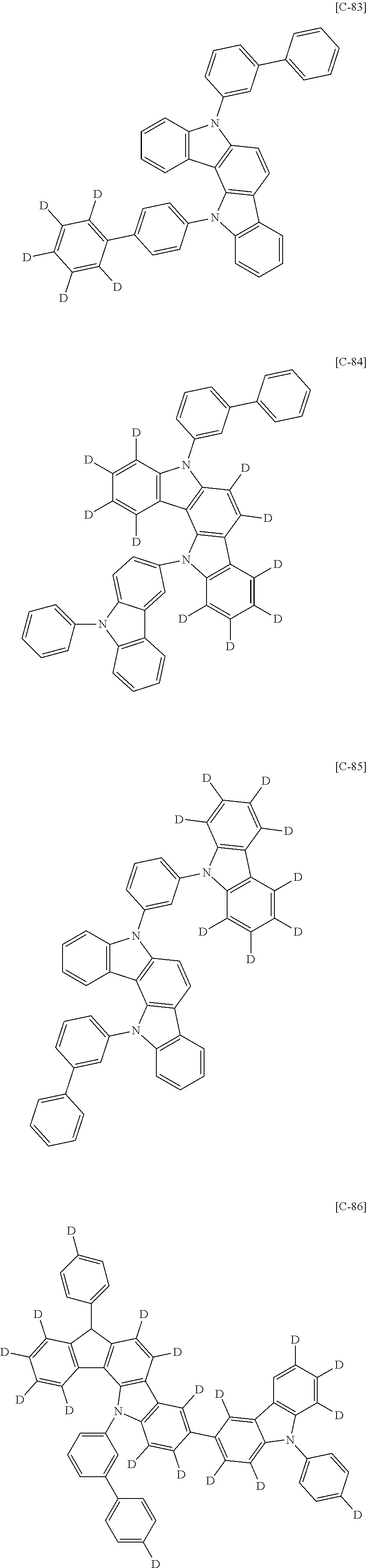

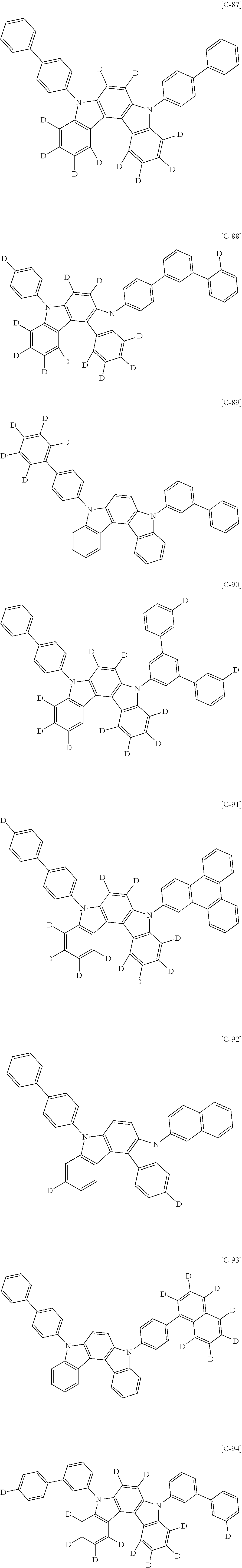

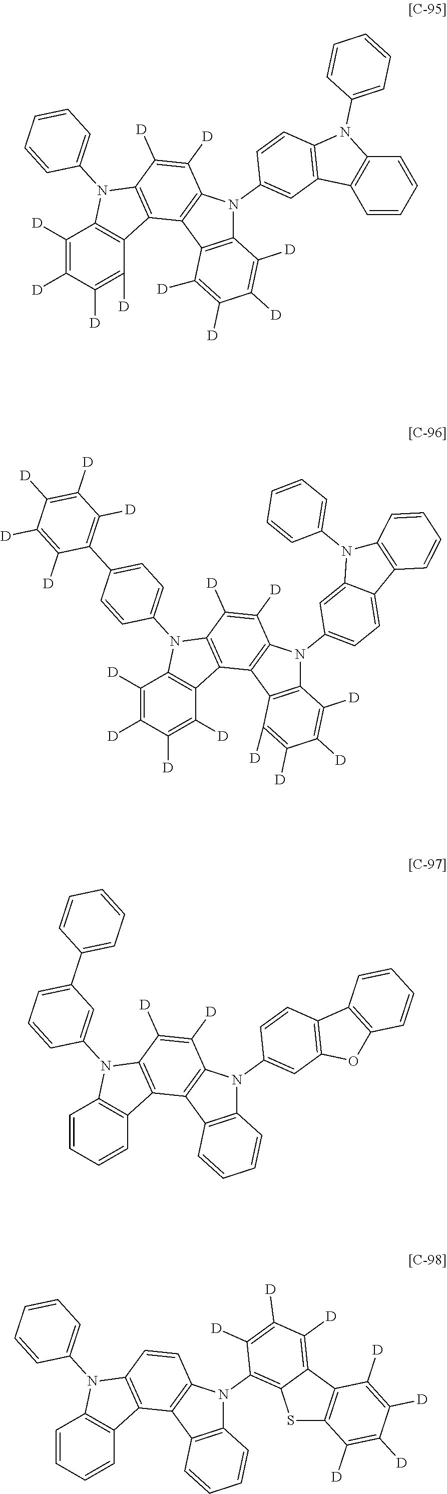 [SMILES: [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1 ; [2H]c1c([2H])c([2H])c(-c2ccc(-n3c4c([2H])c([2H])c([2H])c([2H])c4c4c5c6c([2H])c([2H])c([2H])c([2H])c6n(-c6ccc7c8ccccc8n(-c8ccccc8)c7c6)c5c([2H])c([2H])c43)cc2)c([2H])c1[2H] ; [2H]c1c([2H])c2c(c3ccccc3n2-c2ccc3c(c2)oc2ccccc23)c2c3ccccc3n(-c3cccc(-c4ccccc4)c3)c12 ; [2H]c1c([2H])c([2H])c2c(sc3c(-n4c5ccccc5c5c6c7ccccc7n(-c7ccccc7)c6ccc54)c([2H])c([2H])c([2H])c32)c1[2H]]

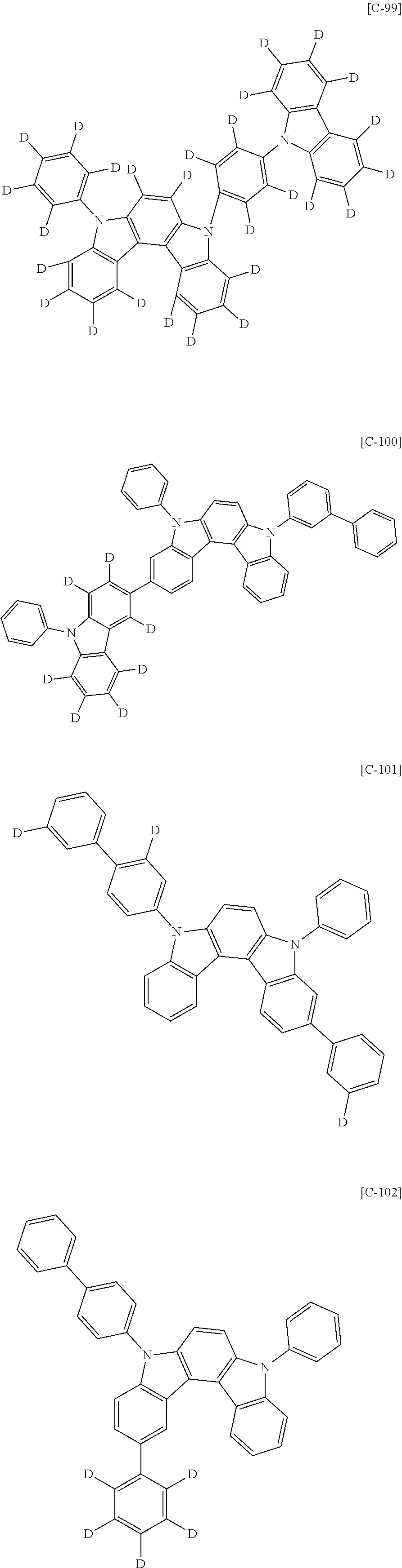

In an implementation, the second compound for organic optoelectronic device may be, e.g., a compound of Group 4.

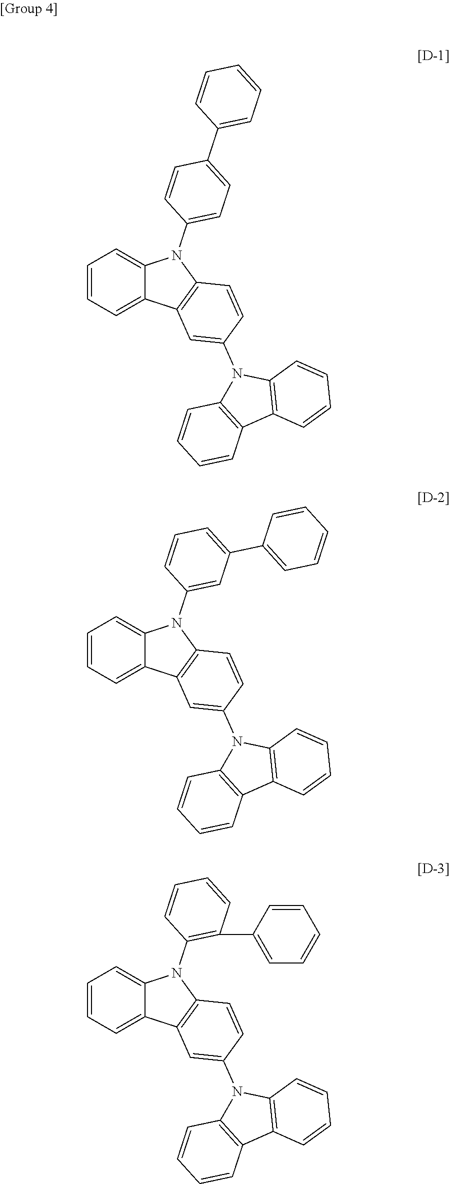

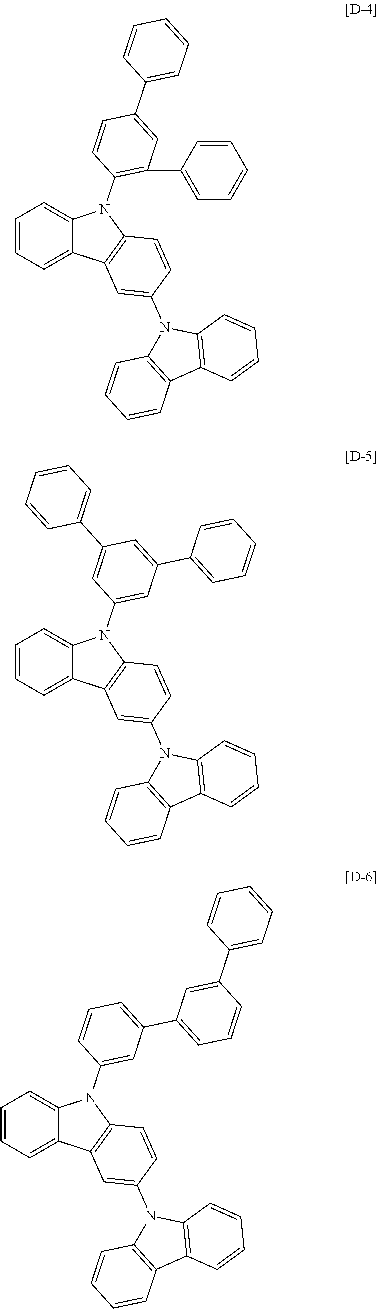 [SMILES: c1ccc(-c2ccc(-n3c4ccccc4c4cc(-n5c6ccccc6c6ccccc65)ccc43)c(-c3ccccc3)c2)cc1 ; c1ccc(-c2cc(-c3ccccc3)cc(-n3c4ccccc4c4cc(-n5c6ccccc6c6ccccc65)ccc43)c2)cc1 ; c1ccc(-c2cccc(-c3cccc(-n4c5ccccc5c5cc(-n6c7ccccc7c7ccccc76)ccc54)c3)c2)cc1]

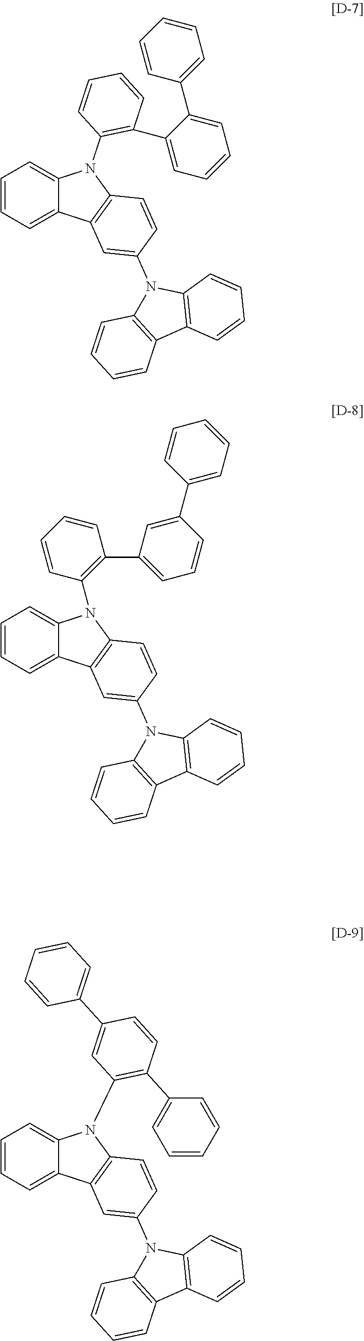 [SMILES: c1ccc(-c2ccccc2-c2ccccc2-n2c3ccccc3c3cc(-n4c5ccccc5c5ccccc54)ccc32)cc1 ; c1ccc(-c2cccc(-c3ccccc3-n3c4ccccc4c4cc(-n5c6ccccc6c6ccccc65)ccc43)c2)cc1 ; c1ccc(-c2ccc(-c3ccccc3)c(-n3c4ccccc4c4cc(-n5c6ccccc6c6ccccc65)ccc43)c2)cc1]

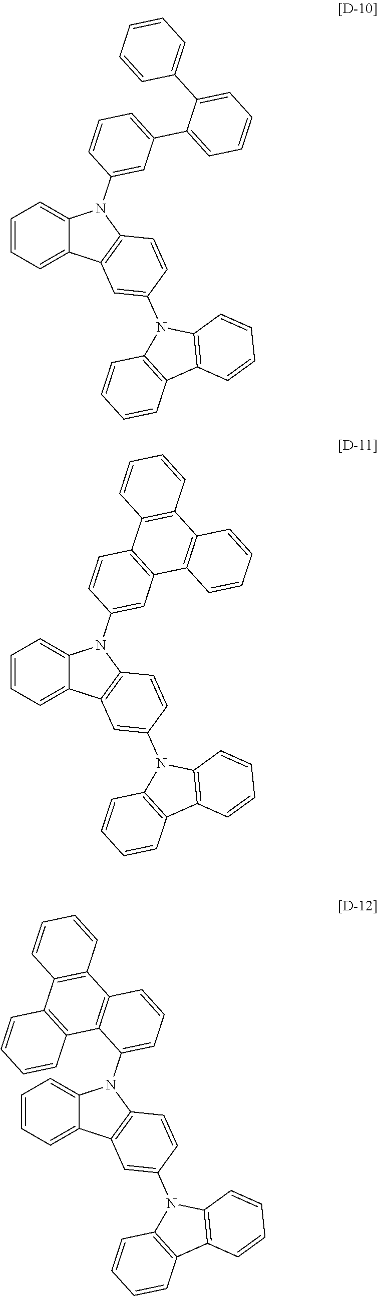

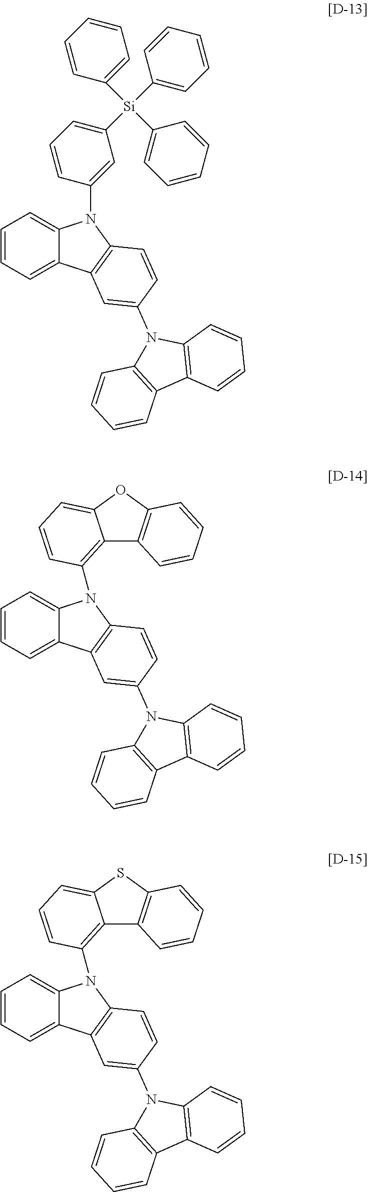 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)oc1cccc(-n3c4ccccc4c4cc(-n5c6ccccc6c6ccccc65)ccc43)c12 ; c1ccc2c(c1)sc1cccc(-n3c4ccccc4c4cc(-n5c6ccccc6c6ccccc65)ccc43)c12]

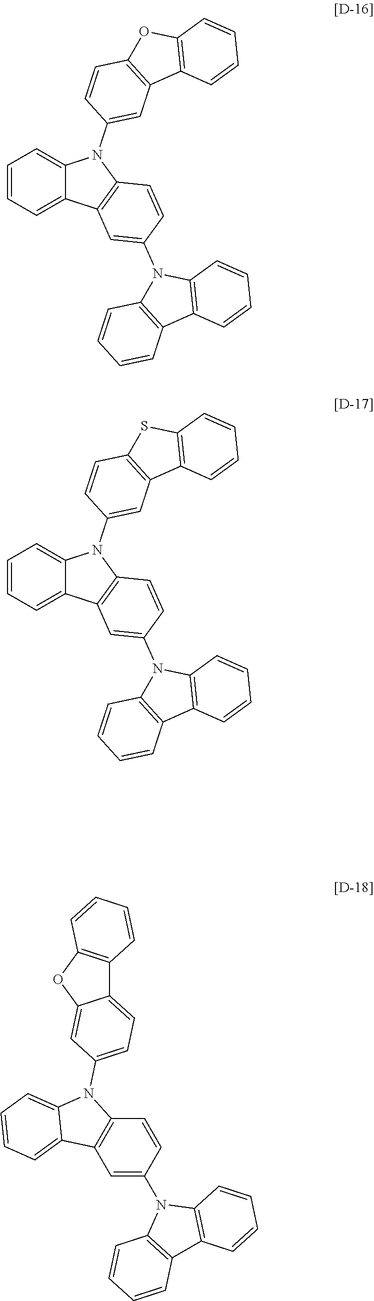 [SMILES: c1ccc2c(c1)oc1ccc(-n3c4ccccc4c4cc(-n5c6ccccc6c6ccccc65)ccc43)cc12 ; c1ccc2c(c1)sc1ccc(-n3c4ccccc4c4cc(-n5c6ccccc6c6ccccc65)ccc43)cc12 ; c1ccc2c(c1)oc1cc(-n3c4ccccc4c4cc(-n5c6ccccc6c6ccccc65)ccc43)ccc12]

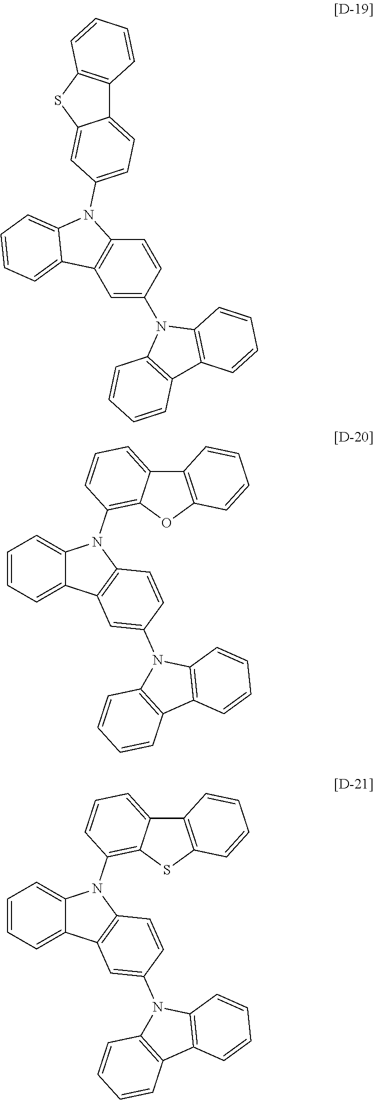

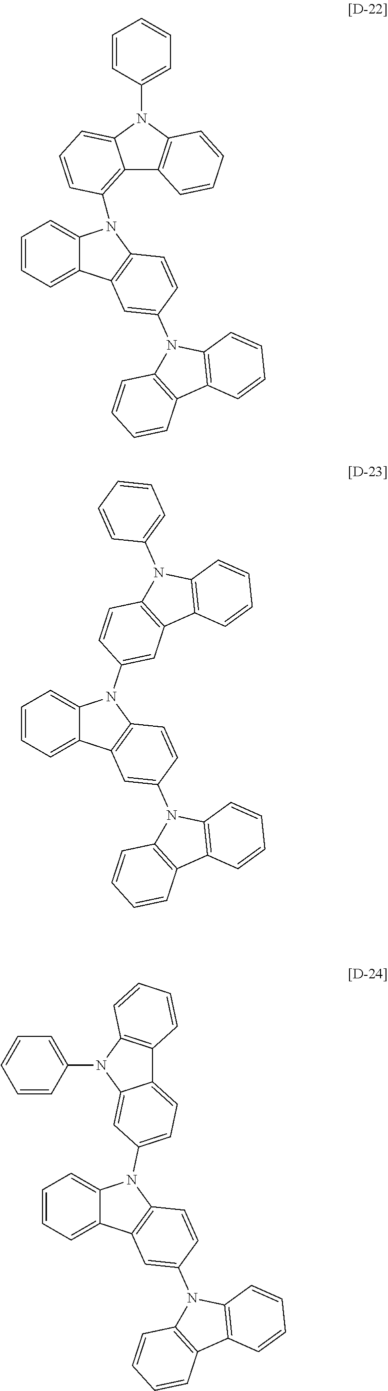 [SMILES: c1ccc(-n2c3ccccc3c3c(-n4c5ccccc5c5cc(-n6c7ccccc7c7ccccc76)ccc54)cccc32)cc1 ; c1ccc(-n2c3ccccc3c3cc(-n4c5ccccc5c5cc(-n6c7ccccc7c7ccccc76)ccc54)ccc32)cc1 ; c1ccc(-n2c3ccccc3c3ccc(-n4c5ccccc5c5cc(-n6c7ccccc7c7ccccc76)ccc54)cc32)cc1]

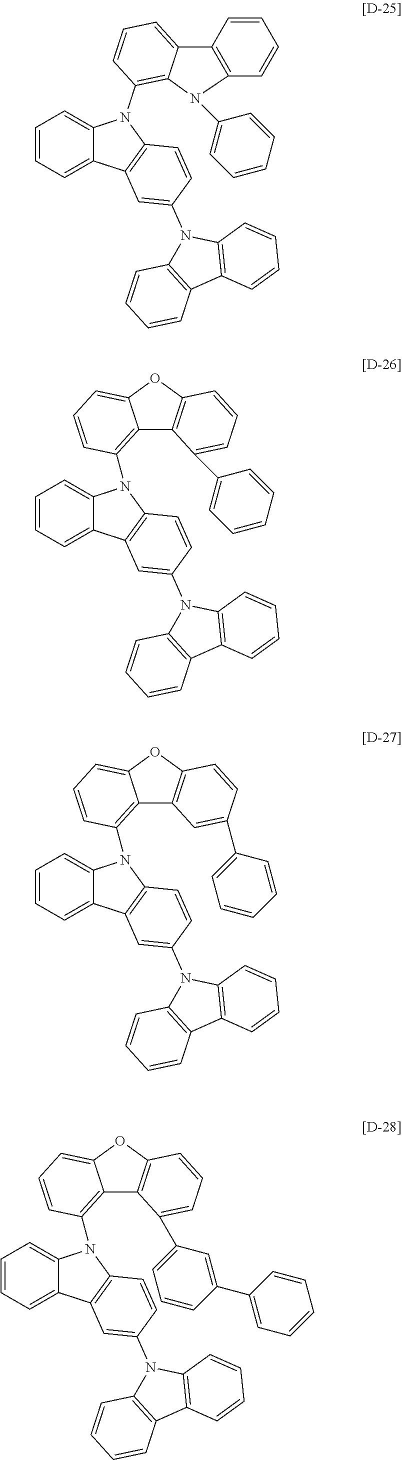 [SMILES: c1ccc(-n2c3ccccc3c3cccc(-n4c5ccccc5c5cc(-n6c7ccccc7c7ccccc76)ccc54)c32)cc1 ; c1ccc(-c2cccc3oc4cccc(-n5c6ccccc6c6cc(-n7c8ccccc8c8ccccc87)ccc65)c4c23)cc1 ; c1ccc(-c2ccc3oc4cccc(-n5c6ccccc6c6cc(-n7c8ccccc8c8ccccc87)ccc65)c4c3c2)cc1 ; c1ccc(-c2cccc(-c3cccc4oc5cccc(-n6c7ccccc7c7cc(-n8c9ccccc9c9ccccc98)ccc76)c5c34)c2)cc1]

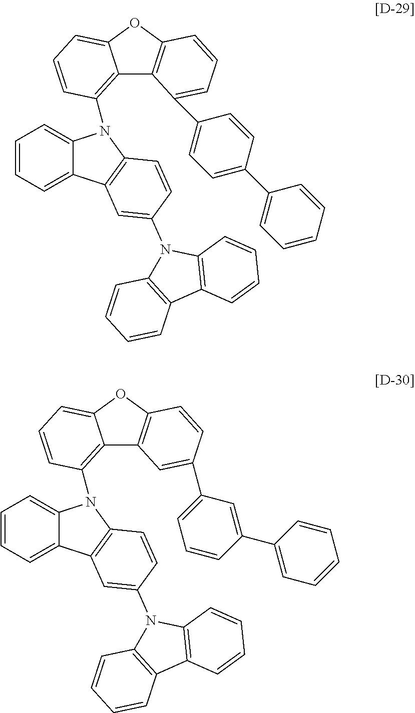 [SMILES: c1ccc(-c2ccc(-c3cccc4oc5cccc(-n6c7ccccc7c7cc(-n8c9ccccc9c9ccccc98)ccc76)c5c34)cc2)cc1 ; c1ccc(-c2cccc(-c3ccc4oc5cccc(-n6c7ccccc7c7cc(-n8c9ccccc9c9ccccc98)ccc76)c5c4c3)c2)cc1]

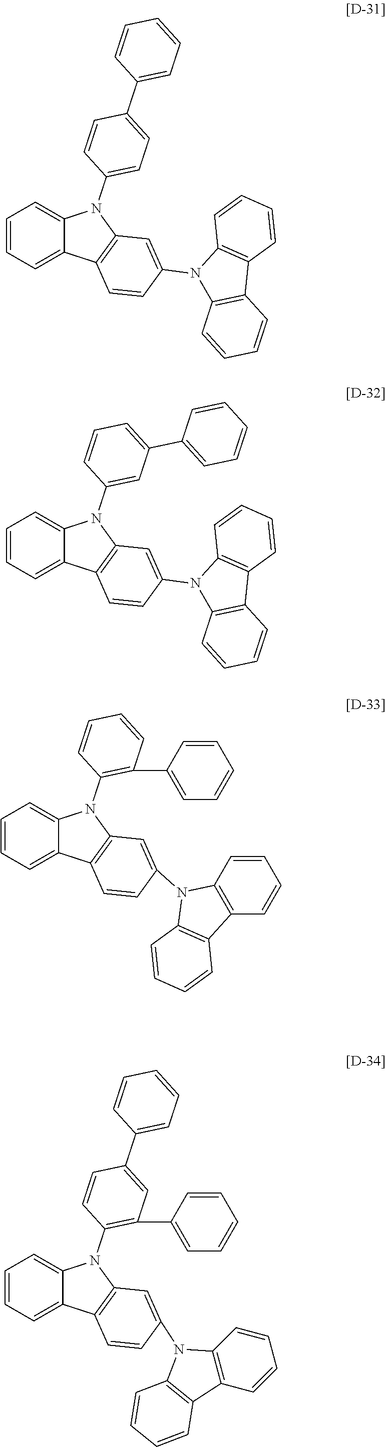

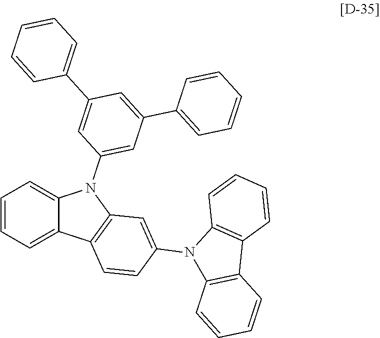 [SMILES: c1ccc(-c2cc(-c3ccccc3)cc(-n3c4ccccc4c4ccc(-n5c6ccccc6c6ccccc65)cc43)c2)cc1]

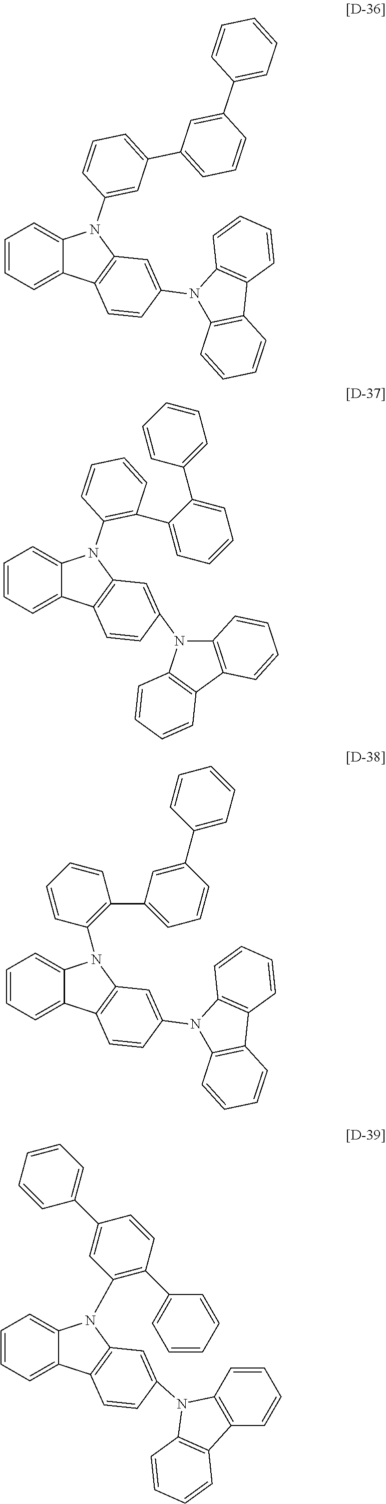

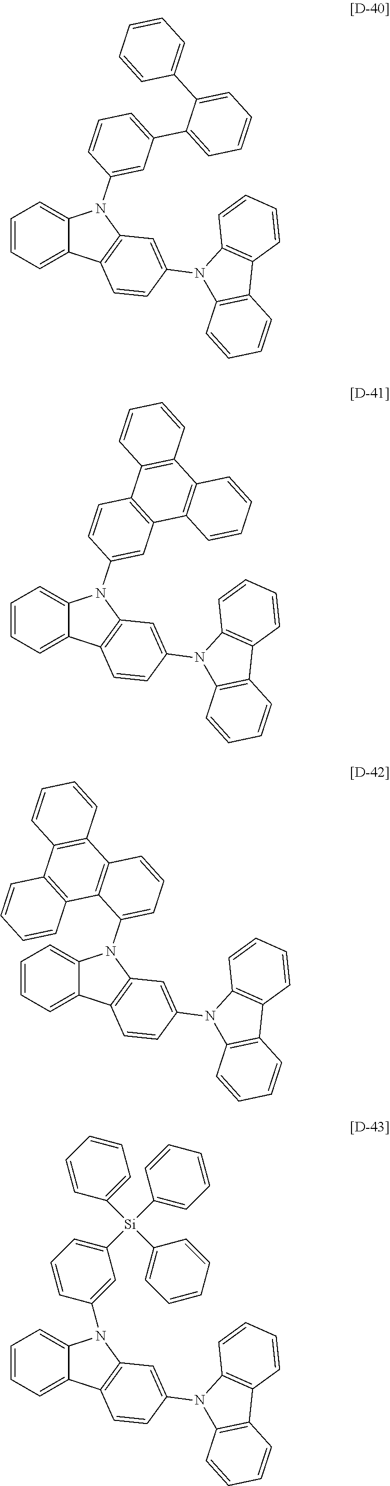 [SMILES: c1ccc(-c2ccccc2-c2cccc(-n3c4ccccc4c4ccc(-n5c6ccccc6c6ccccc65)cc43)c2)cc1 ; c1ccc2c(c1)c1ccccc1c1cc(-n3c4ccccc4c4ccc(-n5c6ccccc6c6ccccc65)cc43)ccc21 ; c1ccc2c(c1)c1ccccc1c1c(-n3c4ccccc4c4ccc(-n5c6ccccc6c6ccccc65)cc43)cccc21 ; c1ccc([Si](c2ccccc2)(c2ccccc2)c2cccc(-n3c4ccccc4c4ccc(-n5c6ccccc6c6ccccc65)cc43)c2)cc1]

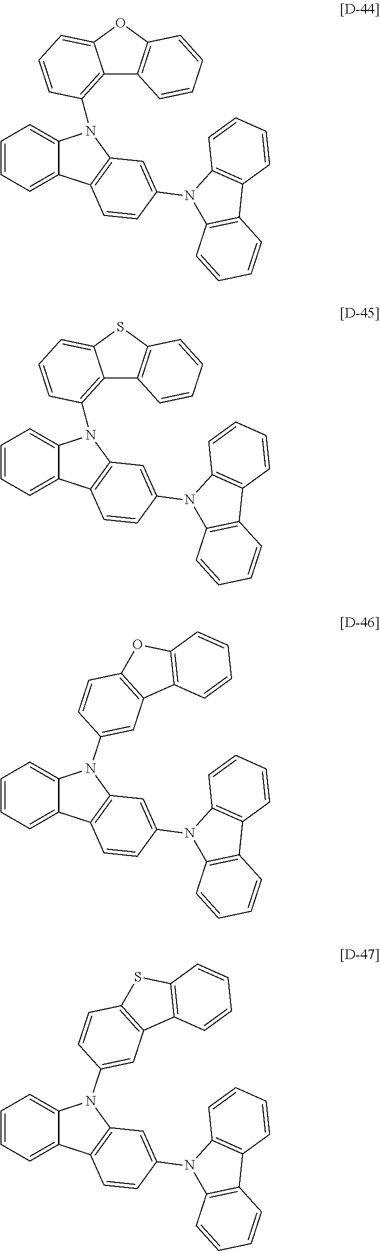 [SMILES: c1ccc2c(c1)oc1cccc(-n3c4ccccc4c4ccc(-n5c6ccccc6c6ccccc65)cc43)c12 ; c1ccc2c(c1)sc1cccc(-n3c4ccccc4c4ccc(-n5c6ccccc6c6ccccc65)cc43)c12 ; c1ccc2c(c1)oc1ccc(-n3c4ccccc4c4ccc(-n5c6ccccc6c6ccccc65)cc43)cc12 ; c1ccc2c(c1)sc1ccc(-n3c4ccccc4c4ccc(-n5c6ccccc6c6ccccc65)cc43)cc12]

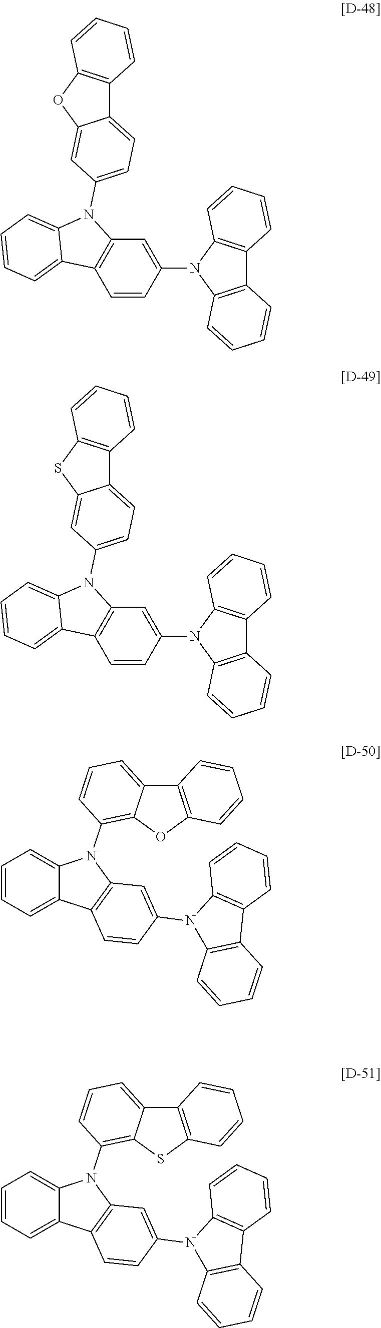 [SMILES: c1ccc2c(c1)oc1cc(-n3c4ccccc4c4ccc(-n5c6ccccc6c6ccccc65)cc43)ccc12 ; c1ccc2c(c1)sc1cc(-n3c4ccccc4c4ccc(-n5c6ccccc6c6ccccc65)cc43)ccc12 ; c1ccc2c(c1)oc1c(-n3c4ccccc4c4ccc(-n5c6ccccc6c6ccccc65)cc43)cccc12 ; c1ccc2c(c1)sc1c(-n3c4ccccc4c4ccc(-n5c6ccccc6c6ccccc65)cc43)cccc12]

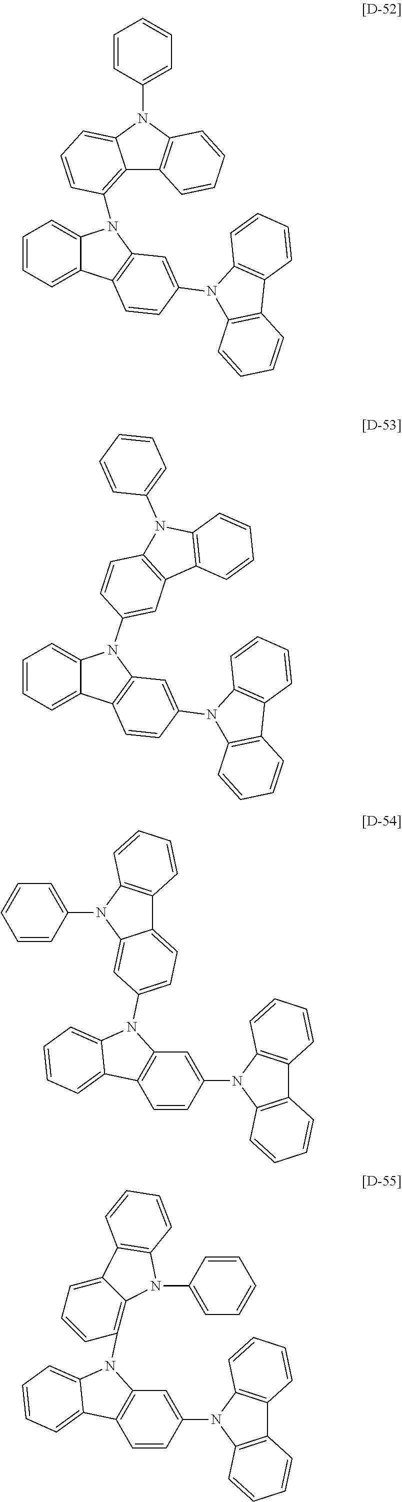 [SMILES: c1ccc(-n2c3ccccc3c3c(-n4c5ccccc5c5ccc(-n6c7ccccc7c7ccccc76)cc54)cccc32)cc1 ; c1ccc(-n2c3ccccc3c3cc(-n4c5ccccc5c5ccc(-n6c7ccccc7c7ccccc76)cc54)ccc32)cc1 ; c1ccc(-n2c3ccccc3c3ccc(-n4c5ccccc5c5ccc(-n6c7ccccc7c7ccccc76)cc54)cc32)cc1 ; c1ccc(-n2c3ccccc3c3cccc(-n4c5ccccc5c5ccc(-n6c7ccccc7c7ccccc76)cc54)c32)cc1]

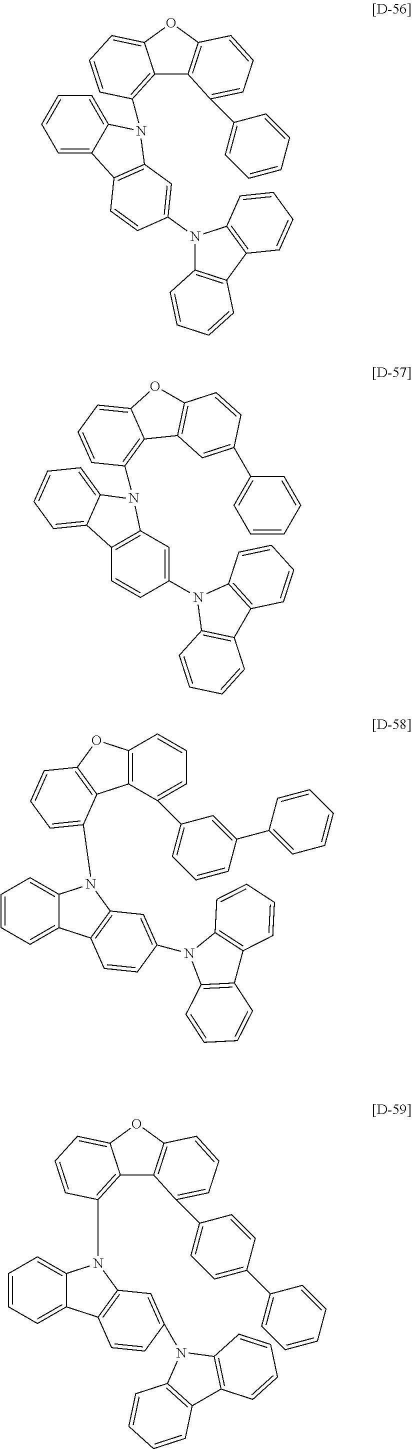 [SMILES: c1ccc(-c2cccc3oc4cccc(-n5c6ccccc6c6ccc(-n7c8ccccc8c8ccccc87)cc65)c4c23)cc1 ; c1ccc(-c2ccc3oc4cccc(-n5c6ccccc6c6ccc(-n7c8ccccc8c8ccccc87)cc65)c4c3c2)cc1 ; c1ccc(-c2cccc(-c3cccc4oc5cccc(-n6c7ccccc7c7ccc(-n8c9ccccc9c9ccccc98)cc76)c5c34)c2)cc1 ; c1ccc(-c2ccc(-c3cccc4oc5cccc(-n6c7ccccc7c7ccc(-n8c9ccccc9c9ccccc98)cc76)c5c34)cc2)cc1]

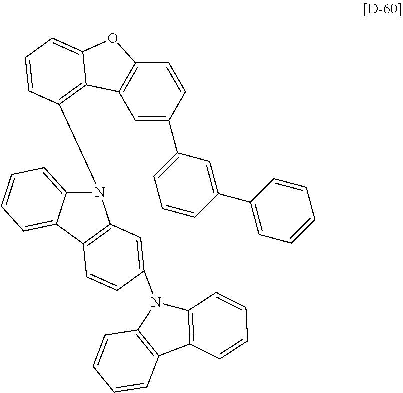 [SMILES: c1ccc(-c2cccc(-c3ccc4oc5cccc(-n6c7ccccc7c7ccc(-n8c9ccccc9c9ccccc98)cc76)c5c4c3)c2)cc1]

Additionally, examples of Compound D-1 to Compound D-60 listed in Group 4 in which at least one hydrogen is replaced with deuterium are given below.

In an implementation, deuterium may be substituted, e.g., as shown in Compound D-61 to Compound D-120 exemplified below.

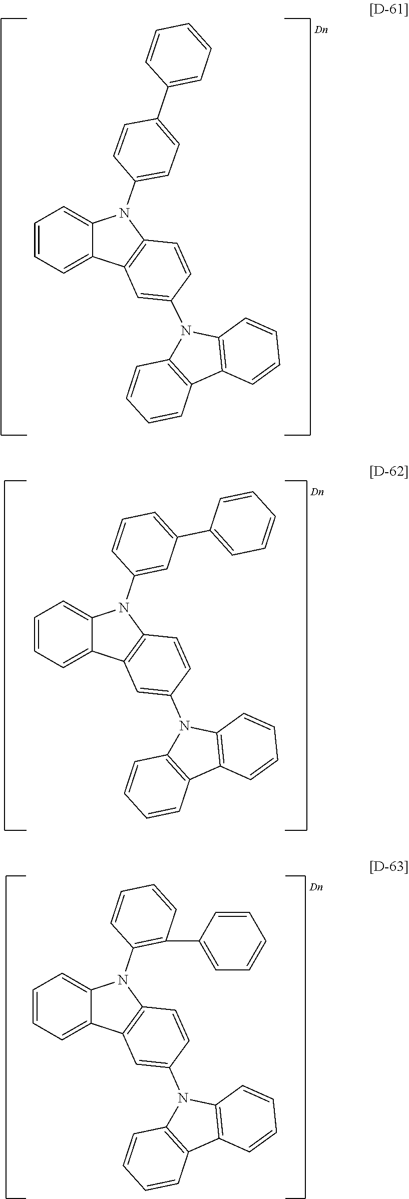

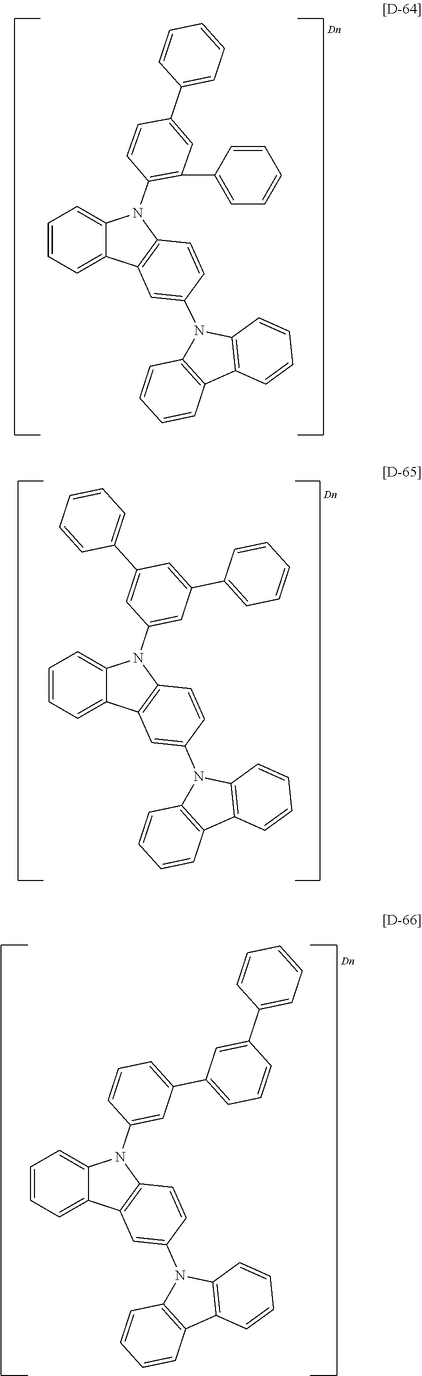

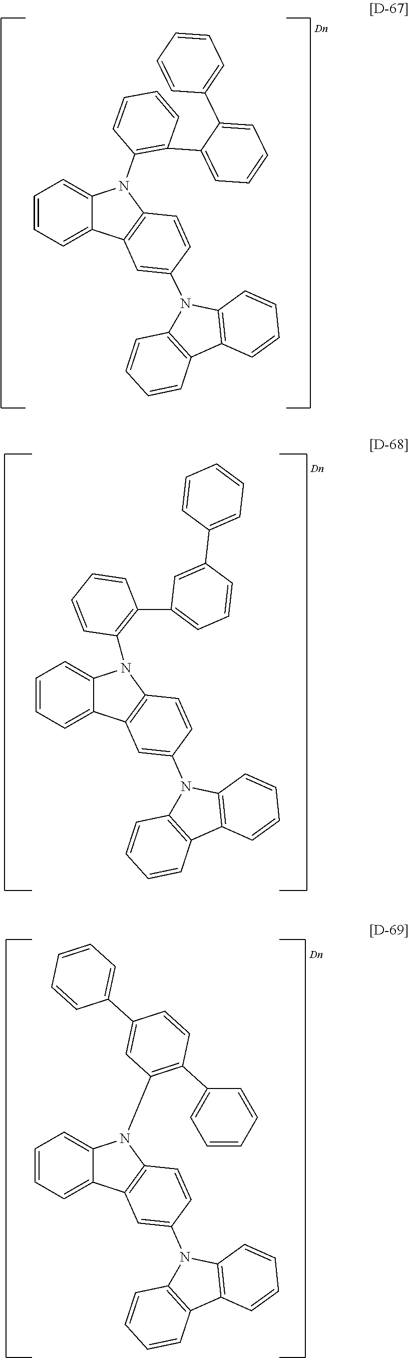

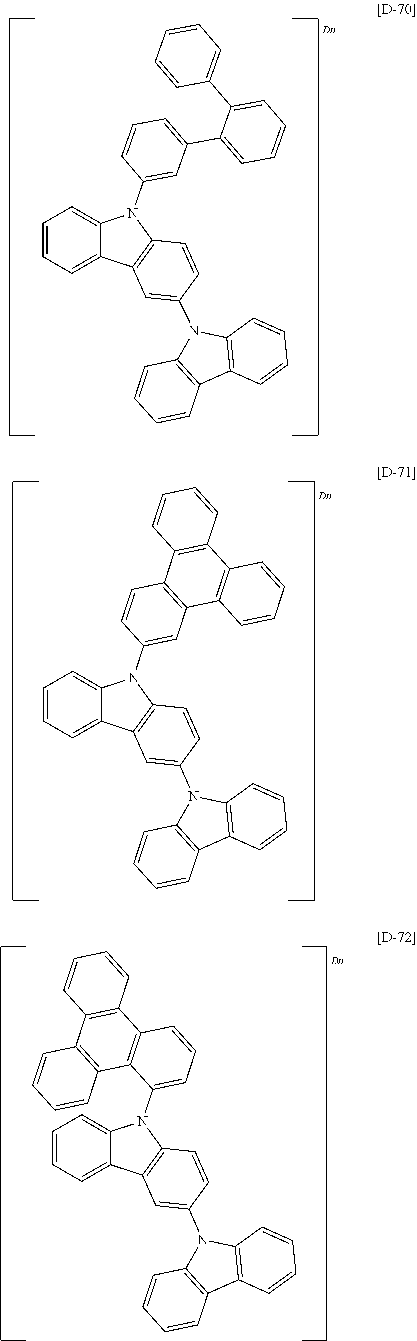

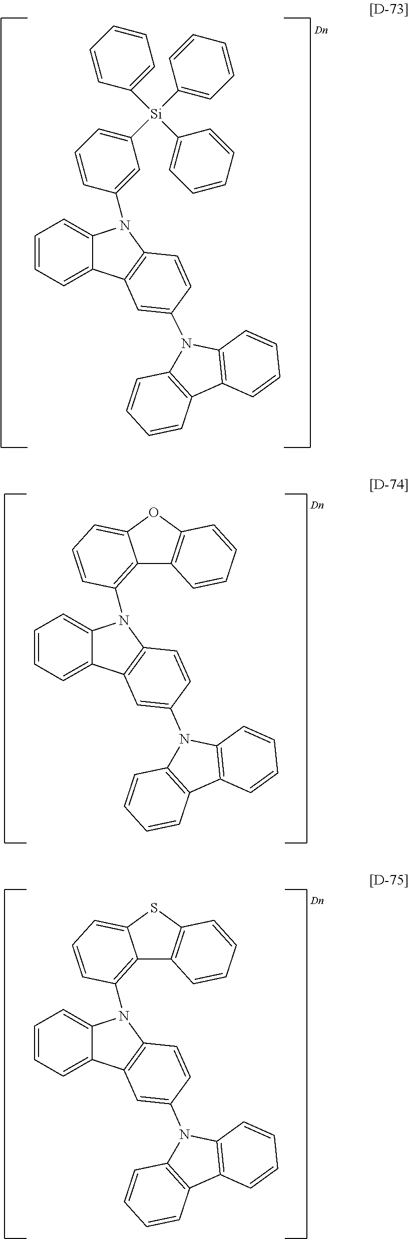

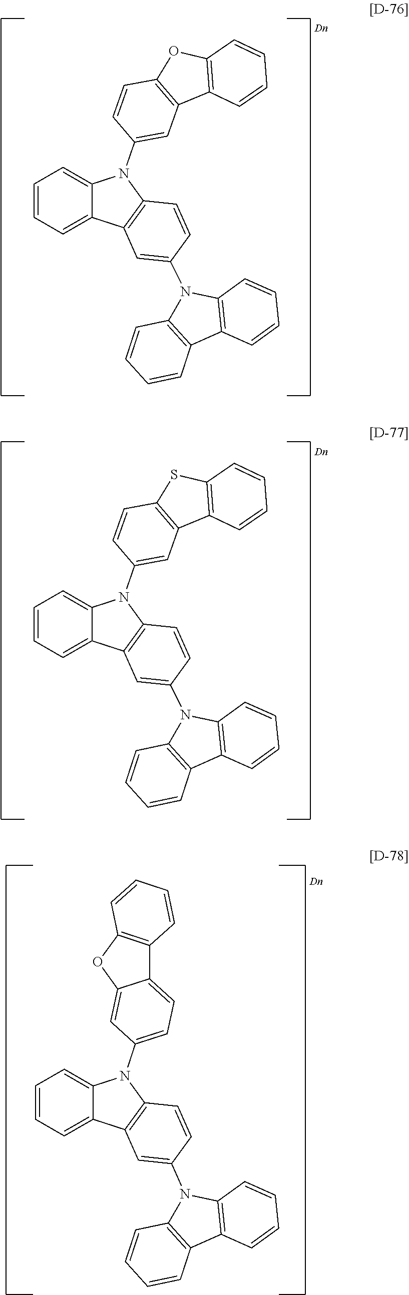

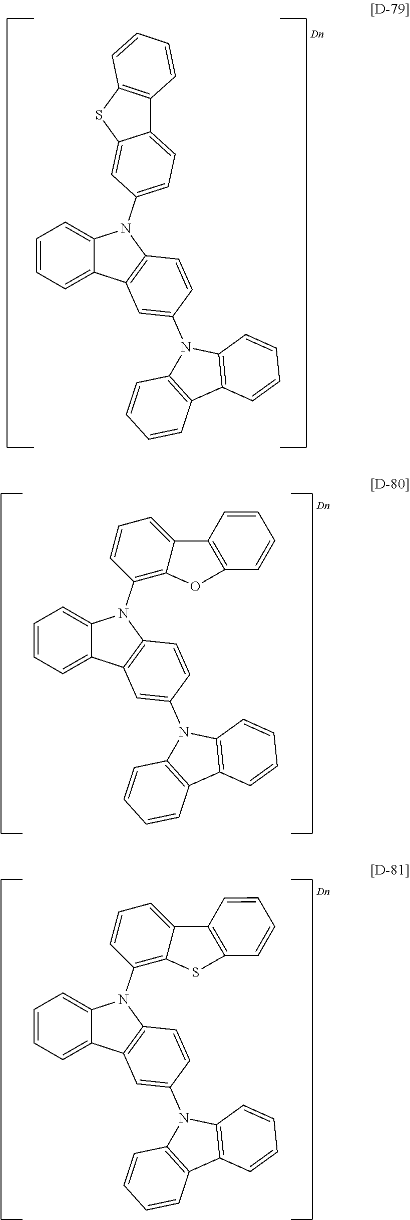

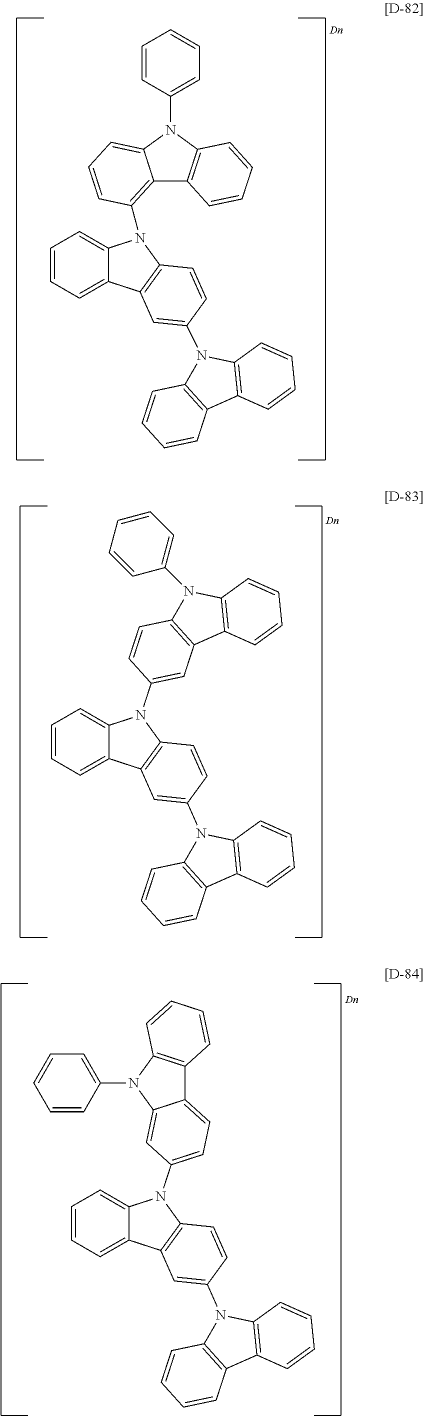

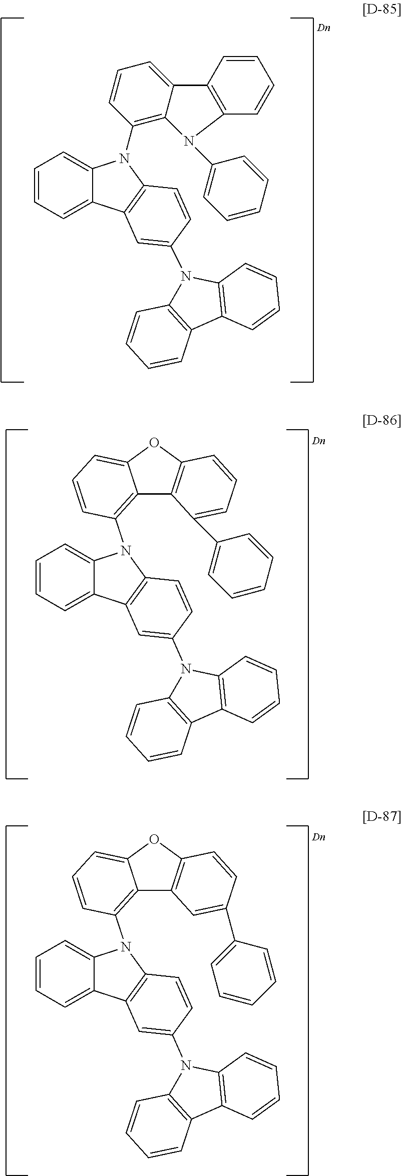

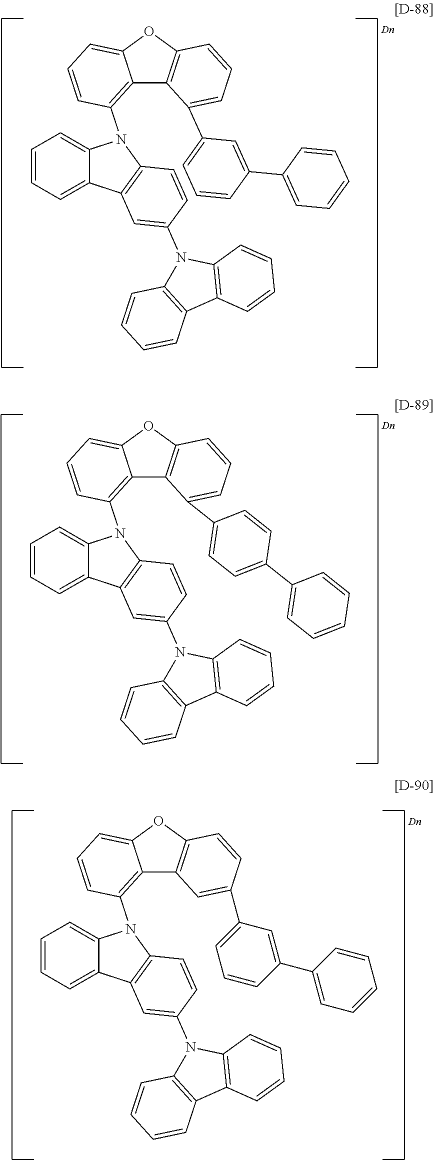

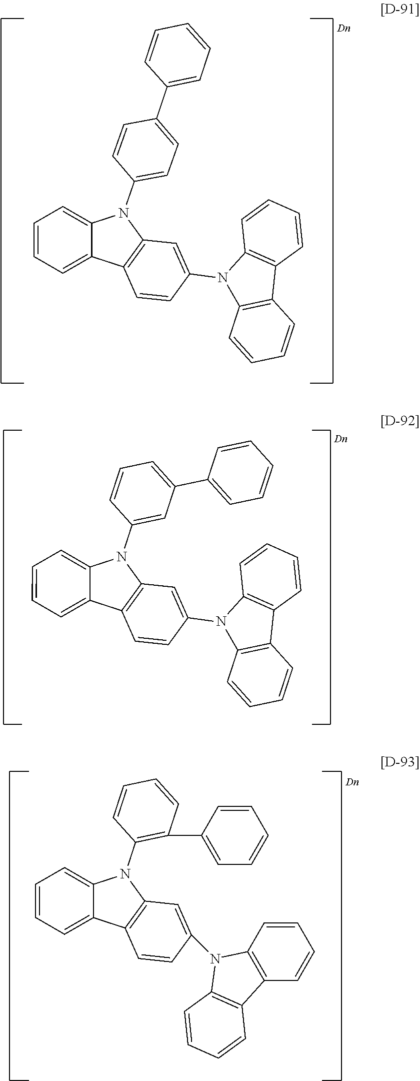

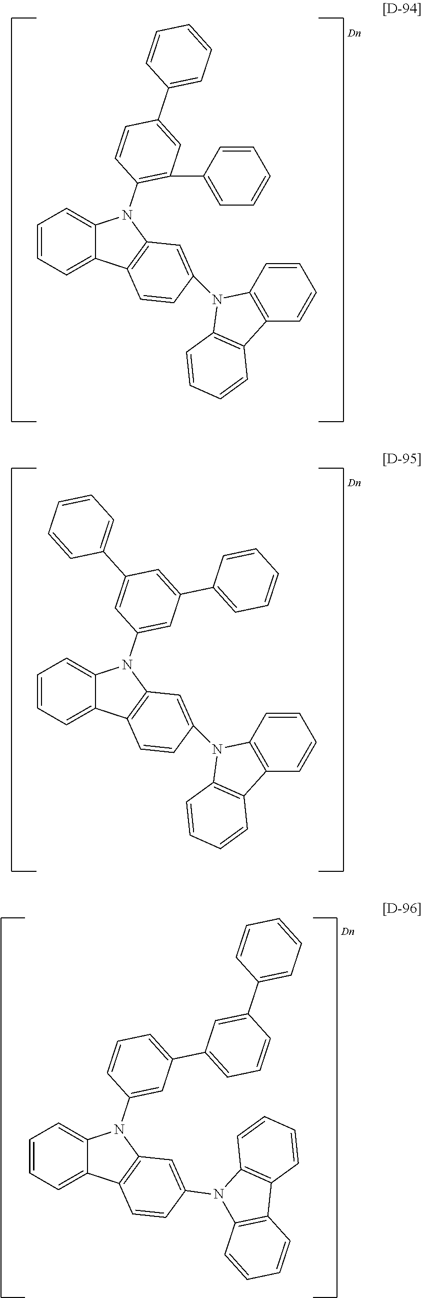

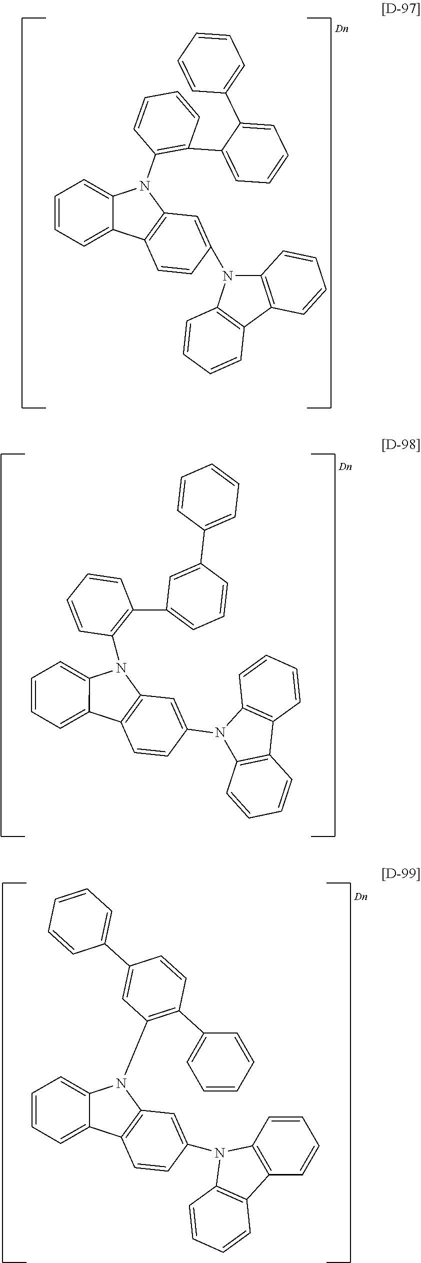

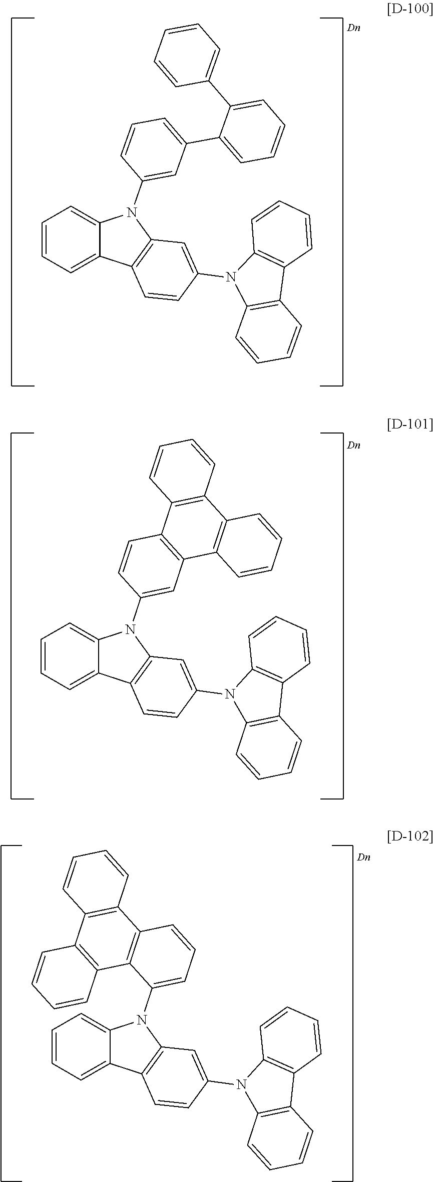

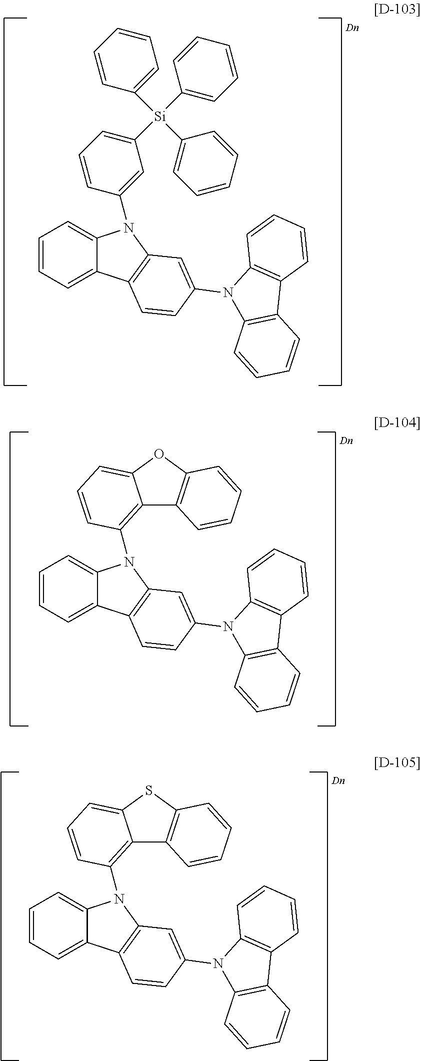

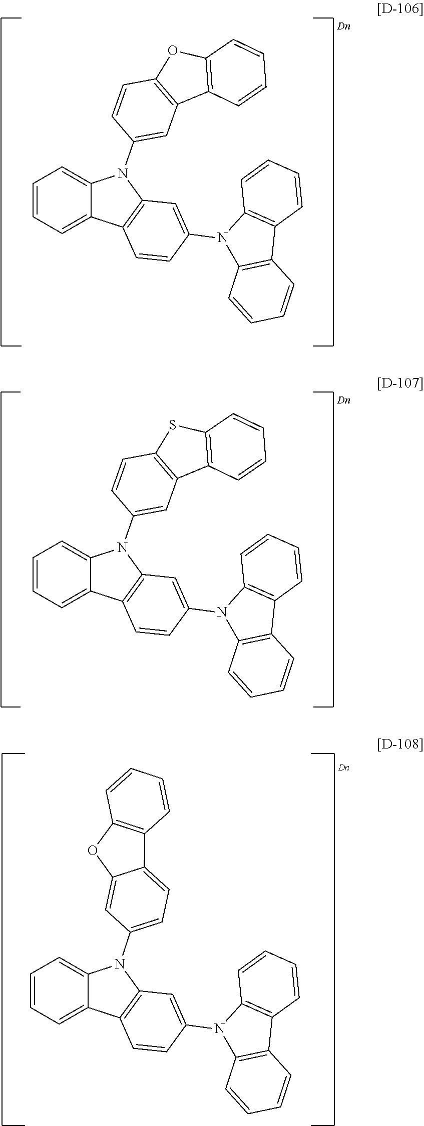

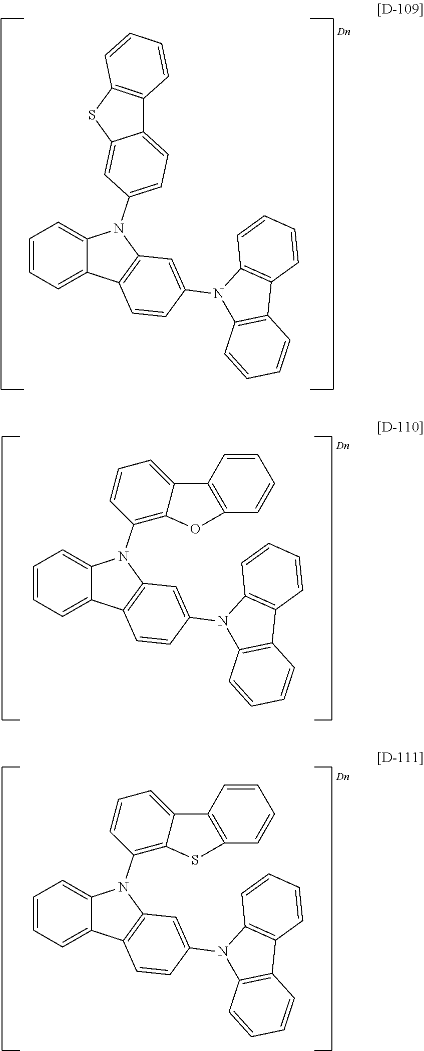

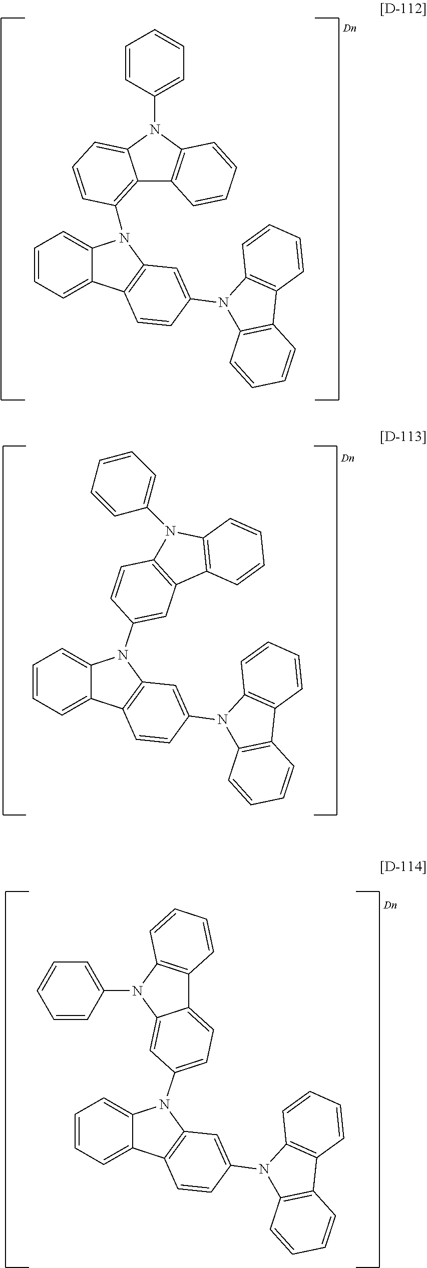

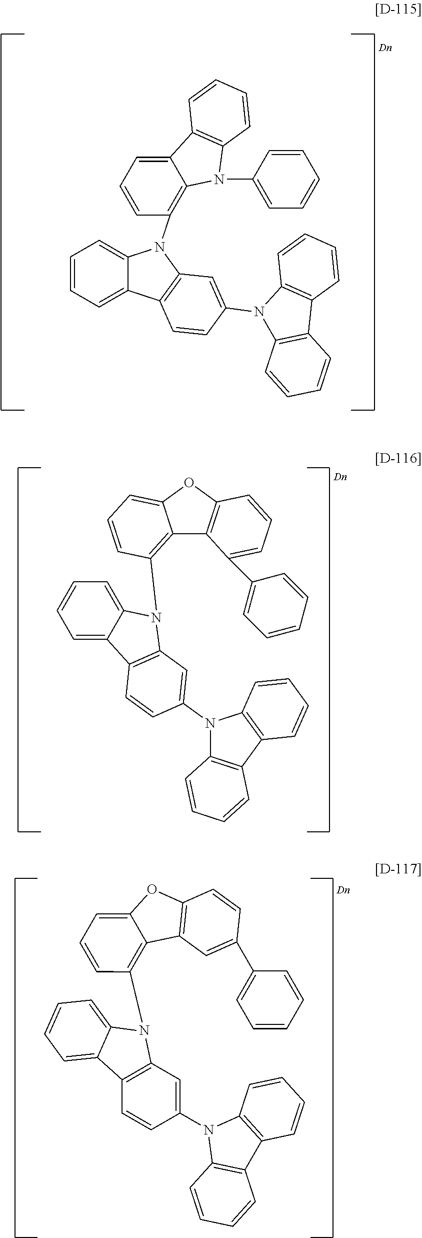

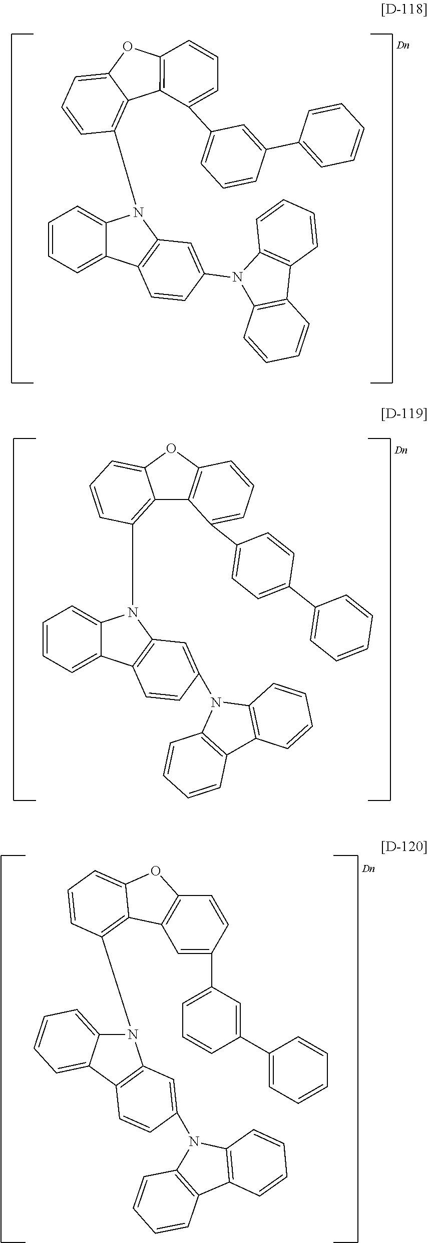

(Dn refers to the number of deuterium substitutions and indicates a structure substituted with one or more deuterium atoms)

The deuterium substitution position and deuterium substitution ratio may include all changeable ranges within the range of Compound D-1 to Compound D-120 (e.g., any hydrogen in any compound may be a protium or a deuterium).

In an implementation, the second compound may be represented by Chemical Formula 2-8.

The first compound and the second compound may be included (e.g., mixed) in a weight ratio of, e.g., about 1:99 to about 99:1. By being included in the above range, efficiency and life-span may be improved by implementing bipolar characteristics by adjusting the appropriate weight ratio using the electron transport capability of the first compound and the hole transport capability of the second compound. Within the above range, they may be included in a weight ratio of, e.g., about 10:90 to about 90:10, about 20:80 to about 80:20, e.g., about 20:80 to about 70:30, about 20:80 to about 60:40, and about 30:70 to about 60:40. In an implementation, they may be included in a weight ratio of about 40:60, about 50:50, or about 60:40.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to the drawing.

The FIGURE is a cross-sectional view showing an organic light emitting diode according to some example embodiments.

Referring to the FIGURE, an organic light emitting diode 100 according to some example embodiments may include an anode 120 and a cathode 110 facing each other and an organic layer 105 disposed between the anode 120 and cathode 110.

The anode 120 may be made of a conductor having a large work function to help hole injection, and may be, e.g., a metal, a metal oxide or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof, or a multi-layer structure material such as LiF/Al, LiO₂/Al, LiF/Ca, or BaF₂/Ca.

The organic layer 105 may include the aforementioned compound for an organic optoelectronic device or composition for an organic optoelectronic device.

The organic layer 105 may include a light emitting layer 130, and the light emitting layer 130 may include a host and a dopant, the host may include the aforementioned compound for an organic optoelectronic device or composition for an organic optoelectronic device, and the dopant may be, e.g., a phosphorescent dopant, e.g., a red, green or blue phosphorescent dopant, e.g., a red or green phosphorescent dopant.

The dopant may be a material mixed with the compound or composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may include a phosphorescent dopant and examples of the phosphorescent dopant may include an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

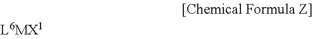

In Chemical Formula Z, M may be, e.g., a metal and L⁶ and X¹ may be the same or different, and may each independently be, e.g., ligands forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁶ and X¹ may be, e.g., a bidentate ligand.

The bidentate ligand represented by L⁶ and X¹ may be represented by Chemical Formula Z-1 or Chemical Formula Z-2.

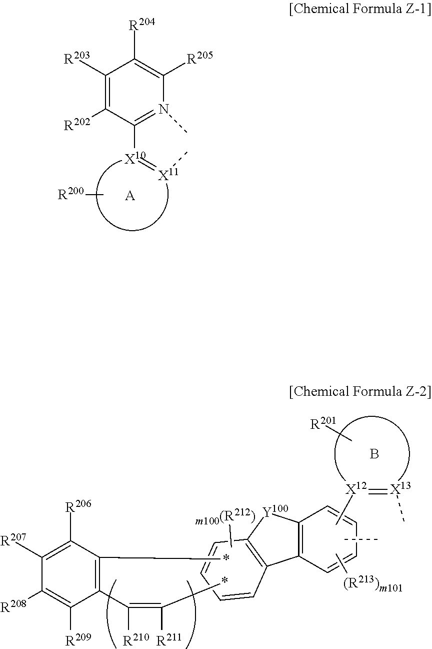

In Chemical Formula Z-1 and Chemical Formula Z-2, ring A and ring B may each independently be, e.g., a monocyclic ring or a polycyclic fused ring, wherein each ring among the monocyclic ring and polycyclic fused ring may be, e.g., a 5- or 6-membered carbocyclic or heterocyclic ring

R²⁰⁰ and R²⁰¹ may each independently represent, e.g., one to a maximum number of monovalent substituents.

In an implementation, R²⁰⁰ and R²⁰¹ may be two or more, and each R²⁰⁰ and R²⁰¹ may be the same or different from each other.

R²⁰² to R²¹³ may each independently be, e.g., hydrogen, deuterium, halogen, a substituted or unsubstituted C1 to C10 alkyl group, a substituted or unsubstituted C6 to C20 aryl group, —SiR²¹⁴R²¹⁵R²¹⁶, —GeR²¹⁴R²¹⁵R²¹⁶, or a combination thereof.

R²¹⁴ to R²¹⁶ may each independently be, e.g., a substituted or unsubstituted C1 to C6 alkyl group.

X¹⁰, X¹¹, X¹², and X¹³ may each independently be carbon or nitrogen.

Y¹⁰⁰ may be, e.g., O or S.

m100 may be an integer of 1 to 3.

m101 may be an integer of 1 to 2.

n100 may be an integer of 0 or 1.

* is a linking point.

When n100 is 0, it is formed with a monovalent substituent.

When n100 is 1, a fusion ring may be formed.

Examples of the ligands represented by L⁶ and X¹ may include ligands of Group A.

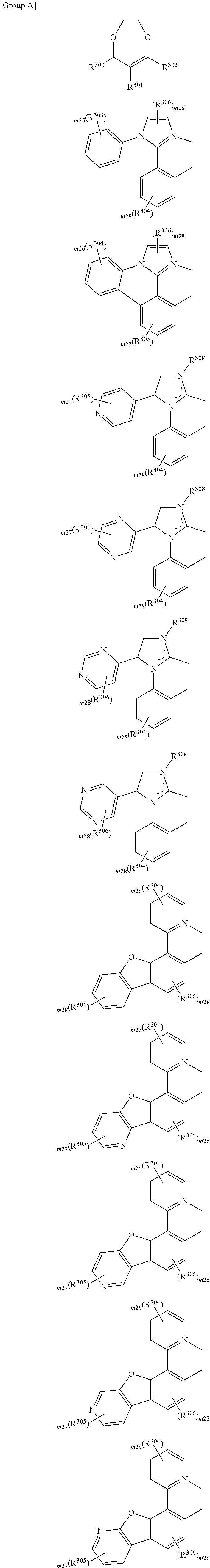

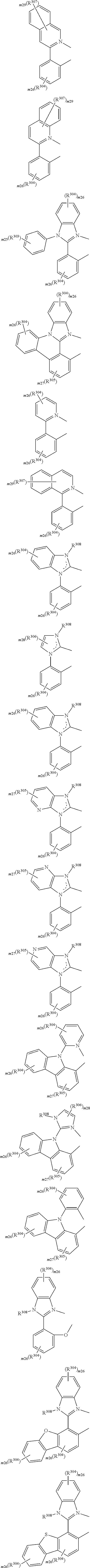

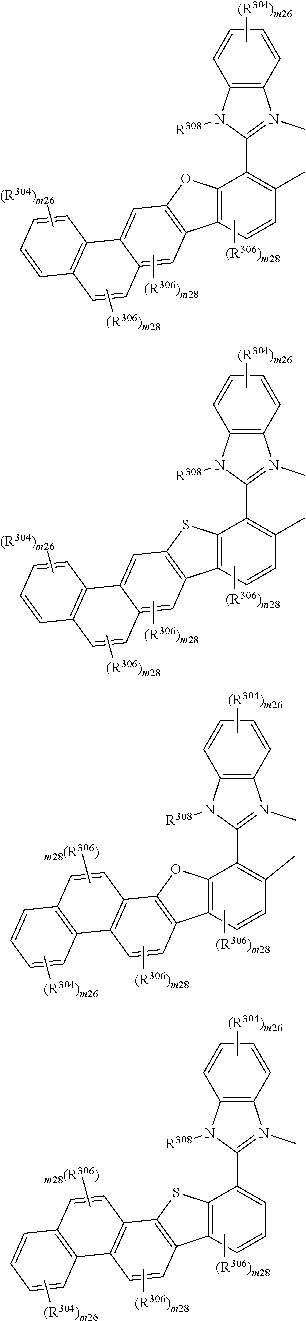

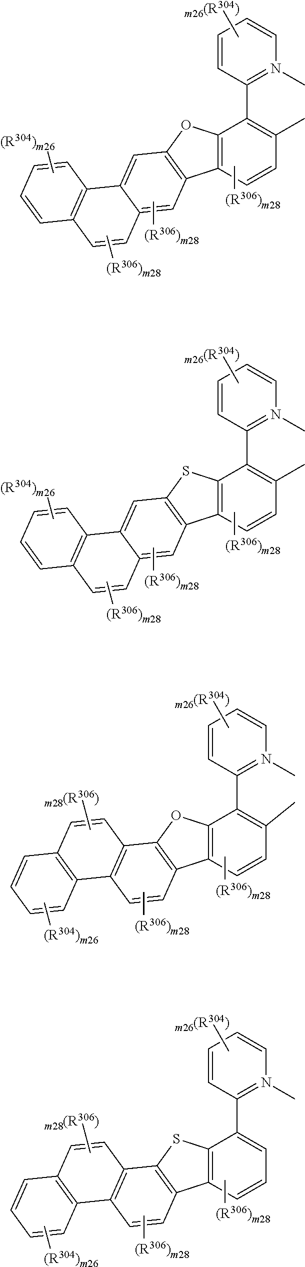

In Group A, R³⁰⁰ to R³⁰² may each independently be, e.g., hydrogen, deuterium, a C1 to C30 alkyl group that may be substituted or unsubstituted with a halogen, a C6 to C30 aryl group that may be substituted or unsubstituted with a C1 to C30 alkyl, or a halogen.

R³⁰³ to R³⁰⁸ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

m25 may be, e.g., an integer of 1 to 5.

m26 may be, e.g., an integer of 1 to 4.

m27 may be, e.g., an integer of 1 to 3.

m28 may be, e.g., an integer of 1 or 2.

m29 may be, e.g., an integer of 1 to 6.

In an implementation, m25 to m29 may be 2 or more, and each R³⁰³ to R³⁰⁷ may be the same or different from each other.

The dopant according to some example embodiments may be an iridium complex, and may be represented, e.g., by Chemical Formula 6-1 to Chemical Formula 6-5.

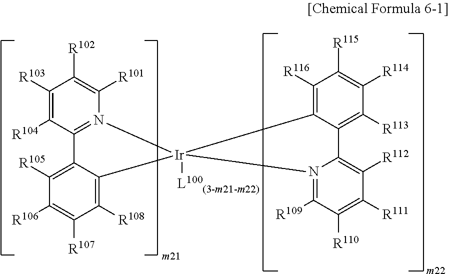

In Chemical Formula 6-1, R¹⁰¹ to R¹¹⁶ may each independently be, e.g., hydrogen, deuterium, halogen, a substituted or unsubstituted C1 to C10 alkyl group, a substituted or unsubstituted C6 to C20 aryl group, —SiR¹³²R¹³³R¹³⁴, or —GeR¹³²R¹³³R¹³⁴.

R¹³² to R¹³⁴ may each independently be, e.g., a substituted or unsubstituted C1 to C6 alkyl group.

At least one of R¹⁰¹ to R¹¹⁶ may be, e.g., a functional group represented by Chemical Formula V-1.

L¹⁰⁰ may be, e.g., a bidentate ligand of a monovalent anion and may be, e.g., a ligand that coordinates to iridium through a lone pair of carbons or heteroatoms.

m21 and m22 may each independently be, e.g., an integer of 0 to 3 and m21+m22 may be, e.g., an integer of 1 to 3.

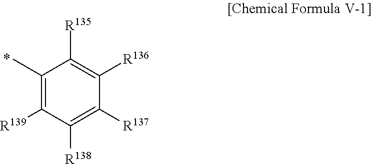

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴. R¹³² to R¹³⁴ may each independently be, e.g., a substituted or unsubstituted C1 to C6 alkyl group.

* refers to a portion linked to a carbon atom.

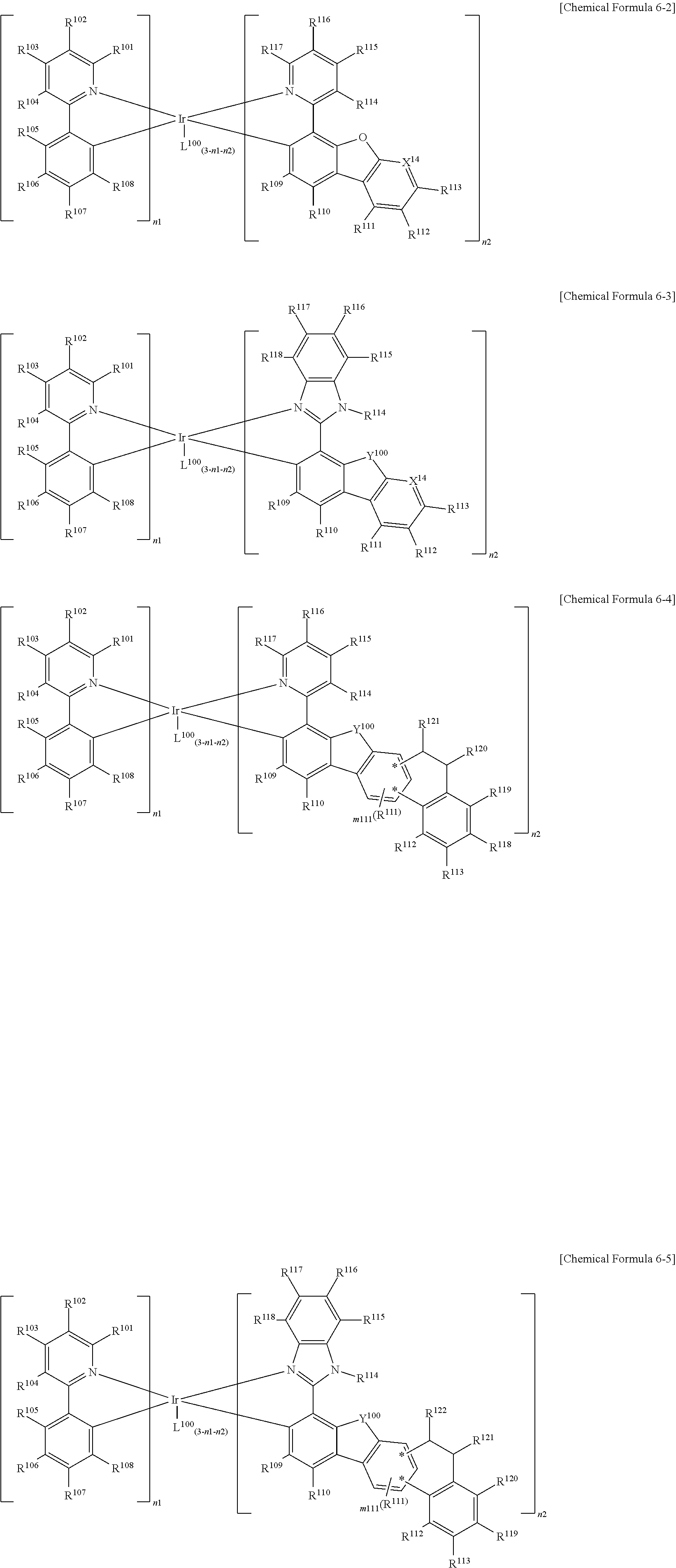

In Chemical Formula 6-2 to Chemical Formula 6-5, X¹⁴ may be, e.g., carbon or nitrogen.

Y¹⁰⁰ may be, e.g., O or S.

R¹⁰¹ to R¹²² may each independently be, e.g., hydrogen, deuterium, halogen, a substituted or unsubstituted C1 to C10 alkyl group, a substituted or unsubstituted C6 to C20 aryl group, —SiR¹³³R¹³⁴R¹³⁵, or —GeR¹³³R¹³⁴R¹³⁵.

R¹³³ to R¹³⁵ may each independently be, e.g., a substituted or unsubstituted C1 to C6 alkyl group.

L¹⁰⁰ may be, e.g., a bidentate ligand of a monovalent anion, and may be, e.g., a ligand that coordinates to iridium through a lone pair of carbons or heteroatoms.

m111 may be, e.g., an integer of 1 to 2.

n1 and n2 may each independently be, e.g., an integer of 0 to 3, and n1+n2 may be, e.g., an integer of 1 to 3.

The dopant according to some example embodiments may be a platinum complex, and may be represented, e.g., by Chemical Formula Z-3.

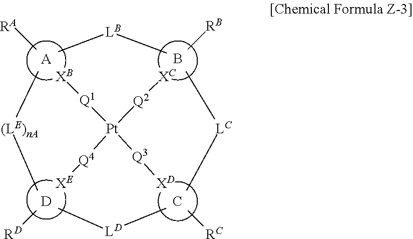

In Chemical Formula Z-3, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution.

L^{B}, L^{C}, and L^{D} may each independently be, e.g., a direct bond, BR, NR, PR, O, S, Se, C═O, S═O, SO₂, CRR′, SiRR′, GeRR′, or a combination thereof.

In an implementation, nA may be 1, L^{E} may be, e.g., a direct bond, BR, NR, PR, O, S, Se, C═O, S═O, SO₂, CRR′, SiRR′, GeRR′, or a combination thereof. In an implementation, nA may be 0 and L^{E} may not exist;

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ may optionally be linked to each other to provide a ring; X^{B}, X^{C}, X^{D}, and X^{E} may each independently be, e.g., carbon or nitrogen; and Q¹, Q², Q³, and Q⁴ may each independently be, e.g., oxygen or a direct bond.

The platinum complex may be represented by, e.g., Chemical Formula 7-1 or Chemical Formula 7-2.

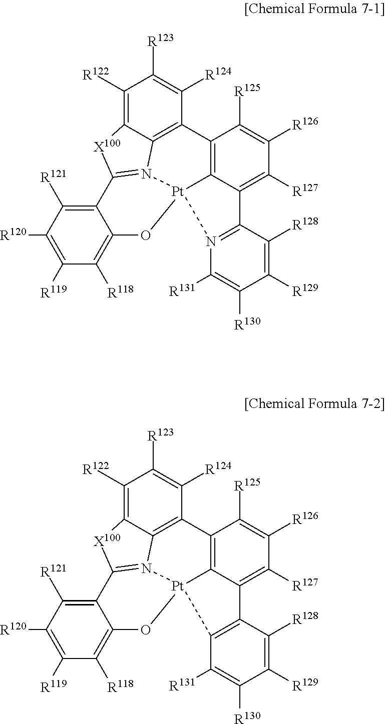

In Chemical Formula 7-1 and Chemical Formula 7-2, X¹⁰⁰ may be, e.g., O, S, or NR¹³².

R¹¹⁸ to R¹³² may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹⁸ to R¹³² may be, e.g., —SiR¹³³R¹³⁴R¹³⁵ or a tert-butyl group wherein R¹³³ to R¹³⁵ may each independently be, e.g., a substituted or unsubstituted C1 to C6 alkyl group.

The organic layer may further include a charge transport region in addition to the light emitting layer.

The charge transport region may be, e.g., the hole transport region 140.

The hole transport region 140 may help further increase hole injection or hole mobility between the anode 120 and the light emitting layer 130 and block electrons.

In an implementation, the hole transport region 140 may include, e.g., a hole transport layer between the anode 120 and the light emitting layer 130, and a hole transport auxiliary layer between the light emitting layer 130 and the hole transport layer, and a compound of Group B may be included in at least one of the hole transport layer and the hole transport auxiliary layer.

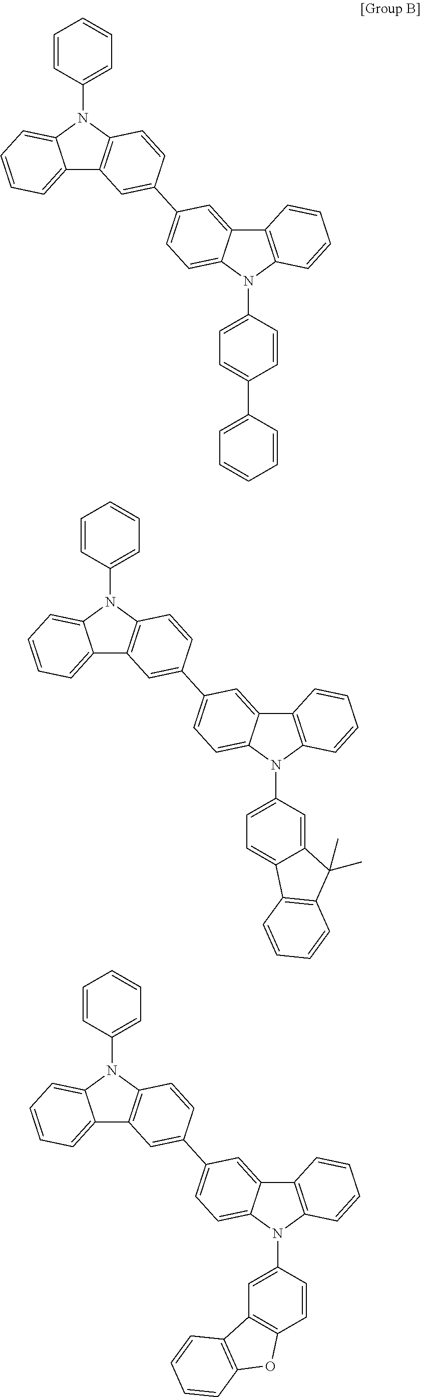 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

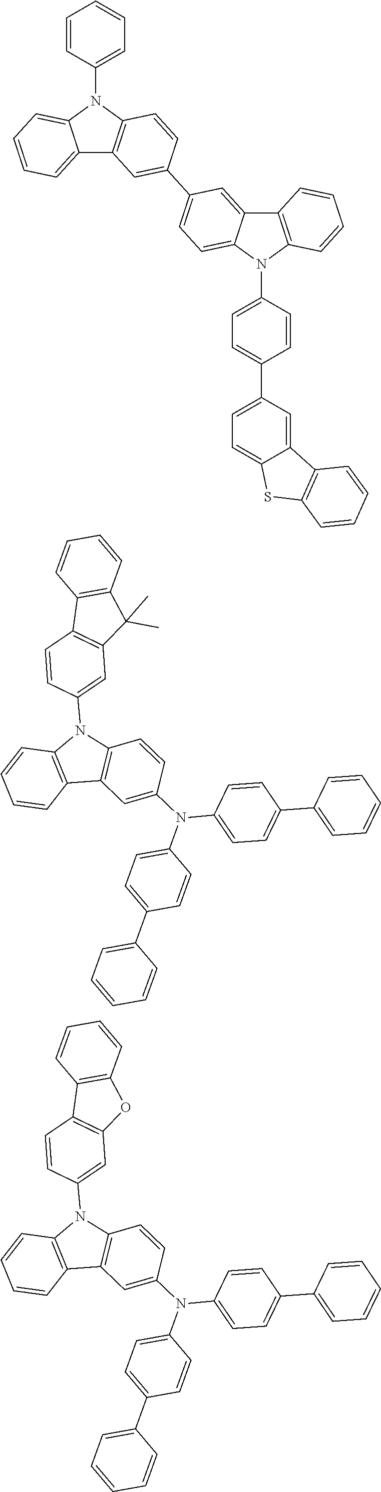

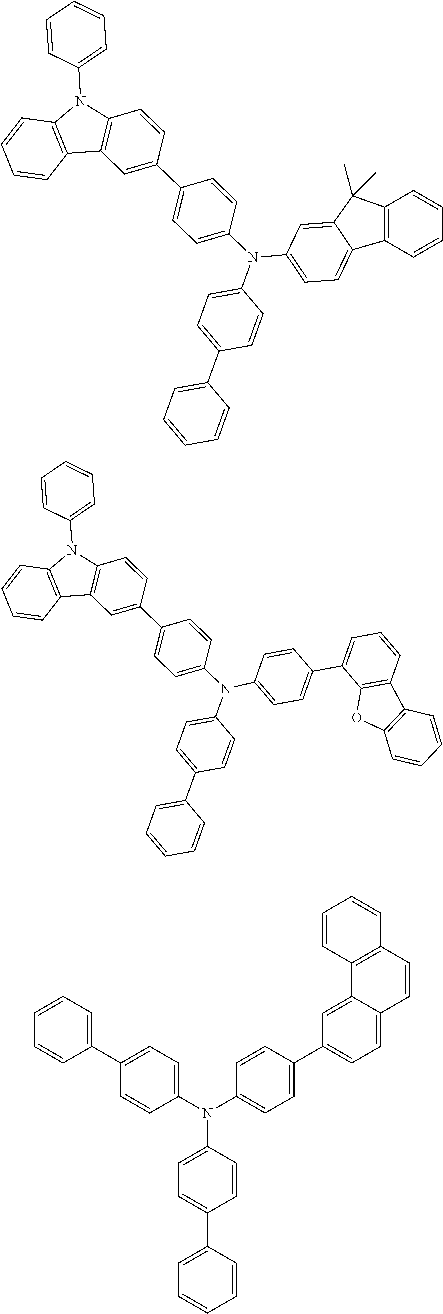 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

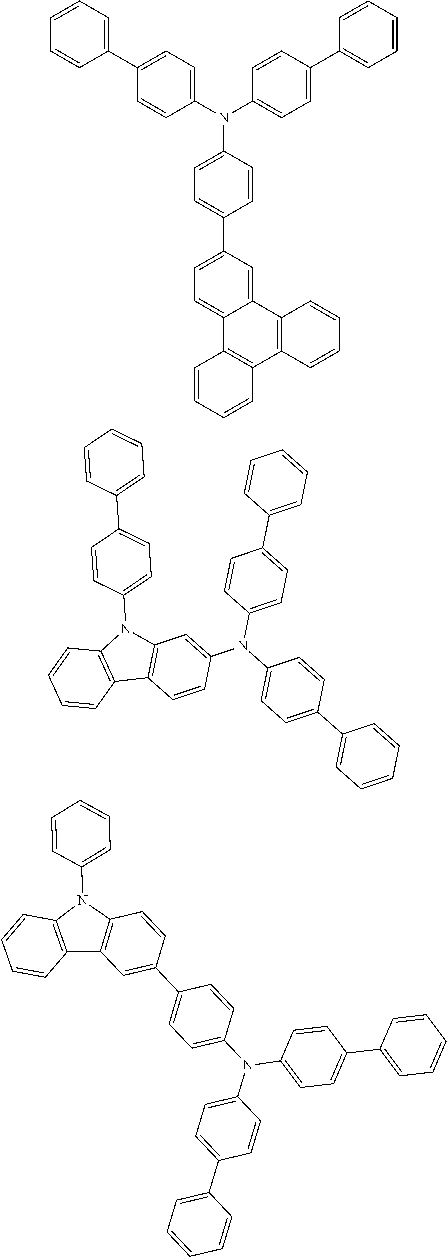 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

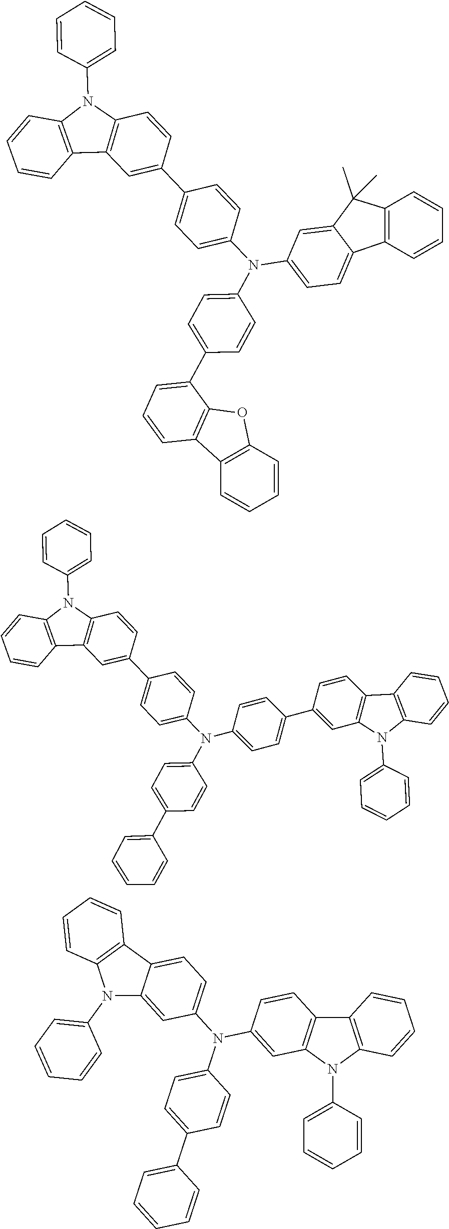

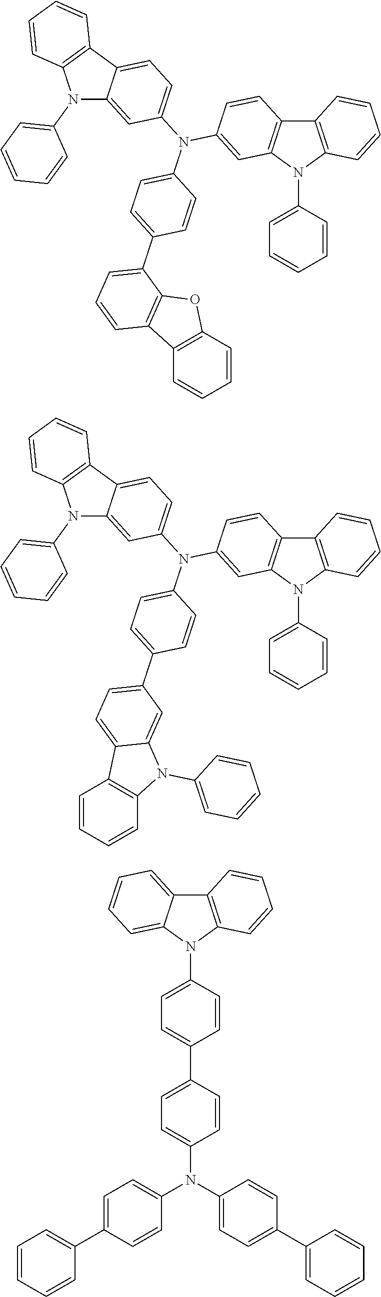

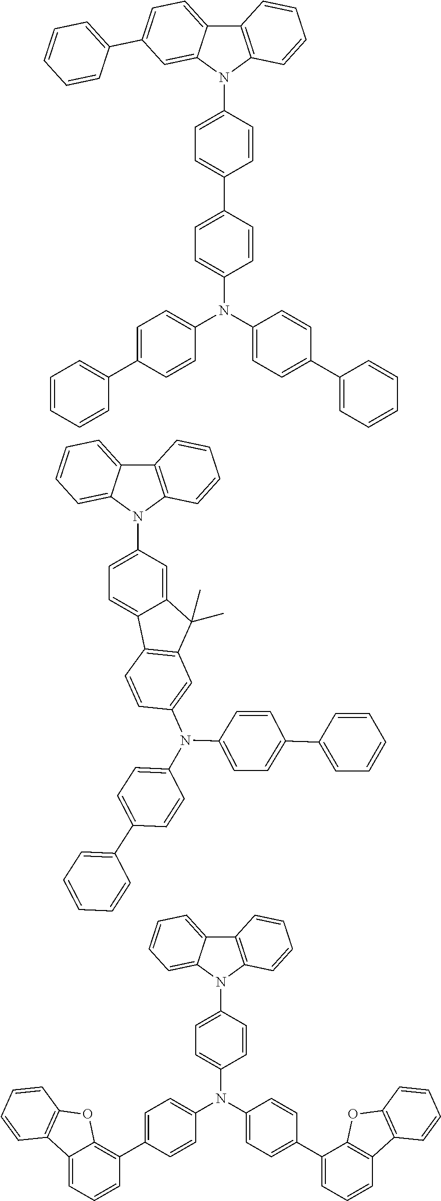

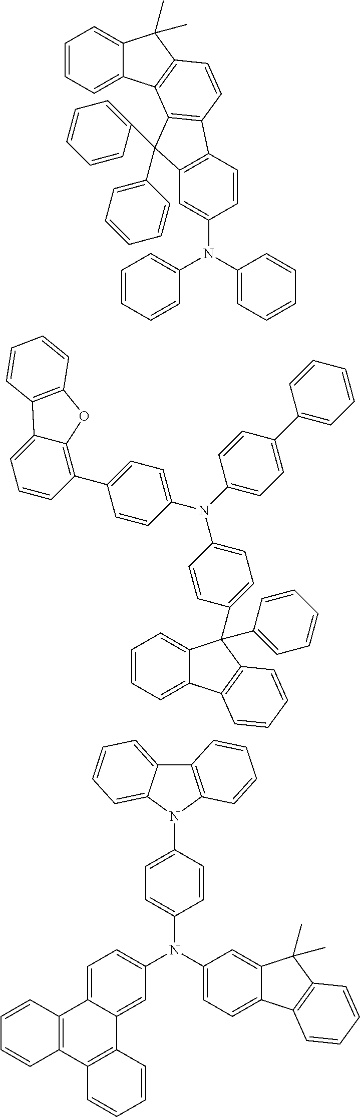

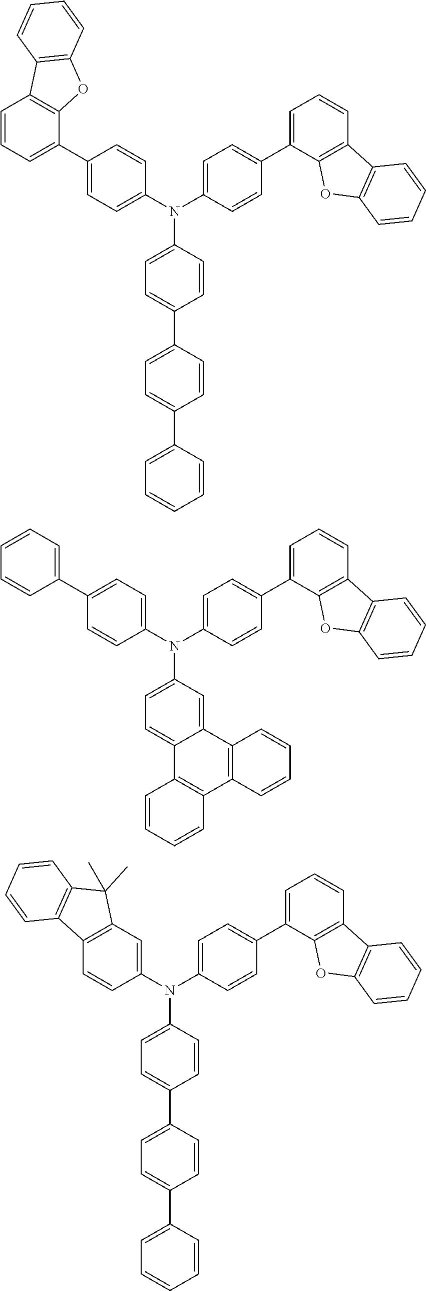

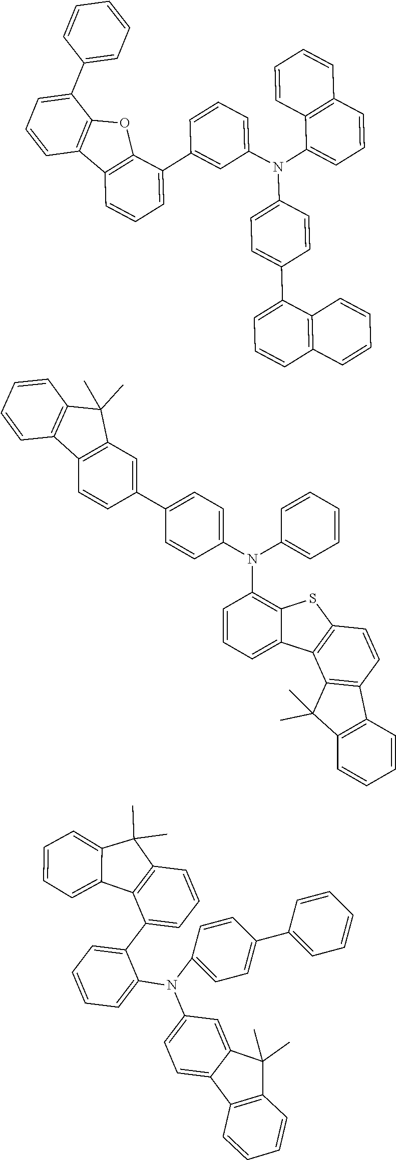 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

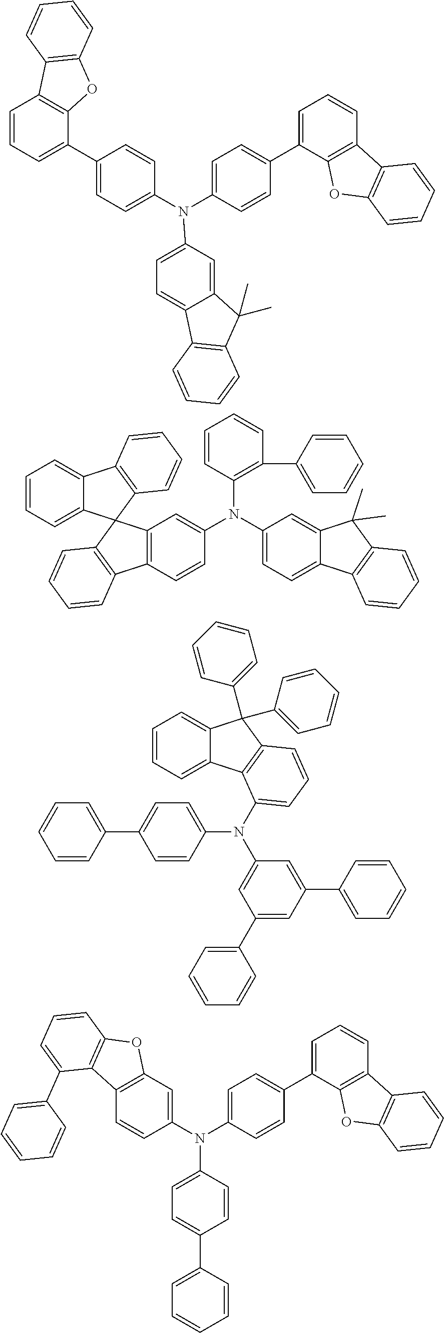

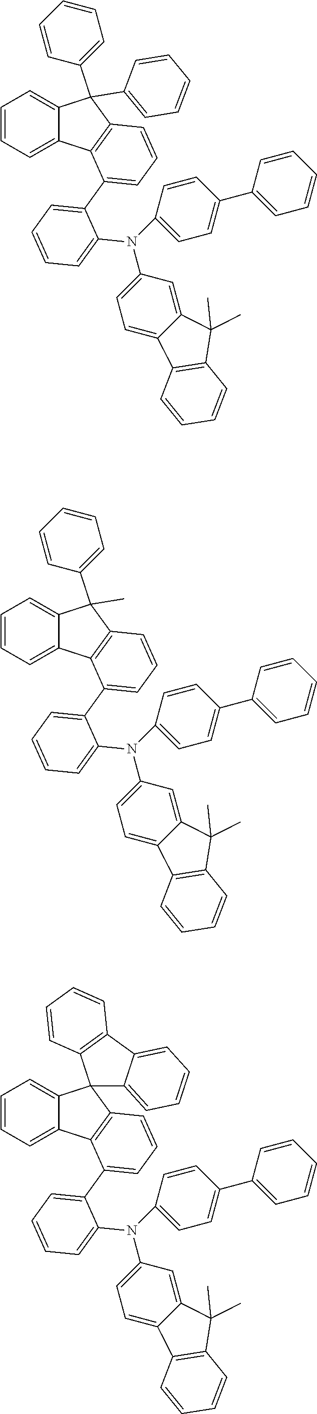 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

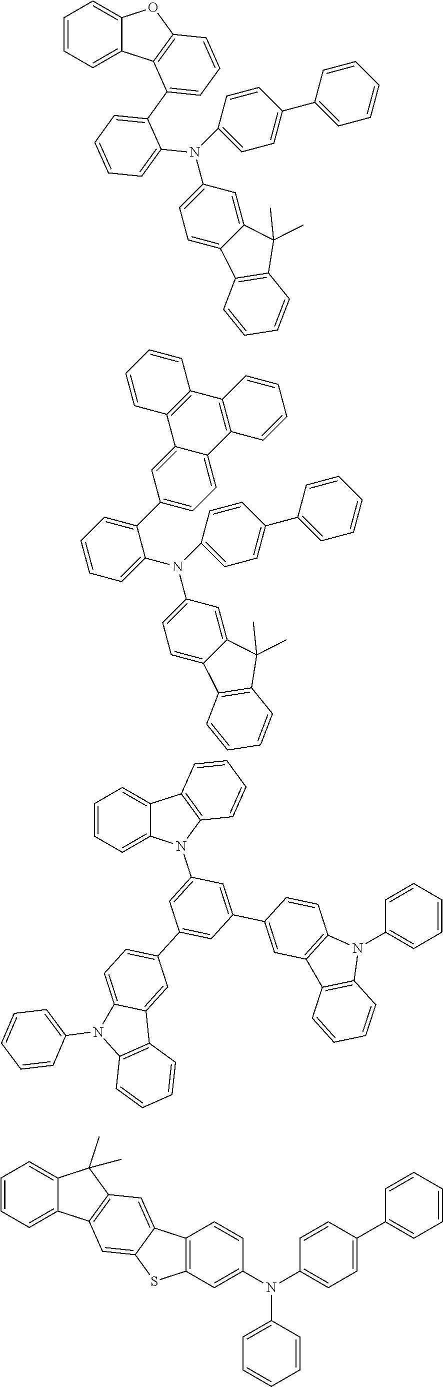

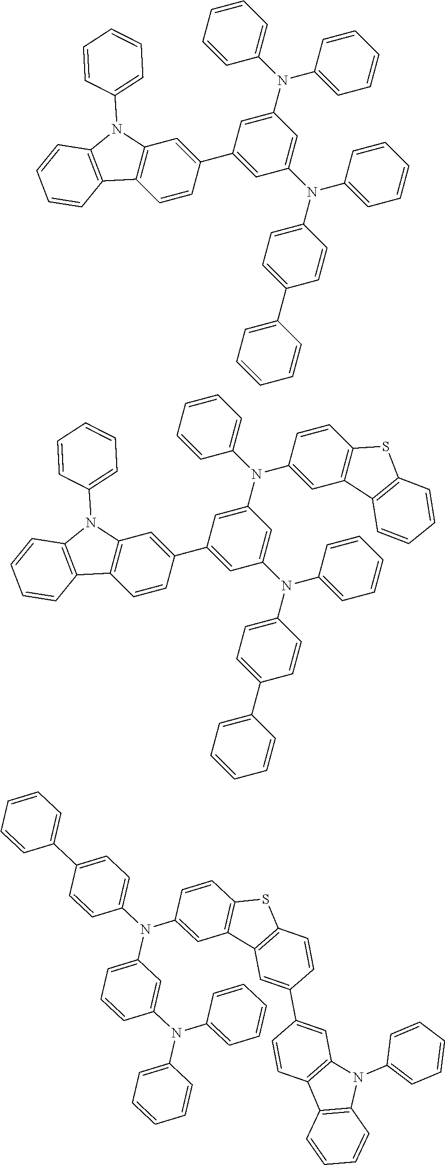

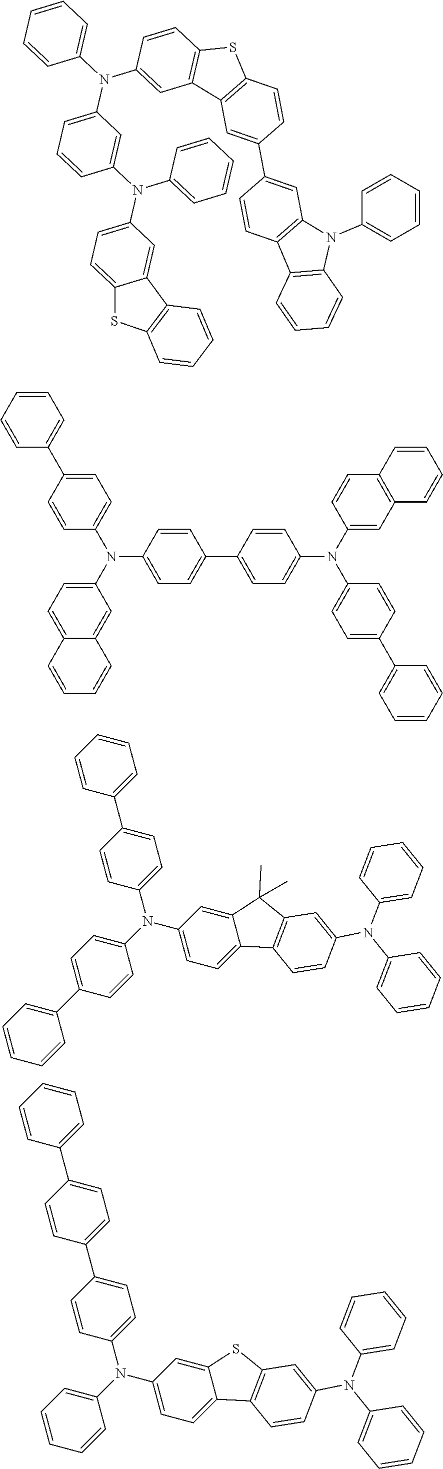

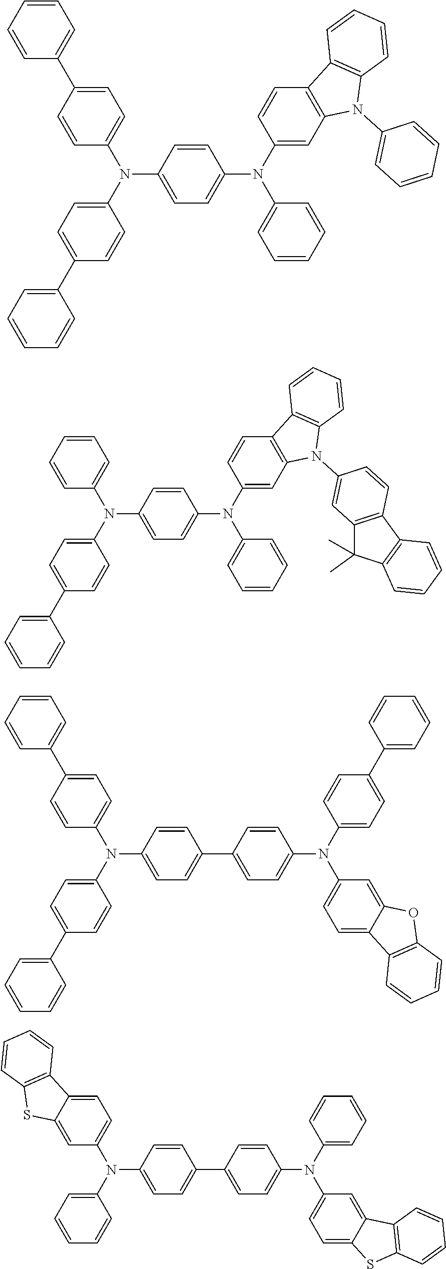

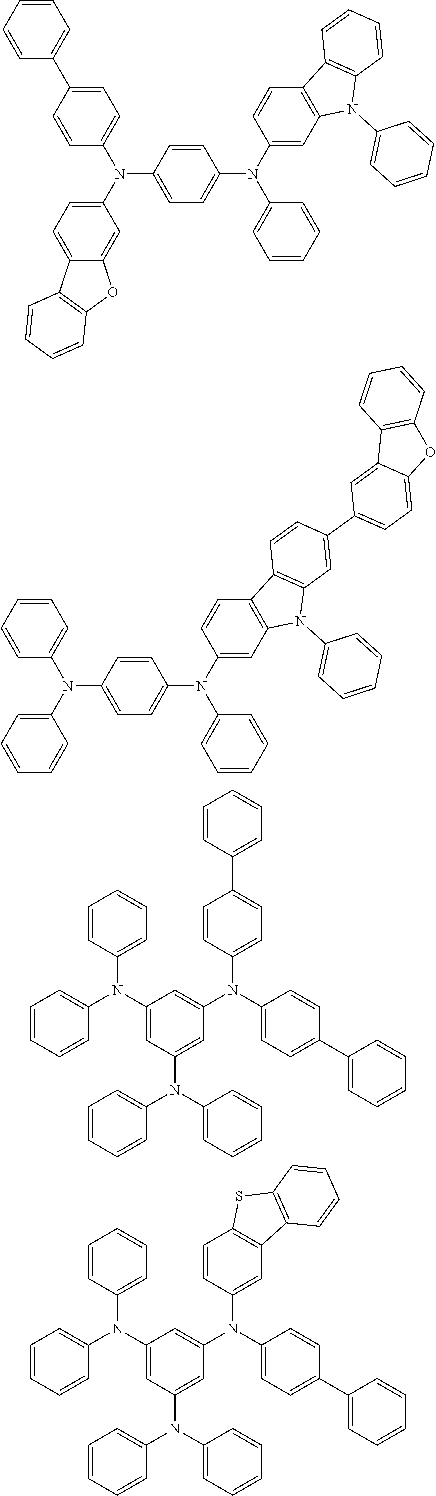

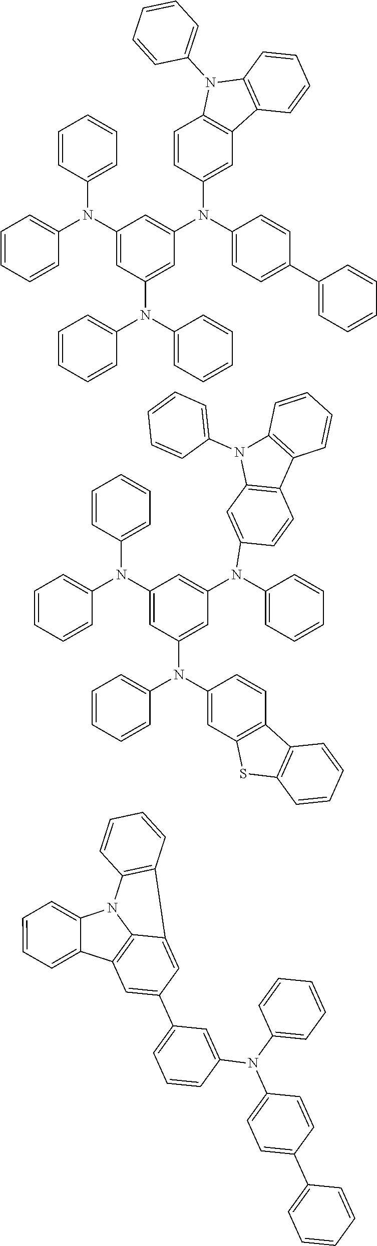

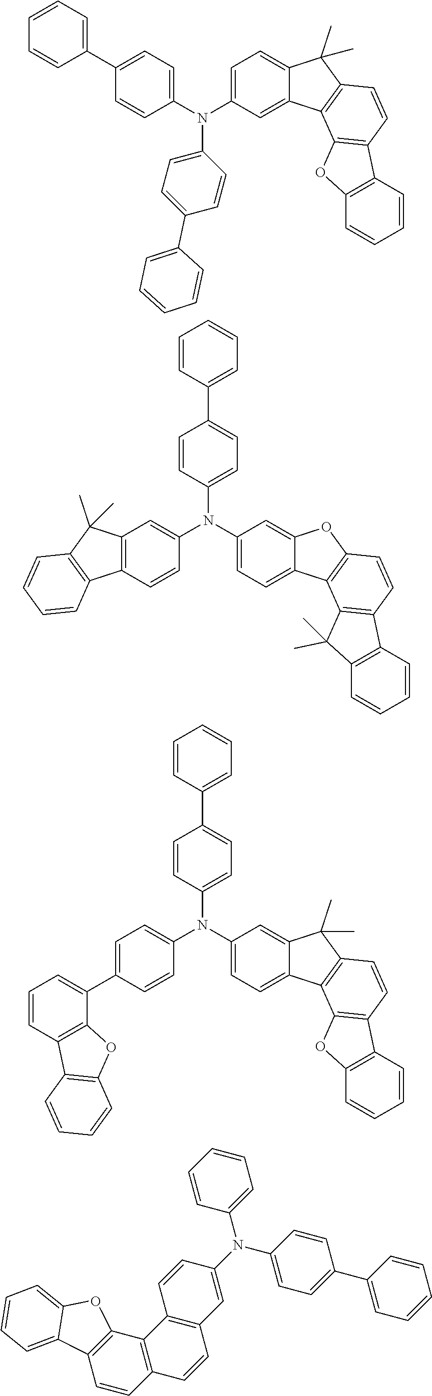

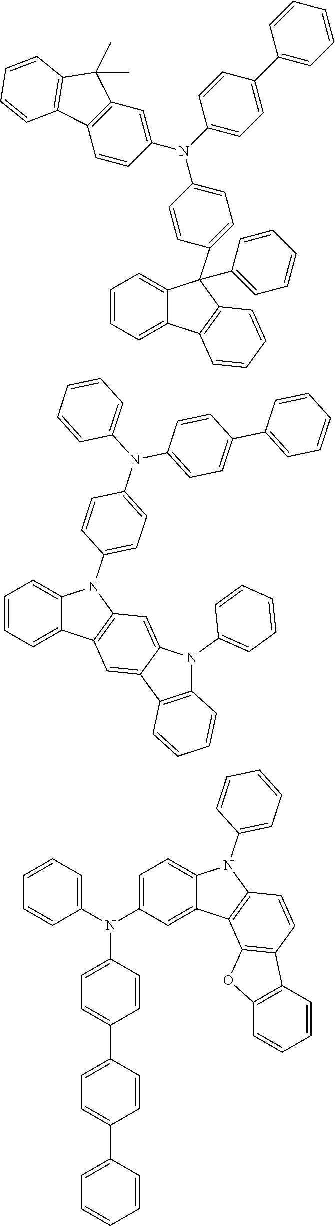

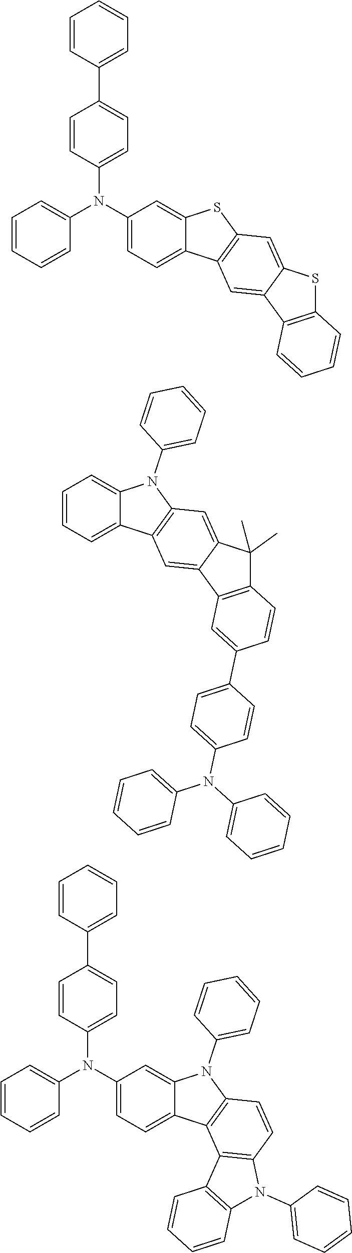 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

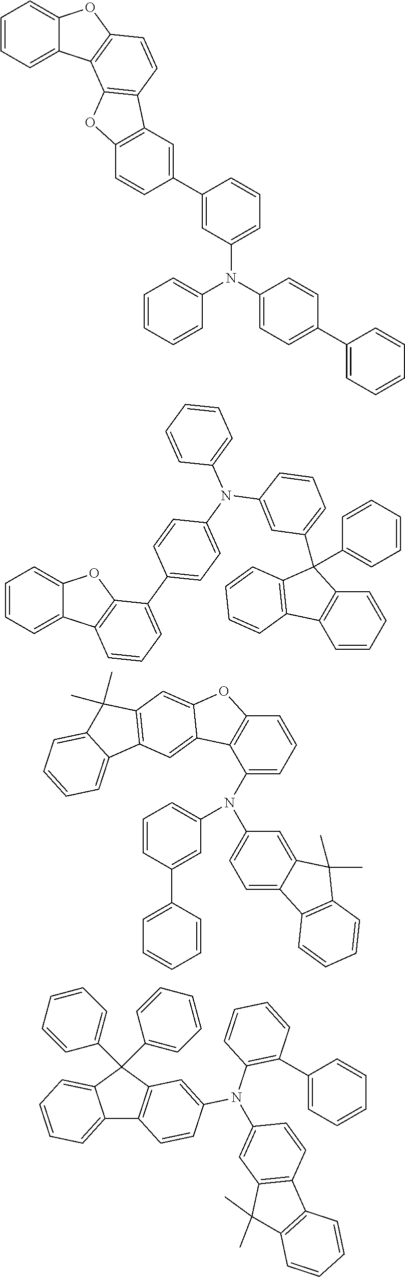

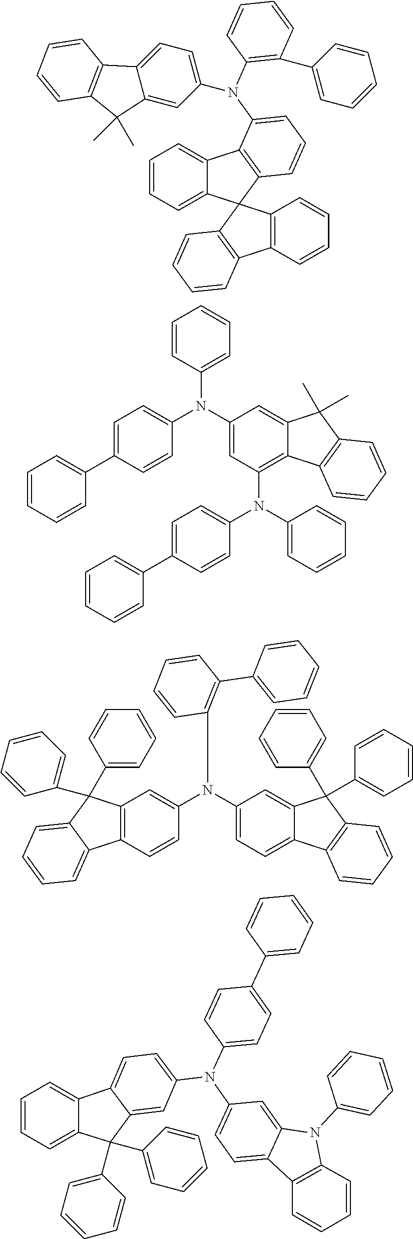

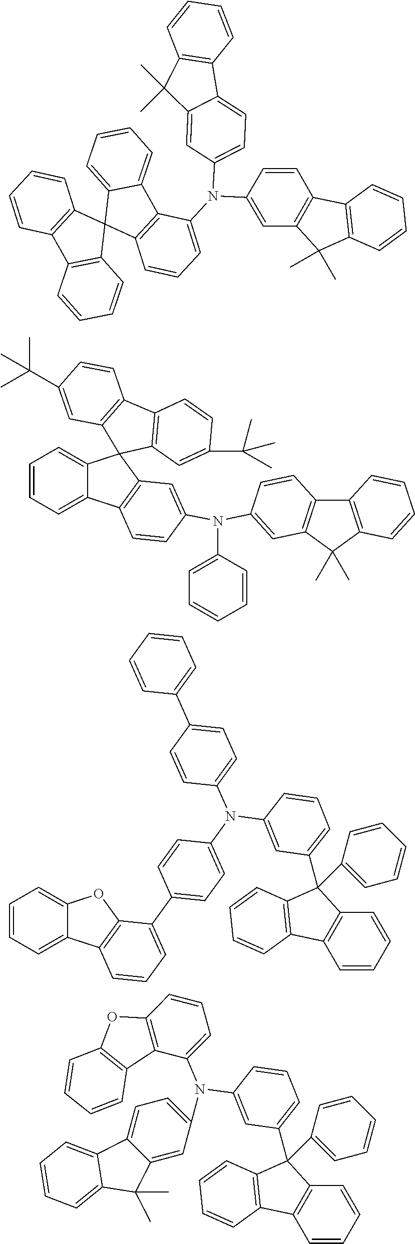

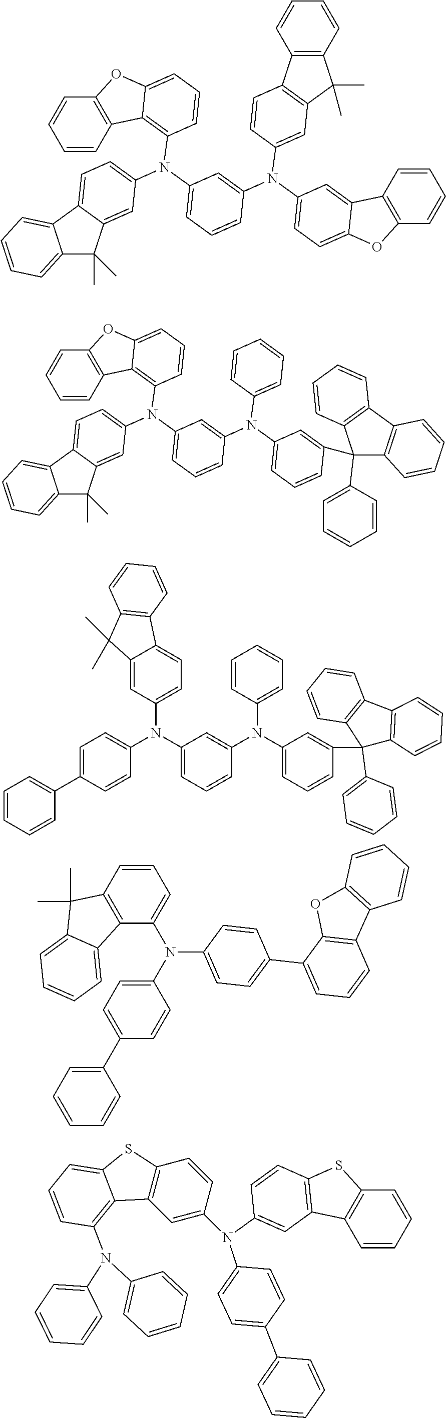

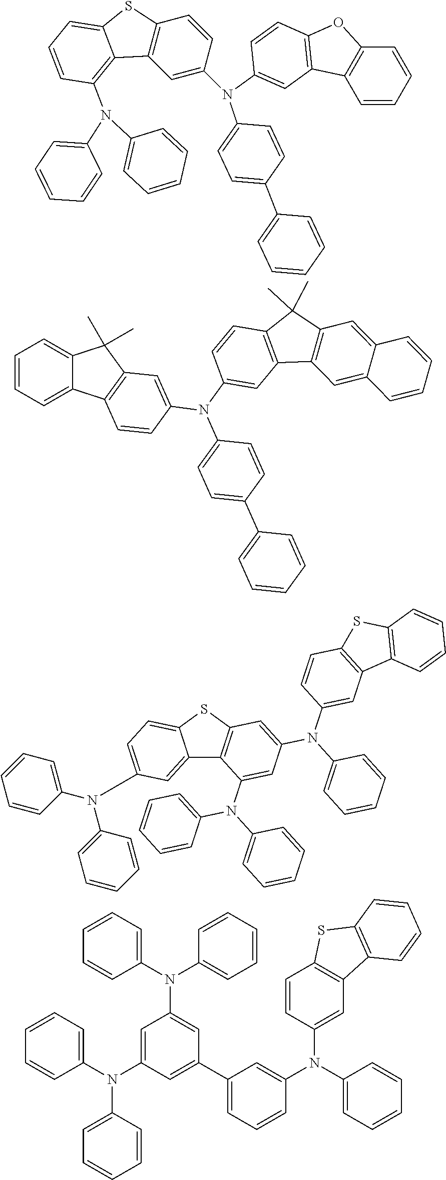

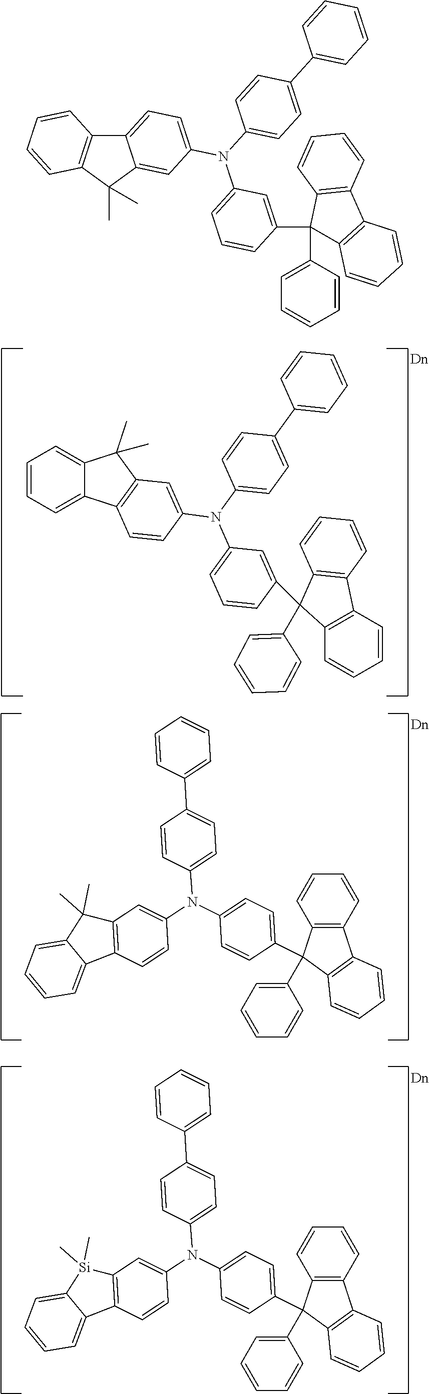

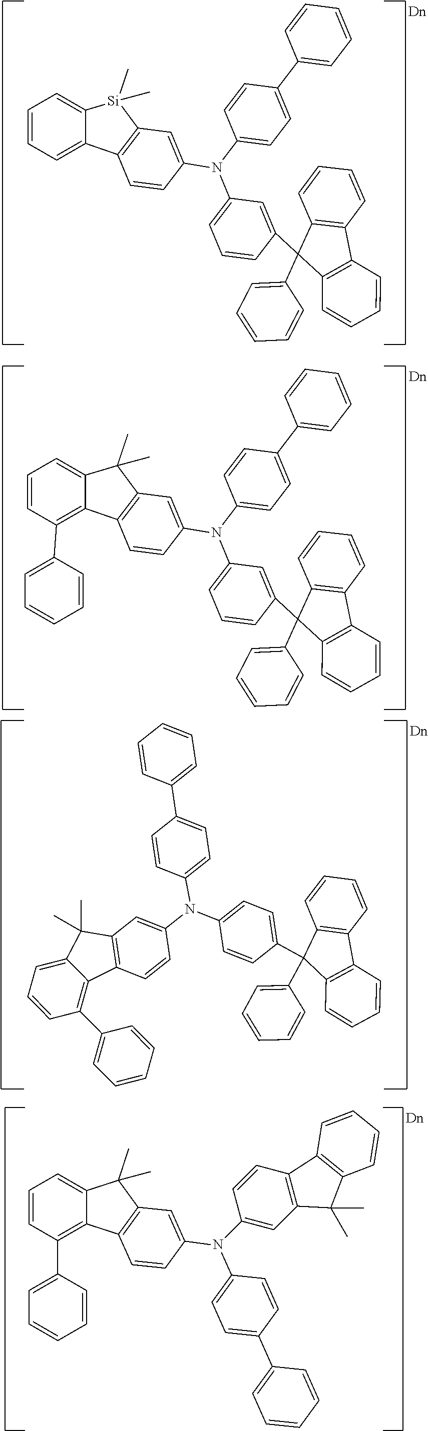

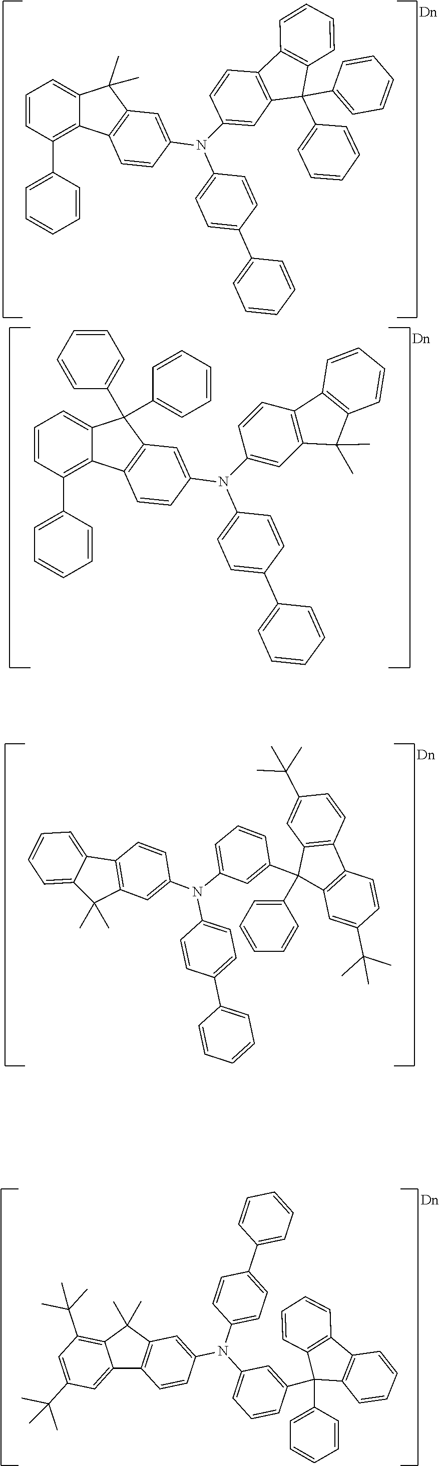

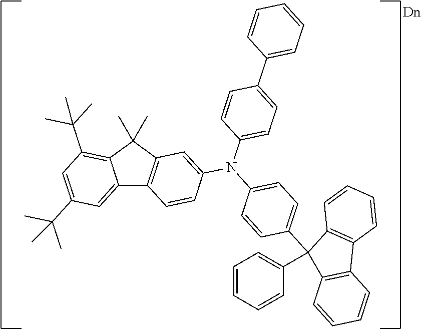

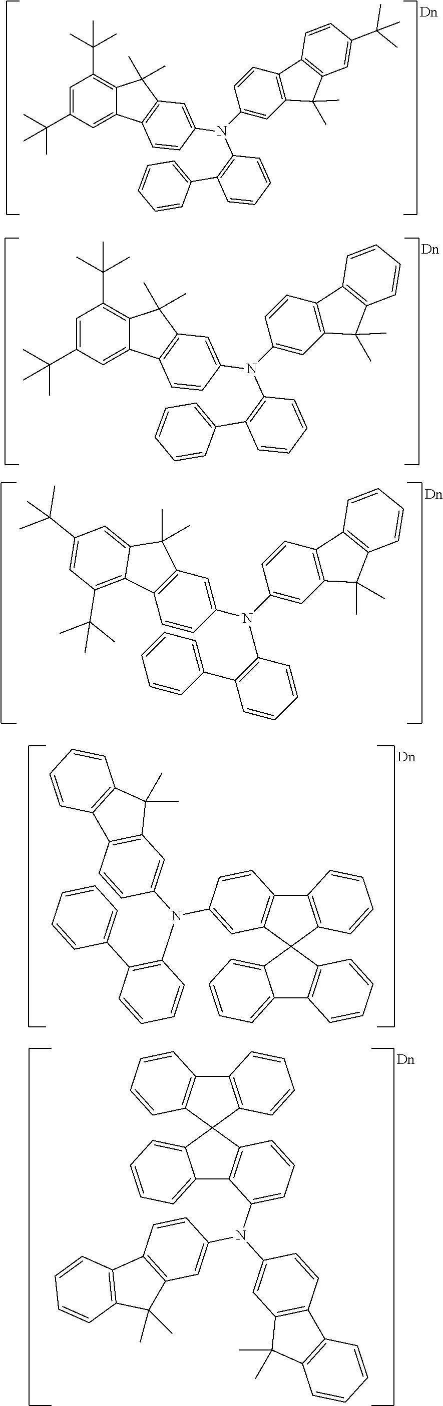

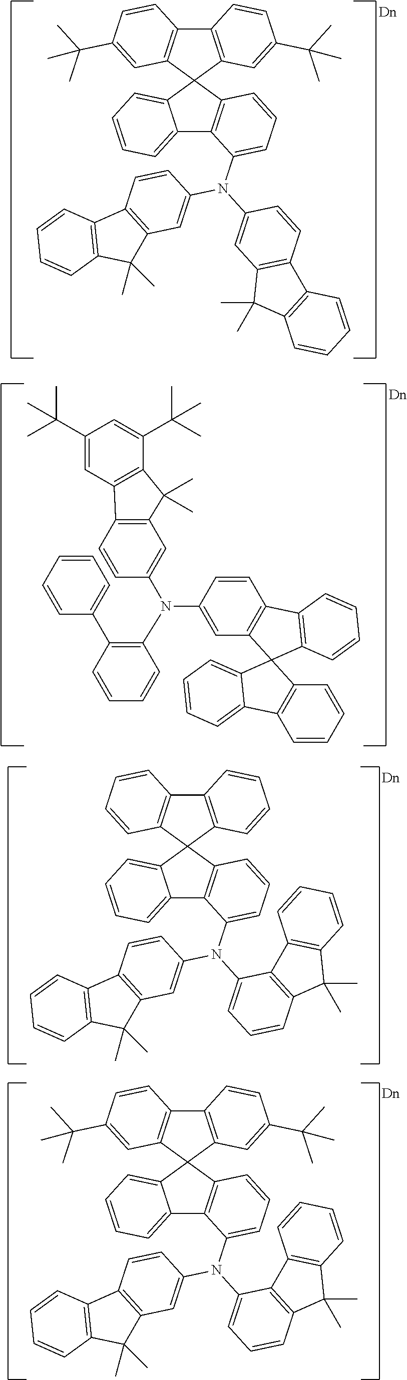

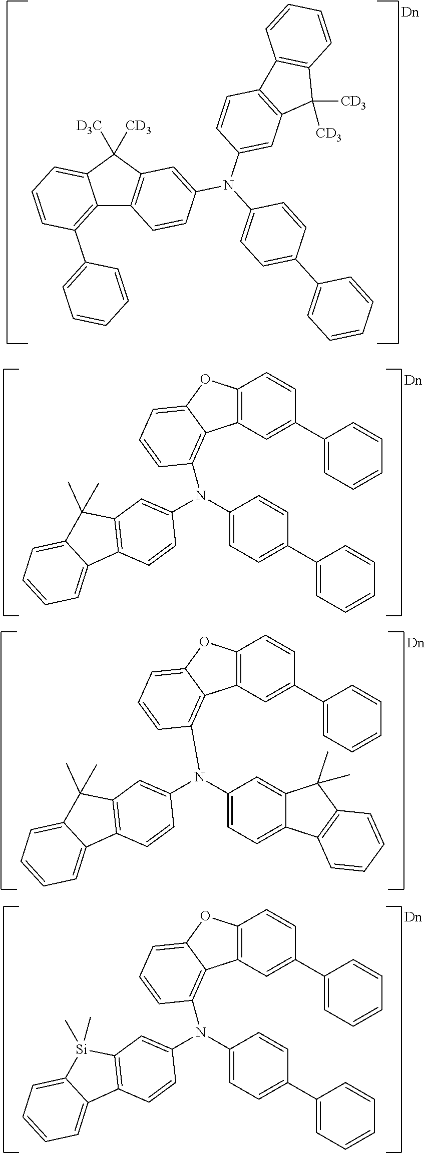

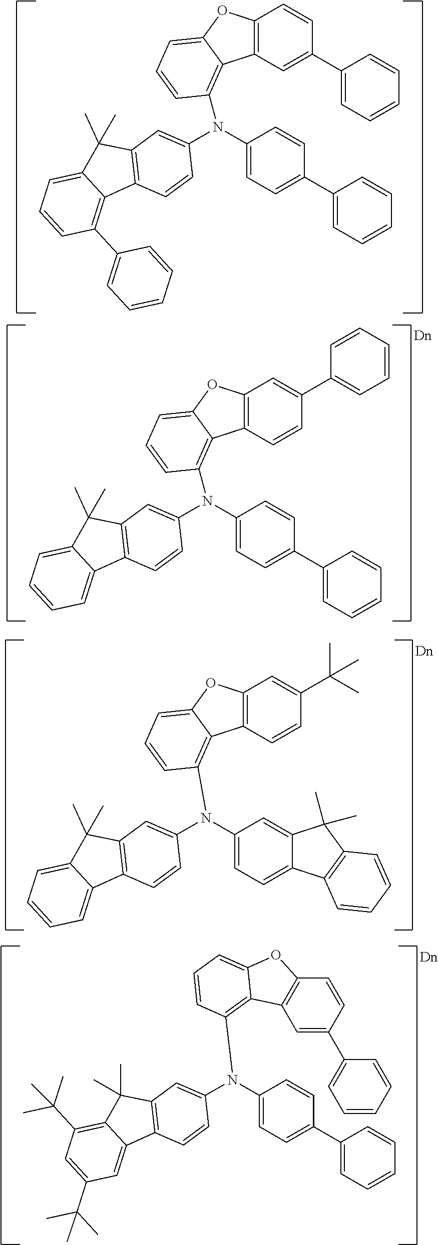

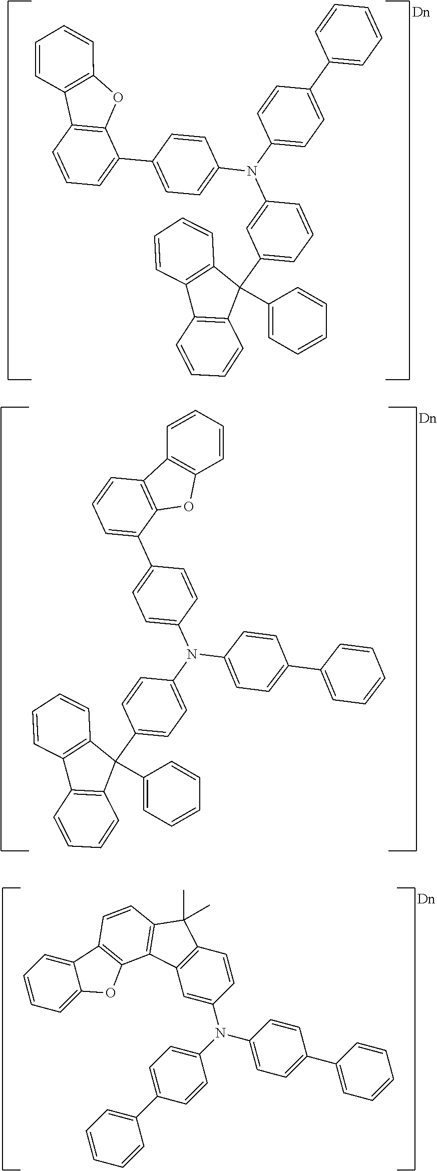

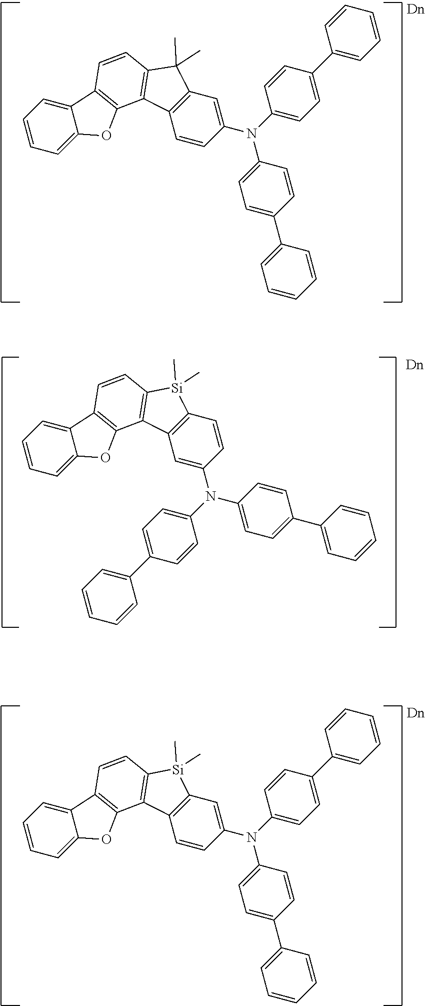

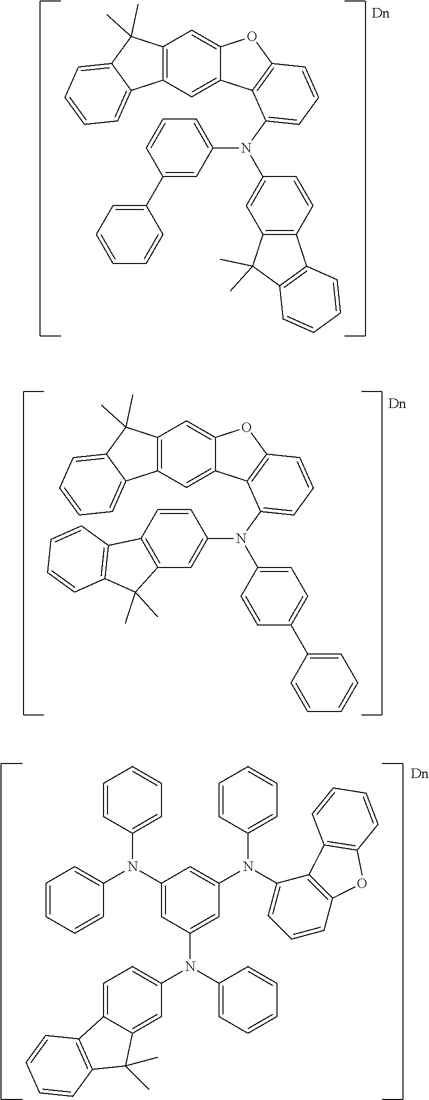

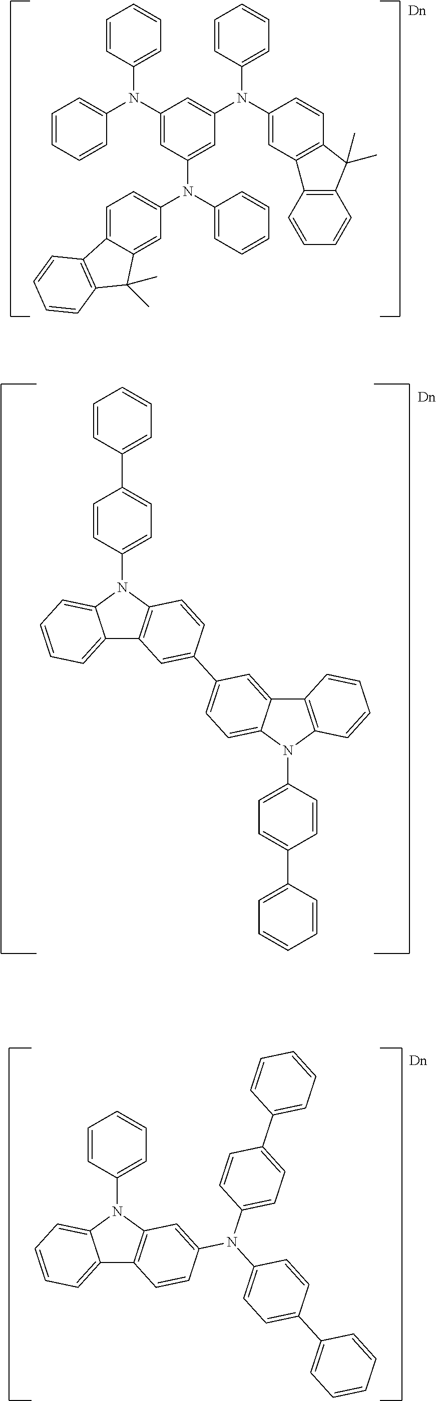

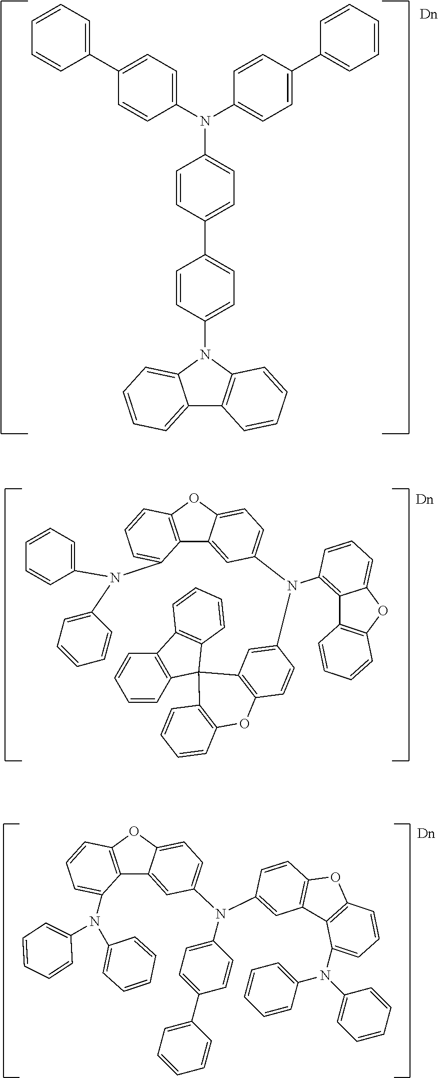

(Dn refers to the number of deuterium substitutions and indicates a structure substituted with one or more deuterium atoms)

In the hole transport region 140, in addition to the compounds described above, other suitable compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

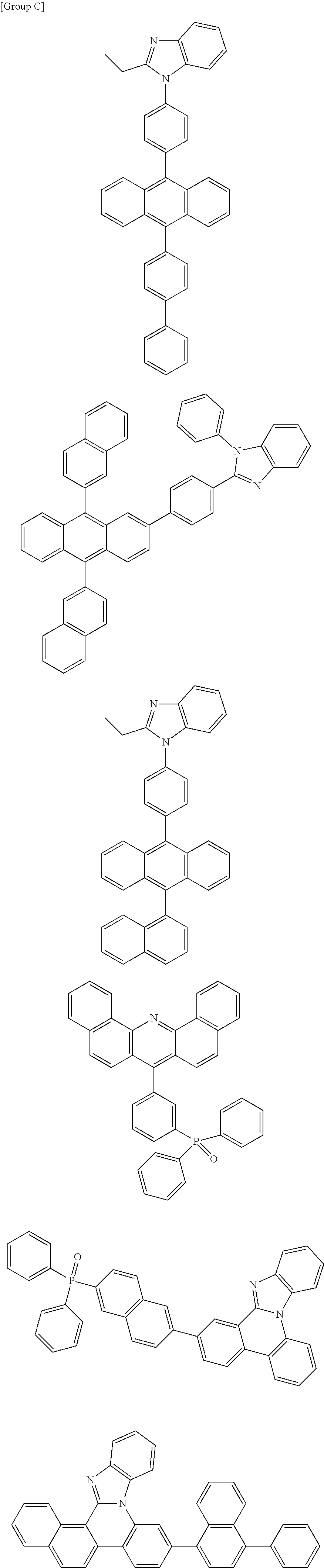

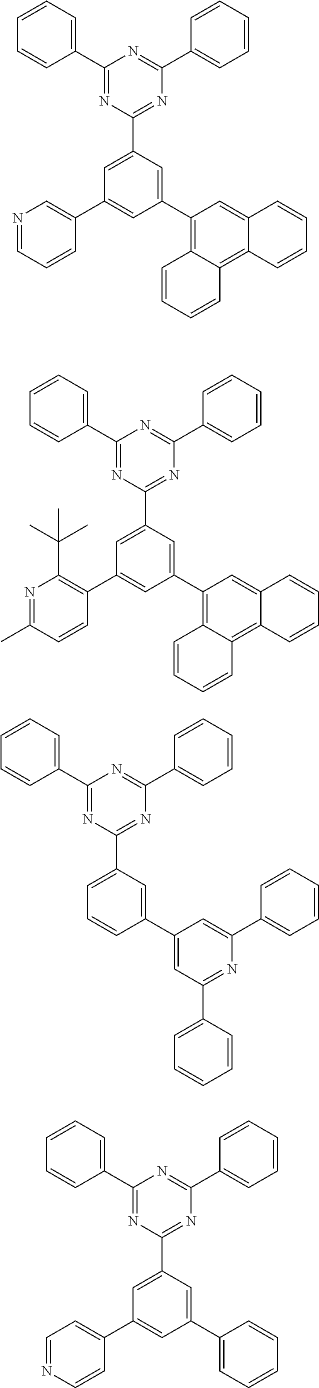

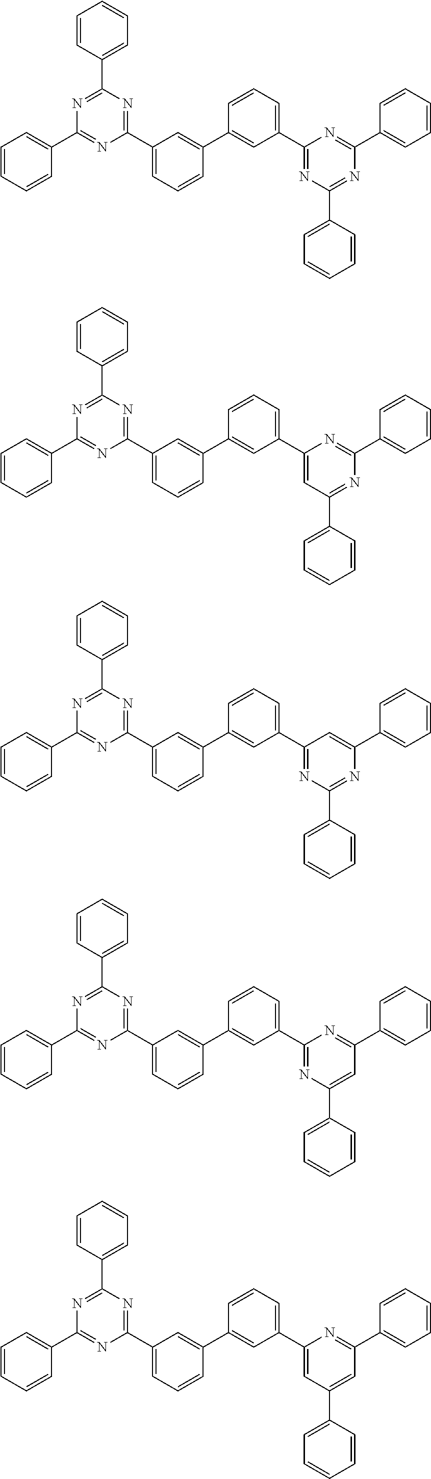 [SMILES: c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1]

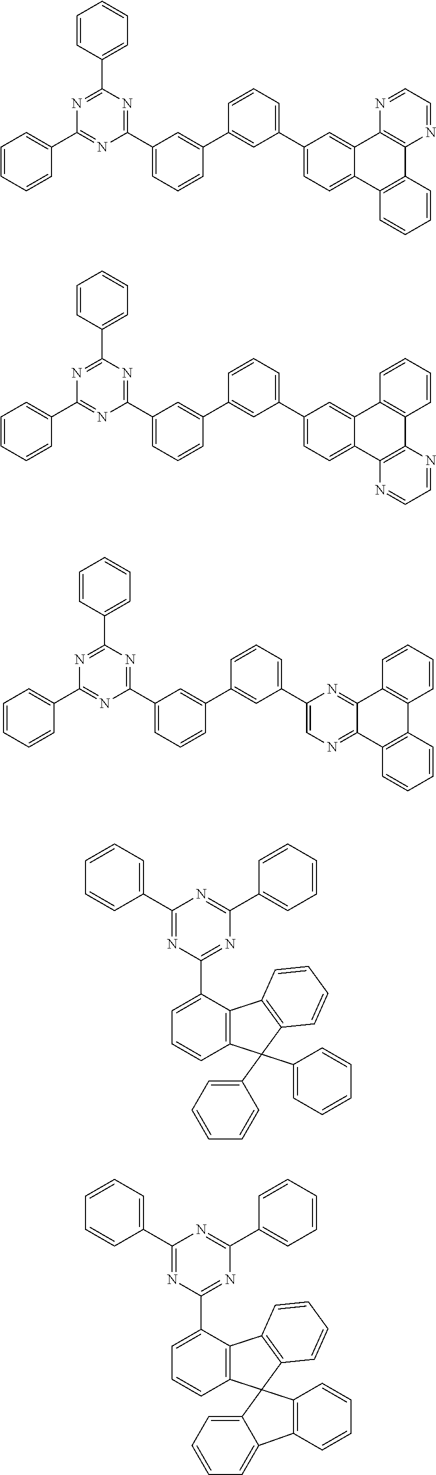 [SMILES: c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1]

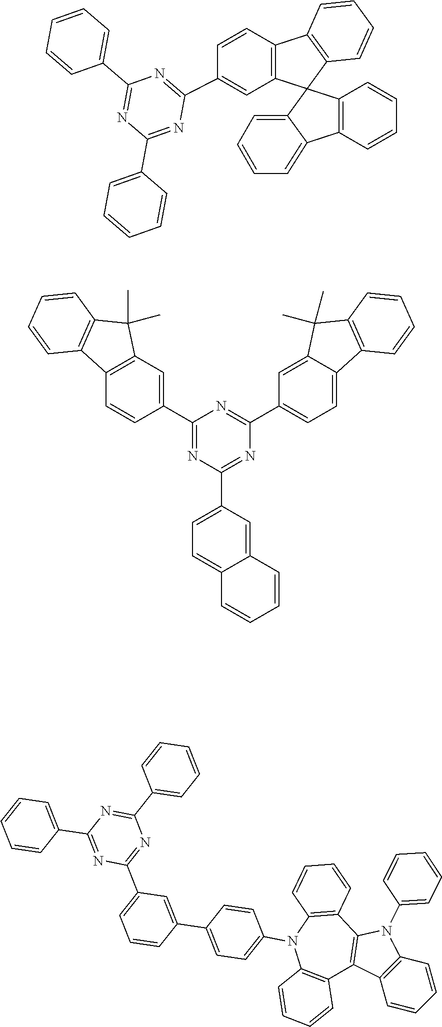 [SMILES: c1ccc(-c2nc(-c3ccccc3)nc(-c3ccc4c(c3)C3(c5ccccc5-c5ccccc53)c3ccccc3-4)n2)cc1 ; CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

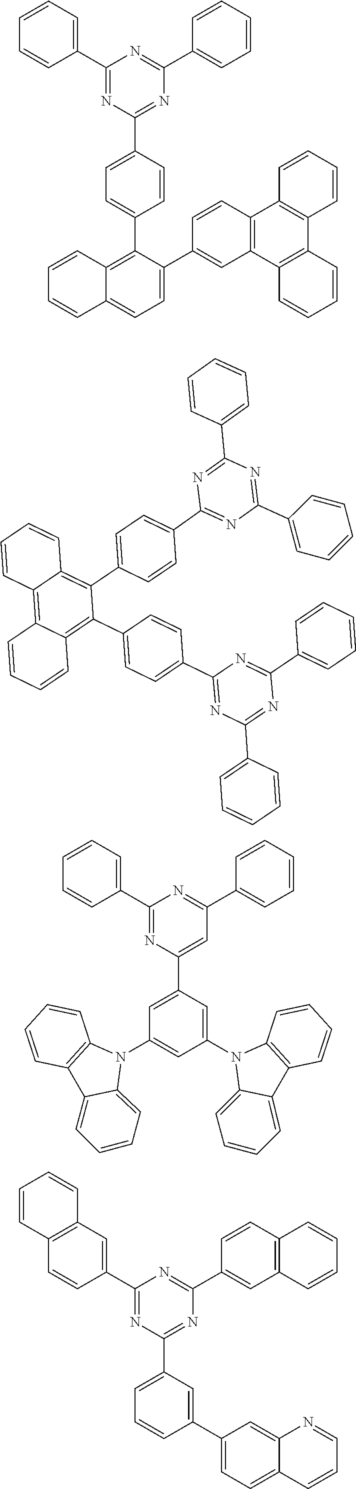

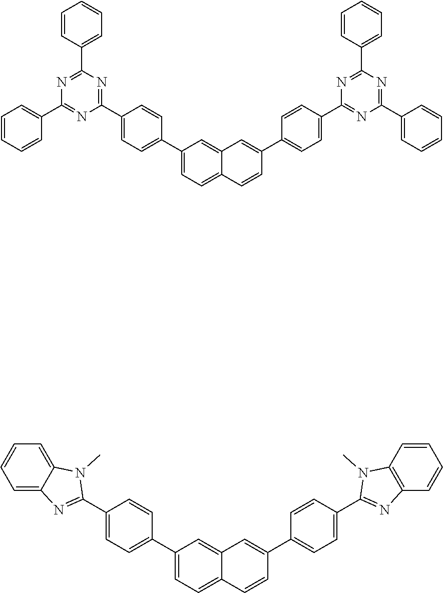 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21]

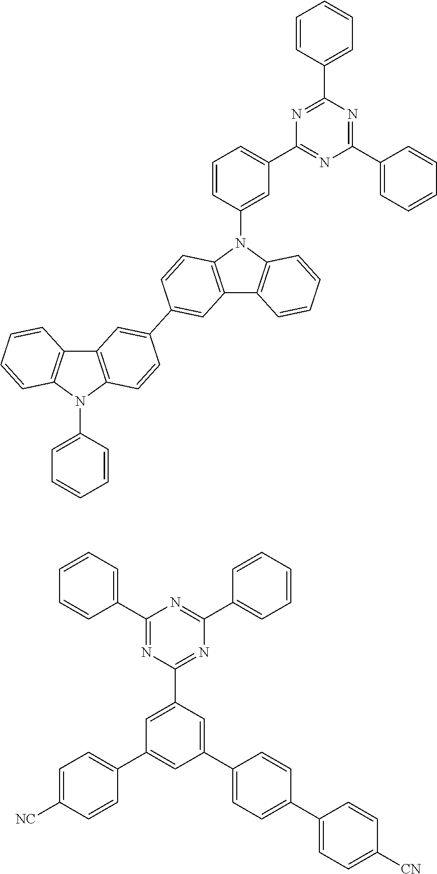 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1]

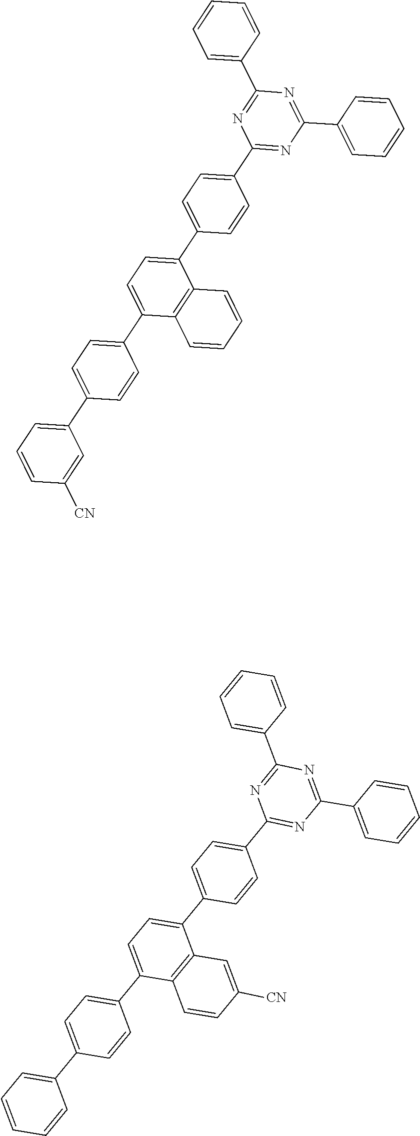 [SMILES: N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

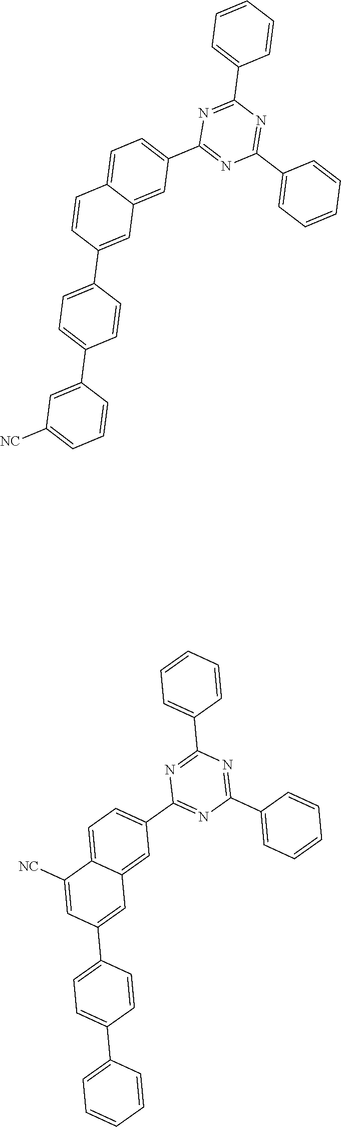 [SMILES: N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

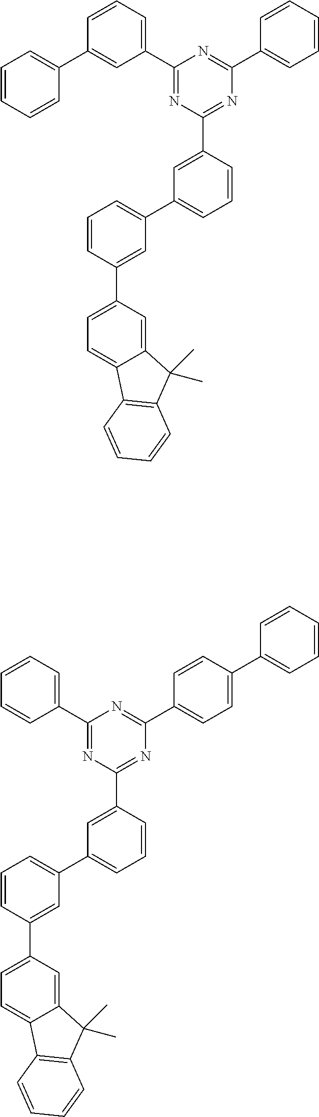 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

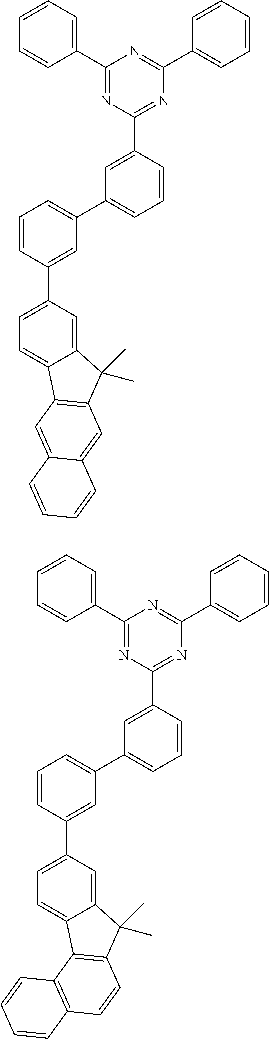

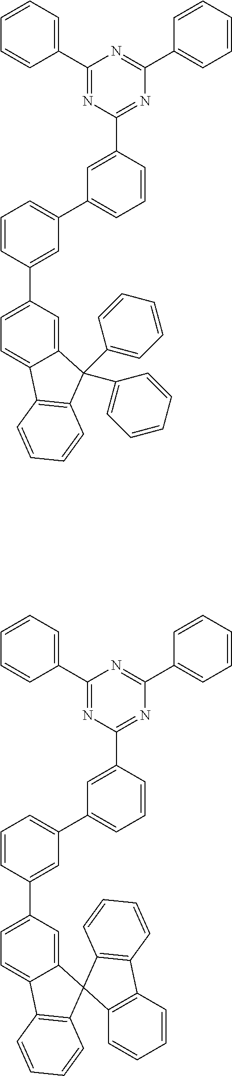 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1]

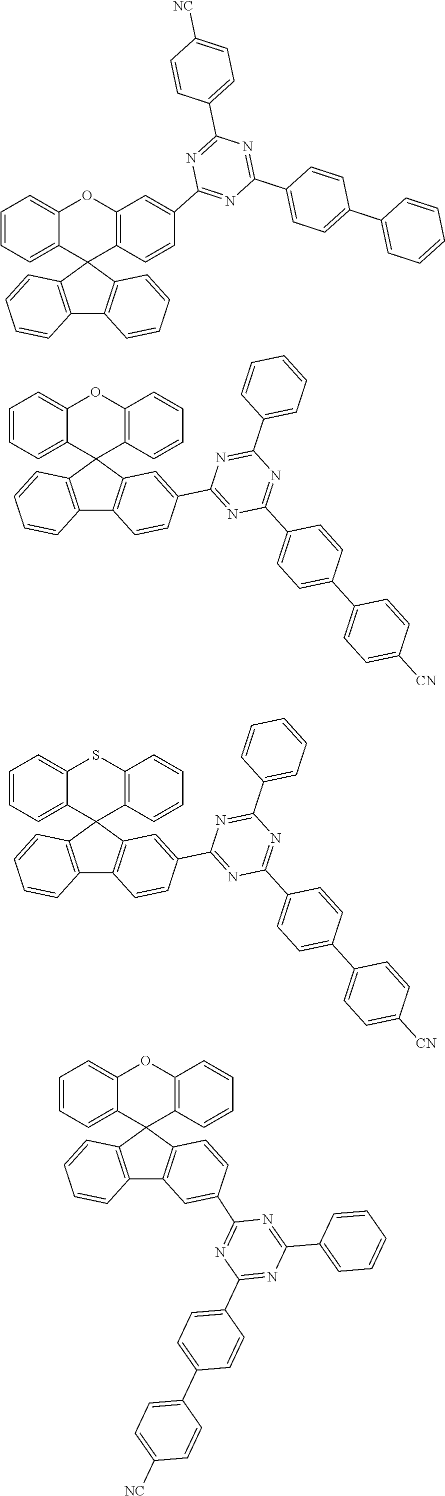 [SMILES: N#Cc1ccc(-c2nc(-c3ccc(-c4ccccc4)cc3)nc(-c3ccc4c(c3)Oc3ccccc3C43c4ccccc4-c4ccccc43)n2)cc1 ; N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1]

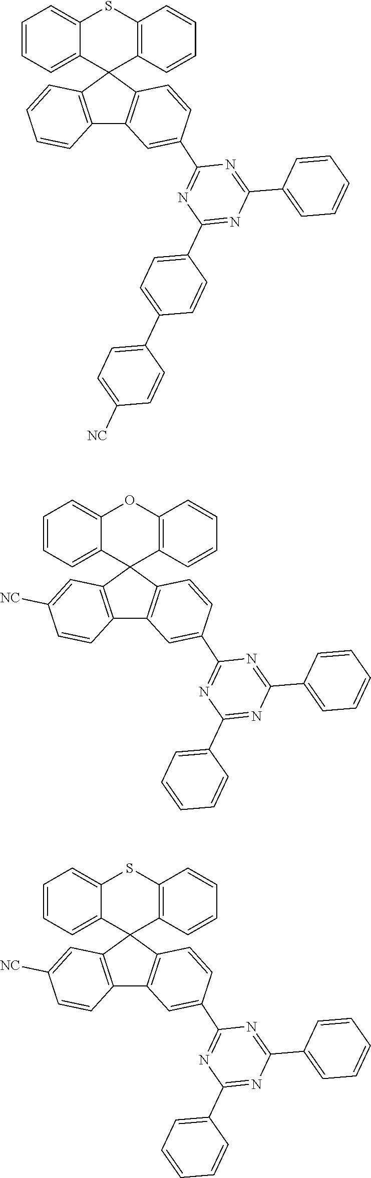 [SMILES: N#Cc1ccc(-c2ccc(-c3nc(-c4ccccc4)nc(-c4ccc5c(c4)-c4ccccc4C54c5ccccc5Sc5ccccc54)n3)cc2)cc1 ; N#Cc1ccc2c(c1)C1(c3ccccc3Oc3ccccc31)c1ccc(-c3nc(-c4ccccc4)nc(-c4ccccc4)n3)cc1-2 ; N#Cc1ccc2c(c1)C1(c3ccccc3Sc3ccccc31)c1ccc(-c3nc(-c4ccccc4)nc(-c4ccccc4)n3)cc1-2]

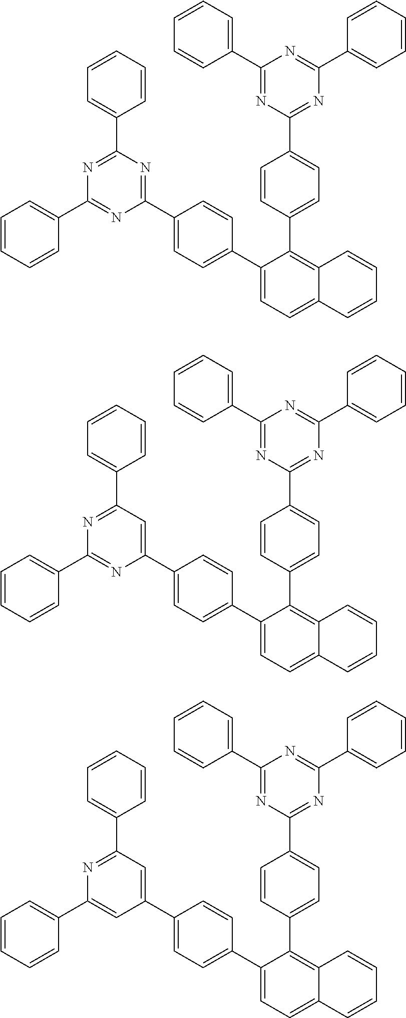

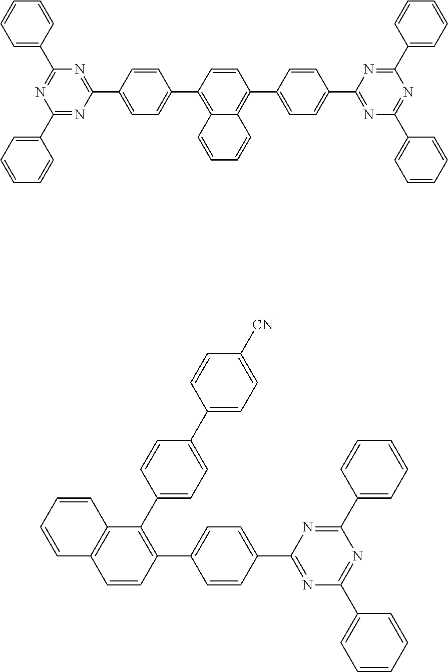 [SMILES: c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1 ; N#Cc1ccc(-c2ccc(-c3c(-c4ccc(-c5nc(-c6ccccc6)nc(-c6ccccc6)n5)cc4)ccc4ccccc34)cc2)cc1]

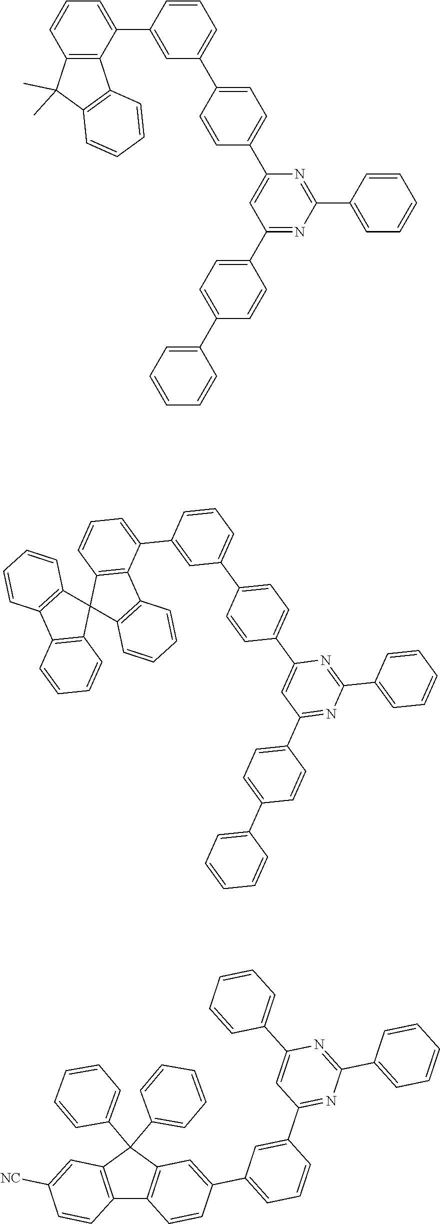 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2]

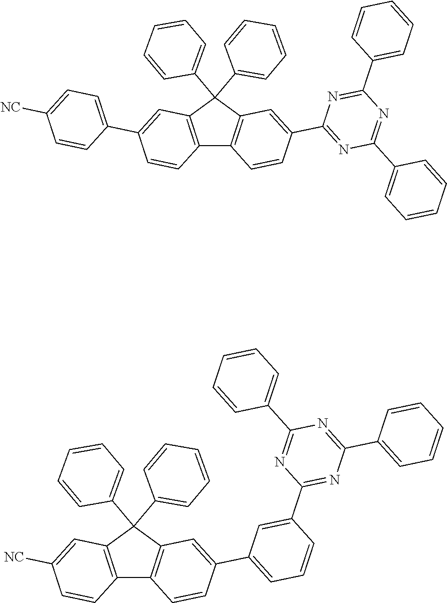 [SMILES: N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

Some example embodiments may provide an organic light emitting diode including the light emitting layer as the organic layer.

Some example embodiments may provide an organic light emitting diode including a light emitting layer and a hole transport region as the organic layer.

Some example embodiments may provide an organic light emitting diode including a light emitting layer and an electron transport region as the organic layer.

An organic light emitting diode according to some example embodiments includes a hole transport region 140 and an electron transport region 150 in addition to the light emitting layer 130 as the organic layer 105, as shown in the FIGURE.

In an implementation, an organic light emitting diode may further include an electron injection layer, a hole injection layer, or the like, in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment, or they were synthesized by suitable methods.

### (Synthesis of Compound for Organic Optoelectronic Device)

### Synthesis Example 1: Synthesis of Compound A-1

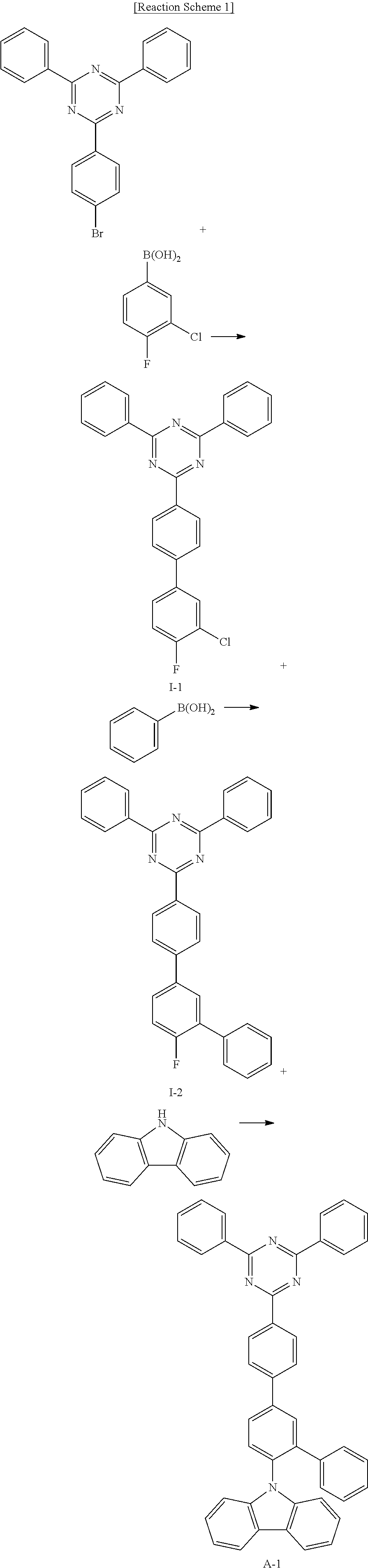

### 1st Step: Synthesis of Intermediate I-1

2-(4-bromophenyl)-4,6-diphenyl-1,3,5-triazine (25.5 g, 65.7 mmol), (3-chloro-4-fluorophenyl)boronic acid (12.0 g, 69.0 mmol), K₂CO₃ (18.2 g, 131.4 mmol), and Pd(PPh₃)₄ (3.8 g, 3.3 mmol) were added to a round-bottomed flask and dissolved in Tetrahydrofuran (THF) (300 ml) and distilled water (70 ml) and then, stirred under reflux at 60° C. for 12 hours. When a reaction was completed, the resultant was cooled to ambient temperature, and an excess amount of methanol was poured thereinto to precipitate a solid, which was filtered and gathered. The solid was dissolved by heating in monochlorobenzene (MCB) and then filtered through a silica pad to collect a filtrate, which was recrystallized to obtain 20.7 g (72%) of Intermediate I-1.

### 2nd Step: Synthesis of Intermediate I-2

Intermediate I-1 (20.7 g, 47.3 mmol), phenylboronic acid (5.8 g, 47.3 mmol), Cs₂CO₃ (23.1 g, 70.9 mmol), tri-tert-butylphosphine (3.8 g, 9.5 mmol), and Pd₂(dba)₃ (1.3 g, 1.4 mmol) were added to a round-bottomed flask and dissolved in 1,4-dioxane (250 ml) and then, stirred under reflux at 120° C. for 8 hours. When a reaction was completed, the resultant was cooled to ambient temperature, and an excess amount of distilled water was poured thereinto to precipitate a solid. The precipitated solid was filtered and gathered. The solid was dissolved by heating in MCB and then passed through a silica pad to collect a filtrate, which was recrystallized to obtain 13.8 g (61%) of Intermediate I-2.

### 3rd Step: Synthesis of Compound A-1

Intermediate I-2 (13.8 g, 28.8 mmol), 9H-carbazole (6.5 g, 38.8 mmol), and K₃PO₄ (12.2 g, 57.6 mmol) were added to a round-bottomed flask and dissolved in DMF (Dimethylformamide) (100 m1) and then, stirred under reflux at 150° C. for 8 hours. When a reaction was completed, the resultant was slowly poured into an excess amount of water to precipitate a solid and then, stirred for 30 minutes. The precipitated solid was filtered and added to MCB to dissolve it by heating and then filtered through a silica pad. A filtrate therefrom was distilled under a reduced pressure to precipitate the solid, obtaining 15.5 g (86%) of Compound A-1.

### Synthesis Example 2: Synthesis of Compound A-40

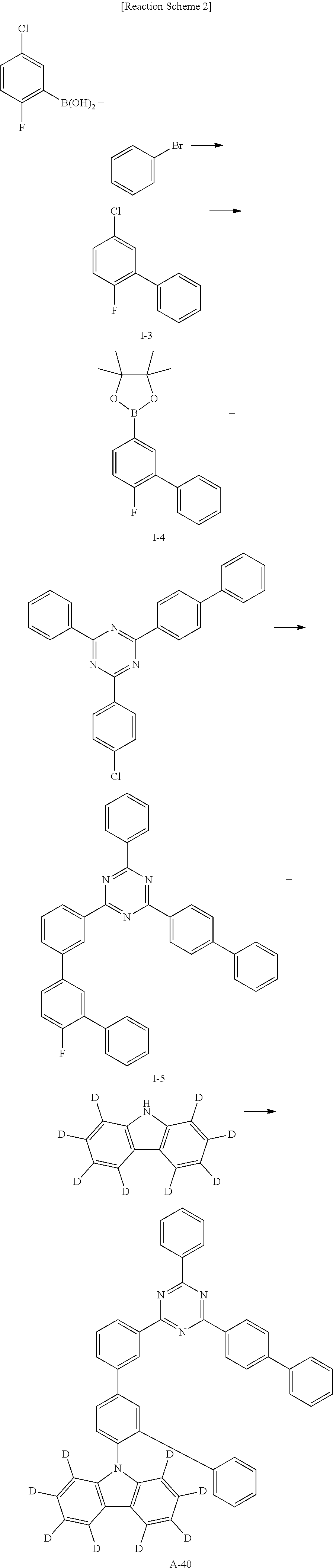

### 1 st Step: Synthesis of Intermediate I-3

(5-chloro-2-fluorophenyl)boronic acid (20.0 g, 114.7 mmol), bromobenzene (21.6 g, 137.6 mmol), K₂CO₃ (31.7 g, 229.4 mmol), and Pd(PPh₃)₄ (6.6 g, 5.7 mmol) were added to a round-bottomed flask and dissolved in THF (450 ml) and distilled water (120 ml) and then, stirred under reflux at 60° C. for 12 hours. When a reaction was completed, the resultant was cooled to ambient temperature and passed through a separatory funnel to separate an aqueous layer and an organic layer. The organic solvent was removed under a reduced pressure and treated through column chromatography (hexane:DCM (10% to 20%) to obtain 16.1 g (68%) of Intermediate I-3.

### 2nd Step: Synthesis of Intermediate I-4

Intermediate I-3 (16.1 g, 77.9 mmol), bis(pinacolato)diboron (24.7 g, 97.4 mmol), tricyclohexylphosphine (4.4 g, 15.6 mmol), potassium acetate (15.3 g, 155.8 mmol), and Pd(dppf)Cl₂ (2.9 g, 3.9 mmol) were added to a round-bottomed flask and dissolved in xylene (350 ml). The mixture was stirred at 120° C. for 8 hours. When a reaction was completed, the resultant was cooled to ambient temperature and filtered to remove a salt, and an excess amount of dichloromethane (DCM) and distilled water were added thereto for extraction. 16.3 g (70%) of Intermediate I-4 was obtained through column chromatography (hexane:DCM (30% to 50%)).

### 3rd Step: Synthesis of Intermediate I-5

2-([1,1′-biphenyl]-4-yl)-4-(3-chlorophenyl)-6-phenyl-1,3,5-triazine (19.1 g, 45.5 mmol), Intermediate I-4 (16.3 g, 54.6 mmol), K₂CO₃ (12.6 g, 91.0 mmol), and Pd(PPh₃)₄ (2.6 g, 2.3 mmol) were added to a round-bottomed flask and dissolved in THF (200 ml) and distilled water (50 ml) and then, stirred under reflux at 70° C. for 12 hours. When a reaction was completed, the resultant was cooled to ambient temperature, and an excess amount of water was added to precipitate a solid. The precipitated solid was filtered and gathered. The solid was dissolved by heating in MCB and then passed through a silica pad to collect a filtrate, which was recrystallized to obtain 22.7 g (90%) of Intermediate I-5.

### 4th Step: Synthesis of Compound A-40

Intermediate I-5 (12.7 g, 22.9 mmol), 9H-carbazole-1,2,3,4,5,6,7,8-d₈ (5.2 g, 29.7 mmol), and K₃PO₄ (9.7 g, 45.7 mmol) were added to a round-bottomed flask and dissolved in DMF (100 ml) and then, stirred under reflux at 150° C. for 8 hours. When a reaction was completed, the resultant was slowly poured into an excess amount of water and then, stirred for 30 minutes. The solid was dissolved by heating in MCB and then passed through a silica pad. A filtrate therefrom was distilled under a reduced pressure to precipitate the solid, which was filtered to obtain 14.9 g (92%) of Compound A-40.

### Synthesis Example 3: Synthesis of Compound C-4

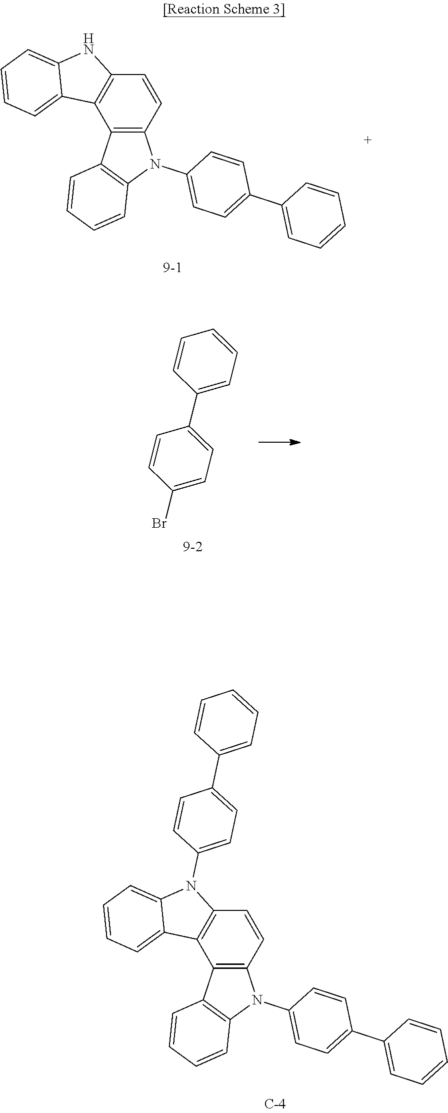 [SMILES: c1ccc(-c2ccc(-n3c4ccccc4c4c5c(ccc43)[nH]c3ccccc35)cc2)cc1 ; Brc1ccc(-c2ccccc2)cc1 ; c1ccc(-c2ccc(-n3c4ccccc4c4c5c6ccccc6n(-c6ccc(-c7ccccc7)cc6)c5ccc43)cc2)cc1]

10.0 g (24.5 mmol) of Intermediate 9-1, 6.3 g (26.9 mmol) of Intermediate 9-2, 1.1 g (1.2 mmol) of Pd₂(dba)₃, 3.5 g (36.7 mmol) of NaOtBu, and 0.7 g (3.7 mmol) of P(t-Bu)₃ were added to a round-bottomed flask and dissolved in xylene (122 ml) and then, stirred under reflux at 140° C. for 8 hours. When a reaction was completed, distilled water was added thereto and then, stirred, and after removing an aqueous layer, an organic layer therefrom was filtered through a silica gel to obtain 10.3 g (75%) of Compound C-4.

(LC/MS theoretical value: 560.23 g/mol, measured value: M+=561.54 g/mol)

### Comparative Synthesis Example 1: Synthesis of Compound R-1

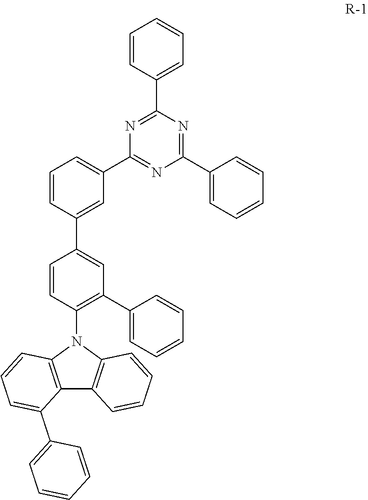 [SMILES: c1ccc(-c2nc(-c3ccccc3)nc(-c3cccc(-c4ccc(-n5c6ccccc6c6c(-c7ccccc7)cccc65)c(-c5ccccc5)c4)c3)n2)cc1]

Compound R-1 was synthesized using the same method as the preparation example of Korean patent document KR10-2447007.

### Comparative Synthesis Example 2: Synthesis of Compound R-2

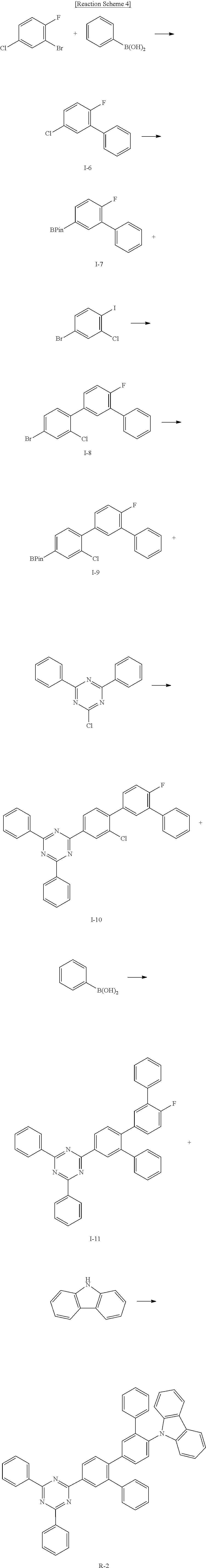

1st Step: Synthesis of Intermediate I-6

2-bromo-4-chloro-1-fluorobenzene (45.0 g, 214.9 mmol), phenylboronic acid (27.5 g, 225.6 mmol), K₂CO₃ (59.4 g, 429.7 mmol), and Pd(PPh₃)₄ (8.7 g, 7.5 mmol) were added to a round-bottomed flask and dissolved in THF (850 ml) and distilled water (220 ml) and then, stirred under reflux at 60° C. for 8 hours. When a reaction was completed, the resultant was cooled to ambient temperature and passed through a separatory funnel to separate an aqueous layer and an organic layer. After removing the organic solvent under a reduced pressure, 32.9 g (74%) of Intermediate I-6 was obtained by using column chromatography (hexane:DCM (10% to 20%)).

### 2nd Step: Synthesis of Intermediate I-7

Intermediate I-6 (32.9 g, 159.2 mmol), bis(pinacolato)diboron (50.5 g, 199.0 mmol), tricyclohexylphosphine (8.9 g, 31.8 mmol), potassium acetate (31.3 g, 318.4 mmol), and Pd(dppf)Cl₂ (5.8 g, 8.0 mmol) were added to a round-bottomed flask and dissolved in xylene (650 ml). The mixture was stirred under reflux at 120° C. for 10 hours. When a reaction was completed, the resultant was cooled to ambient temperature and filtered to remove a salt, and an excess amount of DCM and distilled water were added thereto for extraction. 36.6 g (77%) of Intermediate I-7 was obtained through column chromatography (Hexane:DCM (30% to 50%)).

### 3rd Step: Synthesis of Intermediate I-8

4-bromo-2-chloro-1-iodobenzene (39.0 g, 122.8 mmol), Intermediate I-7 (36.6 g, 122.8 mmol), K₂CO₃ (33.9 g, 245.5 mmol), and Pd(PPh₃)₄ (5.7 g, 4.9 mmol) were added to a round-bottomed flask and dissolved in THF/Toluene (1:1, 600 ml) and distilled water (150 ml) and then, stirred under reflux at 55° C. for 12 hours. When a reaction was completed, the resultant was cooled to ambient temperature and filtered through a separatory funnel to separate an aqueous layer and an organic layer. After removing the organic solvent under a reduced pressure, 25.7 g (58%) of Intermediate I-8 was obtained through column chromatography (hexane:DCM (20% to 30%)).

### 4th Step: Synthesis of Intermediate I-9

Intermediate I-8 (25.7 g, 71.7 mmol), bis(pinacolato)diboron (22.6 g, 88.8 mmol), potassium acetate (10.5 g, 106.6 mmol), and Pd(dppf)Cl₂ (2.6 g, 3.6 mmol) were added to a round-bottomed flask and dissolved in 1,4-dioxane (250 ml). The mixture was stirred under reflux at 120° C. for 10 hours. When a reaction was completed, the resultant was cooled to ambient temperature and filtered to remove a salt, and an excess amount of DCM and distilled water were added thereto for extraction. 17.4 g (60%) of Intermediate I-9 was obtained by using column chromatography (hexane:DCM (30% to 50%)).

### 5th Step: Synthesis of Intermediate I-10

2-chloro-4,6-diphenyl-1,3,5-triazine (10.8 g, 40.4 mmol), Intermediate I-9 (17.4 g, 42.6 mmol), K₂CO₃ (11.8 g, 85.1 mmol), and Pd(PPh₃)₄ (2.5 g, 2.1 mmol) were added to a round-bottomed flask and dissolved in THF (200 ml) and distilled water (50 ml) and then, stirred under reflux at 80° C. for 8 hours. When a reaction was completed, the resultant was cooled to ambient temperature and filtered to remove a salt, and an excess amount of methanol was poured thereinto and then, stirred for 30 minutes. The precipitated solid was filtered and dissolved by heating in MCB. The mixture was passed through a silica pad to collect a filtrate, which was recrystallized to obtain 13.6 g (62%) of Intermediate I-10.

### 6th Step: Synthesis of Intermediate I-11

Intermediate I-10 (13.6 g, 26.5 mmol), phenylboronic acid (6.5 g, 52.9 mmol), Cs₂CO₃ (12.9 g, 39.7 mmol), tri-tert-butylphosphine (2.1 g, 5.3 mmol), and Pd₂(dba)₃ (0.7 g, 0.8 mmol) were added to a round-bottomed flask and dissolved in 1,4-dioxane (150 ml) and then, stirred under reflux at 120° C. for 10 hours. When a reaction was completed, the resultant was cooled to ambient temperature and filtered, and an excess amount of distilled water was added thereto to precipitate a solid. The precipitated solid was filtered and gathered. After dissolving it by heating in MCB, a filtrate was collected therefrom through a silica pad and recrystallized to obtain 10.1 g (69%) of Intermediate I-11.

### 7th Step: Synthesis of Compound R-2

Intermediate I-11 (10.1 g, 18.2 mmol), 9H-carbazole (4.0 g, 23.6 mmol), and K₃PO₄ (7.7 g, 36.4 mmol) were added to a round-bottomed flask and dissolved in DMF (100 ml) and then, stirred under reflux at 150° C. for 8 hours. When a reaction was completed, the resultant was cooled to ambient temperature and filtered, and an excess amount water was slowly poured thereinto and then, stirred for 30 minutes. The precipitated solid was filtered and then dissolved by heating in MCB and then, filtered through a silica pad. A filtrate therefrom was distilled under a reduced pressure to obtain 12.0 g (94%) of Compound R-2.

### Comparative Synthesis Example 3: Synthesis of Compound R-3

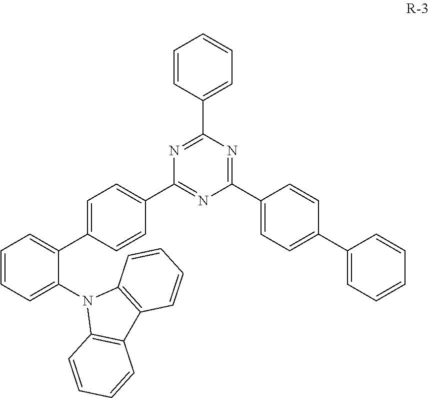 [SMILES: c1ccc(-c2ccc(-c3nc(-c4ccccc4)nc(-c4ccc(-c5ccccc5-n5c6ccccc6c6ccccc65)cc4)n3)cc2)cc1]

Compound R-3 was synthesized using the same method as the preparation example of United States patent document US2023/0200226.

### Comparative Synthesis Example 4: Synthesis of Compound R-4

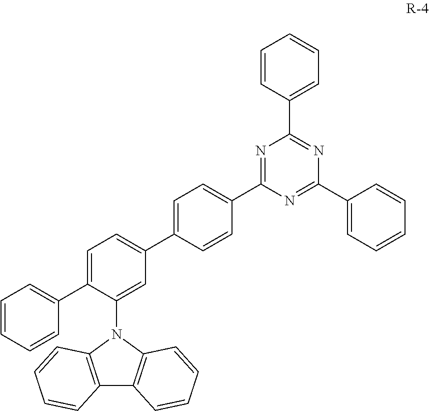 [SMILES: c1ccc(-c2nc(-c3ccccc3)nc(-c3ccc(-c4ccc(-c5ccccc5)c(-n5c6ccccc6c6ccccc65)c4)cc3)n2)cc1]

Compound R-4 was synthesized using the same method as the synthesis method of the Chinese patent document CN117384143.

### Example 1: Manufacturing of Green Organic Light Emitting Diode (Single Host)

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. Compound B was deposited on the hole transport layer to a thickness of 350 Å to form a hole transport auxiliary layer. On the hole transport auxiliary layer, Compound A-1 was used as a host and PhGD was doped at 7 wt % as a dopant to form a 400 Å-thick light emitting layer by vacuum deposition. Then, Compound C was deposited on the light emitting layer to a thickness of 50 Å to form an electron transport auxiliary layer, and Compound D and LiQ were simultaneously vacuum-deposited in a weight ratio of 1:1 to form a 300 Å-thick electron transport layer. An organic light emitting diode was manufactured by sequentially vacuum-depositing 15 Å of LiQ and 1,200 Å of Al on the electron transport layer to form a cathode.

The organic light emitting diode was manufactured to have a structure of ITO/Compound A (3% NDP-9 doping, 100 Å)/Compound A (1,350 Å)/Compound B (350 Å)/EML [Host (Compound A-1): PhGD=93 wt %: 7 wt %](400 Å)/Compound C (50 Å)/Compound D: LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-dibenzofuranyl)phenyl]-N-[4-(9-phenyl-9H-fluoren-9-yl)phenyl][1,1′-biphenyl]-4-amine
Compound C: 2,4-diphenyl-6-(4′,5′,6′-triphenyl [1,1′: 2′,1″: 3″,1″: 3″,1″-quinquephenyl]-3″. yl)-1,3,5-triazine
Compound D: 2-(1, l′-biphenyl-4-yl)-4-(9,9-diphenylfluoren-4-yl)-6-phenyl-1,3,5-triazine

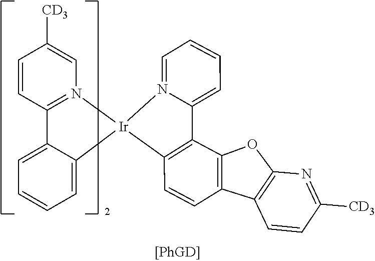

### Example 2 and Comparative Examples 1 to 4

Each organic light emitting diode was manufactured in the same manner as Example 1, except that the compositions were changed to those shown in Table 1.

### Example 3: Manufacturing of Green Organic Light Emitting Diode (Mixed Host)

A glass substrate coated with a thin film of ITO was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound E doped with 3% NDP-9 (Novaled GmbH) was vacuum-deposited on the ITO substrate to form a 100 Å-thick hole injection layer, and Compound E was deposited on the hole injection layer to a thickness of 1,350 Å to form a hole transport layer. Compound F was deposited on the hole transport layer to a thickness of 320 Å to form a hole transport auxiliary layer. On the hole transport auxiliary layer, Compound A-1 and Compound C-4 were simultaneously used as hosts in a weight ratio of 4:6, and PhGD was doped at 10 wt % as a dopant to form a 380 Å-thick light emitting layer by vacuum deposition. Subsequently, Compound G was deposited on the light emitting layer to a thickness of 50 Å to form an electron transport auxiliary layer, and Compound H and LiQ were simultaneously vacuum-deposited in a weight ratio of 1:1 to form a 300 Å-thick electron transport layer. An organic light emitting diode was manufactured by sequentially vacuum-depositing 15 Å of LiQ and 1,200 Å of Al on the electron transport layer to form a cathode.

The organic light emitting diode was manufactured to have a structure of ITO/Compound E (3% NDP-9 doping, 100 Å)/Compound E (1,350 Å)/Compound F (320 Å)/EML [Host (Compound A-1: Compound C-4=4:6 wt %/wt %): PhGD=90 wt %: 10 wt %] (380 Å)/Compound G (50 Å)/Compound H: LiQ (300 Å)/LiQ (15 Å)/AI (1,200 Å).
Compound E: N-(9,9-diphenyl-9H-fluoren-2-yl)-N,9-diphenyl-9H-carbazol-2-amine
Compound F: 9,9-dimethyl-N-[3-(9-phenyl-9H-fluoren-9-yl)phenyl]-4-(4-phenylphenyl)-9H-fluoren-2-amine
Compound G: 4-{4-[4-(9,9-dimethyl-9H-fluoren-4-yl)phenyl]phenyl}-2-phenyl-6-(4-phenylphenyl)pyrimidine
Compound H: 2-(4-{1-[4-(diphenyl-1,3,5-triazin-2-yl)phenyl]naphthalene-2-yl}-4,6-diphenyl-1,3,5-triazine

### Example 4 and Comparative Examples 5 to 8

Each organic light emitting diode was manufactured in the same manner as Example 3, except that the compositions were changed to those shown in Table 2.

### Evaluation

The driving voltage, luminous efficiency, and life-span characteristics of the organic light emitting diodes according to Examples 1 to 4 and Comparative Examples 1 to 8 were evaluated.

The specific measurement methods are as follows, and the results are as shown in Tables 1 to 2.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Current efficiency (cd/A) at the same current density (10 mA/cm²) was calculated by using the luminance and current density from (1) and (2) above.

The luminous efficiency values of Example 1, Example 2, and Comparative Examples 1 to 4 were calculated as relative values based on Comparative Example 1 and are shown in Table 1.

The luminous efficiency values of Example 3, Example 4, and Comparative Examples 5 to 8 were calculated as relative values based on Comparative Example 5 and are shown in Table 2.

### (4) Measurement of Life-Span

Time when each current efficiency (cd/A) was reduced to 97%, while maintaining luminance (cd/m²) at 24000 cd/m², was measured as a life-span.

The life-spans of Example 1, Example 2, and Comparative Examples 1 to 4 were calculated as relative values based on Comparative Example 1 and are shown in Table 1.

The life-spans of Example 3, Example 4, and Comparative Examples 5 to 8 were calculated as relative values based on Comparative Example 5 and are shown in Table 2.

### (5) Measurement of Driving Voltage

The driving voltage of each diode at 15 mA/cm² using a current-voltage meter (Keithley 2400) were measured to obtain the results.

The driving voltages of Examples 1, 2, and Comparative Examples 1 to 4 were calculated as relative values based on Comparative Example 1 and are listed in Table 1.

TABLE 1

| | | | Luminous | |
|---|---|---|---|---|
| | | Driving voltage | efficiency | Life-span |
| No. | Compound | (%) | (%) | (%) |
| Example 1 | A-1 | 97 | 105 | 115 |
| Example 2 | A-42 | 95 | 108 | 132 |
| Comparative | R-1 | 100 | 100 | 100 |
| Example 1 | | | | |
| Comparative | R-2 | 106 | 99 | 85 |
| Example 2 | | | | |
| Comparative | R-3 | 102 | 102 | 88 |
| Example 3 | | | | |
| Comparative | R-4 | 100 | 94 | 90 |
| Example 4 | | | | |

TABLE 2

| | | | Luminous | |
|---|---|---|---|---|
| | First | Second | efficiency | Life-span |
| No. | compound | compound | (%) | (%) |
| Example 3 | A-1 | C-4 | 106 | 118 |
| Example 4 | A-42 | C-4 | 108 | 135 |
| Comparative | R-1 | C-4 | 100 | 100 |
| Example 5 | | | | |
| Comparative | R-2 | C-4 | 95 | 88 |
| Example 6 | | | | |
| Comparative | R-3 | C-4 | 99 | 92 |
| Example 7 | | | | |
| Comparative | R-4 | C-4 | 94 | 90 |
| Example 8 | | | | |

Referring to Tables 1 and 2, the driving voltage, luminous efficiency, and life-span characteristics of the organic light emitting diodes according to Examples 1 to 4 are significantly improved compared to the organic light emitting diodes according to Comparative Examples 1 to 8.

By way of summation and review, some example embodiments may provide a compound for an organic optoelectronic device that can lower the driving voltage and realize an organic optoelectronic device with high efficiency and long life-span.

Some example embodiments may provide a composition for an organic optoelectronic device including the compound for an organic optoelectronic device.

Some example embodiments may provide an organic optoelectronic device including the compound for an organic optoelectronic device or the composition for an organic optoelectronic device.

Some example embodiments may provide a display device including the organic optoelectronic device.

Through some example embodiments high-efficiency, long life-span organic optoelectronic devices can be realized while lowering the required operating voltage.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device represented by a combination of Chemical Formula 1a and Chemical Formula 1b: 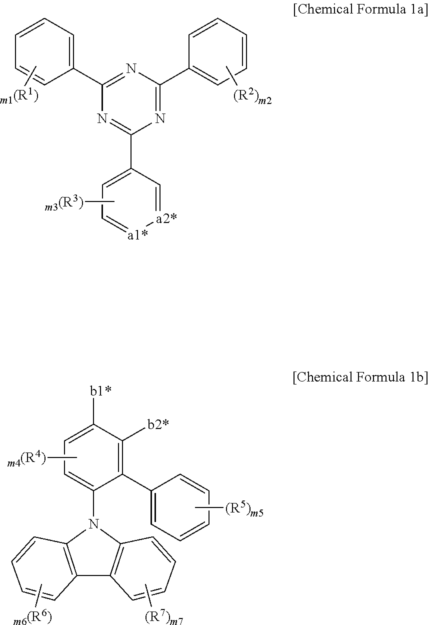
wherein, in Chemical Formula 1a and Chemical Formula 1b,
a1* and a2* are each independently a linking carbon or CR^{a},
b1* and b2* are each independently linking points, that link at a1* or a2*, or R^{b},
R¹ and R² are each independently hydrogen, deuterium, or a phenyl group substituted with deuterium,
R^{a}, R^{b}, and R³ to R⁷ are each independently hydrogen or deuterium,
m1, m2, and m5 are each independently an integer of 1 to 5,
m3 is an integer of 1 to 3,
m4 is an integer of 1 or 2,
m6 and m7 are each independently an integer of 1 to 4,
when m1 is 2, 3, 4, or 5, each R¹ is the same or different from each other,
when m2 is 2, 3, 4, or 5, each R² is the same or different from each other,
when m3 is 2 or 3, each R³ is the same or different from each other,
when m4 is 2, each R⁴ is the same or different from each other,
when m5 is 2, 3, 4, or 5, each R⁵ is the same or different from each other,
when m6 is 2, 3, or 4, each R⁶ is the same or different from each other, and
when m7 is 2, 3, or 4, each R⁷ is the same or different from each other.

2. The compound for an organic optoelectronic device as claimed in claim 1, wherein:
the combination of Chemical Formula 1a and Chemical Formula 1b is represented by one of Chemical Formula 1-1 to Chemical Formula 1-4: 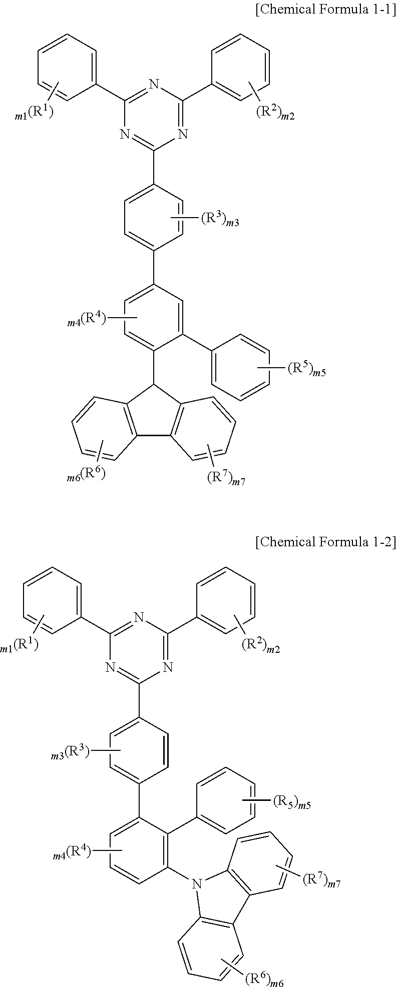 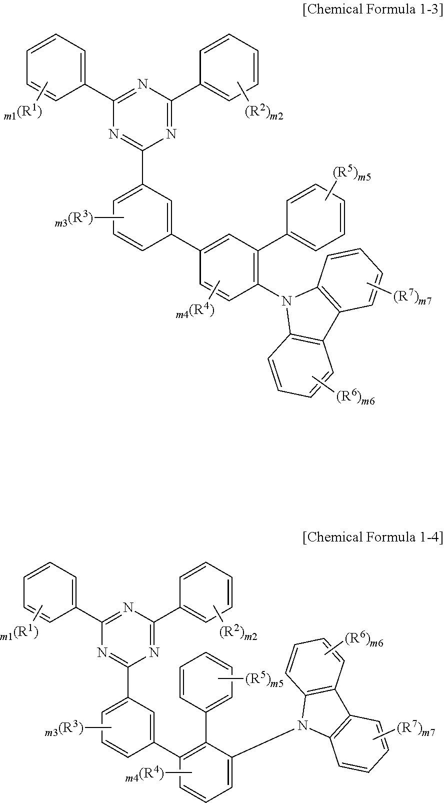
in Chemical Formula 1-1 to Chemical Formula 1-4, R¹ to R⁷ and m1 to m7 are defined the same as those of Chemical Formula 1a and Chemical Formula 1b.

3. The compound for an organic optoelectronic device as claimed in claim 1, wherein:
the compound is a compound of Group 1, or Group 1-1: 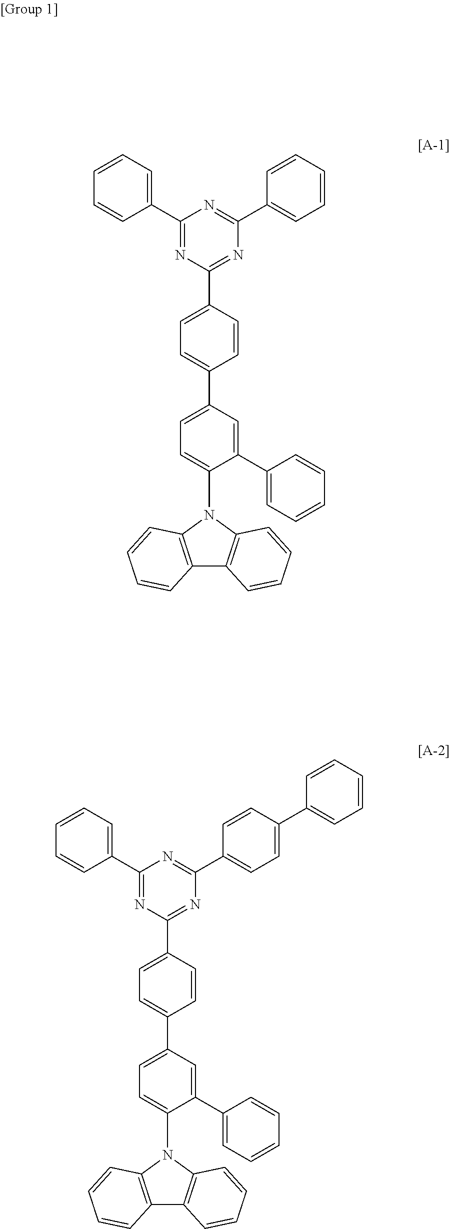 [SMILES: c1ccc(-c2nc(-c3ccccc3)nc(-c3ccc(-c4ccc(-n5c6ccccc6c6ccccc65)c(-c5ccccc5)c4)cc3)n2)cc1 ; c1ccc(-c2ccc(-c3nc(-c4ccccc4)nc(-c4ccc(-c5ccc(-n6c7ccccc7c7ccccc76)c(-c6ccccc6)c5)cc4)n3)cc2)cc1] 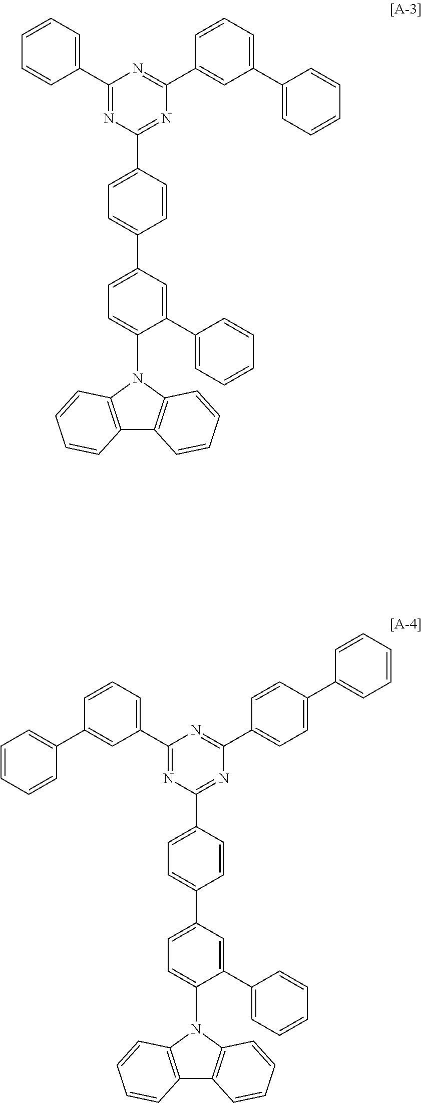 [SMILES: c1ccc(-c2cccc(-c3nc(-c4ccccc4)nc(-c4ccc(-c5ccc(-n6c7ccccc7c7ccccc76)c(-c6ccccc6)c5)cc4)n3)c2)cc1 ; c1ccc(-c2ccc(-c3nc(-c4ccc(-c5ccc(-n6c7ccccc7c7ccccc76)c(-c6ccccc6)c5)cc4)nc(-c4cccc(-c5ccccc5)c4)n3)cc2)cc1] 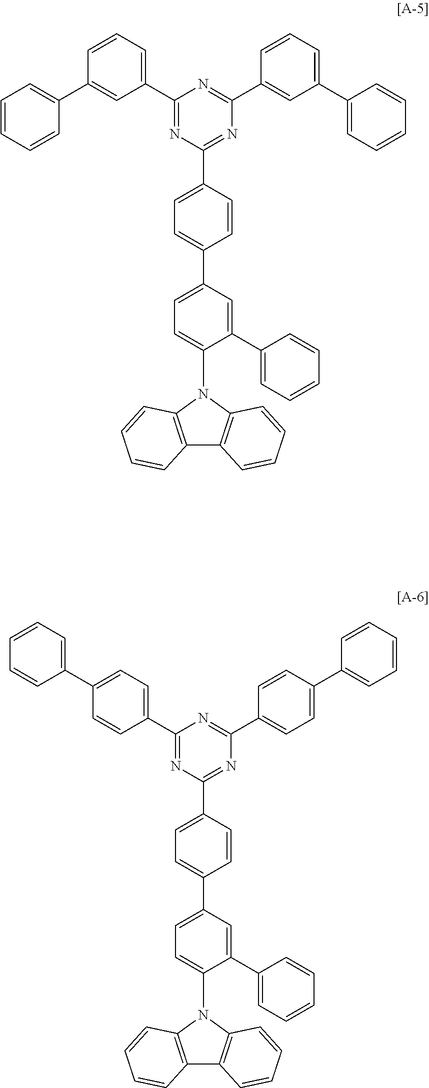 [SMILES: c1ccc(-c2cccc(-c3nc(-c4ccc(-c5ccc(-n6c7ccccc7c7ccccc76)c(-c6ccccc6)c5)cc4)nc(-c4cccc(-c5ccccc5)c4)n3)c2)cc1 ; c1ccc(-c2ccc(-c3nc(-c4ccc(-c5ccccc5)cc4)nc(-c4ccc(-c5ccc(-n6c7ccccc7c7ccccc76)c(-c6ccccc6)c5)cc4)n3)cc2)cc1] 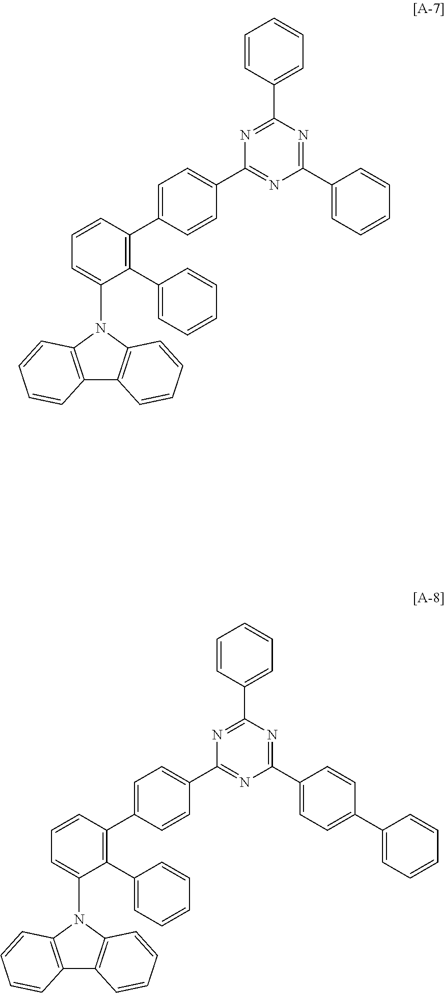 [SMILES: c1ccc(-c2nc(-c3ccccc3)nc(-c3ccc(-c4cccc(-n5c6ccccc6c6ccccc65)c4-c4ccccc4)cc3)n2)cc1 ; c1ccc(-c2ccc(-c3nc(-c4ccccc4)nc(-c4ccc(-c5cccc(-n6c7ccccc7c7ccccc76)c5-c5ccccc5)cc4)n3)cc2)cc1] 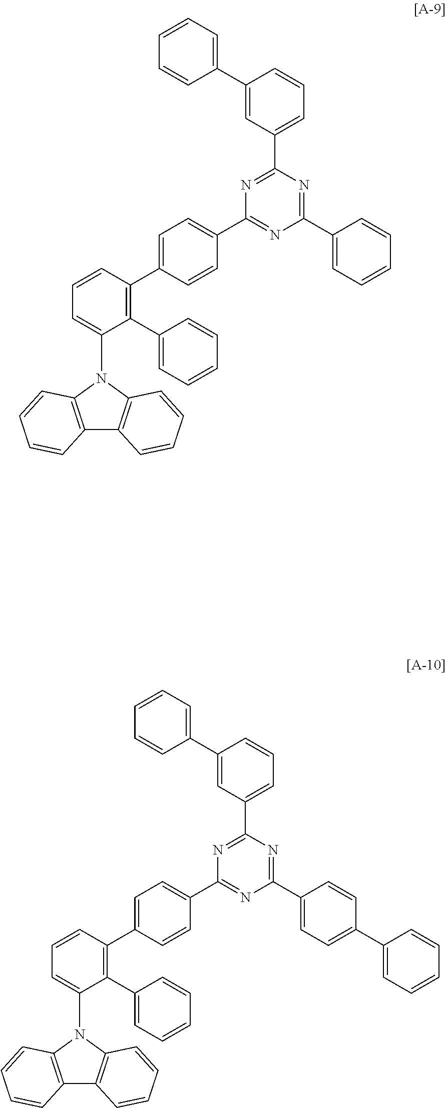 [SMILES: c1ccc(-c2cccc(-c3nc(-c4ccccc4)nc(-c4ccc(-c5cccc(-n6c7ccccc7c7ccccc76)c5-c5ccccc5)cc4)n3)c2)cc1 ; c1ccc(-c2ccc(-c3nc(-c4ccc(-c5cccc(-n6c7ccccc7c7ccccc76)c5-c5ccccc5)cc4)nc(-c4cccc(-c5ccccc5)c4)n3)cc2)cc1] 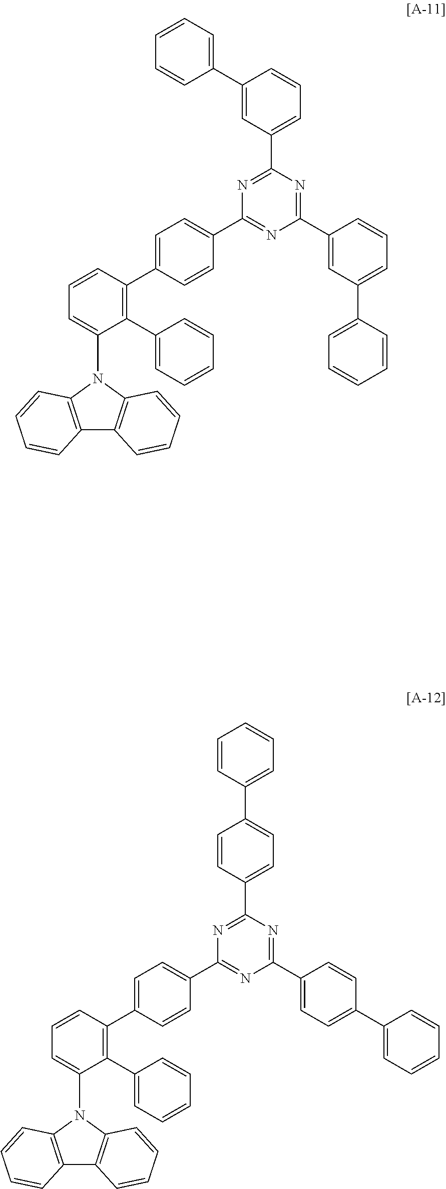 [SMILES: c1ccc(-c2cccc(-c3nc(-c4ccc(-c5cccc(-n6c7ccccc7c7ccccc76)c5-c5ccccc5)cc4)nc(-c4cccc(-c5ccccc5)c4)n3)c2)cc1 ; c1ccc(-c2ccc(-c3nc(-c4ccc(-c5ccccc5)cc4)nc(-c4ccc(-c5cccc(-n6c7ccccc7c7ccccc76)c5-c5ccccc5)cc4)n3)cc2)cc1] 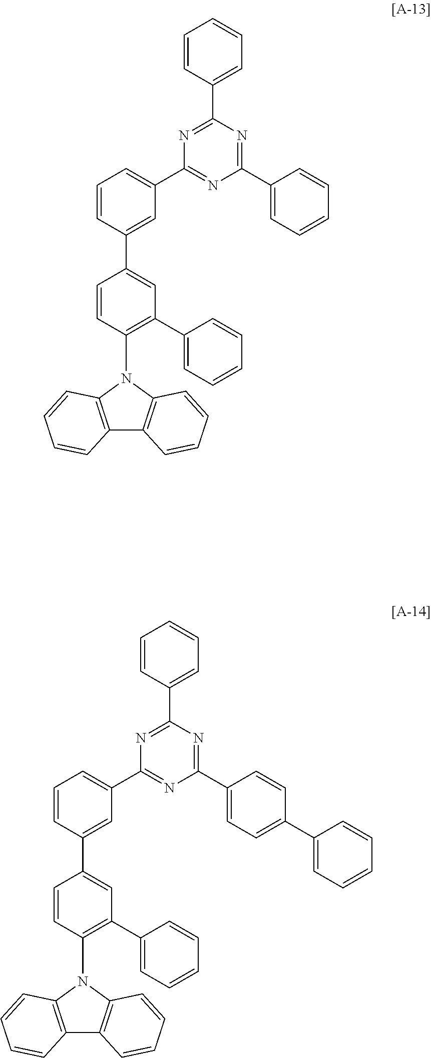 [SMILES: c1ccc(-c2nc(-c3ccccc3)nc(-c3cccc(-c4ccc(-n5c6ccccc6c6ccccc65)c(-c5ccccc5)c4)c3)n2)cc1 ; c1ccc(-c2ccc(-c3nc(-c4ccccc4)nc(-c4cccc(-c5ccc(-n6c7ccccc7c7ccccc76)c(-c6ccccc6)c5)c4)n3)cc2)cc1] 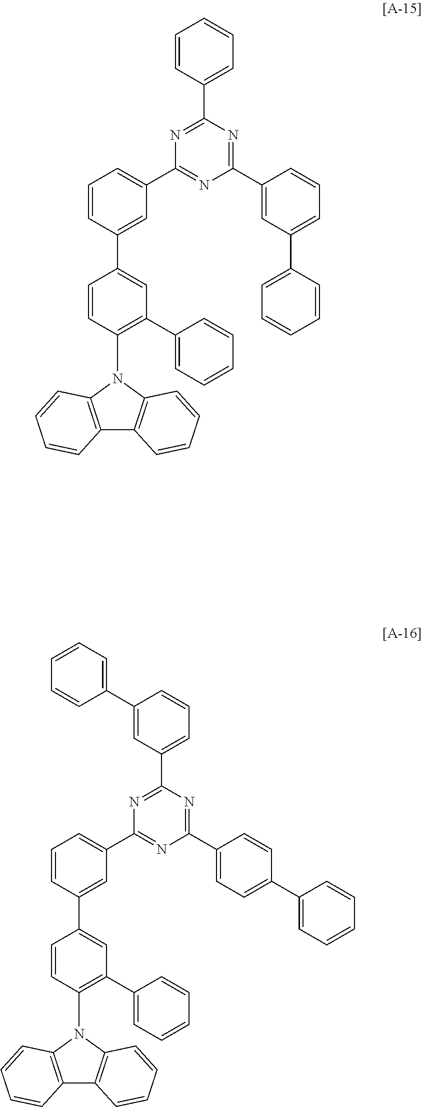 [SMILES: c1ccc(-c2cccc(-c3nc(-c4ccccc4)nc(-c4cccc(-c5ccc(-n6c7ccccc7c7ccccc76)c(-c6ccccc6)c5)c4)n3)c2)cc1 ; c1ccc(-c2ccc(-c3nc(-c4cccc(-c5ccccc5)c4)nc(-c4cccc(-c5ccc(-n6c7ccccc7c7ccccc76)c(-c6ccccc6)c5)c4)n3)cc2)cc1] 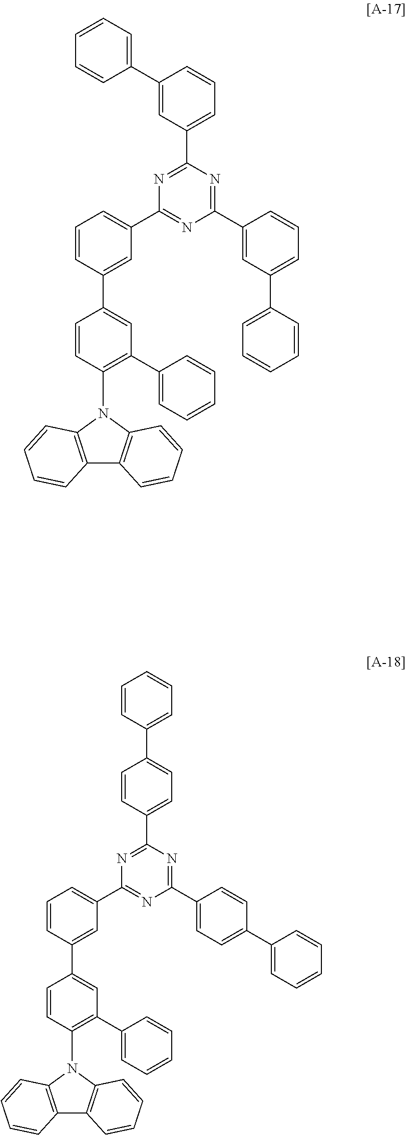 [SMILES: c1ccc(-c2cccc(-c3nc(-c4cccc(-c5ccccc5)c4)nc(-c4cccc(-c5ccc(-n6c7ccccc7c7ccccc76)c(-c6ccccc6)c5)c4)n3)c2)cc1 ; c1ccc(-c2ccc(-c3nc(-c4ccc(-c5ccccc5)cc4)nc(-c4cccc(-c5ccc(-n6c7ccccc7c7ccccc76)c(-c6ccccc6)c5)c4)n3)cc2)cc1] 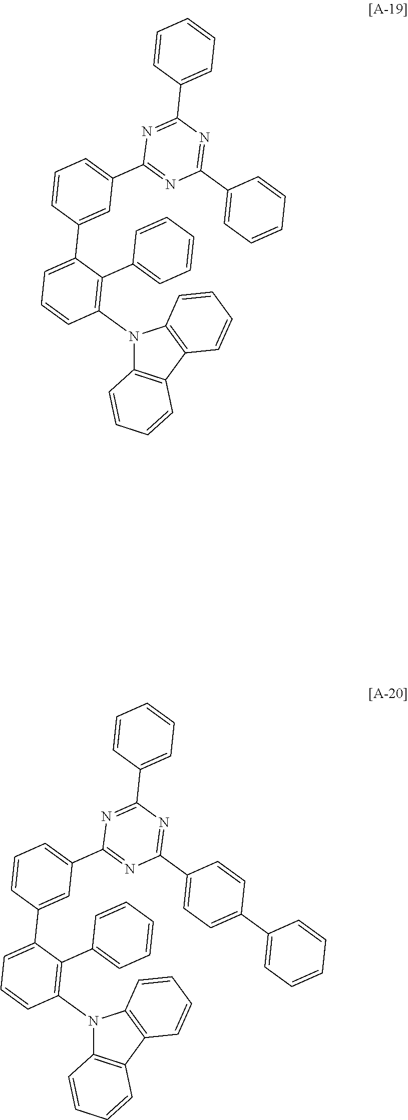 [SMILES: c1ccc(-c2nc(-c3ccccc3)nc(-c3cccc(-c4cccc(-n5c6ccccc6c6ccccc65)c4-c4ccccc4)c3)n2)cc1 ; c1ccc(-c2ccc(-c3nc(-c4ccccc4)nc(-c4cccc(-c5cccc(-n6c7ccccc7c7ccccc76)c5-c5ccccc5)c4)n3)cc2)cc1] 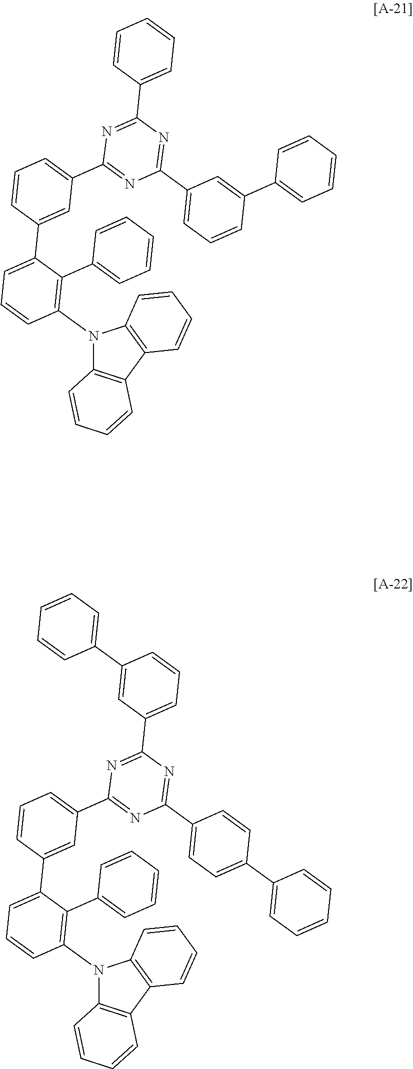 [SMILES: c1ccc(-c2cccc(-c3nc(-c4ccccc4)nc(-c4cccc(-c5cccc(-n6c7ccccc7c7ccccc76)c5-c5ccccc5)c4)n3)c2)cc1 ; c1ccc(-c2ccc(-c3nc(-c4cccc(-c5ccccc5)c4)nc(-c4cccc(-c5cccc(-n6c7ccccc7c7ccccc76)c5-c5ccccc5)c4)n3)cc2)cc1] 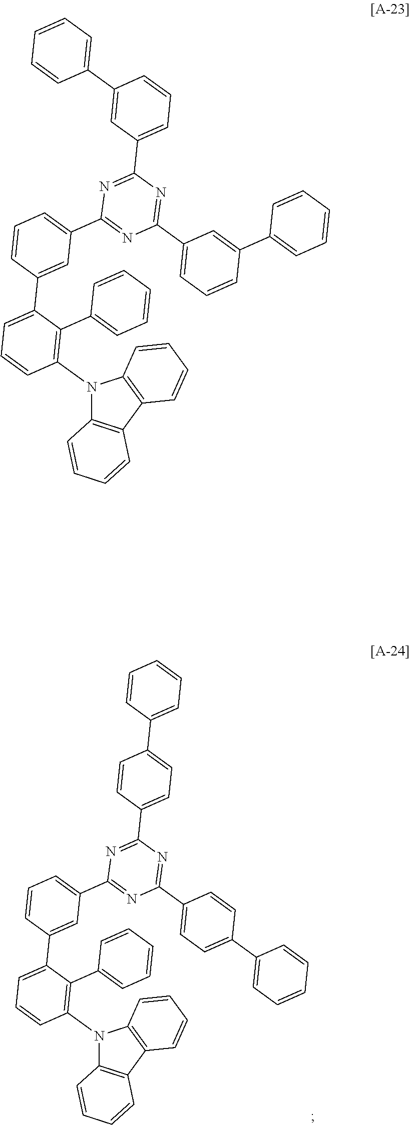 [SMILES: c1ccc(-c2cccc(-c3nc(-c4cccc(-c5ccccc5)c4)nc(-c4cccc(-c5cccc(-n6c7ccccc7c7ccccc76)c5-c5ccccc5)c4)n3)c2)cc1 ; c1ccc(-c2ccc(-c3nc(-c4ccc(-c5ccccc5)cc4)nc(-c4cccc(-c5cccc(-n6c7ccccc7c7ccccc76)c5-c5ccccc5)c4)n3)cc2)cc1] 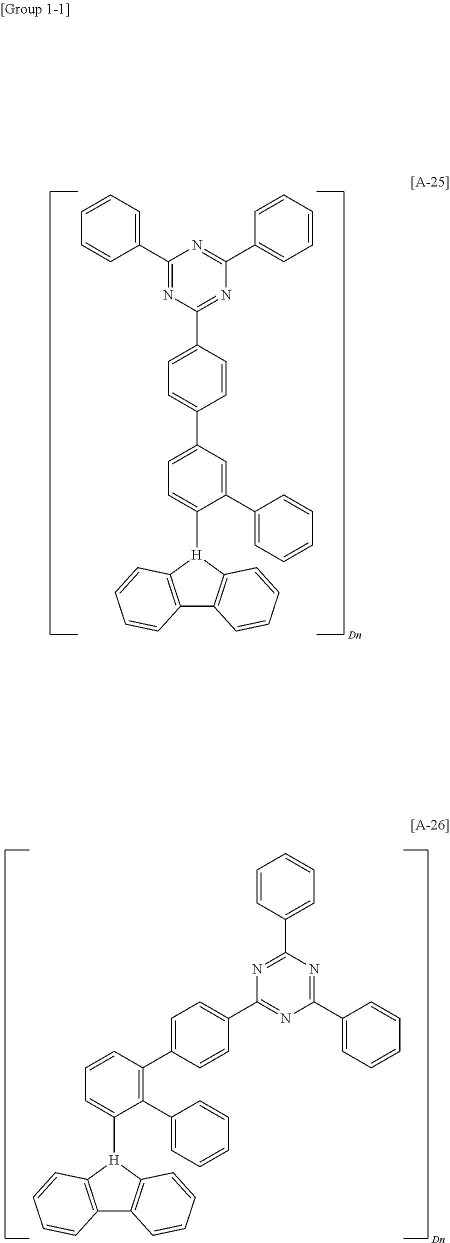 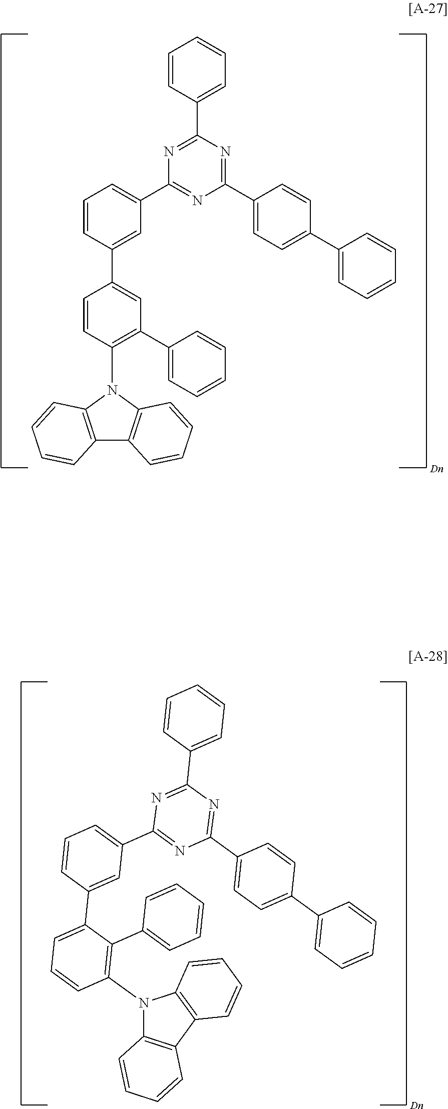 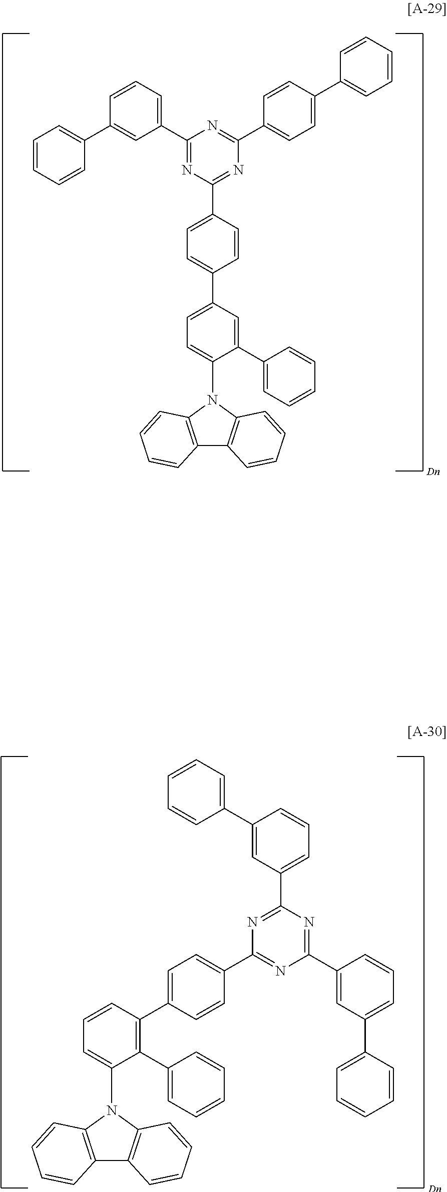 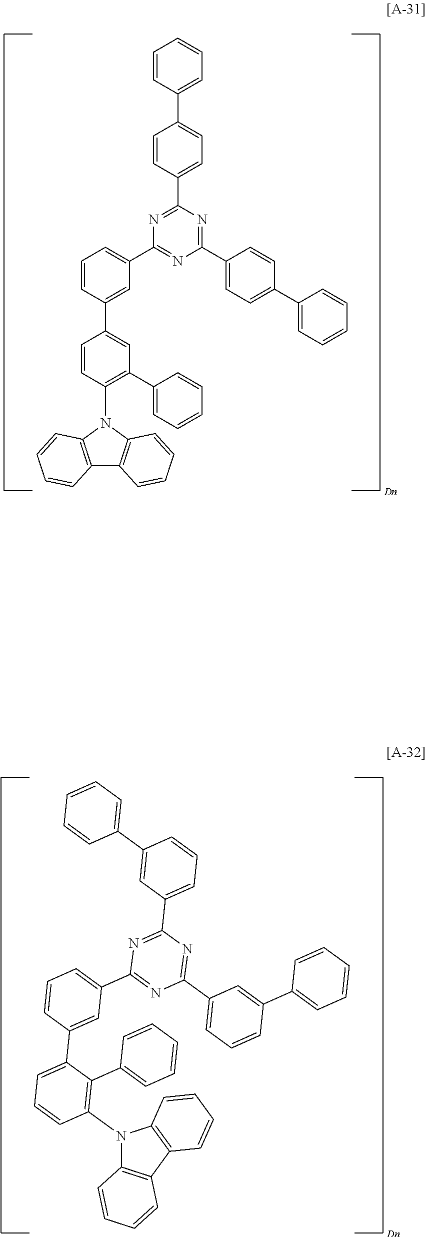
in Group 1-1, Dn refers to the number of deuterium substitutions and indicates a structure substituted with one or more deuterium atoms.

4. The compound for an organic optoelectronic device as claimed in claim 1, wherein:
the compound is a compound of Group 1-2

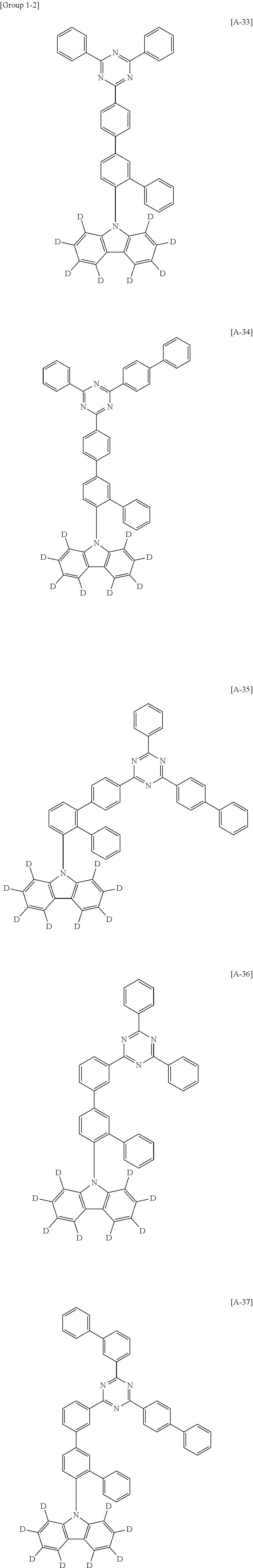

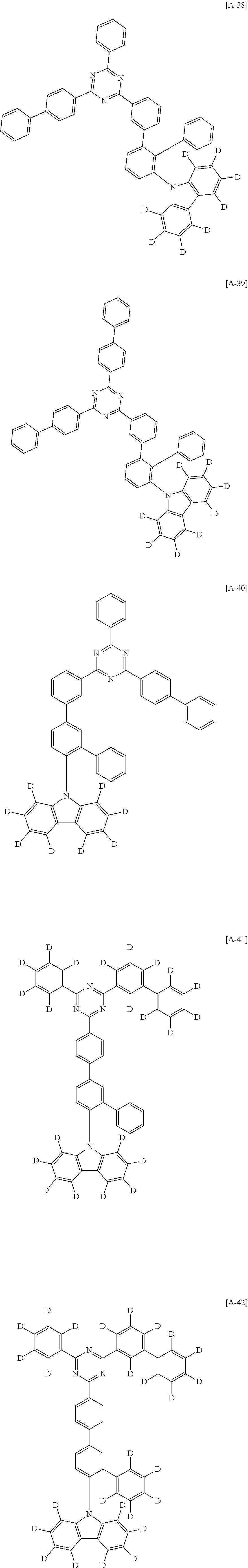

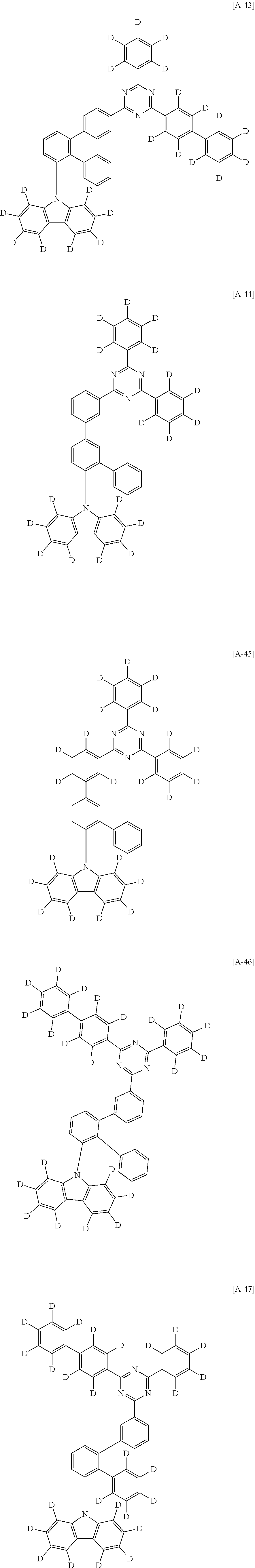

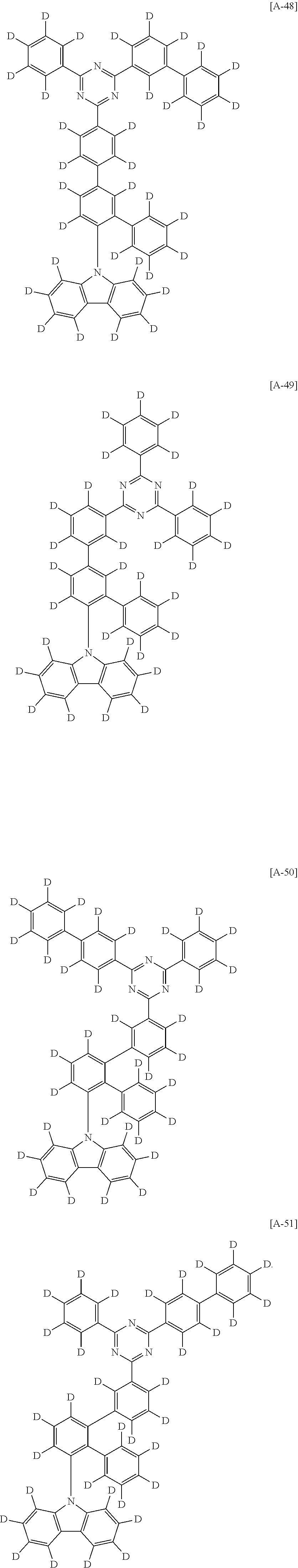

5. A composition for an organic optoelectronic device, comprising:
a first compound; and
a second compound,
wherein:
the first compound is the compound for an organic optoelectronic device as claimed in claim 1, and
the second compound is represented by:
Chemical Formula 2,
a combination of Chemical Formula 3 and Chemical Formula 4, or
Chemical Formula 5: 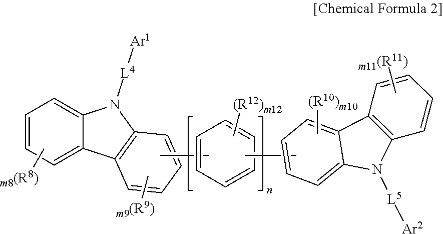
in Chemical Formula 2,
R⁸ to R¹² are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar¹ and Ar² are each independently a substituted or unsubstituted C6 to C20 aryl group or a substituted or unsubstituted C2 to C30 heterocyclic group,
L¹ and L² are each independently a single bond or a substituted or unsubstituted C6 to C20 arylene group,
m8, m11, and m12 are each independently an integer of 1 to 4,
m9 and m10 are each independently an integer of 1 to 3,
when m8 is 2, 3, or 4, each R⁸ is the same or different from each other,
when m9 is 2 or 3, each R⁹ is the same or different from each other,
when m10 is 2 or 3, each R¹⁰ is the same or different from each other,
when m11 is 2, 3, or 4, each R¹¹ is the same or different from each other,
when m12 is 2, 3, or 4, each R¹² is the same or different from each other, and
n is an integer of 0 to 2; 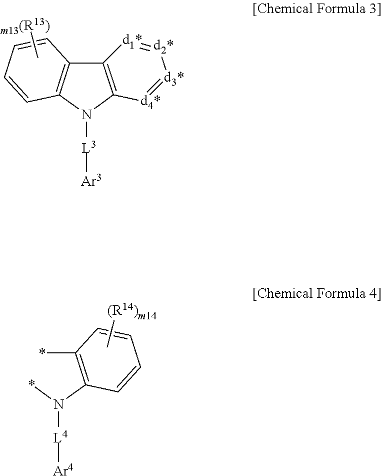
in Chemical Formula 3 and Chemical Formula 4,
two adjacent ones of d₁* to d₄* in Chemical Formula 3, are each linking carbons linked to * of Chemical Formula 4,
the remaining two of d₁* to d₄* in Chemical Formula 3, not linked at * of Chemical Formula 4, are C-L^{a}-R^{c},
L^{a}, L³, and L⁴ are each independently a single bond or a substituted or unsubstituted C6 to C20 arylene group,
R^{c}, R¹³, and R¹⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar³ and Ar⁴ are each independently a substituted or unsubstituted C6 to C20 aryl group or a substituted or unsubstituted C2 to C30 heterocyclic group,
m13 and m14 are each independently an integer of 1 to 4,
when m13 is 2, 3, or 4, each R¹³ is the same or different from each other, and
when m14 is 2, 3, or 4, each R¹⁴ is the same or different from each other; 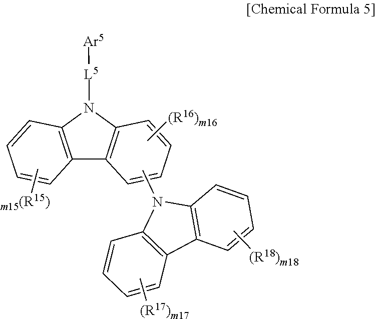
in Chemical Formula 5,
L⁵ is single bond or a substituted or unsubstituted C6 to C20 arylene group,
R¹⁵ to R¹⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁵ is a substituted or unsubstituted C6 to C20 aryl group or a substituted or unsubstituted C2 to C30 heterocyclic group,
m15, m17, and m18 are each independently an integer of 1 to 4,
m16 is an integer of 1 to 3,
when m15 is 2, 3, or 4, each R¹⁵ is the same or different from each other,
when m16 is 2 or 3, each R¹⁶ is the same or different from each other,
when m17 is 2, 3, or 4, each R¹⁷ is the same or different from each other, and
when m18 is 2, 3, or 4, each R¹⁸ is the same or different from each other.

6. The composition for an organic optoelectronic device as claimed in claim 5, wherein:
the second compound is represented by Chemical Formula 2, and
Chemical Formula 2 is represented by Chemical Formula 2-8: 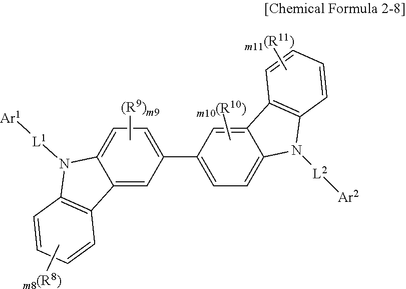
in Chemical Formula 2-8,
R⁸ to R¹¹ are each independently hydrogen, deuterium, or a substituted or unsubstituted C6 to C12 aryl group,
m8 and m11 are each independently an integer of 1 to 4,
m9 and m10 are each independently an integer of 1 to 3, and
moieties L¹-Ar¹ and L²-Ar² are each independently a moiety of Group I, 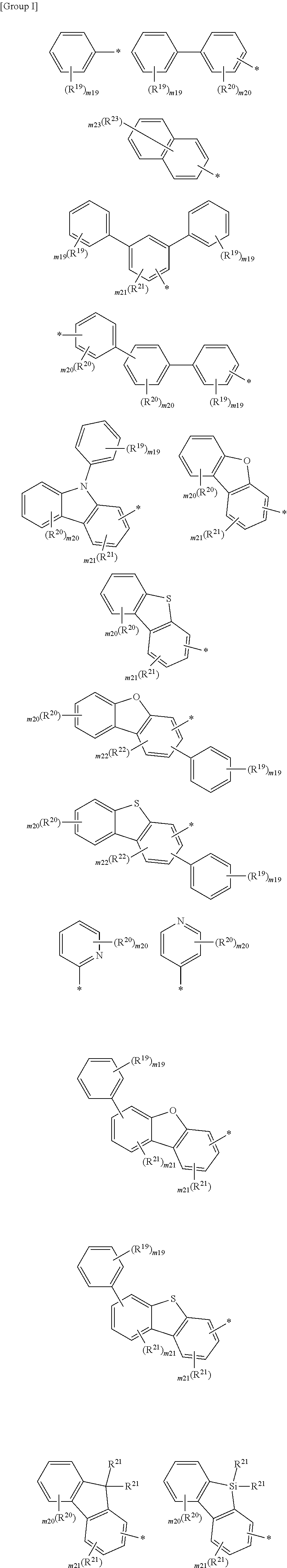 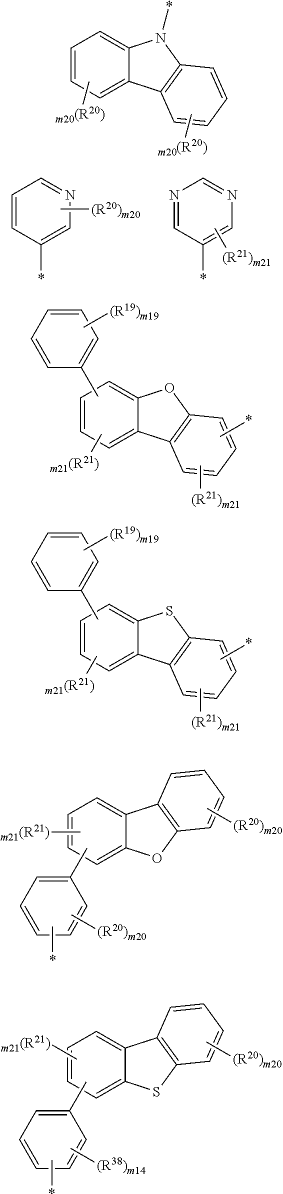 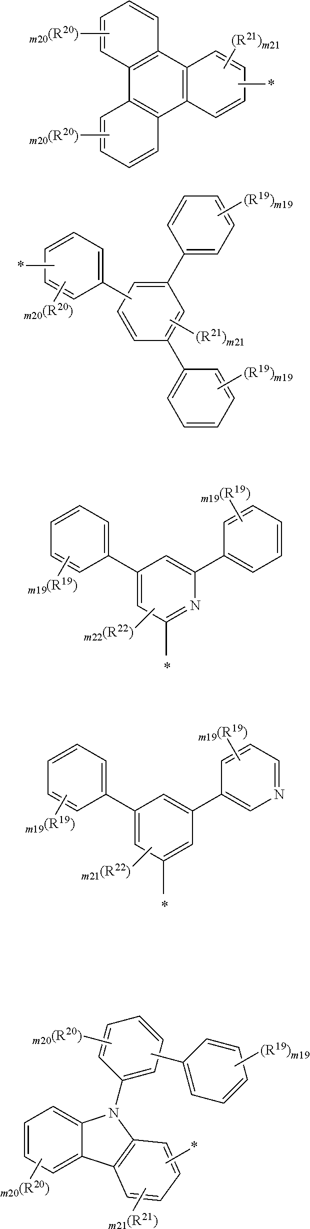 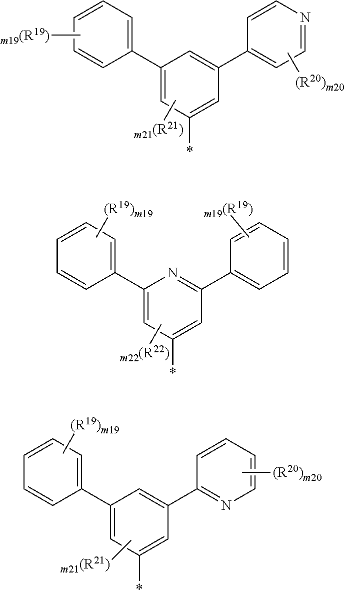
in Group I,
R¹⁹ to R²³ are each independently hydrogen, deuterium, a cyano group, a substituted or unsubstituted C1 to C10 alkyl group or a substituted or unsubstituted C6 to C12 aryl group,
m19 is an integer of 1 to 5,
m20 is an integer of 1 to 4,
m21 is an integer of 1 to 3,
m22 is an integer of 1 or 2,
m23 is an integer of 1 to 7,
* is a linking point,
when m19 is 2, 3, 4, or 5, each R¹⁹ is the same or different from each other,
when m20 is 2, 3, or 4, each R²⁰ is the same or different from each other,
when m21 is 2 or 3, each R²¹ is the same or different from each other,
when m22 is 2, each R²² is the same or different from each other, and
when m23 is 2, 3, 4, 5, 6, or 7, each R²³ is the same or different from each other.

7. The composition for an organic optoelectronic device as claimed in claim 5, wherein:
the second compound is represented by a combination of Chemical Formula 3 and Chemical Formula 4, and
the combination of Chemical Formula 3 and Chemical Formula 4 is represented by Chemical Formula 3C: 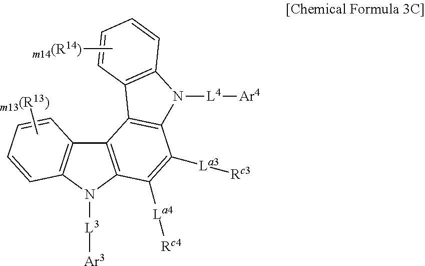
in Chemical Formula 3C,
L^{a3} and L^{a4} are each a single bond,
R¹³, R¹⁴, R^{c3}, and R^{c4} are each independently hydrogen, deuterium, or a substituted or unsubstituted C6 to C12 aryl group,
m13 and m14 are each independently an integer of 1 to 4,
moieties L³-Ar³ and L⁴-Ar⁴ are each independently a moiety of Group I,
when m13 is 2, 3, or 4, each R¹³ is the same or different from each other, and
when m14 is 2, 3, or 4, each R¹⁴ is the same or different from each other, 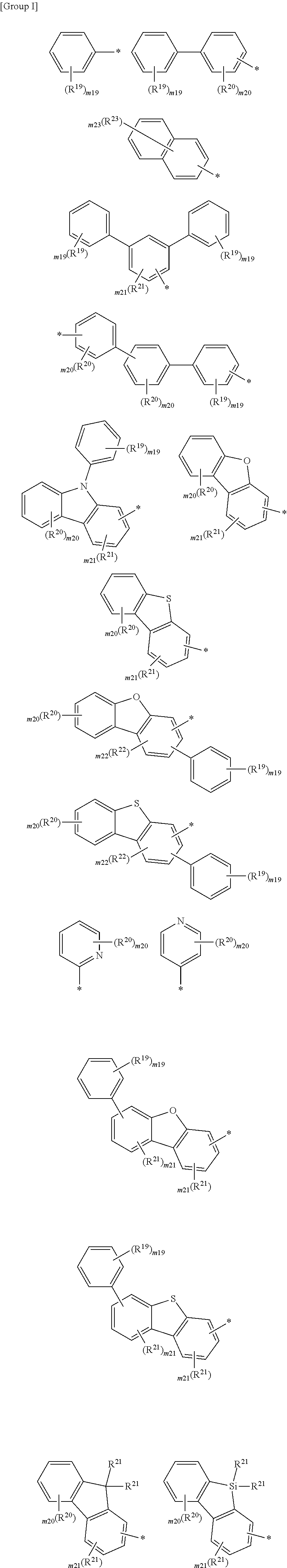 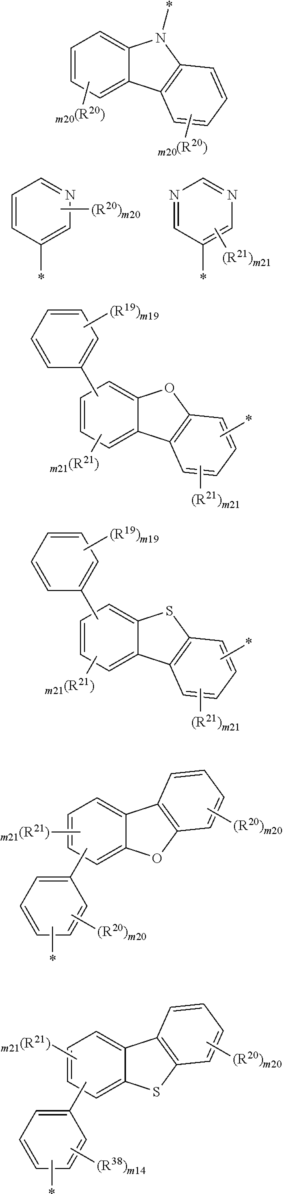 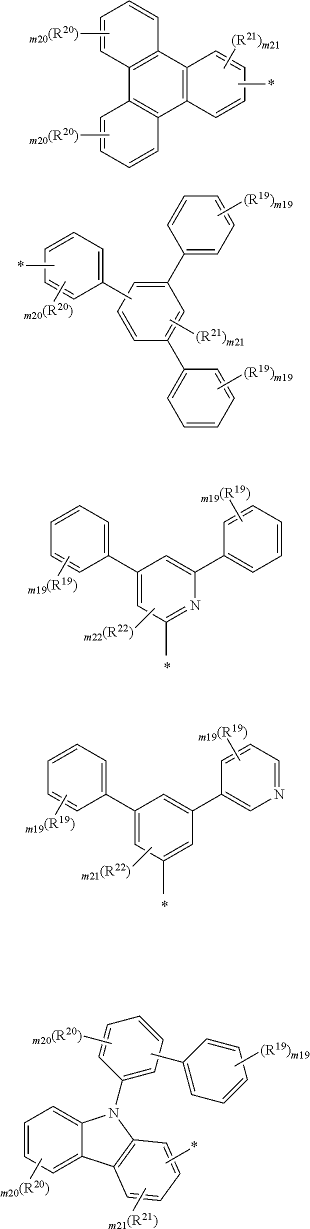 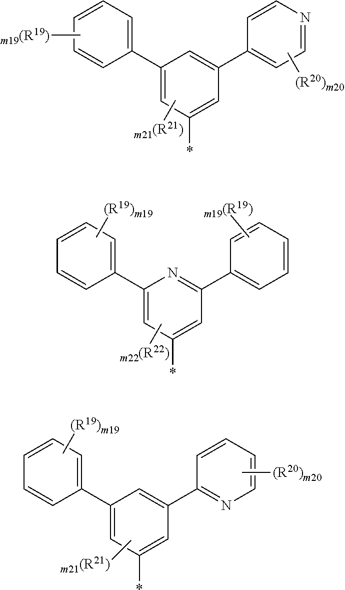
in Group I,
R¹⁹ to R²³ are each independently hydrogen, deuterium, a cyano group, a substituted or unsubstituted C1 to C10 alkyl group, or a substituted or unsubstituted C6 to C12 aryl group,
m19 is an integer of 1 to 5,
m20 is an integer of 1 to 4,
m21 is an integer of 1 to 3,
m22 is an integer of 1 or 2,
m23 is an integer of 1 to 7,
* is a linking point,
when m19 is 2, 3, 4, or 5, each R¹⁹ is the same or different from each other,
when m20 is 2, 3, or 4, each R²⁰ is the same or different from each other,
when m21 is 2 or 3, each R²¹ is the same or different from each other,
when m22 is 2, each R²² is the same or different from each other, and
when m23 is 2, 3, 4, 5, 6, or 7, each R²³ is the same or different from each other.

8. An organic optoelectronic device, comprising:
an anode and a cathode facing each other, and
at least one organic layer between the anode and the cathode,
wherein the at least one organic layer includes the compound for the organic optoelectronic device of claim 1.

9. The organic optoelectronic device as claimed in claim 8, wherein:
the at least one organic layer includes a light emitting layer, and
the light emitting layer includes the compound.

10. A display device comprising the organic optoelectronic device as claimed in claim 8.

11. An organic optoelectronic device, comprising:
an anode and a cathode facing each other, and
at least one organic layer between the anode and the cathode,
wherein the at least one organic layer includes the composition for the organic optoelectronic device of claim 5.

12. The organic optoelectronic device as claimed in claim 11, wherein:
the at least one organic layer includes a light emitting layer, and
the light emitting layer includes the composition.

13. A display device comprising the organic optoelectronic device as claimed in claim 11.
